(19) United States
(12) Patent Application Publication
(10) Pub. No.: US 2023/0117831 A1
(43) Pub. Date: Apr. 20, 2023

(54) MULTI-FUNCTIONAL CHIMERIC MOLECULES

(71) Applicants: THE BROAD INSTITUTE, INC., CAMBRIDGE, MA (US); THE BRIGHAM AND WOMEN'S HOSPITAL, INC., BOSTON, MA (US)

(72) Inventors: Amit CHOUDHARY, Boston, MA (US); Sophia LAI, Boston, MA (US); Veronika SHOBA, Cambridge, MA (US); Praveen KOKKONDA, Boston, MA (US); Dhanushka MUNKANATTA GODAGE, Cambridge, MA (US); Sachini SIRIWARDENA, Cambridge, MA (US)

(21) Appl. No.: 17/791,811

(22) PCT Filed: Jan. 8, 2021
(86) PCT No.: PCT/US21/12816

§ 371 Date: Jul. 8, 2022

Related U.S. Application Data
(60) Provisional application No. 62/958,696, filed on Jan. 8, 2020.
(60) Provisional application No. 63/057,879, filed on Jul. 28, 2020.
(60) Provisional application No. 63/069,655, filed on Aug. 24, 2020.

(51) Int. Cl. A61K 47/55; A61K 31/551

(52) U.S. Cl. CPC A61K 47/55; A61K 31/551

(57) ABSTRACT

The present disclosure relates to multifunctional chemical conjugation molecules, which find utility as modifiers of target substrates. The present disclosure includes multifunctional compounds comprising a localizing moiety, a chemical linker moiety, an activator moiety, a first orienting adaptor interconnecting the chemical linker moiety on one end to the activator moiety, and optionally a second orienting adaptor interconnecting the chemical linker molecule on a different end to the localizing moiety. Molecules according to the present invention find use making post-translational modifications to macromolecules that are not the natural substrate of the activator moiety. Diseases or disorders may be treated or prevented with molecules of the present disclosure.

### CROSS-REFERENCE TO RELATED APPLICATIONS

This application is the U.S. National Stage Application under 35 U.S.C. § 371 of PCT Application No. PCT/US2021/012816, which claims the benefit of U.S. Provisional Application No. 62/958,696, filed Jan. 8, 2020, U.S. Provisional Application No. 63/057,879, filed Jul. 28, 2020, and U.S. Provisional Application No. 63/069,655, filed Aug. 24, 2020. The entire contents of the above-identified applications are hereby fully incorporated herein by reference.

### STATEMENT REGARDING FEDERALLY SPONSORED RESEARCH

This invention was made with government support under Grant No. AI154099 awarded by the National Institutes of Health, and Grant No. N66001-17-2-4055 awarded by the Defense Advanced Research Projects Agency. The government has certain rights in the invention.

### REFERENCE TO AN ELECTRONIC SEQUENCE LISTING

The contents of the electronic sequence listing (“BROD-4850WP_ST25.txt”; Size is 14,782 bytes and it was created on Jan. 5, 2021) is herein incorporated by reference in its entirety.

### TECHNICAL FIELD

The subject matter disclosed herein is generally directed to multifunctional chemical conjugation molecules utilized to induce modifications in target substrates.

### BACKGROUND

Protein kinases regulate critical cellular functions like cell cycle, metabolism, differentiation, proliferation, and apoptosis. Kinase dysfunction is also connected to a variety of human diseases including cancer, inflammatory conditions, autoimmune disorders, and cardiac diseases.

An ongoing need exists in the art for effective treatments for diseases associated with enzymatic dysfunctions as well as modifications such as post-translational modifications. However, obstacles such as non-specific effects remain as obstacles to the development of effective modifications and treatments. As an example, small molecules that induce phosphorylation of any given protein do not exist, and phosphorylation of any protein on demand using small molecules would be advantageous. As such, new small molecules that endow new functions to enzymes via proximity-mediated effects could be useful in the study and treatment of critical cellular functions and diseases.

### SUMMARY

In certain example embodiments, multi-functional chemical conjugation molecules are provided, comprising a localizing moiety, a chemical linker moiety, an activator moiety, a first orienting adaptor interconnecting the chemical linker moiety on one end to the activator moiety, and optionally a second orienting adaptor interconnecting the chemical linker molecule on a different end to the localizing moiety.

Molecules in certain embodiment can be represented by

Formula I-A

Loc-L-(V₁-Act)₁ (I-A),

wherein Loc comprises the localizing moiety, L is the chemical linker moiety, V₁ is the first orienting adaptor, and Act is the activator moiety and wherein n is at least one; or

Formula I-B

Loc-V₂-L-(V₁-Act)ₙ (I-B)

wherein: Loc comprises the localizing moiety, L is the chemical linker moiety, V₁ is the first orienting adaptor, V₂ is the second orienting adaptor, and Act is the activator moiety. The molecule can include a first and second orienting adaptor independently selected from Table 1.

In an aspect, the activator moiety binds and activates an enzyme that modifies a target substrate associated with the localizing moiety. In certain embodiments, the target substrate is not a natural substrate of the enzyme, or wherein activation of the enzyme by the activator molecule results in modification of the target substrate by the enzyme at one or more new modification sites that would otherwise remain unmodified by the enzyme when not activated by binding to the activator moiety.

The linker may comprise a PEG molecule, alkyl, heterocycloalkyl, cycloalkyl, aryl, alkylene, alkenyl, heteroaryl, amide, amine, thiol or derivatives thereof. The linker can be a multifunctional linker, in an aspect, the linker is a multifunctional PEG linker. The multifunctional molecule may contain between about 2 and 5 activator molecules. The activator moiety is capable of finding and activating an enzyme in an aspect, the enzyme can be a kinase, phosphatase, transferase or ligase. In embodiments, the kinase a serine/threonine kinase, a tyrosine kinase, or a dual-specificity protein kinase that phosphorylates protein serine/threonine and protein tyrosine.

The kinase may comprise a AMP-activated protein kinase (AMPK), a Glucokinase (GK), or an AGC kinase. In embodiments, the activator moiety binds and activates a protein kinase C (PKC). The activator moiety, in embodiments, binds and activates a PKC isoform selected from: PKC-α, PKC-βI, PKC-βII, PKC-γ, PKC-ε, PKC-δ, PKC-η, or PKC-ξ. The activator moiety is selected from Table 1.

The molecule can comprise a localizing moiety that targets a nucleic acid, polypeptide, or polysaccharide. In embodiments, the localizing moiety is a target polypeptide binding moiety. In an aspect, the target polypeptide binding moiety binds a target polypeptide comprising a bromodomain and an extra-terminal motif (BET), which may be a bromodomain-containing protein 4 (BRD4), BRD3, BRD2, BRDT. The target polypeptide binding moiety can comprise (+)-JQ1.

The molecule can be according to the formula

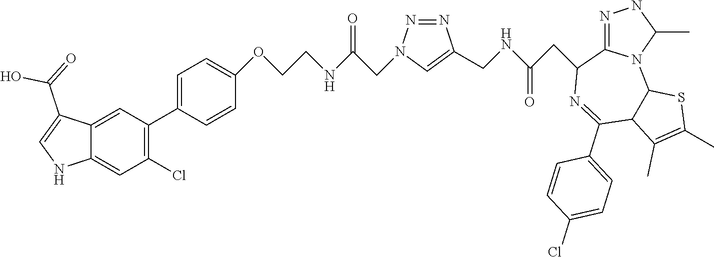

where n=3, 5, 7, 9, 11

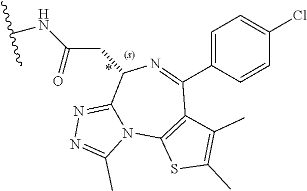 [SMILES: *NC(=O)C[C@@H]1N=C(c2ccc(Cl)cc2)c2c(sc(C)c2C)-n2c(C)nnc21]

[1.1-1.5].

The molecule can be according to the formula

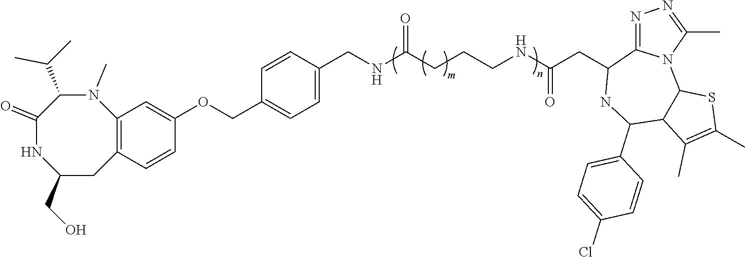

wherein n=0 and m=0, n=1 and m=1, n=2 and m=3, n=2 and m=1, n=2 and m=3 [PHICS 2.1-2.5].

The molecule can be selected from

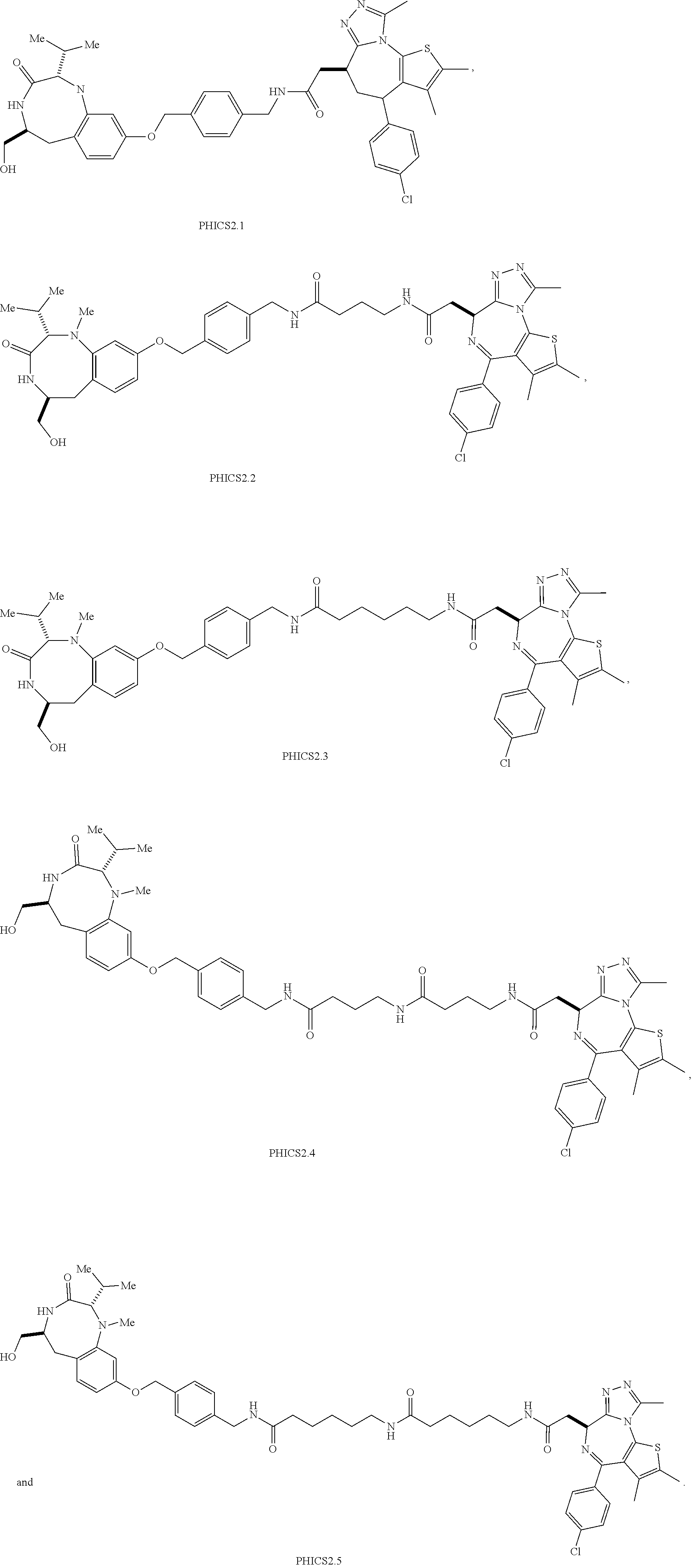

In an aspect, the molecule is according to the formula

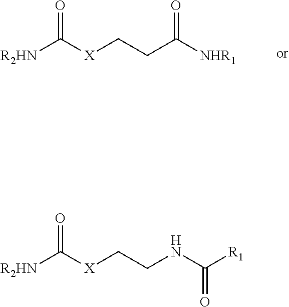

wherein R₁ is selected from JQ1, Ibrutinib, Dasatinib, MRTX, MI-1061, Gefitinib, Palbociclib, or Foretinib, and R₂ is selected from PF-06409577, Benzolactam or DPH, wherein X is CH₂ or (CH₂)₂O, and when X=CH₂, n=1 or 5 or m=0 or 4 and wherein when X=(CH₂)₂O, n=3, or m=3.

The molecule can be according to the formula

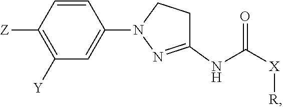

wherein Y and Z are a halogen, in preferred embodiments, the formula is according to

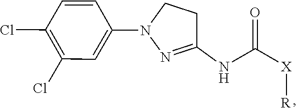

wherein X is selected from (CH₂)ₙ, (CH₂)ₙNHC(O)(CH₂)ₙ, (CH₂)ₙ(OC₂H₄)ₙ, (CH₂)ₙNHC(O)CH₂(OC₂H₄)ₙ, and (CH₂)ₙNHC(O)(CH₂)ₙ(OC₂H₄)ₙ, wherein each n is independently selected from 0, 1, 2, 3, 4, 5, 6 or 7 and R is

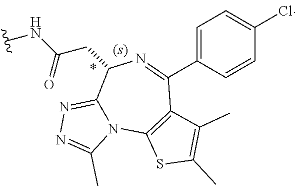 [SMILES: *NC(=O)C[C@@H]1N=C(c2ccc(Cl)cc2)c2c(sc(C)c2C)-n2c(C)nnc21]

The molecules disclosed herein may comprise independently selecting any of the localizing moieties as disclosed herein, the activating moiety is independently selected from each of the activators disclosed herein, the first and second orienting adaptor are independently selected from Table 2 and the linker is independently selected from the linker moieties as disclosed herein.

Pharmaceutical compositions can be provided comprising the molecule according to any one of the preceding claims and one or more pharmaceutically acceptable salts, carriers, or diluents. Compounds of the present invention may be provided with AMP.

Methods of modifying a target substrate in a cell, comprising contacting the cell with one or more of the multifunctional molecules described herein. In certain embodiments, the modifying comprises a post translational modification, which may comprise phosphorylation, hydroxylation, acetylation, methylation, glycosylation, prenylation, amidation, eliminylation, lipidation, acylation, lipoylation, deacetylation, formylation, S-nitrosylation, S-sulfenylation, sulfonylation, sulfinylation, succinylation, sulfation, carbonylation, or alkylation.

In an aspect, the modifying comprises inducing phosphorylation of a protein in the cell. Methods of phosphorylating a protein comprises contacting the protein with a molecule disclosed herein, wherein the protein is in proximity to a kinase specific to the activator moiety of the molecule. Phosphorylating of the protein may comprise phosphorylation of a plurality of proteins that are not a substrate of the kinase. The protein may be BRD4. In certain methods, the protein is phosphorylated between BD1 and BD2 of the BRD4. Methods of modifying a target substrate in a subject in need thereof are also provided, the method comprising administering a molecule as disclosed herein. In an aspect, the subject has a condition to be treated, which may be cancer.

Methods for modifying a protein of interest are also provided, the method comprising contacting the protein of interest with a compound disclosed herein in an environment comprising one or more activators. Methods for the treatment of a disease, disorder, or condition in a subject in need thereof can comprise administering a molecule disclosed herein to a subject.

Methods of making multifunctional conjugation molecules are also provided, comprising binding a localizing moiety and an activator moiety to different ends of a linker molecule, the localizing moiety and activator moiety optionally bound to the linker molecule via orienting adaptors wherein the linker molecule links the activator molecule such that both the activator molecule and localizing moiety is active in a cell.

These and other aspects, objects, features, and advantages of the 91 embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1C—An illustration of PHICS-induced ternary complex formation between FIG. 1A AMPK and BRD4 (PHICS1.2) or FIG. 1B PKC and BRD4 (PHICS2.3) and FIG. 1C general illustration of the proximity induced phosphorylation promoted by the chimeric molecules.

FIG. 2A-2H—Biochemical characterization of PHICS1.2-induced BRD4 phosphorylation by AMPK. (FIG. 2A) Ternary complex formation of BRD4, PHICS, and AMPK observed by AlphaScreen (normalized to DMSO). (R)-PHICS1.2 is the inactive analog with a low affinity for BRD4. (FIG. 2B) ADP-Glo™ assay for AMPK-catalyzed phosphorylation of BRD4 by PHICS1.2 compared to (R)-PHICS1.2. (FIG. 2C) Western blot analysis of BRD4 phosphorylation by PHICS1.2 using phospho-AMPK substrate motif antibody. (FIG. 2D) Bell-shaped dependence of BRD4 phosphorylation as a function of PHICS1.2 concentration analyzed via western blot. (FIG. 2E) ADP-Glo™ assay for AMPK-mediated phosphorylation of different peptide sequences from BRD4 or the peptide derived from AMPK substrate ACC (SAMS peptide). (FIG. 2F) Effect of AMPK isoforms on PHICS1.2-mediated BRD4 phosphorylation. (FIG. 2G) AlphaScreen for the ternary complex formation between AMPK, PHICS1.2, and different BRD proteins. (FIG. 2H) Detection of PHICS1.2-catalyzed phosphorylation of different BRD proteins by western blot. The loading level of BRD proteins was determined by coomassie gel.

FIG. 3A-3E—Biochemical characterization of PHICS2.3-induced BRD4 phosphorylation by PKC. (FIG. 3A) Formation of BRD4, PHICS, and PKC ternary complex observed by AlphaScreen normalized to the DMSO control. (FIG. 3B) ADP-Glo assay for PKC-catalyzed phosphorylation of BRD4 by PHICS2.3 compared to that of (R)-PHICS2.3. (FIG. 3C) Detection of BRD4 phosphorylation with phospho-PKC substrate motif antibody in western blot. (FIG. 3D) Different level of BRD4 phosphorylation observed between PKC isoforms in the presence of PHICS2.3. (FIG. 3E) Western blot analysis of PHICS2.3-mediated phosphorylation of different BRD proteins.

FIG. 4A-4B—(FIG. 4A) 3-D co-crystal structure of activator PF-06409577 bound to AMPK with key interactions K29, K31, and D88 in 2-D ligand map. (FIG. 4B) Docking of benzolactam activator to PKC with key interactions T242, L251, and G253 in 2-D ligand map. The solvent-accessible site where the linker were attached is highlighted in blue.

FIG. 5—Click-chemistry platform for the synthesis of PHICS1 with different linkers.

FIG. 6—Biochemical validation of AMPK activators, PHICS1 intermediates, and PHICS1.2 by ADP Glo assay with SAMS peptide as the substrate.

FIG. 7A-7D—Biochemical validation of PHICS1 with different linkers for identification of the optimal molecule for further studies. (FIG. 7A) Structures of PHICS1 analogs with varying linker length. (FIG. 7B) Schematic of Alpha screen assay for BRD4-PHICS-AMPK ternary complex formation. (FIG. 7C) Alpha screen assay for PHICS1 with different linkers normalized to DMSO. (FIG. 7D) Western blot analysis of AMPK catalyzed BRD4 phosphorylation with different concentrations of PHICS1 analogs.

FIG. 8A-8E—Validation of the inactive analog. (FIG. 8A) Structures of the PHICS1.2 and its inactive analog (R)-PHICS1.2. (FIG. 8B) ADP-Glo with SAMStide peptide as the substrate to compare the AMPK activation by PHICS1 molecules. (FIG. 8C) PHICS1.2 induced ternary complex formation of AMPK and BRD4 observed by pulldown assay. (FIG. 8D) Western blot analysis of His-tagged BRD4 (49-460) phosphorylation by AMPK in the presence of PHICS1.2. (FIG. 8E) Effect of AMPK concentration on PHICS1.2 or (R)-PHICS1.2 mediated BRD4 phosphorylation observed by western blot.

FIG. 9A-9E—Mass spectrometry identification of BRD4 phosphorylation by AMPK in the presence of PHICS1.2. Spectra for the peptides with phosphorylated (FIG. 9A) T169, (FIG. 9B) T186, (FIG. 9C) T221, (FIG. 9D) S324 and (FIG. 9E) S325. The fragmentation pattern is shown in each spectrum.

FIG. 10—Amino acid sequence alignment of truncated BRD4, BRD3 and BRD2 used in the experiments. Clustal Omega was used to generate the alignment. The phosphorylated residues of BRD4 identified by mass spectrometry analysis are marked with red (AMPK mediated) or blue (PKC mediated) asterisks.

FIG. 11—Synthesis of PHICS2 bifunctional molecules with different linkers.

FIG. 12—ADP-Glo assay using SAMStide peptide as the substrate to determine PKC-α activation by PKC activator and PHICS2 analogs.

FIG. 13A-13C—Identification of the optimal PHICS2 for BRD4 phosphorylation by PKC. (FIG. 13A) Alpha screen assay with different PHICS2 analogs for ternary complex formation. (FIG. 13B) Western blot analysis to compare BRD4 phosphorylation mediated by different PHICS2 molecules. (FIG. 13C) Effect of PKC concentration on BRD4 phosphorylation in the presence of PHICS2.3 or (R)-PHICS2.3.

FIG. 14A-14C—Mass spectrometry identification of BRD4 phosphorylation by PKC in the presence of PHICS2.3. Spectra for the peptides with phosphorylated (FIG. 14A) T229, (FIG. 14B) S324 and (FIG. 14C) S338. The fragmentation pattern is shown in each spectrum.

FIG. 15A-15B—NRM spectra: FIG. 15A ¹H NMR spectrum of Compound 3. FIG. 15B. ¹H NMR spectrum of 4.

FIG. 16A-16B—FIG. 16A ¹³C NMR of 4. FIG. 16B ¹H NMR of PHICS1.1.

FIG. 17A-17B—FIG. 17A ¹³C NMR of PHICS1.1. FIG. 17B ¹H NMR of PHICS1.2.

FIG. 18A-FIG. 18B—FIG. 18A ¹³C NMR of PHICS1.2. FIG. 18B ¹H NMR of (R)-PHICS1.2.

FIG. 19A-19B FIG. 19A—¹³C NMR of (R)-PHICS1.2. FIG. 19B ¹H NMR of PHICS1.3.

FIG. 20A-20B—FIG. 20A ¹³C NMR of PHICS1.3. FIG. 20B ¹H NMR of PHICS1.4.

FIG. 21A-21B—FIG. 21A ¹³C NMR of PHICS1.3; FIG. 21B ¹H NMR of PHICS1.5.

FIG. 22A-22B—FIG. 22A ¹³C NMR of PHICS1.5. FIG. 22B ¹H NMR spectrum of 21.

FIG. 23A-23B—FIG. 23A ¹³C NMR spectrum of 21. FIG. 23B ¹H NMR spectrum of 22.

FIG. 24—¹³C NMR spectrum of 22.

FIG. 25A-25B—FIG. 25A ¹H NMR spectrum of 23. FIG. 25B ¹³C NMR spectrum of 23.

FIG. 26A-26B—FIG. 26A ¹H NMR spectrum of 24. FIG. 26B ¹³C NMR spectrum of 24.

FIG. 27A-27B—FIG. 27A ¹H NMR spectrum of 25. FIG. 27B ¹³C NMR spectrum of 25.

FIG. 28A-28B—FIG. 28A ¹H NMR spectrum of 26. FIG. 28B. ¹³C NMR spectrum of 26.

FIG. 29A-29B—FIG. 29A ¹H NMR spectrum of 27. FIG. 29B. ¹³C NMR spectrum of 27.

FIG. 30A-30B—FIG. 30A ¹H NMR spectrum of 28. FIG. 30B ¹³C NMR spectrum of 28.

FIG. 31A-31B—FIG. 31A ¹H NMR spectrum of 29. FIG. 31B¹³C NMR spectrum of 29.

FIG. 32A-32B—FIG. 32A ¹H NMR spectrum of 30; FIG. 32B ¹³C NMR spectrum of 30.

FIG. 33A-33B—FIG. 33A ¹H NMR spectrum of PHICS2.1. FIG. 33B. ¹³C NMR spectrum of PHICS2.1.

FIG. 34A-34B—FIG. 34A ¹H NMR spectrum of PHICS2.2. FIG. 34B. ¹³C NMR spectrum of PHICS 2.2.

FIG. 35A-35B—FIG. 35A ¹H NMR spectrum of PHICS2.3. FIG. 35B. ¹³C NMR spectrum of PHICS2.3

FIG. 36A-36B—FIG. 36A ¹H NMR spectrum of PHICS2.4. FIG. 36B. ¹³C NMR spectrum of PHICS2.4.

FIG. 37A-37B—FIG. 37A ¹H NMR spectrum of PHICS2.5. FIG. 37B. ¹³C NMR spectrum of PHICS2.5.

FIG. 38A-38B—FIG. 38A ¹H NMR spectrum of (R)-PHICS2.3. FIG. 38B. ¹³C NMR spectrum of (R)-PHICS2.3.

FIG. 39A-39B—FIG. 39A—identification of kinase binder and linker optimization to generate PHICS molecules; FIG. 39B—PHICS-Kinase-BRD4 display ‘hook effect’ observed for ternary complexes.

FIG. 40—Mass spectrometry points to neo-phosphorylation sites on BRD4. Identified phosphorylated residues in kinase substrate recognition motif: T186, S324, S325, phosphorylated residues not in kinase substrate recognition motif: T169, T221. Spectra for the peptide with phosphorylated T169, left.

FIG. 41—Gel showing phosphatases can remove PICS mediated BRD4 phosphorylation.

FIG. 42—Imaging shows PHICS fail to phosphorylated BRD4 in cells with kinase isoform (cytosolic) and BRD4 (nuclear).

FIG. 43—Rational design of PHICS and controls for Bruton's Tyrosine Kinase BTK).

FIG. 44A-44B—explores PHICS mediated phosphorylation of BTK. FIG. 44A PHICS mediated phosphorylation of BTK in vitro, FIG. 44B PHICS mediated phosphorylation of BTK in cellulo

FIG. 45—PHICS mediated co-immunoprecipitation of the kinase and BTK.

FIG. 46—Phosphatase inhibitors increase levels of PHICS-mediated phosphorylation of BTK.

FIG. 47—Depiction of BTK pocket showing T474 forms H-bond; D539 and K430 form the base of the pocket (left); gel exhibiting lack of phosphorylation in BTK mutants T474A, K430R, ad D539N.

FIG. 48—Installation of bulky group on PHICS leads to loss of activity

FIG. 49—PHICS for FKBP12

FIG. 50—PHICS for c-Abl tyrosine kinase and BTK using Dasatinib

FIGS. 51A-51D—(A) Detection of BRD4 phosphorylation by immunoblotting using phospho-PKC substrate motif antibody. (B-C) ADP-Glo assay for BRD4 phosphorylation with (B) PHICS1 or (C) PHICS2 compared to their respective iPHICS. (D) Western blot analysis of PHICS1-mediated BRD4 phosphorylation using phosphor-Ser484/488 antibody.

FIGS. 52A-52B—(A) Effect of AMPK isoforms on PHICS1-mediated BRD4 phosphorylation. (B) Effect of PKC isoforms on BRD4 phosphorylation.

FIGS. 53A-53D—PHICS mediate BTK phosphorylation by AMPK in cells. (A) Structures of PHICS3 for phosphorylation of BTK via AMPK and Piv-PHICS3 inactive control. (B) Detection of ternary complex formation in HEK293T cells by co-immunoprecipitation of AMPK and BTK-Flag in the presence of PHICS3. WCL: Whole Cell Lysate (C-D) Western blot analysis of BTK phosphorylation by PHICS3 in HEK293T cells transfected with WT BTK-Flag (C) and BTK-Flag S180A mutant (D). See SI for structures of AMPK activator and BTK inhibitor.

FIGS. 54A-54D—Target engagement of PHICS3 in cells. (A) Competition experiment with covalent BTK inhibitor, Ibrutinib. (B) Key interactions of Ibrutinib with BTK (PDB ID: 5P9I). (C) Western blot analysis of PHICS3-induced phosphorylation of WT BTK and T474 mutant. (C) Western blot analysis of BTK phosphorylation with PHICS3 and its inactive analog Piv-PHICS3.

FIG. 55A-55B—Molecular docking. (55A) Co-crystal structure of activator PF-06409577 bound to AMPK (PDB ID: 5KQ5). The solvent-exposed site was modified for linker attachment is highlighted in blue. (55B) The 2-D ligand map showing key interactions of the molecule with Lys29, Lys31, and Asp88.

FIG. 56A-56B—Biochemical validation of modified activators and PHICS intermediates by ADP Glo assay. (56A) Biochemical validation of AMPK activation by potent activator PF-06409577, AMPK activator and AMPK activator with linker by ADP-Glo assay with SAMS peptide as the substrate. (56B) ADP-Glo assay using CREBtide peptide as the substrate to determine PKCα activation by modified PKC activator.

FIG. 57—Syntheses of the AMPK activator, AMPK activator with linker, bifunctional AMPK-PHICS with different linker lengths (PHICS1, the lead compound derived from n=5), and the inactive analog iPHICS1.

FIG. 58—Syntheses of the PKC activator and bifunctional PKC-PHICS with different linker lengths (PHICS2, the lead compound derived from n=1, m=3), and the inactive analog iPHICS2.

FIG. 59A-59D—Identification of the optimal PHICS for BRD4 phosphorylation by AMPK. (59A) Structures of AMPK-PHICS analogs with varying linker length. (59B) Schematic of Alpha screen assay for BRD4-PHICS-AMPK ternary complex formation. (59C) AlphaScreen assay for AMPK-PHICS with different linkers normalized to DMSO. (59D) Western blot analysis of AMPK catalyzed BRD4 phosphorylation with different concentrations of AMPK-PHICS analogs.

FIG. 60A-60C—Identification of the optimal PHICS for BRD4 phosphorylation by PKC. (60A) Structures of PKC-PHICS analogs with varying linker length. (60B) AlphaScreen assay with different PKC-PHICS analogs for ternary complex formation normalized to DMSO. (60C) Western blot analysis to compare BRD4 phosphorylation mediated by different PKC-PHICS molecules.

FIG. 61A-61G—Validation of the inactive analog. (61A) Structures of the PHICS1, PHICS2 and their inactive analogs iPHICS1 and iPHICS2. (61B) ADP-Glo with SAMStide peptide as the substrate to compare the AMPK activation by PHICS1 and iPHICS1. (61C) ADP-Glo with CREBtide peptide as the substrate to compare the PKC activation by PHICS2 and iPHICS2. (61D) PHICS1 induced ternary complex formation of AMPK and BRD4 observed by pulldown assay. (61E) Western blot analysis of His-tagged BRD4 (49-460) phosphorylation by AMPK in the presence of PHICS1. (61F) Effect of AMPK concentration on PHICS1 or iPHICS1 mediated BRD4 phosphorylation observed by western blot. (61G) Effect of PKC concentration on BRD4 phosphorylation in the presence of PHICS2 or iPHICS2.

FIG. 62—Bell-shaped dependence of BRD4 phosphorylation as a function of PHICS1 concentration analyzed via western blot.

FIG. 63A-63B—Addition of AMP enhances phosphorylation. (63A) In vitro kinase assay for BRD4 phosphorylation by AMPK (1 uM compound MS231, VS804, VS806); (63B) ADP glo for BRD4 phosphorylation by AMPK, with and without AMP.

FIG. 64A-64C—(64A) Structures of Halo- and BRD4-targeting PHICS molecules based on known Abl kinase activator. L-linker. (64B-64C) Western blot analysis of PHICS-induced phosphorylation of Halo-tag (64B) and BRD4 (64C).

FIG. 65—ADP-Glo assay for AMPK-mediated phosphorylation of different peptide sequences from BRD4 or the peptide derived from AMPK substrate ACC (SAMS peptide).

FIG. 66—ADP-Glo assay for the activation of different AMPK isoforms by PF-06409577.

FIG. 67—AlphaScreen assay for the ternary complex formation between AMPK, PHICS1, and different BRD proteins.

FIG. 68—Synthesis of noncovalent BTK inhibitor, PHICS3, its analogs with different linkers and inactive control Piv-PHICS3.

FIG. 69—In-vitro biochemical validation of BTK phosphorylation by AMPK in the presence of PHICS. Western blot analysis for BTK phosphorylation using 10 μM BTK targeting AMPK-PHICS with different linkers for identification of the optimal molecule for further studies. Molecule with 8 carbon alkyl linker (PHICS3) was identified as the most optimal molecule for phosphorylation.

FIG. 70A-70D—Cell base studies for PHICS mediated BTK phosphorylation by AMPK. (70A) Western blot analysis for phosphorylation of Flag-BTK overexpressed in HEK293 cells using PHICS with different linkers. Compounds were treated for 4 hrs and phosphorylation was detected by phospho-BTK (Ser180) antibody. Molecule with 8 carbon alky linker (PHICS3) was identified as the optimal molecule for phosphorylation. (70B) Western blot analysis to compare BTK Ser180 phosphorylation in the presence of 5 μM PHICS3, 5 μM activator, or increasing concentrations of a potent AMPK activator, PF-06409577. (70C) Time-dependent and (70D) PHICS3 dose-dependent BTK phosphorylation observed by western blot. For the time-dependent study, 5 μM of PHICS3 was used.

FIG. 71A-71C—Validation of PHICS3 target engagement with BTK in cells. (71A) Structure of Ibrutinib and the covalent binding of Ibrutinib to BTK. Pretreatment of cells with Ibrutinib leads to covalent irreversible modification of Cys481 and blocks binding of PHICS3 to BTK. (71B) Crystal structure of Ibrutinib bound to BTK (PDB ID: 5P91) with key interactions. Thr474 forms hydrogen bond with the 4-amino group of Ibrutinib. Asp539 (cyan) forms H— bond interactions (Magenta) with Ibrutinib and Lys430 (green). (71C) Detection of S180 phosphorylation in T474A, K430R and D539N mutants of BTK compared to the WT.

FIG. 72—¹H NMR spectrum of 37.

FIG. 73—¹³C NMR spectrum of 37.

FIG. 74—¹H NMR spectrum of PHICS3 recorded in DMSO-d6.

FIG. 75—¹³C NMR spectrum of PHICS3 recorded in DMSO-d6.

FIG. 76—¹H NMR spectrum of Piv-PHICS3 recorded in CDCl3:CD3OD (1:1) solvent mixture.

FIG. 77—¹³C NMR spectrum of Piv-PHICS3 recorded in CDCl3:CD3OD (1:1) solvent mixture.

FIG. 78—Shown are two concepts for using N-acyl N-alkyl sulfonamide (NASA) for activating PKC. Top panel shows concept 1: labeling activator (PKC) with a localizing moiety, e.g., binder of protein of interest using a NASA warhead. Bottom panel shows concept 2: label PKC with trans-cyclooctene (TCO) using a NASA warhead. It can then be attached to a localizing moiety, e.g. binder of any protein of interest, with tetrazine click chemistry.

FIGS. 79A-79B—Phosphorylation of the transcription factor will disrupt its (79A) protein-DNA and (79B) protein-protein binding.

FIG. 80—Modular synthesis of exemplary PHICS molecules for kinase evaluation, with exemplary activator moieties ABL activator, IR activator, MEK inhibitor and AKT inhibitor, and localizing moieties identified as AR binder and BRD4 binder.

FIGS. 81A-81D—Binders of transcription factors targeted by PHICS.

FIGS. 82A-82B—(82A) Cellular localization of PHICS targets. (82B) Timeline and workflow.

FIGS. 83A-83C—Structures of (83A) DPH and (83B) DPH-L6-azide. (83C) ADP-Glo assay for DPH and DPH-L6-azide.

FIGS. 84A-84D—Binders of selected (84A) protein target (localizing moieties) or (84B) kinases (activator moiety) functionalized with a reactive handle (red and green for A and B, respectively). (84C-84D) Synthetic schemes for the construction of PHICS molecules. Note: name under the structure represents parent binder with corresponding protein provided in parentheses.

FIG. 85—Cooperativity in three-body equilibria.

FIG. 86—Schematic of the kinetic process of drug action, from administration of the drug to disease progression. Definitions: compound concentration in plasma (Cₚ); in target vicinity (C); in complex with target (CT); free target concentration (T); kᵢₙ is production rate; kₒᵤₜ is dissipation rate; kₒₙ is the on-rate constant; k_{off} is the off-rate constant.

FIG. 87—Chemical structures of the CDK8 inhibitors initially considered in this study and their experimental residence times. The common 1-(3-tert-butyl-1-p-tolyl-1H-pyrazol-5-yl)urea scaffold is depicted in blue.

FIG. 88—Active site of the crystal structure of human CDK8 in complex with compounds 1-7 in FIG. 87. The interactions of the urea group of the crystallized inhibitors with Glu66 and Asp173 are shown by dashed lines. Protein carbon atoms are represented in white. The protein backbone is represented as a white cartoon, with the exception of the hinge region (gray cartoon).

FIG. 89-2D representation of eight p38α inhibitors, SB5, SB6, SB7, B12, B96, BR5, BR8 and BMU. Common substructures are highlighted.

FIG. 90—Kinetics of inhibition of various inhibitors of Abl1, Dasatinib, Imatinib, Ponatinib and Nilotinib.

FIG. 91—Evaluation of tyrosine phosphorylation Abl-BRD4 (pTyr Millipore) compounds according to exemplary embodiments.

FIG. 92—Evaluation of tyrosine phosphorylation Abl-BRD4 (DPH Activator) compounds according to exemplary embodiments.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

“Diastereoisomers” are stereoisomers that have at least two asymmetric atoms, but which are not mirror-images of each other. The absolute stereochemistry is specified according to the Cahn-Ingold-Prelog R-S system When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. In some chemical structures, stereocenters may be identified with “wavy” bonds indicating that the stereocenter may be in the R or S configuration, unless otherwise specified. However, stereocenters without a wavy bond (i.e., a “straight” bond) may also be in the (R) or (S) configuration, unless otherwise specified. Compositions comprising compounds may comprise stereocenters which each may independently be in the (R) configuration, the (S) configuration, or racemic mixtures.

Optically active (R)- and (S)-isomers can be prepared, for example, using chiral synthons or chiral reagents, or resolved using conventional techniques. Enantiomers can be isolated from racemic mixtures by any method known to those skilled in the art, including chiral high pressure liquid chromatography (HPLC), the formation and crystallization of chiral salts, or prepared by asymmetric syntheses.

Optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid. The separation of the mixture of diastereoisomers by crystallization followed by liberation of the optically active bases from these salts affords separation of the isomers. Another method involves synthesis of covalent diastereoisomeric molecules by reacting disclosed compounds with an optically pure acid in an activated form or an optically pure isocyanate. The synthesized diastereoisomers can be separated by conventional means such as chromatography, distillation, crystallization or sublimation, and then hydrolyzed to deliver the enantiomerically enriched compound.

Optically active compounds can also be obtained by using active starting materials. In some embodiments, these isomers can be in the form of a free acid, a free base, an ester or a salt.

In certain embodiments, a disclosed compound can be a tautomer. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). Tautomerization includes prototropic or proton-shift tautomerization, which is considered a subset of acid-base chemistry. Prototropic tautomerization or proton-shift tautomerization involves the migration of a proton accompanied by changes in bond order. The exact ratio of the tautomers depends on several factors, including temperature, solvent, and pH. Where tautomerization is possible (e.g., in solution), a chemical equilibrium of tautomers can be reached. Tautomerizations (i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

As used herein, a bond substitution coming out of a ring, e.g,

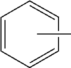

means that the substitution can be at any of the available position on the ring.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide compounds that induce, effect or promote a modification of a target substrate. The compounds are multi-functional conjugation molecules comprising a localizing moiety, a chemical linker moiety, an activator moiety, and a first orienting adaptor interconnecting the chemical linker moiety on one end to the activator moiety. Optionally, a second orienting adaptor interconnecting the chemical linker moiety on a different end to the localizing moiety is present. The terms “multi-functional conjugation compound”, “multi-functional molecule”, “chimeric molecule”, and “chimeric conjugation molecule” are used interchangeably herein.

The multi-functional chemical conjugation molecules can be utilized to modify a target substrate. In one embodiment, the modification is a post-translational modification. Modification as used herein can include addition of a functionality or removal of a functionality. Exemplary embodiments disclosed herein provide small molecule compounds that induce phosphorylation and/or are capable of phosphorylating proteins.

The compounds of the present invention preferably modify a macromolecular substrate, for example polypeptides, oligosaccharides and polysaccharides, and nucleic acids such as DNA or RNA. Associations with macromolecule may comprise covalent bonding, non-covalent bonding, electrophilic association or other association with the macromolecule. Binding may comprise reversible or non-reversible binding

The multi-functional chimeric molecules can induce modifications and can be designed to allow for temporal and dose-dependent control, as detailed herein.

Modifications effected by the activator associated with the activator moiety of the multifunctional compound, either directly or indirectly, can be reversible or irreversible. In embodiments, the modification may include the addition of a chemical group, such as phosphorylation, hydroxylation, acetylation or methylation. In one aspect, the modification is the addition of complex molecules, such as prenylation, glycosylation, ADP-ribosylation, and AMPylation. In another aspect, the modification may include cleavage, e.g. proteolysis. Amino acid modification such as deamidation and eliminylation is effected.

In one example embodiment, the multifunctional chimeric molecule, or multi-functional chemical conjugation molecule, comprise a localizing moiety, a chemical linker moiety, an activator moiety, a first orienting adaptor interconnecting the chemical linker moiety on one end to the activator moiety, and optionally a second orienting adaptor interconnecting the chemical linker molecule on a different end to the localizing moiety. One or more activator moiety may be utilized in the multifunctional chimeric molecules.

In certain example embodiments, the molecule can be represented by Formula I-A

Loc-L-(V₁-Act)₁ (I-A),

wherein: Loc comprises the localizing moiety, L is the chemical linker moiety, VI is the first orienting adaptor, and Act is the activator moiety and wherein n is between 1 and 5;

In certain other example embodiments, the molecule be represented by Formula I-B

Loc-V₂-L-(V₁-Act)ₙ (I-B)

wherein: Loc comprises the localizing moiety, L is the chemical linker moiety, V₁ is the first orienting adaptor, V₂ is the second orienting adaptor, and Act is the activator moiety, wherein n is between 1 and 5.

Use of the multifunctional compounds disclosed herein include use in proximity induced modifications of a macromolecular substrate. Using phosphorylation as an exemplary modification, the compounds can advantageously induce phosphorylation for a protein target of interest. For example, proteins that are non-substrates of the activator moiety of the multifunctional compound can be phosphorlyate by example embodiments of the multifunctional compounds disclosed herein. The multifunctional compounds disclosed herein offer a new class of molecules capable of inducing modification of a target substrate, e.g. phosphorylation of a target protein (e.g., bromodomain family proteins) that does not require use with a natural substrate of the activator moiety. Using principles of proximity-induced reactivity, kinases such as AMPK or PKC can phosphorylate a non-substrate protein (e.g., BRD4).

The multifunctional chimeric molecules can be designed and tailored according to localizing moiety, the activator moiety and necessary proximity to allow for the desired modification of a target substrate, as detailed further herein.

### Localizing Moiety

The localizing moiety, represented in formula I-A and I-B as Loc, provides for the targeting, binding, or association with a macromolecule. In embodiments, the macromolecule is a polypeptide, a nucleic acid, such as DNA or RNA, or a sugar molecule. In embodiments, the polypeptide is a protein, for example, an enzyme.

In one embodiment, the localizing moiety binds to a target substrate to be modified. The localizing moiety's function is to bind a substrate to be modified by the activator bound by the activator moiety, bringing the target substrate into proximity with the activator moiety. The reaction can allow the activator to modify a larger number of substrates, non-natural target substrates of the activator, and to increase the kinetics/efficiency of such substrate modifications. For that purpose, the localizing moiety should be able to bind the specific substrate and may include a large number of molecules suitable for that purpose and capable of being linked to the activator moiety, as further described herein, to allow for modification of the substrate. Provided herein are different example classes of localizing moiety based on the substrate targeted for modification.

In embodiments, the localizing moiety is chosen based on the desired association and modification to be effected. Accordingly, the modifications desired, which may be tailored based on a particular condition, disease, treatment, or other desired effect, will be a design consideration when choosing the localizing moiety. By way of example, DNA binding domains, which are positively charged, interaction with negatively charged DNA backbone. Phosphorylation converts a neutral residue to a negatively charged residue, with charge neutralization has lower DNA binding affinity. See, e.g. Gallagher, et al. Nature Methods, Broad specificity profiling of TALENs results in engineered nucleases with improved DNA-cleavage specificity; Slaymaker et al., Science, Rationally engineered Cas9 nucleases with improved specificity.

### Localizing Moieties for Binding Polypeptide Substrates

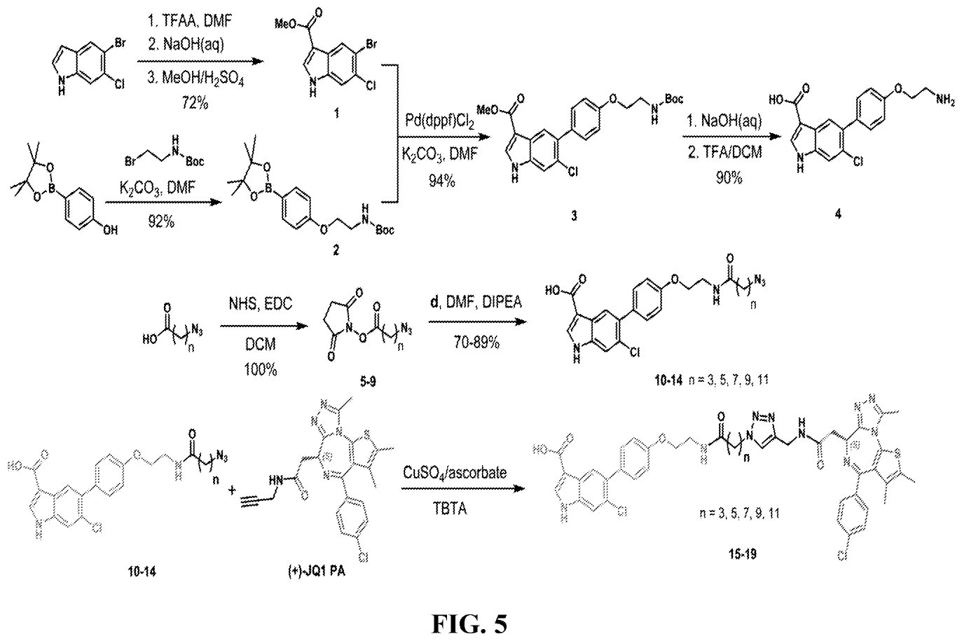

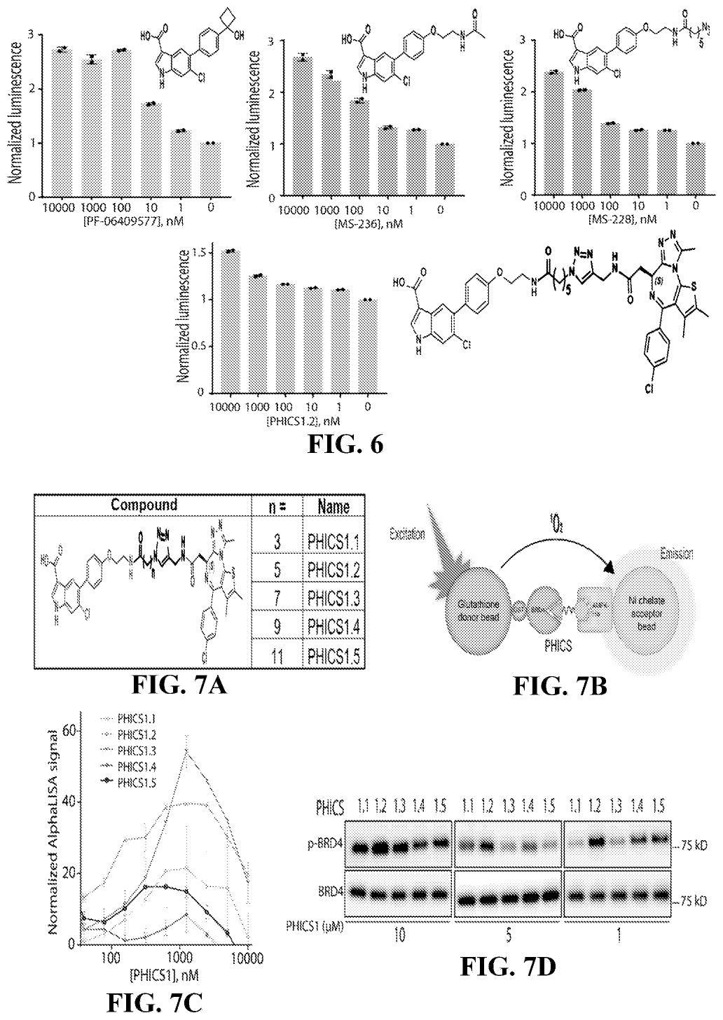

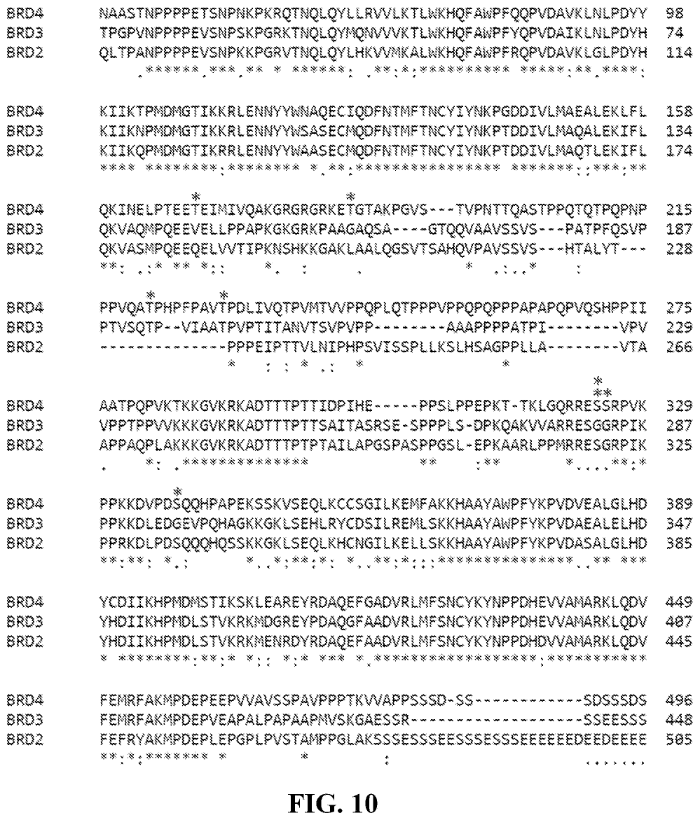

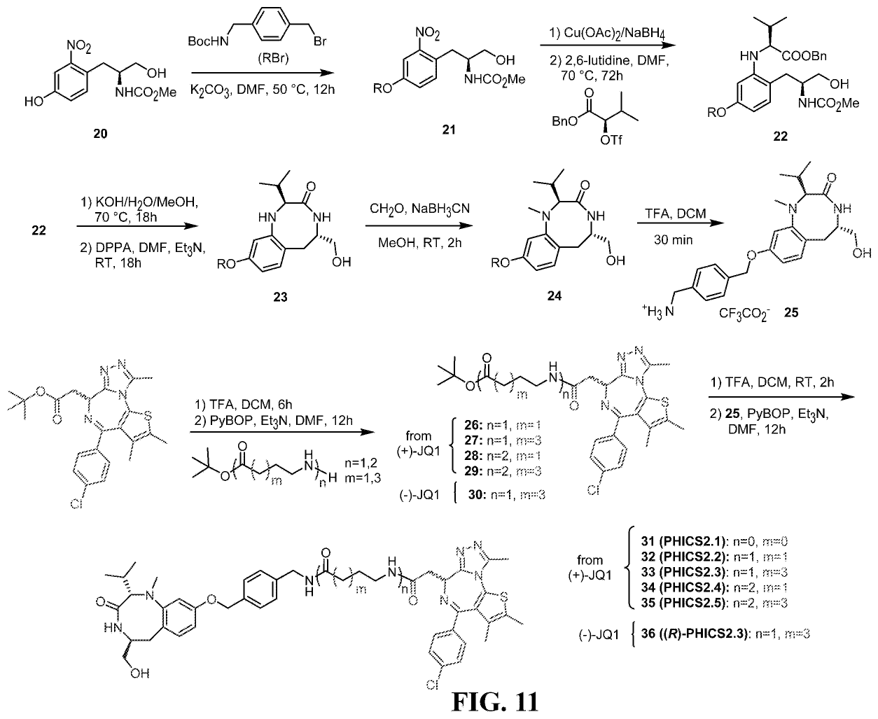

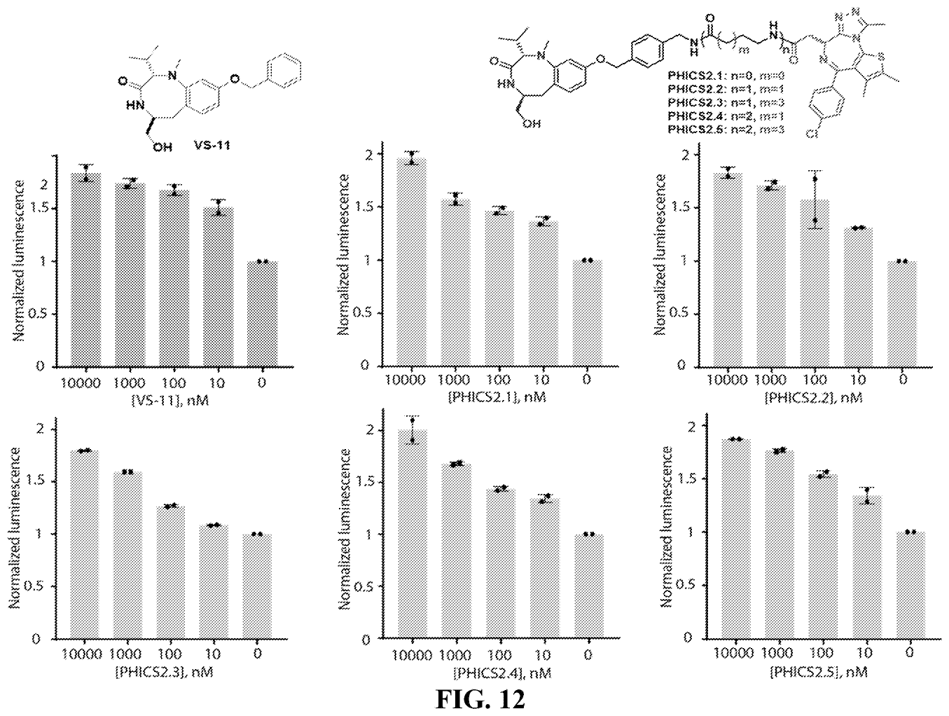

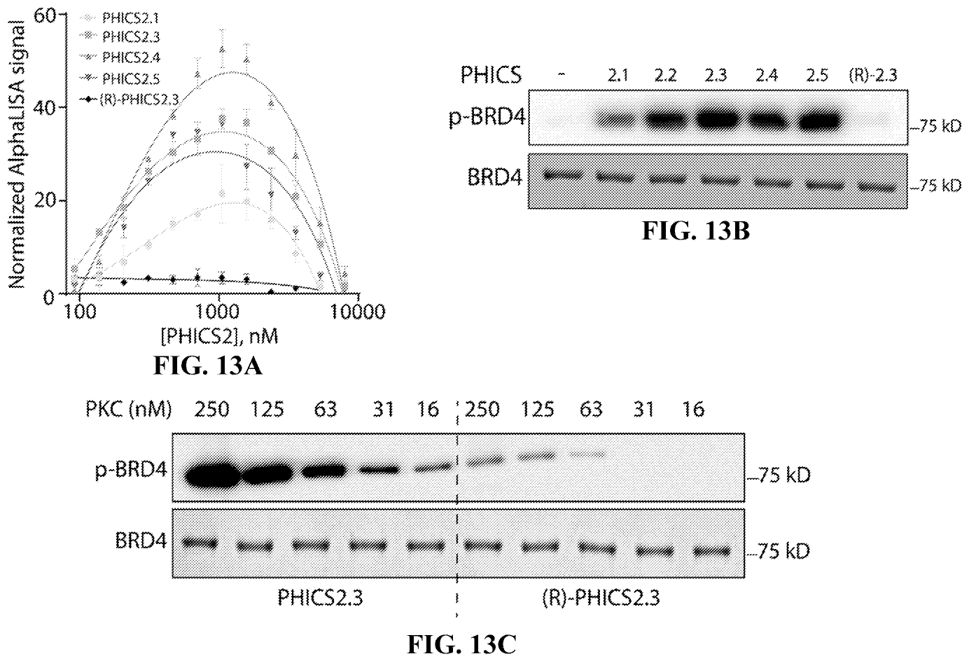

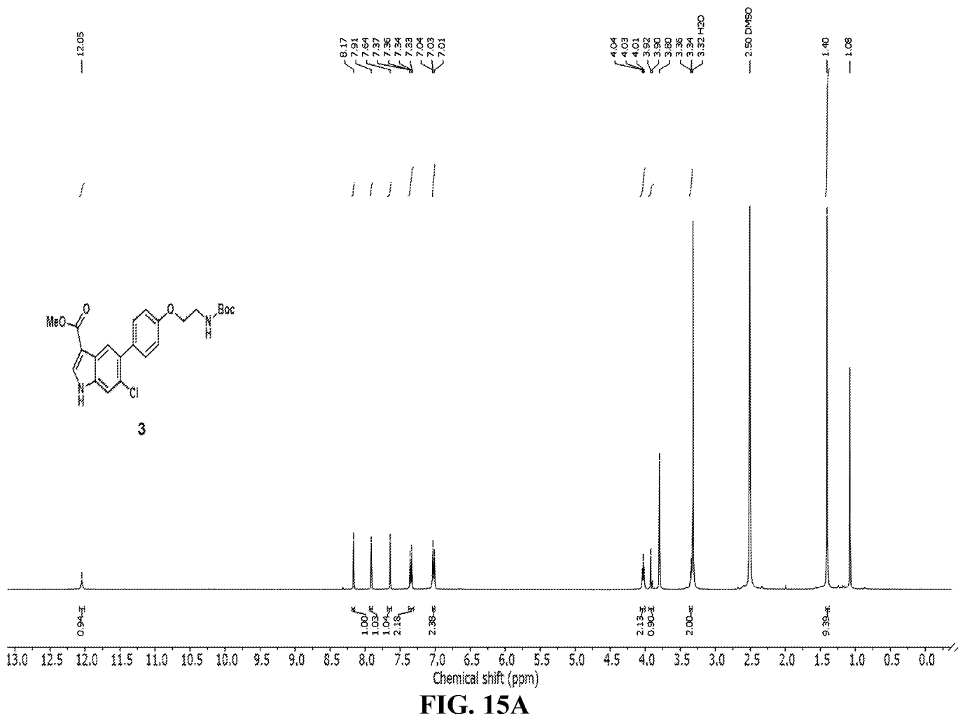

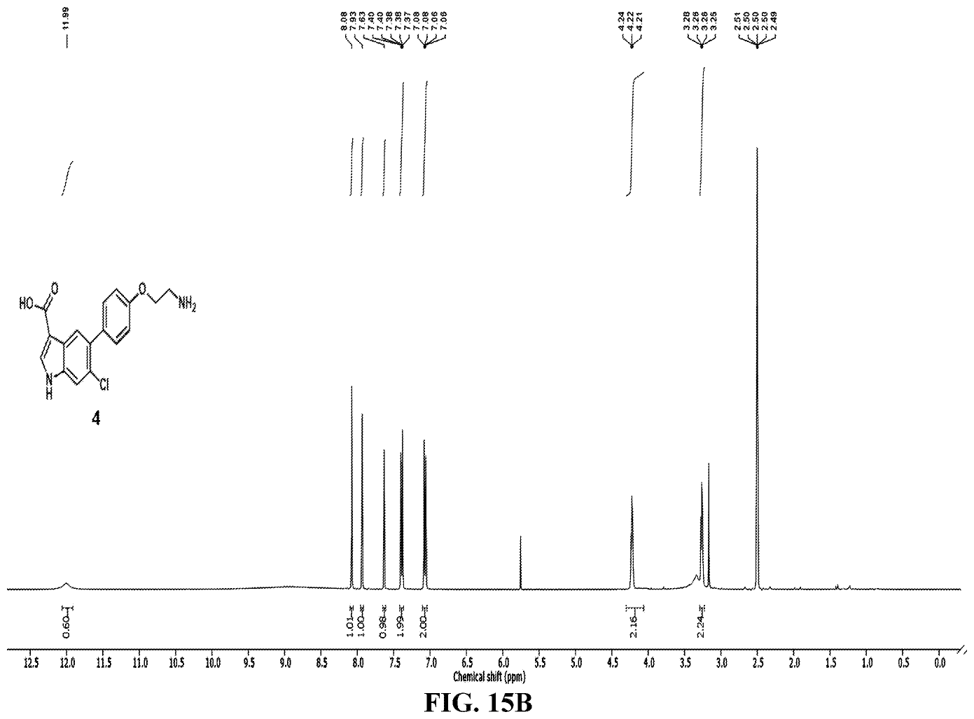

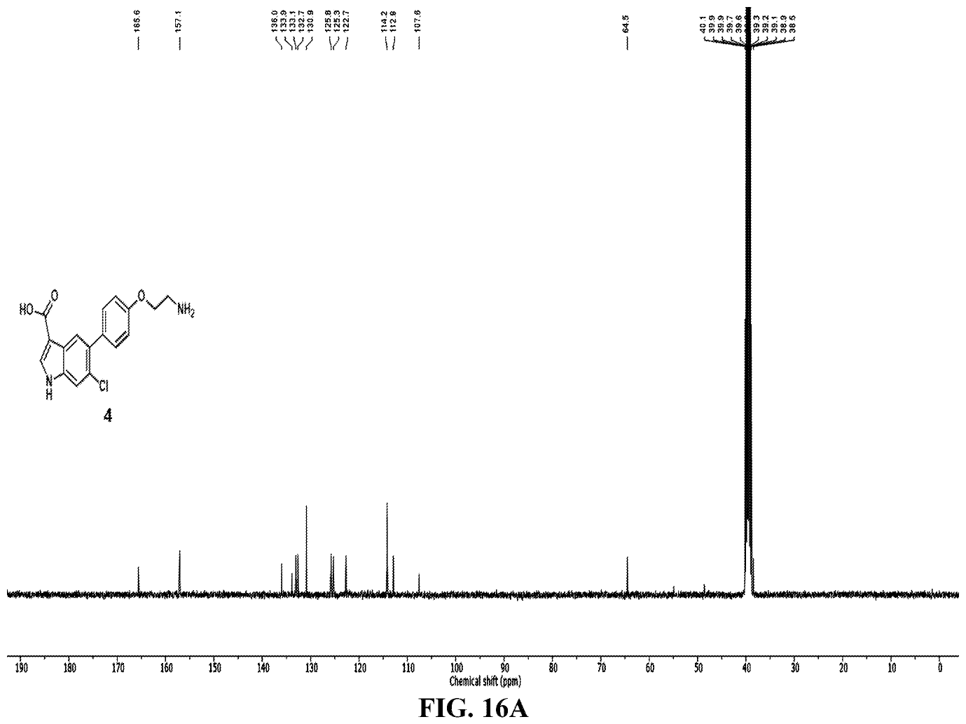

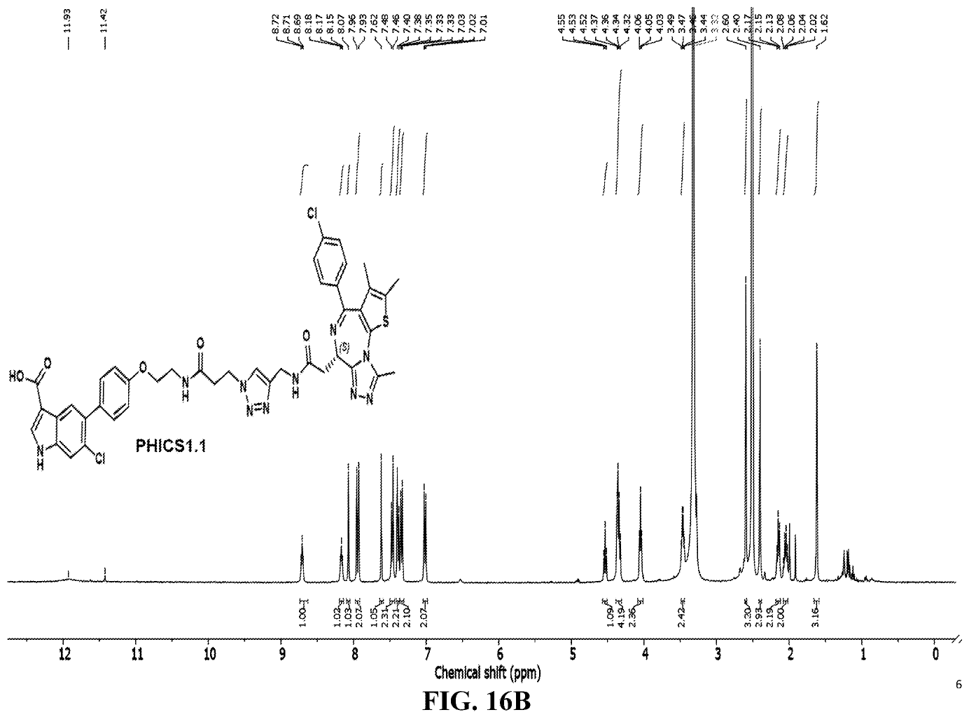

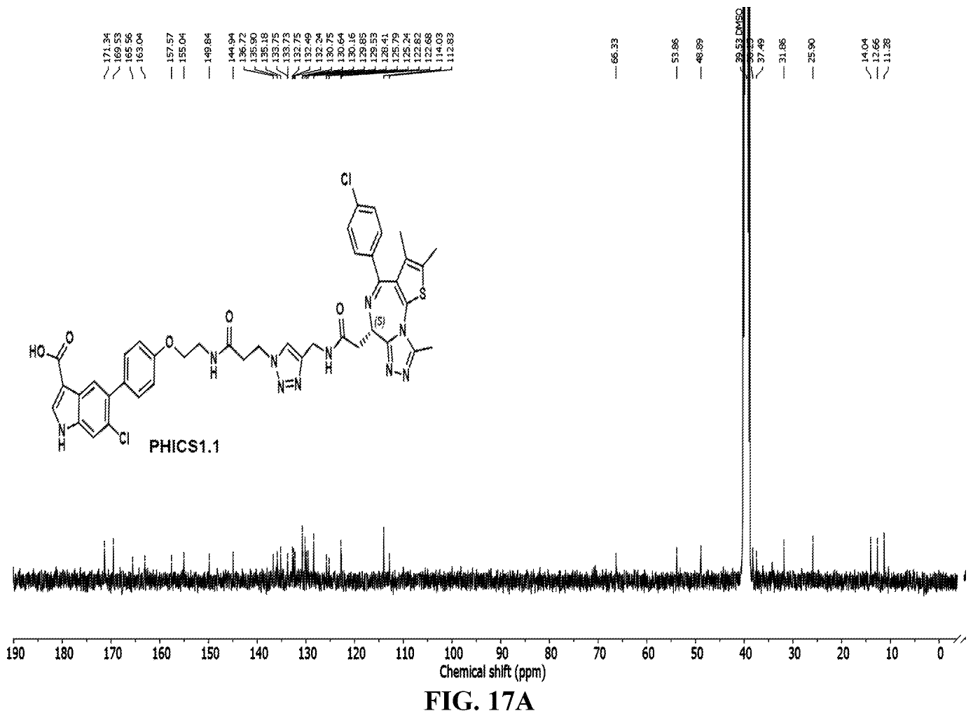

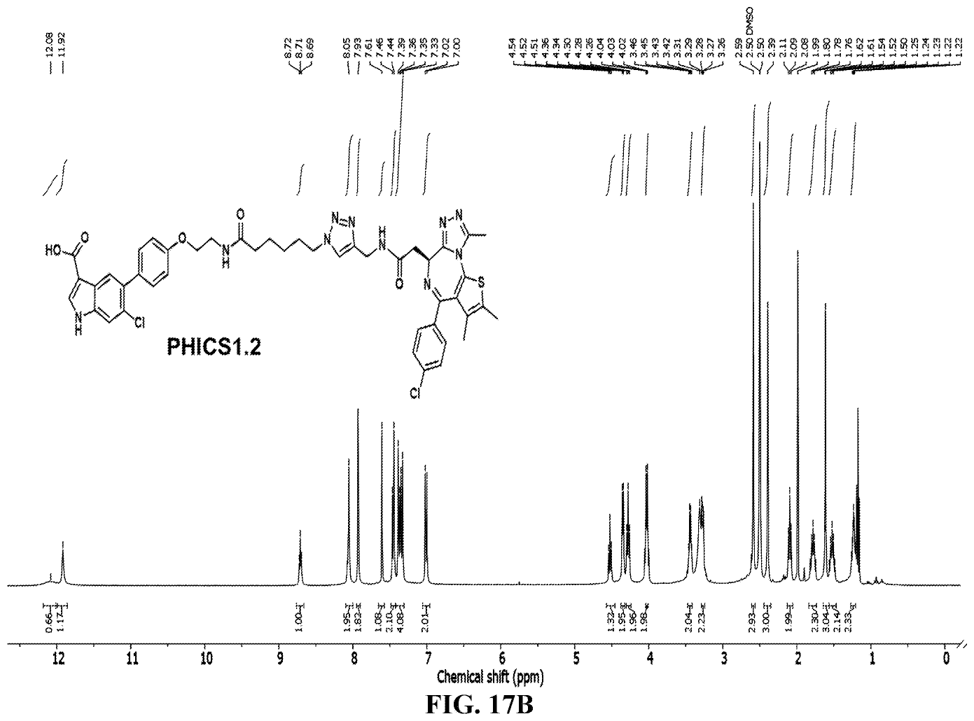

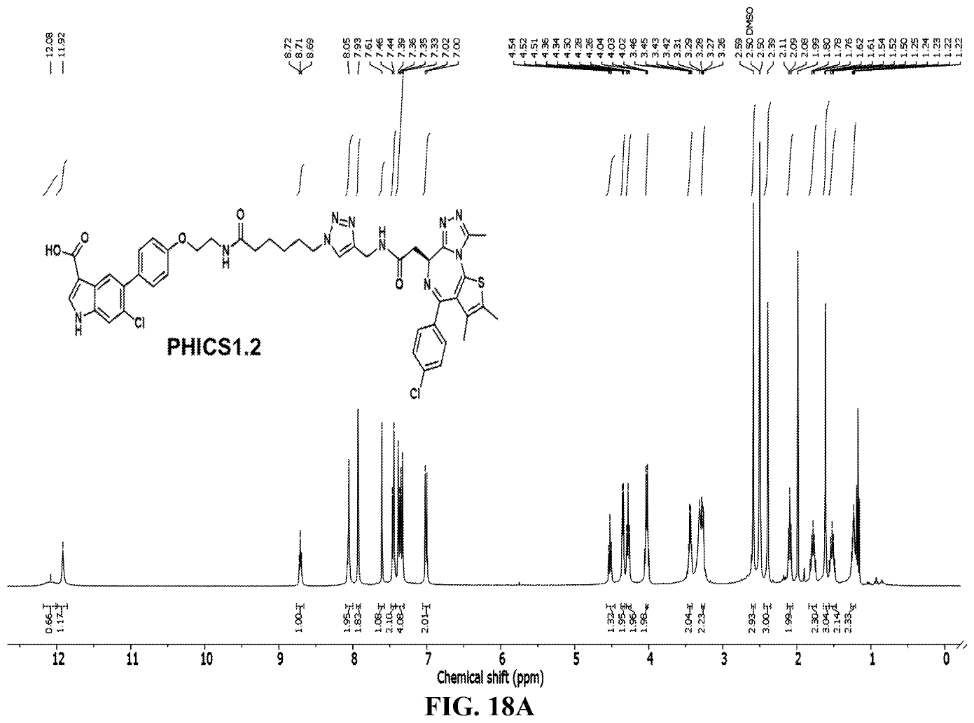

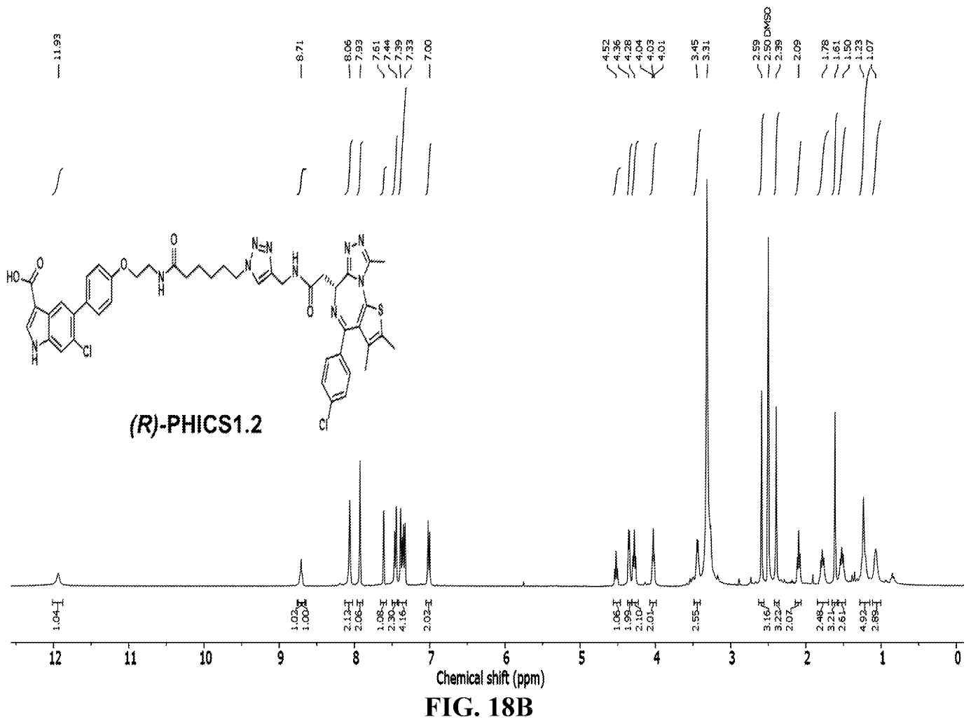

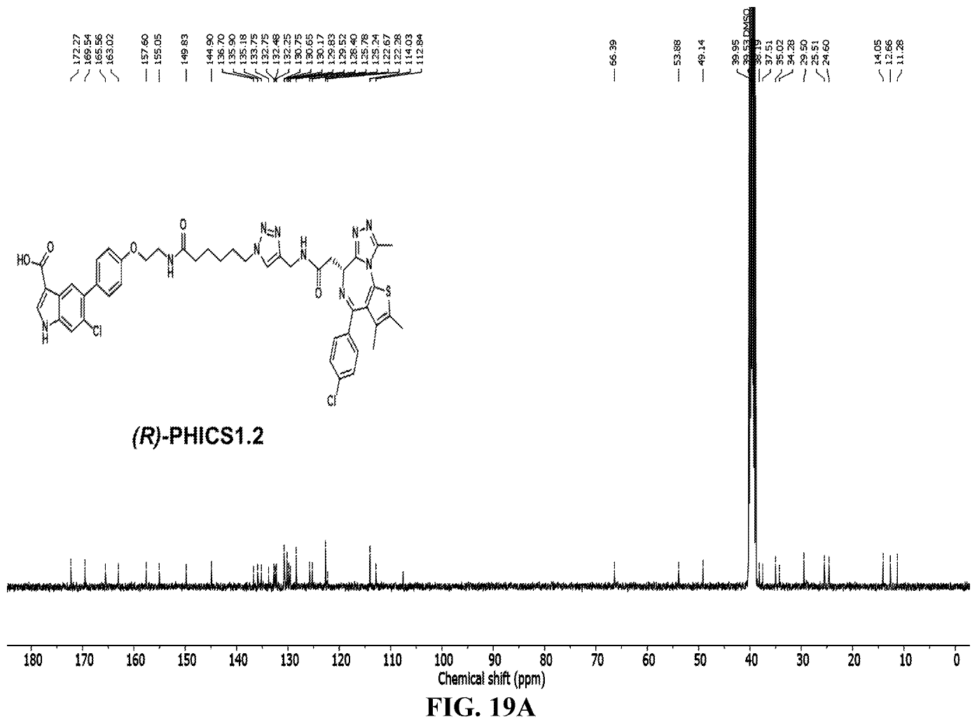

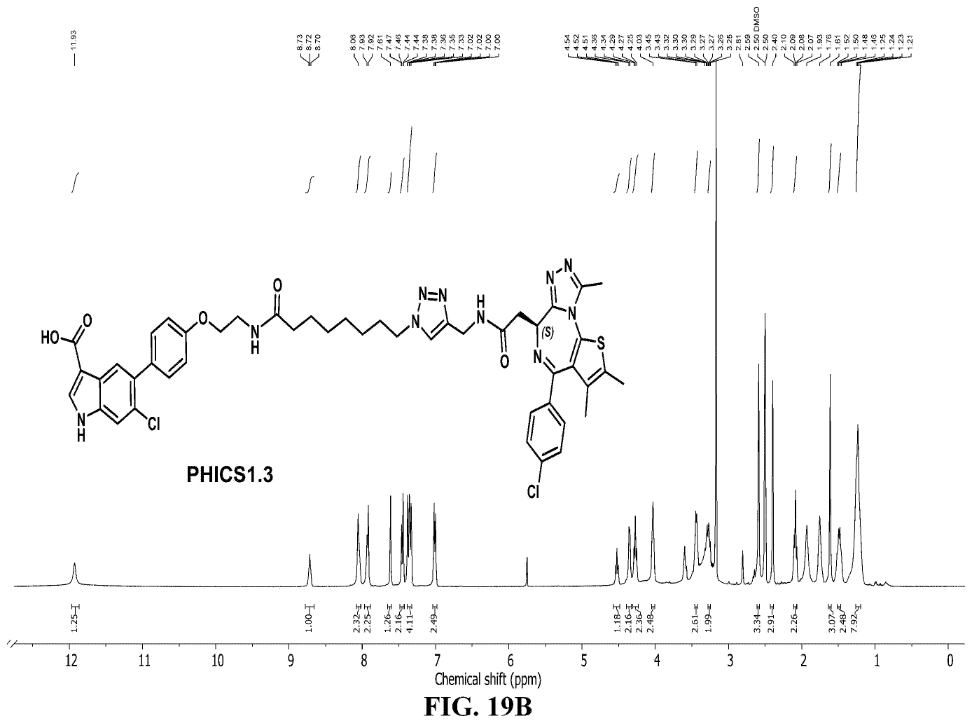

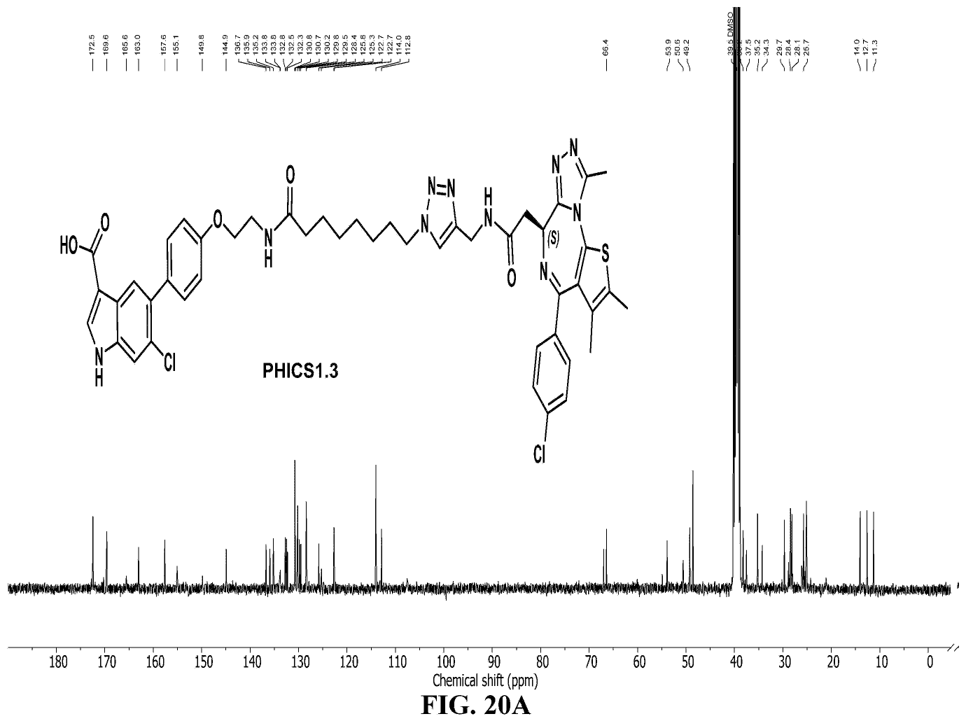

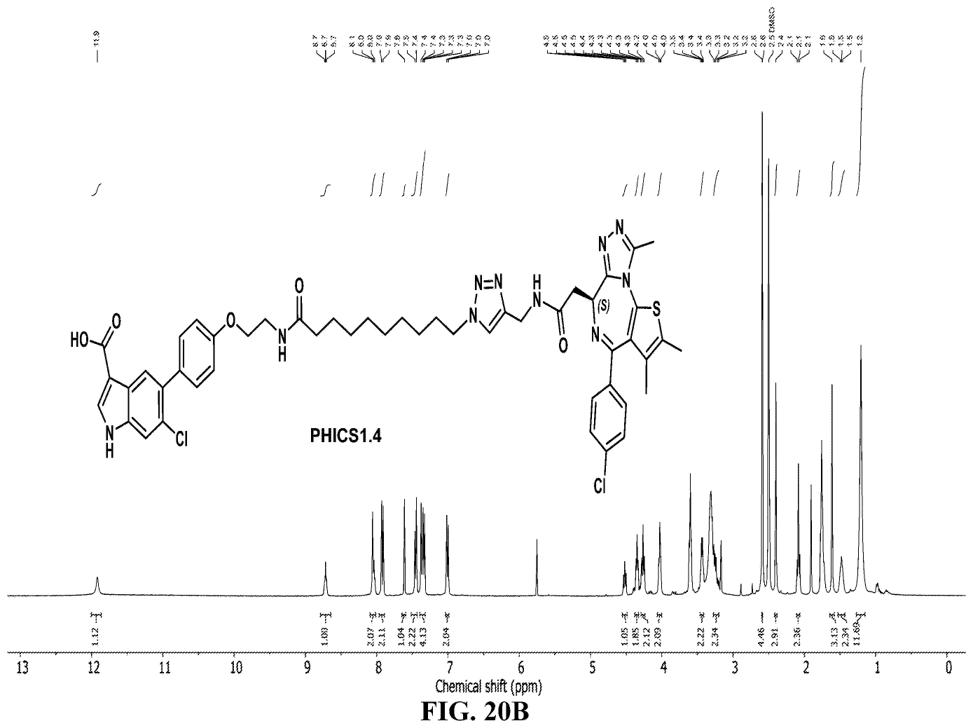

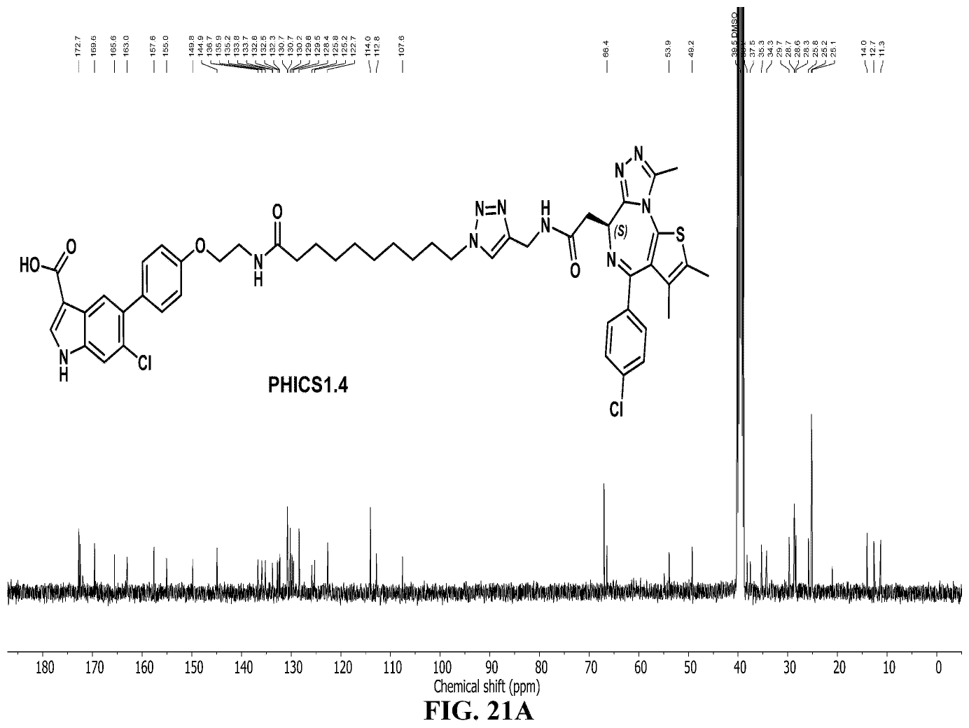

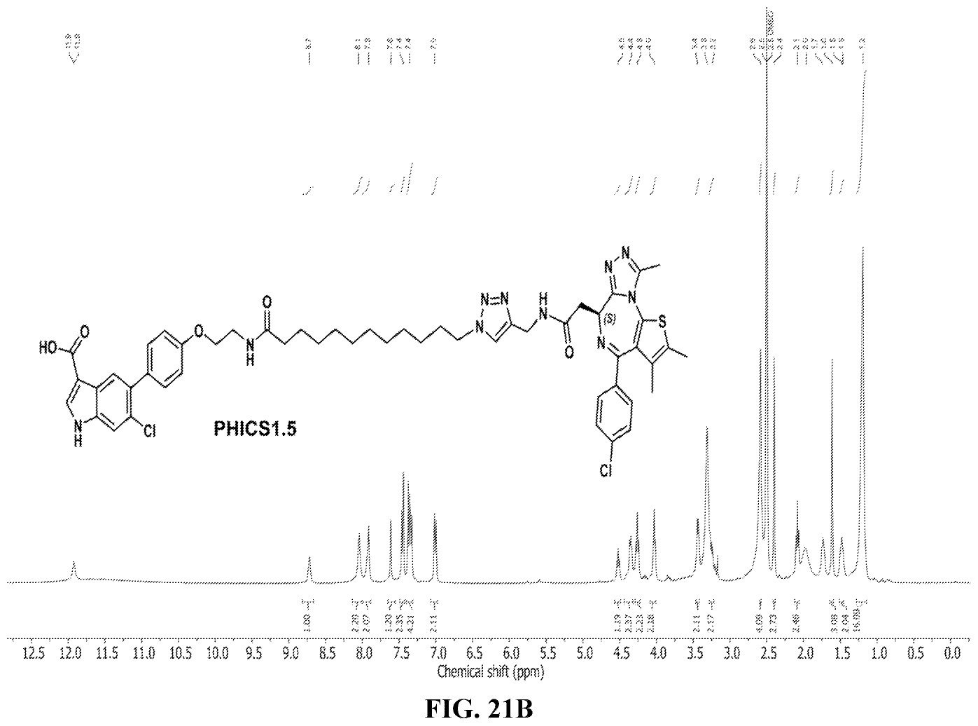

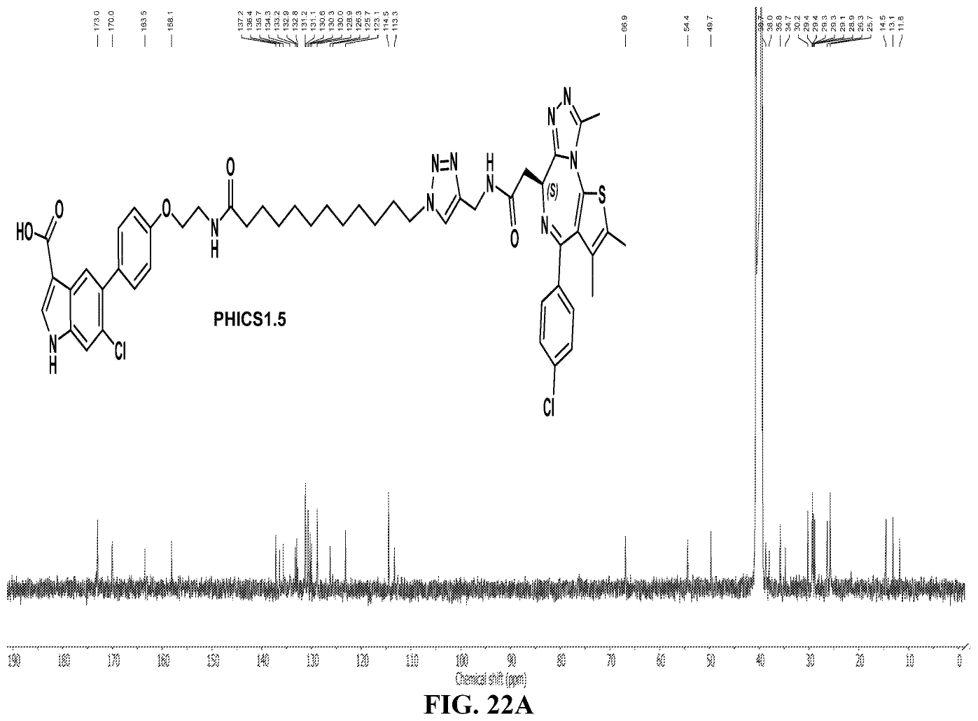

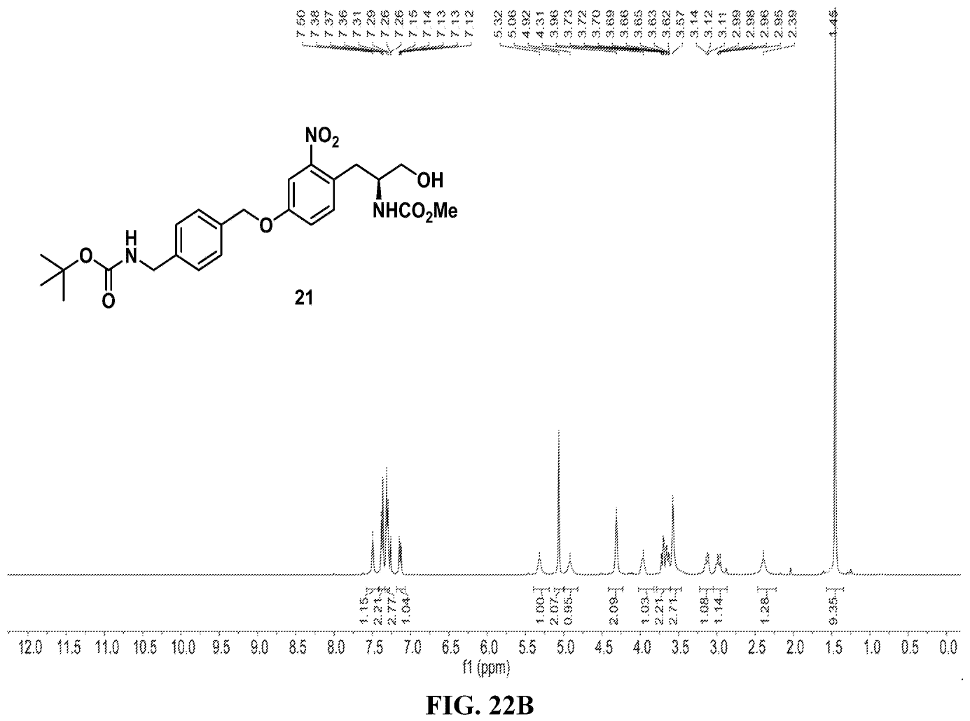

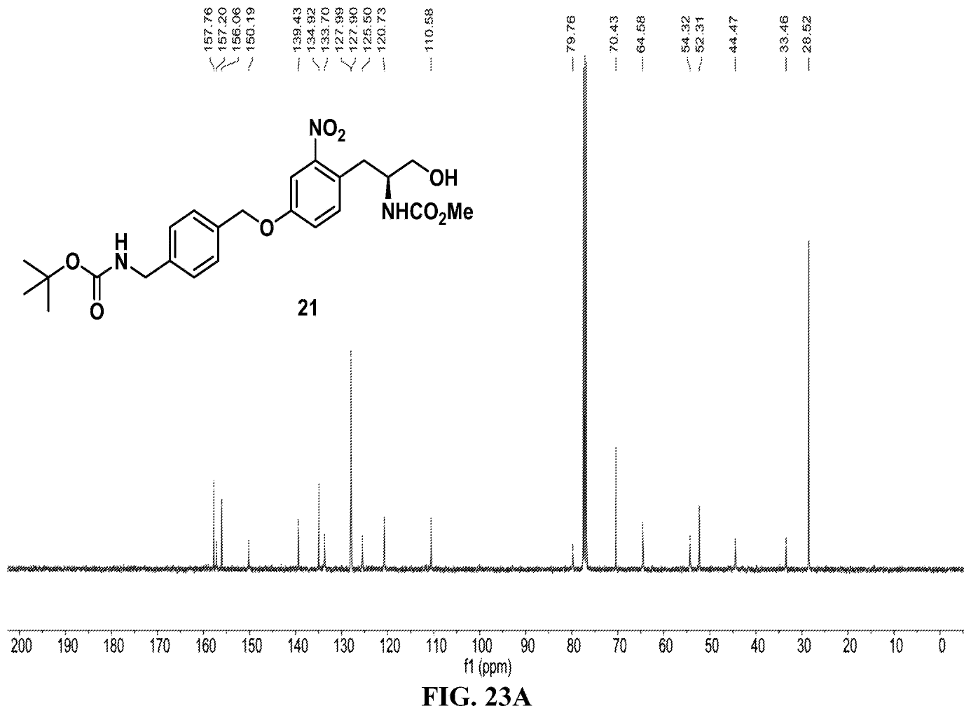

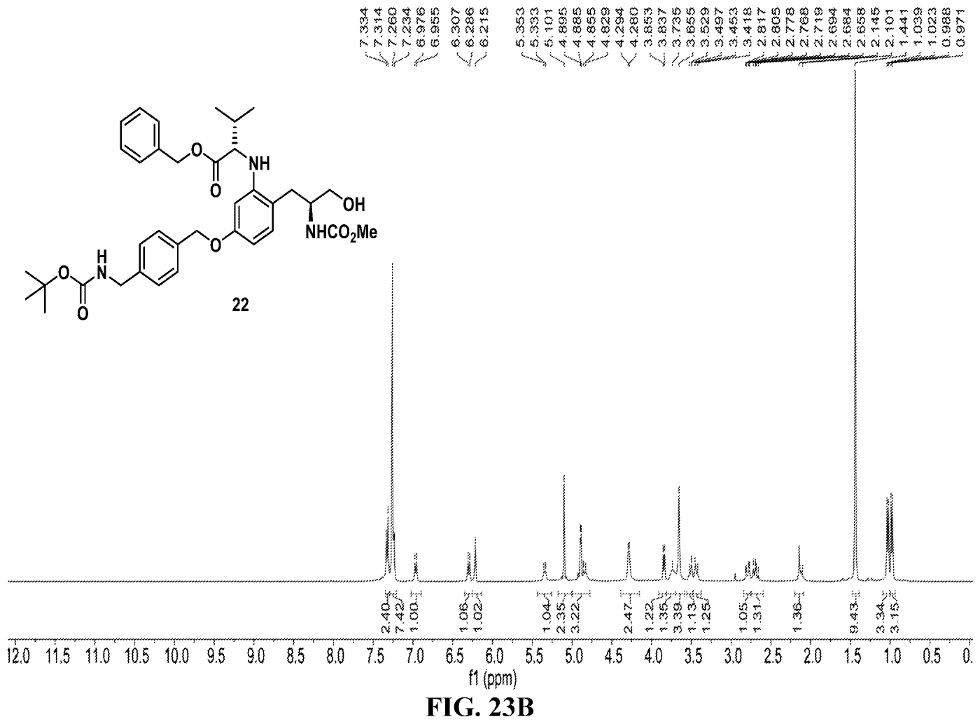

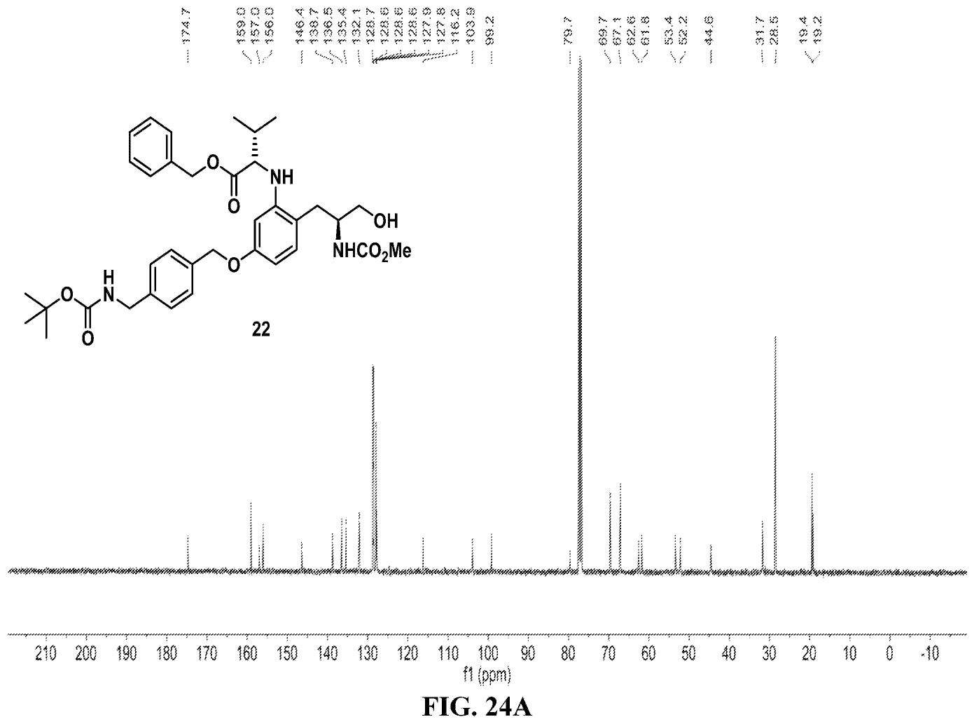

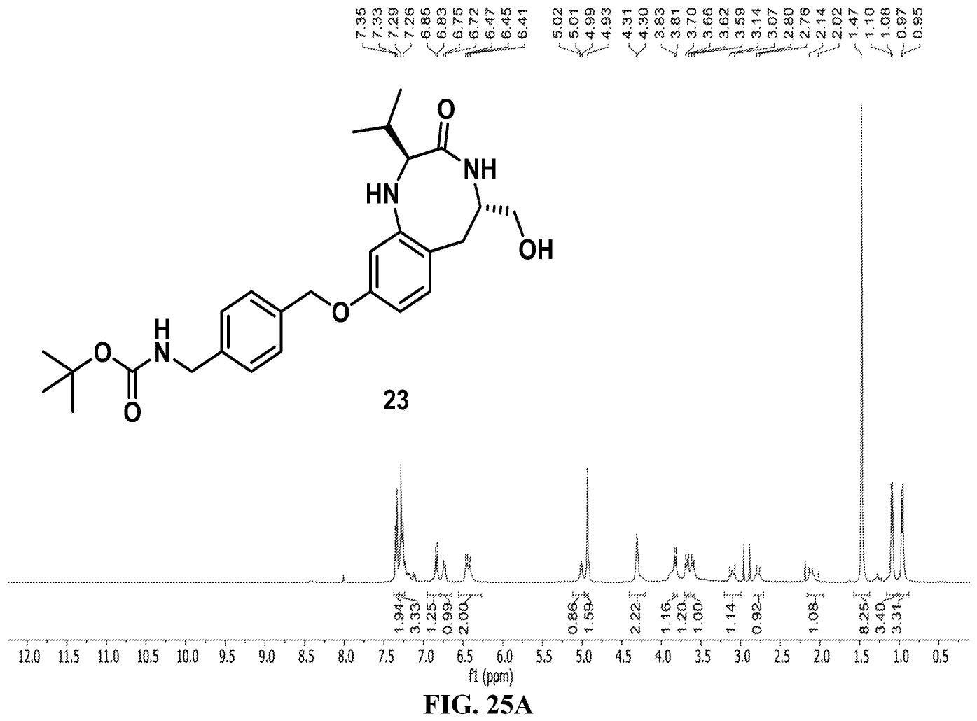

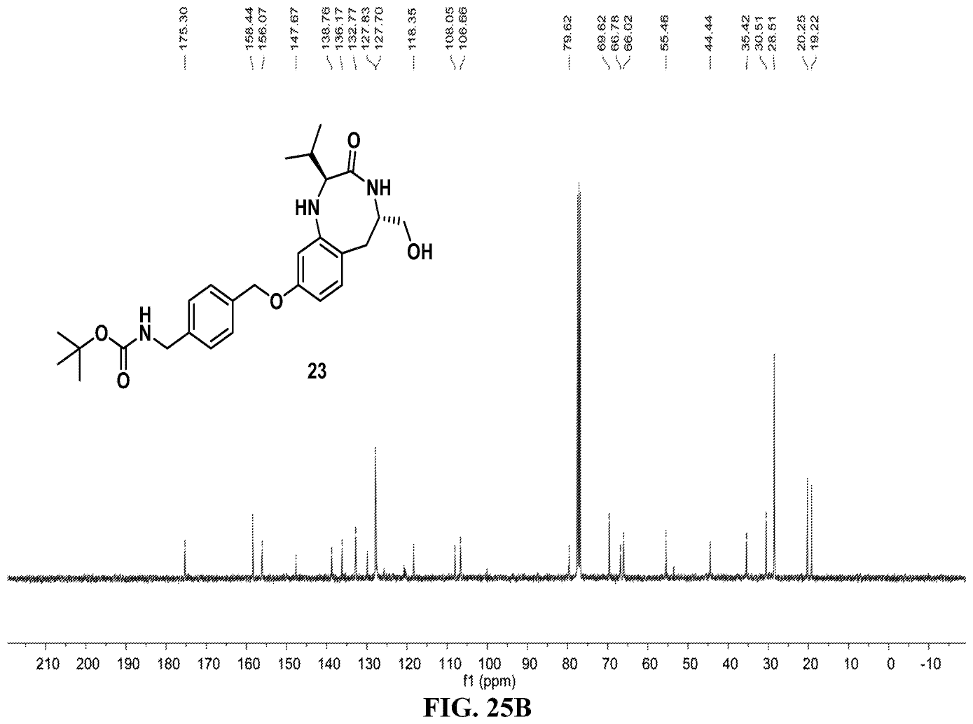

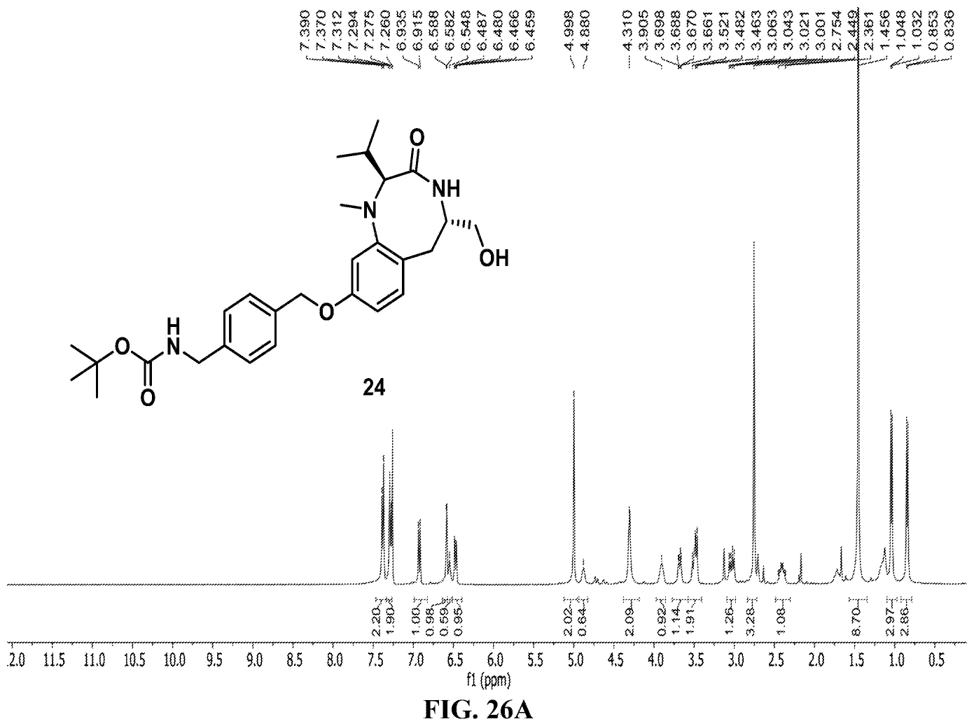

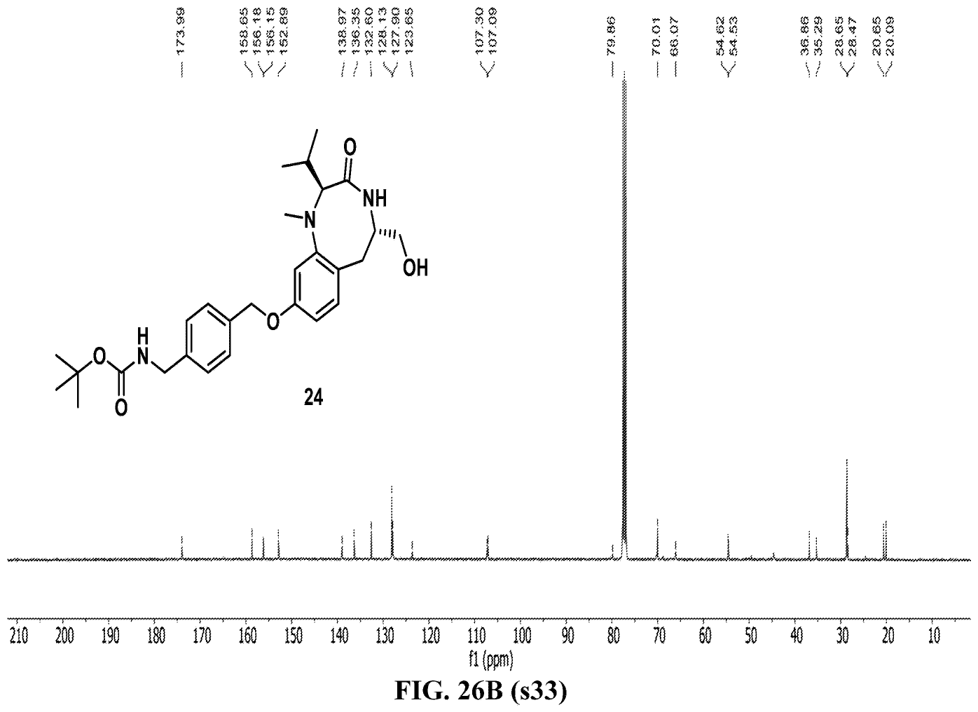

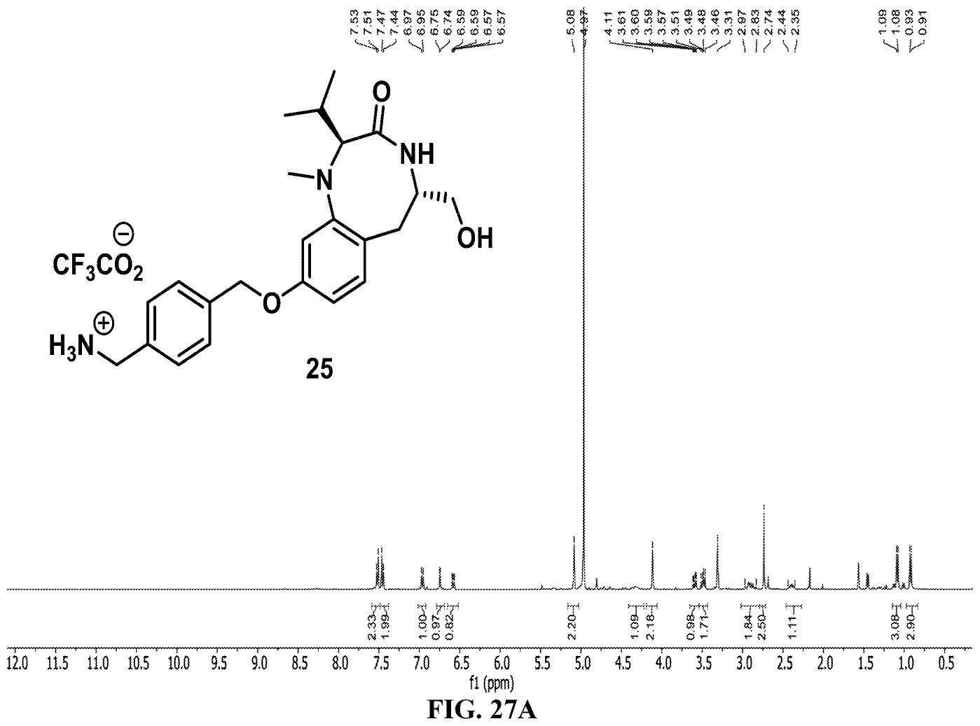

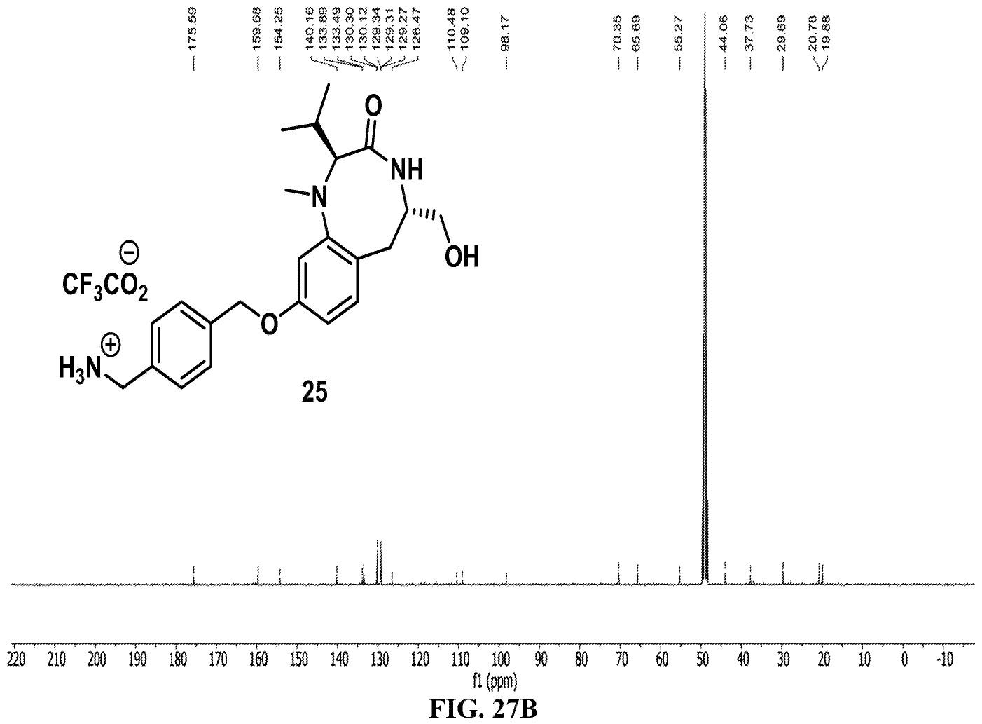

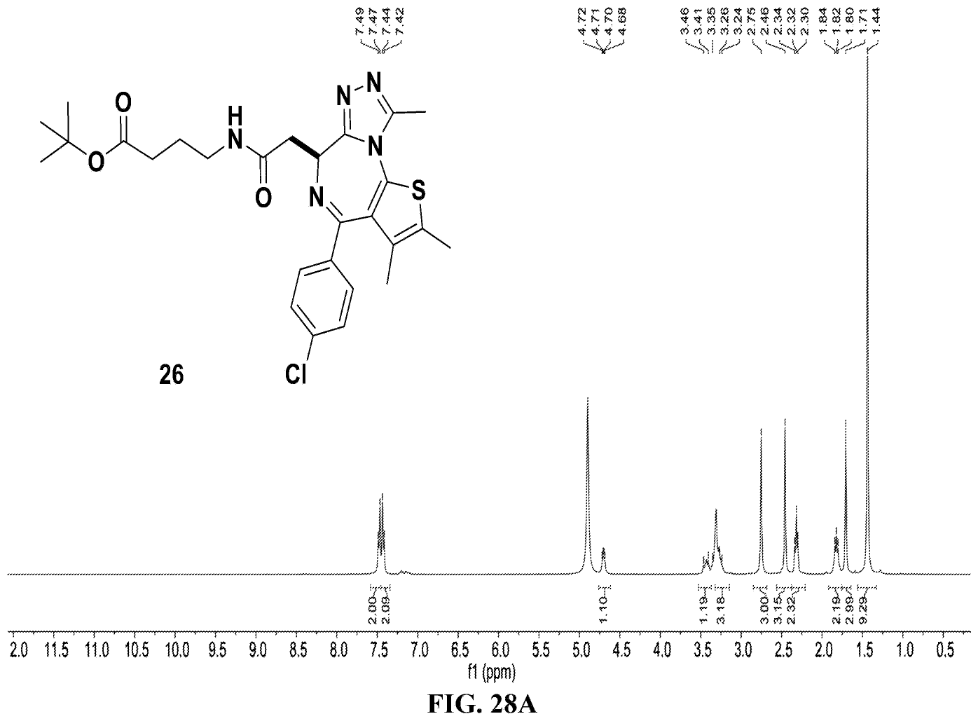

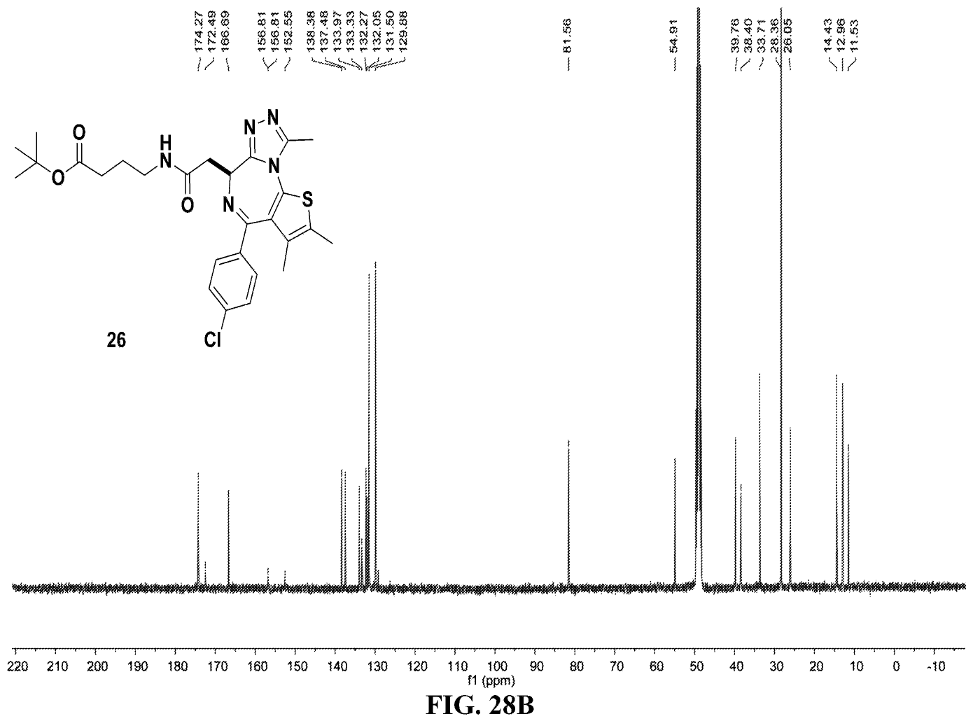

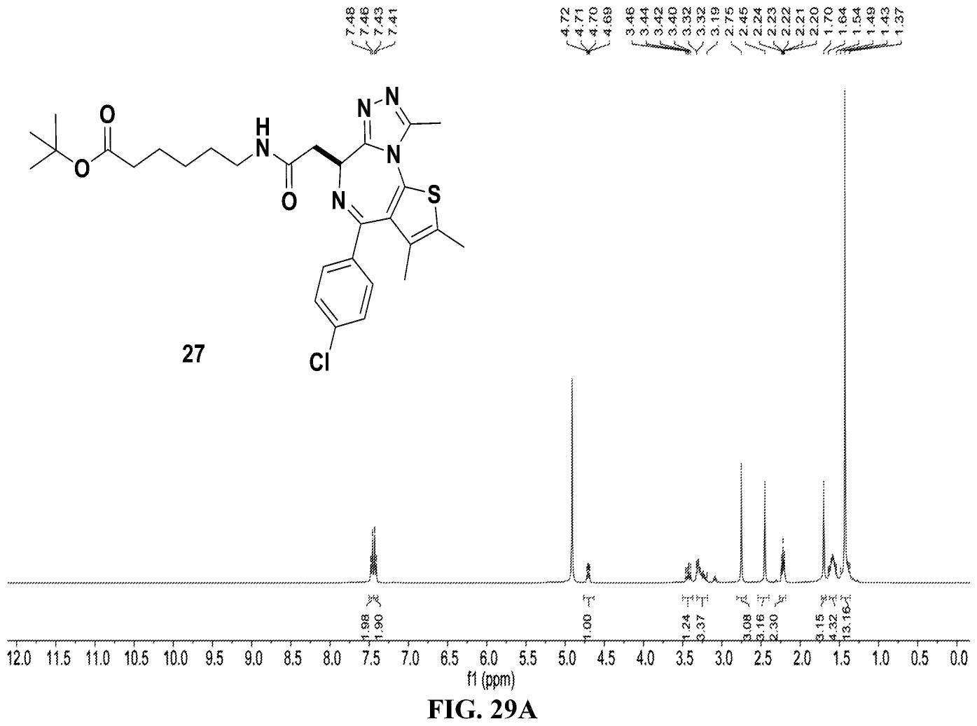

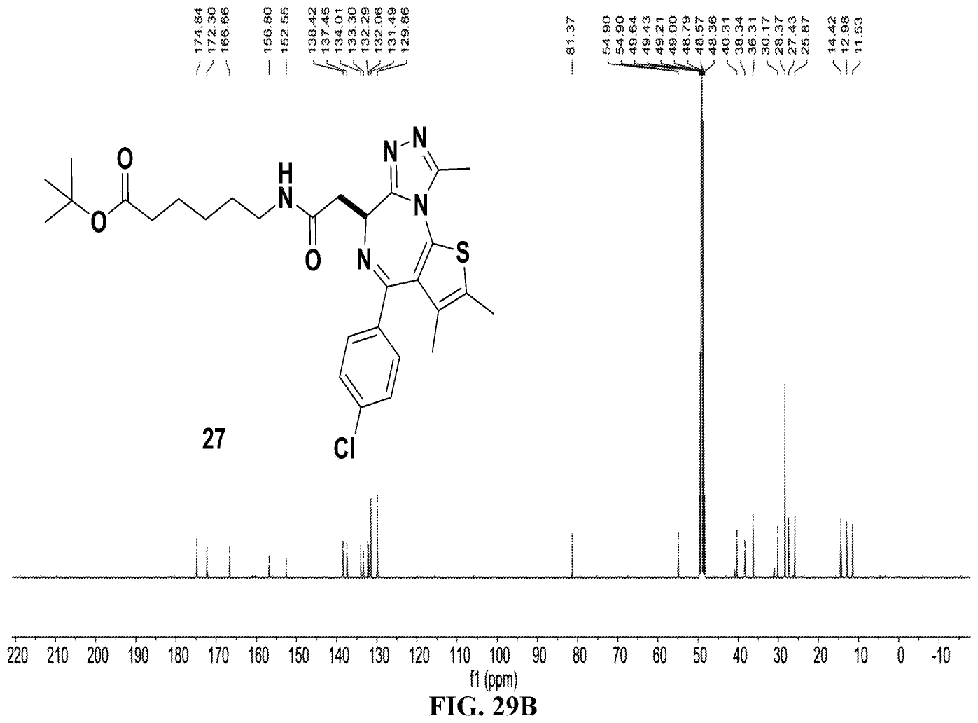

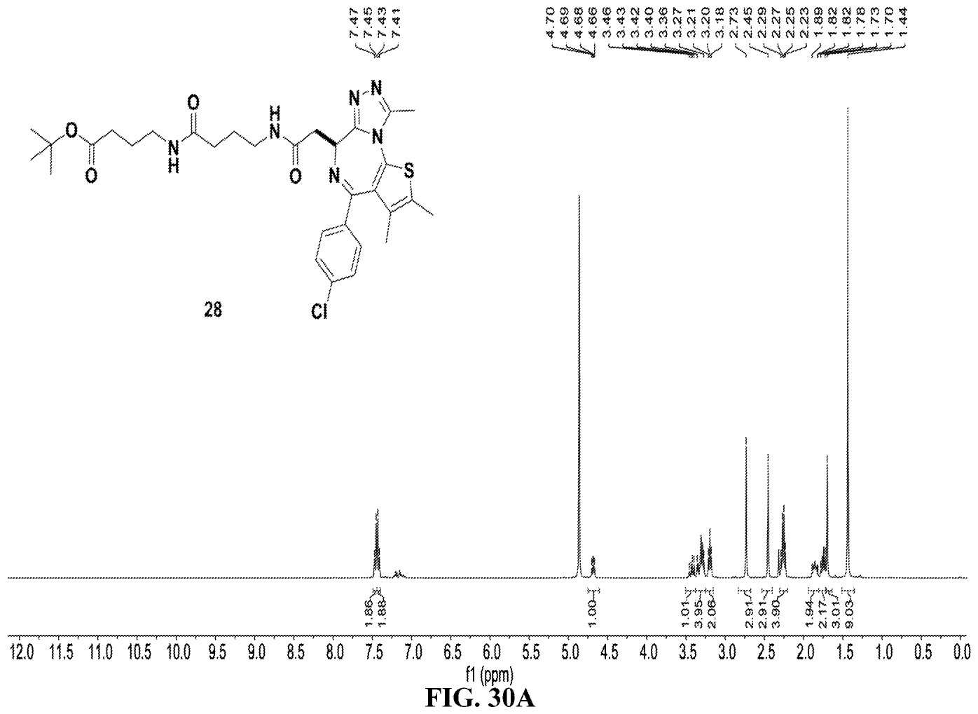

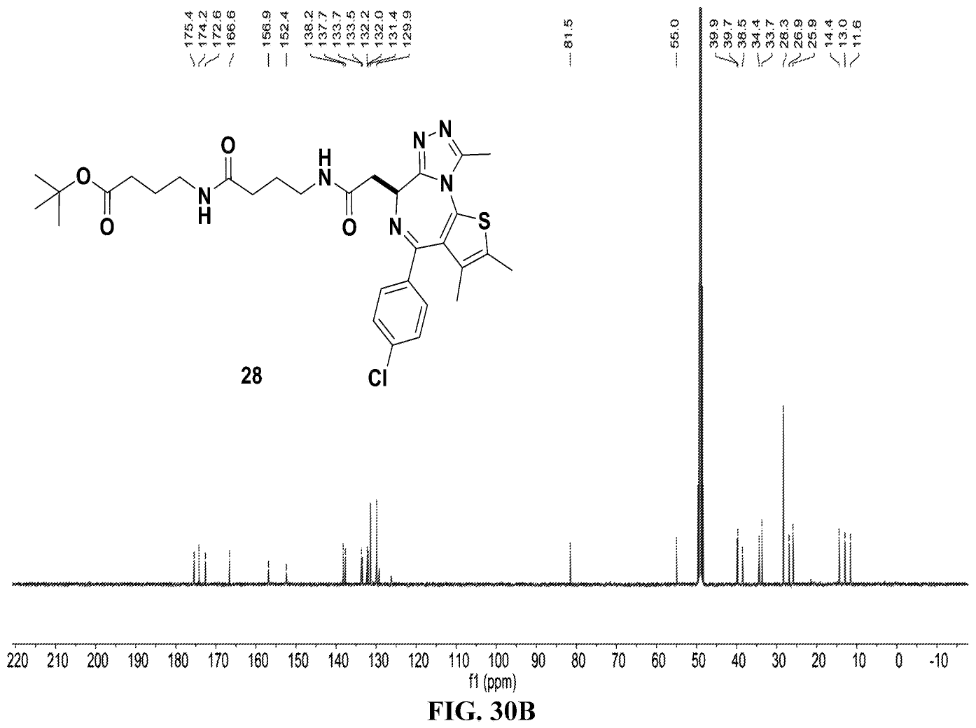

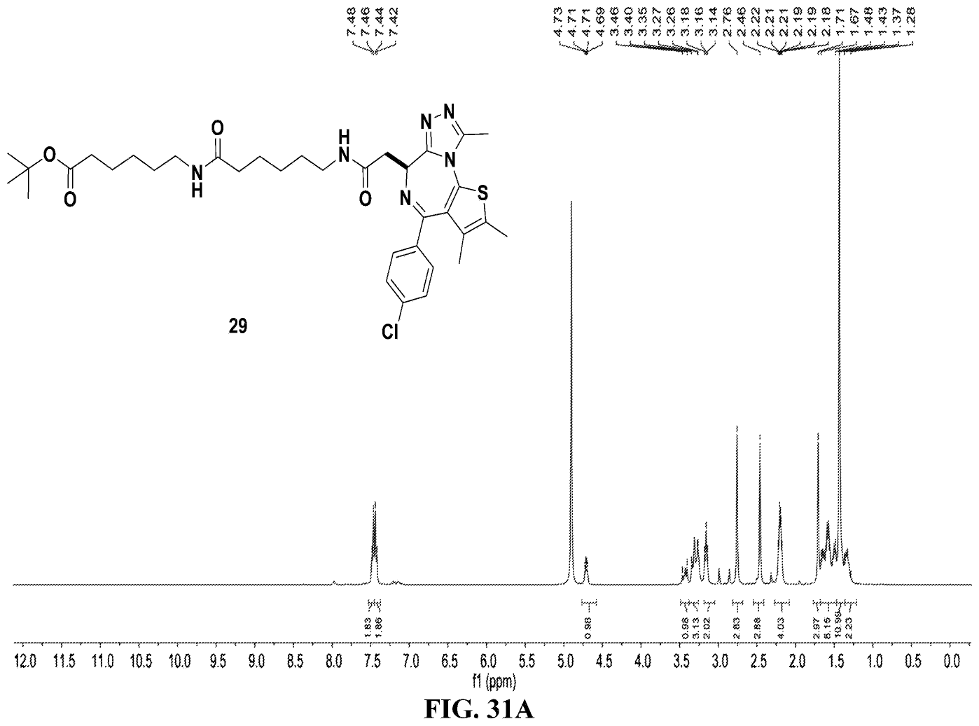

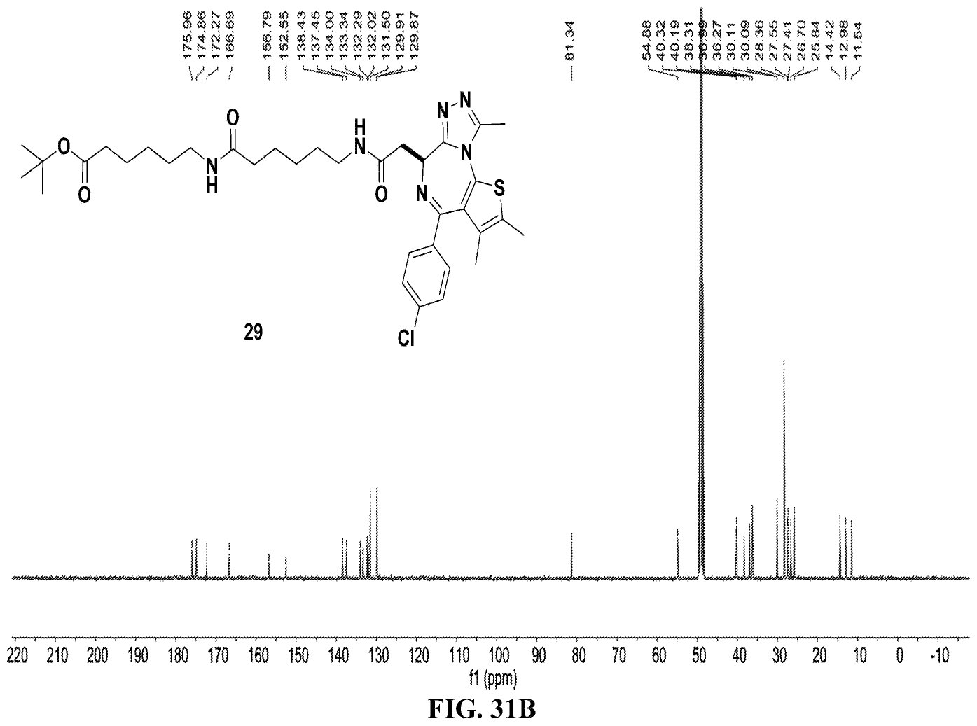

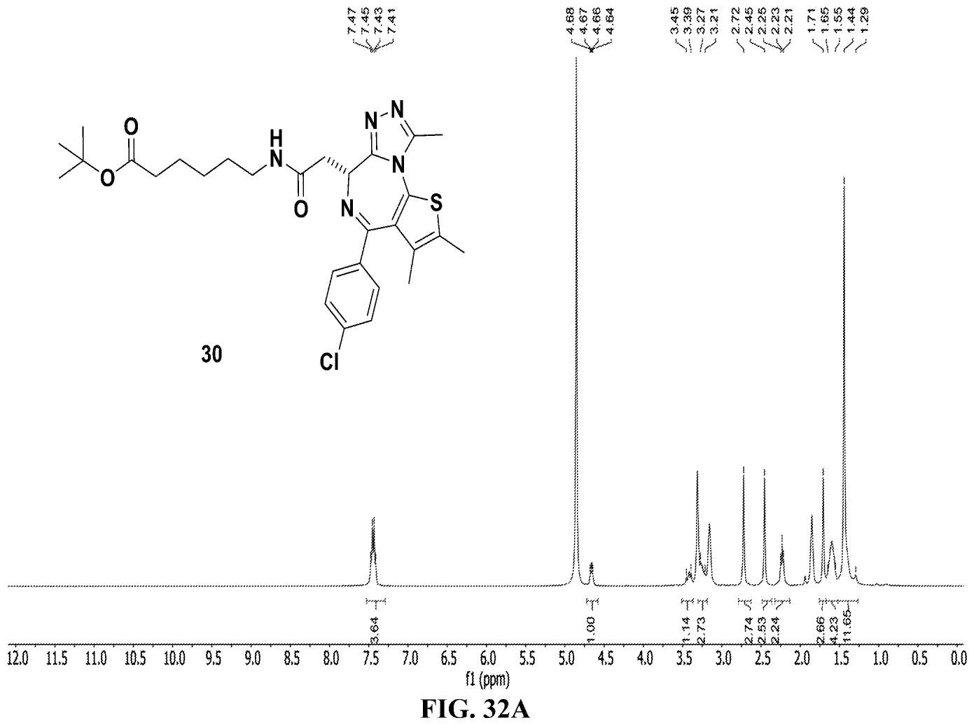

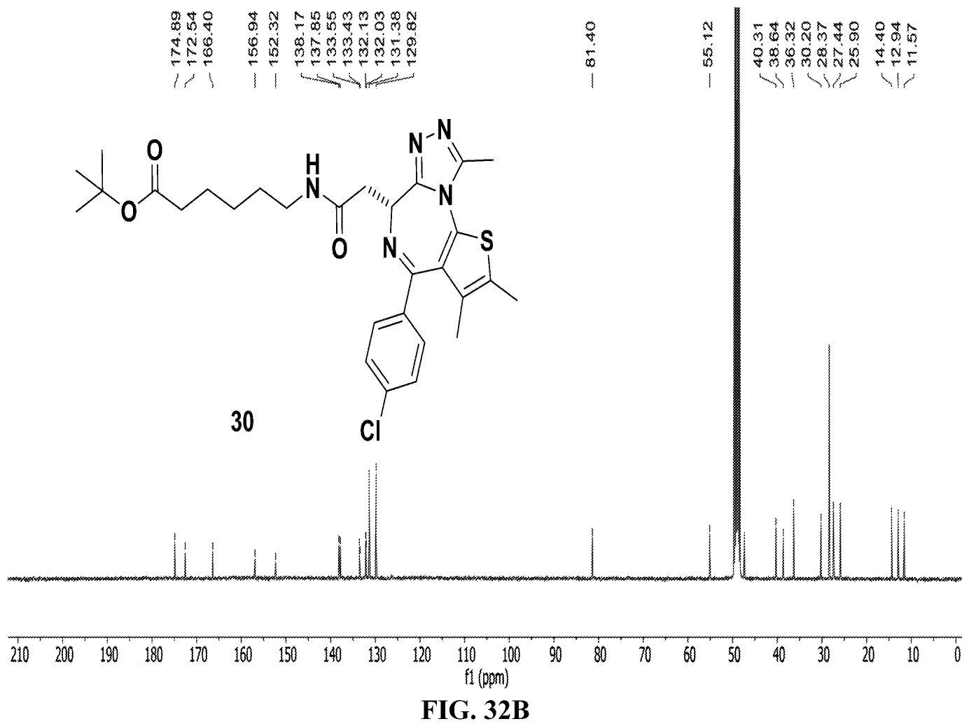

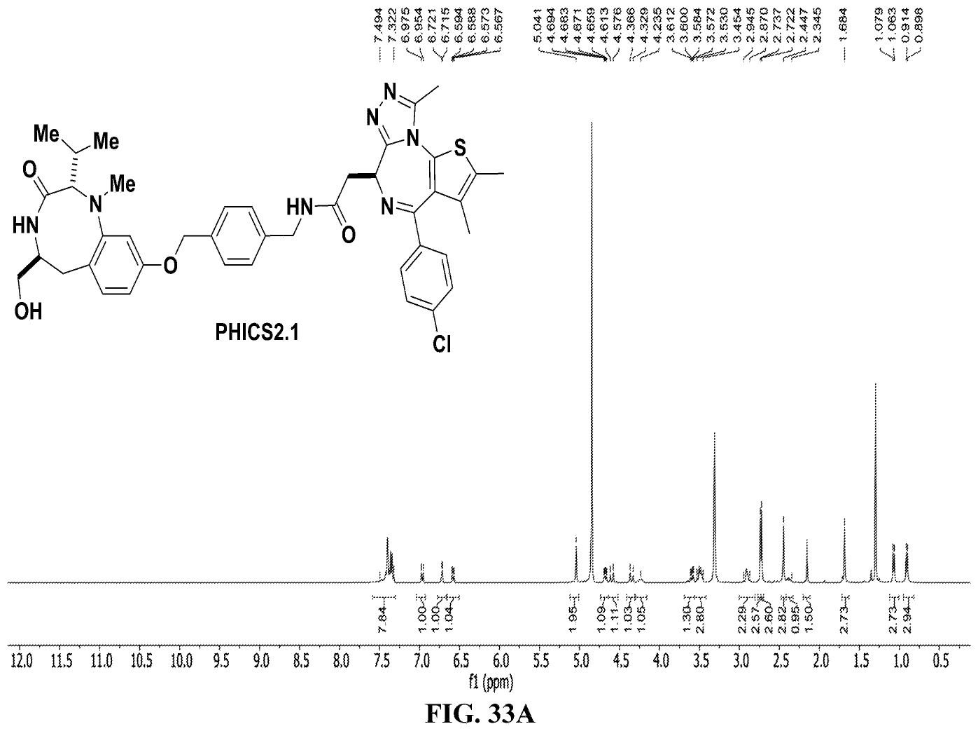

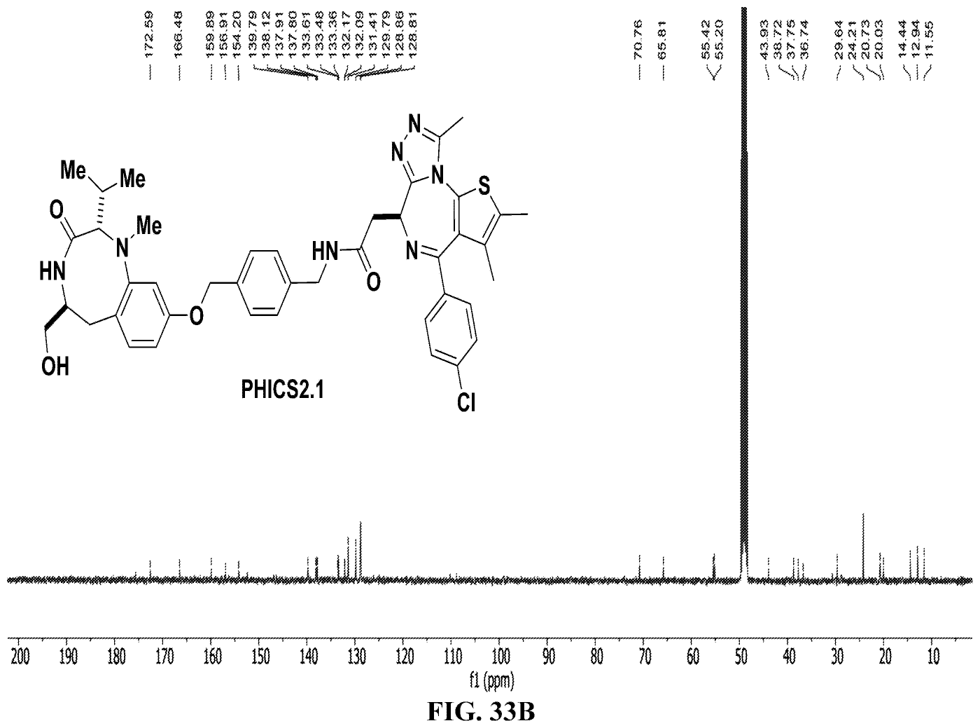

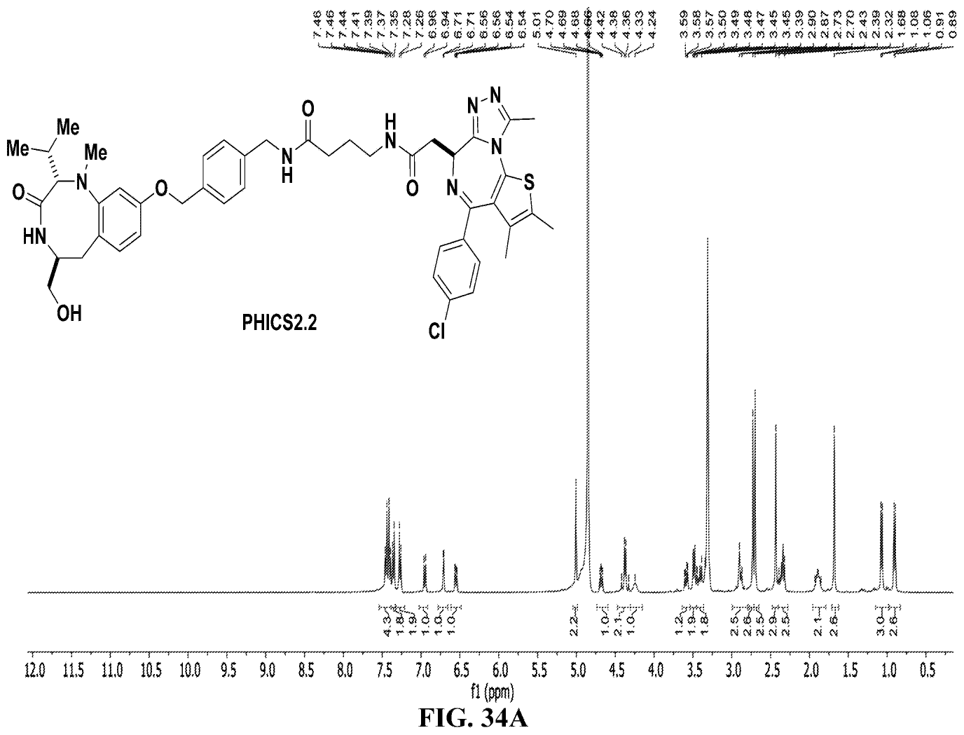

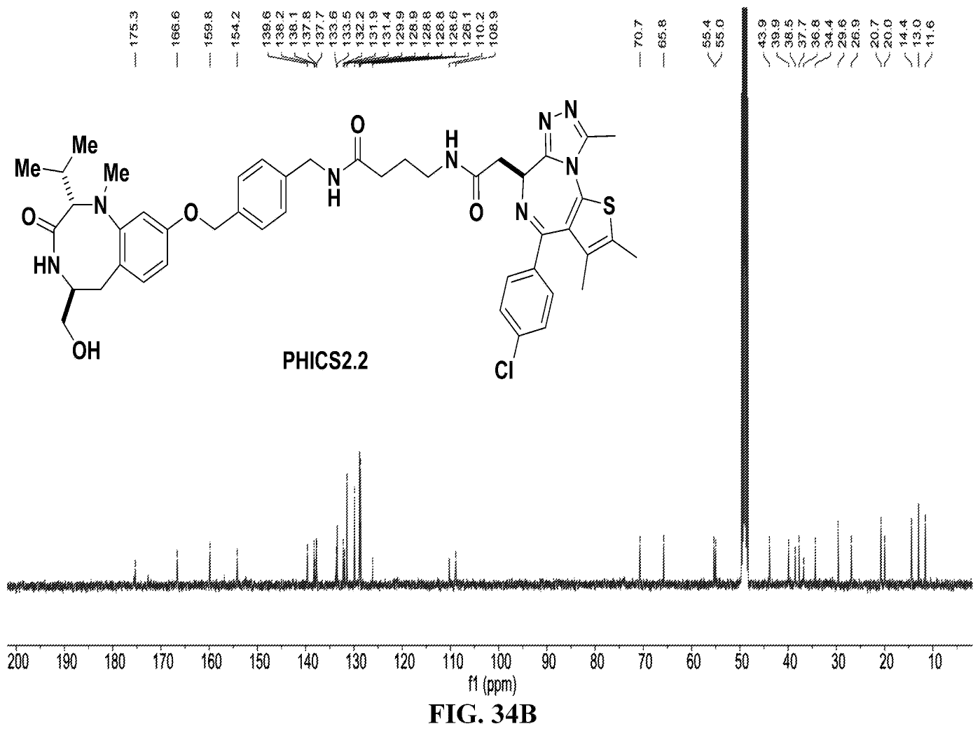

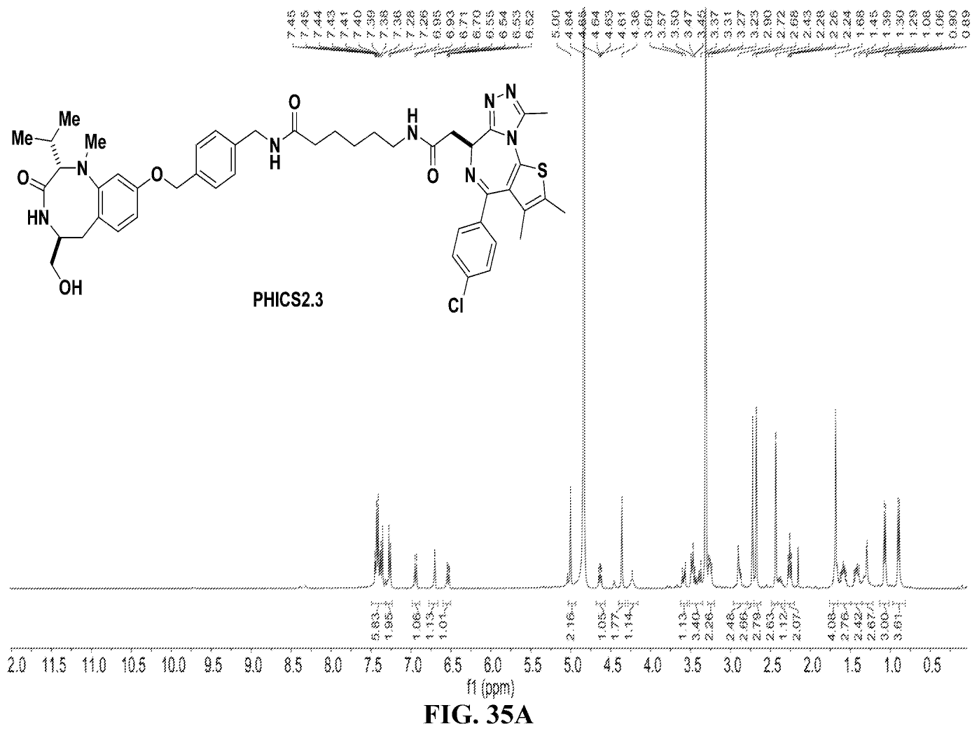

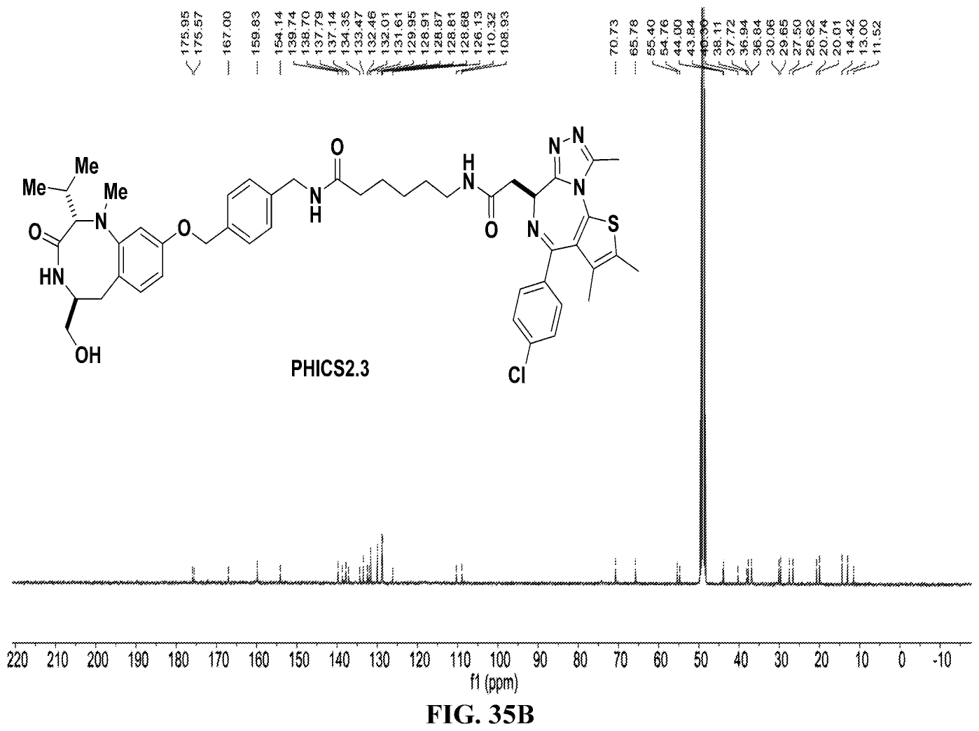

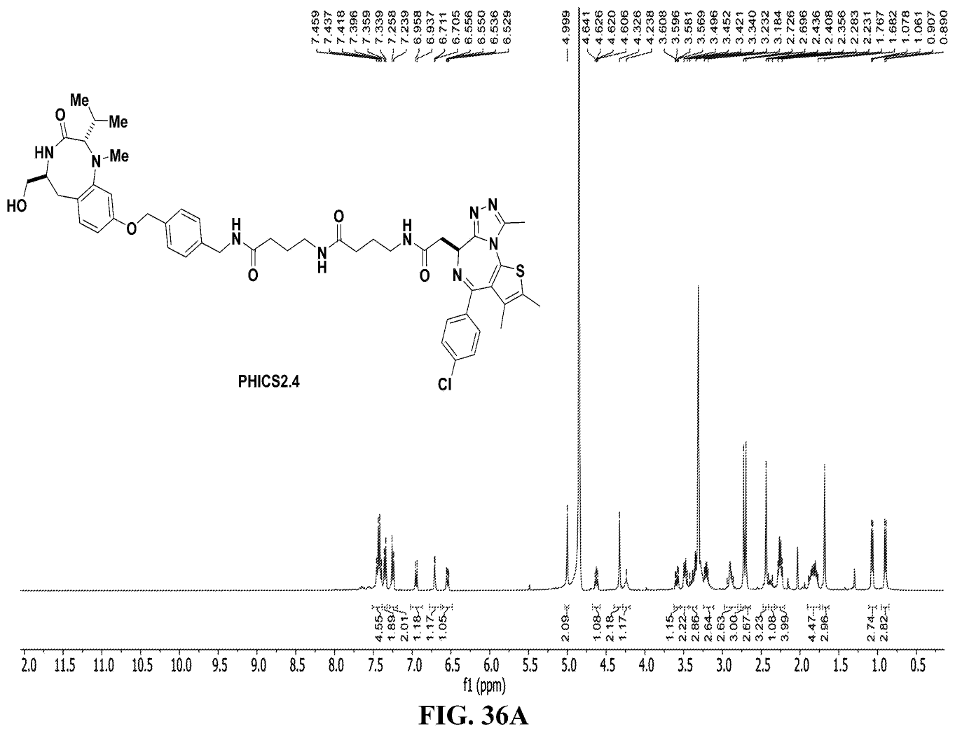

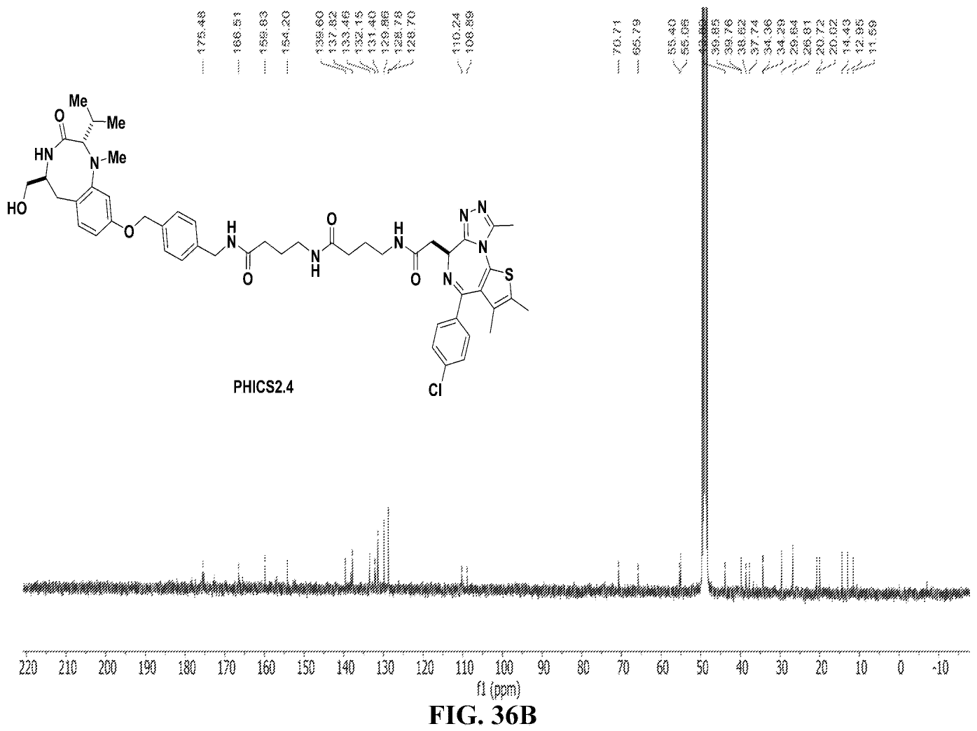

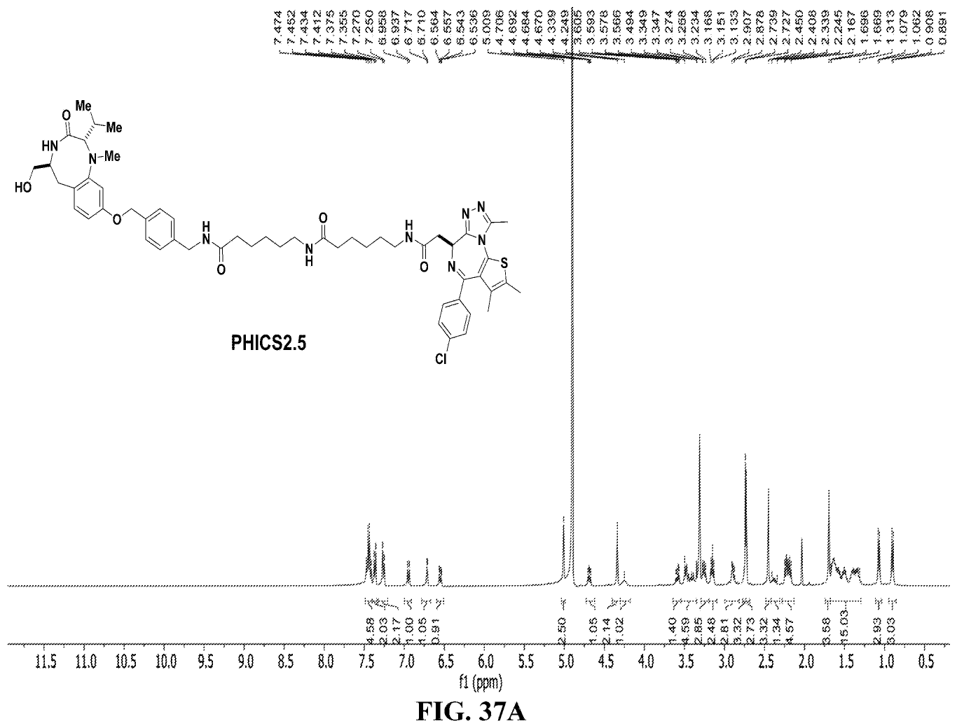

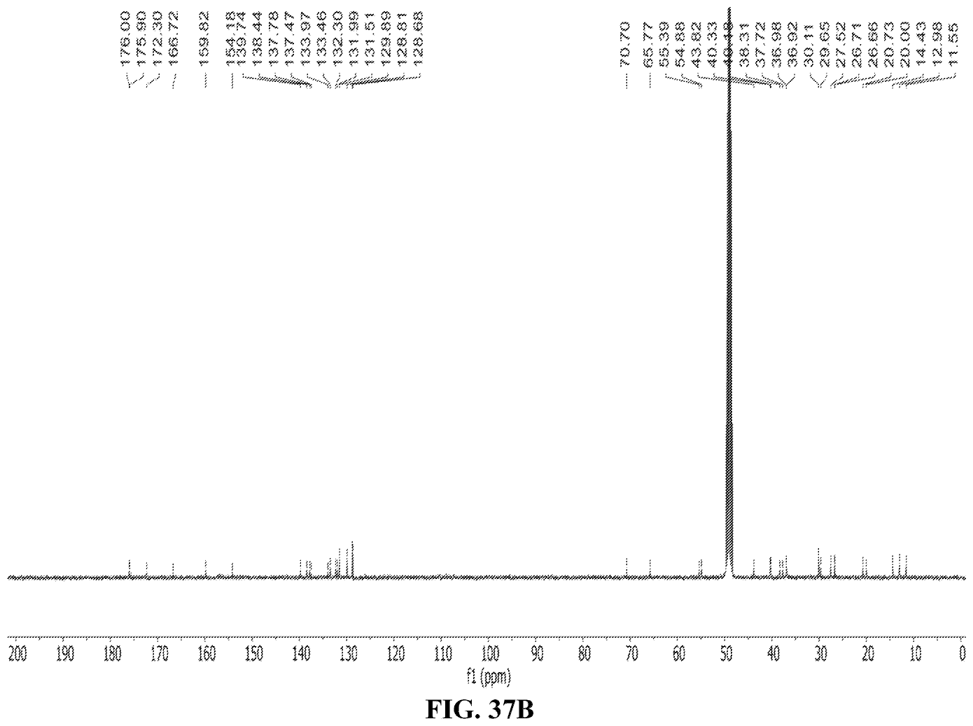

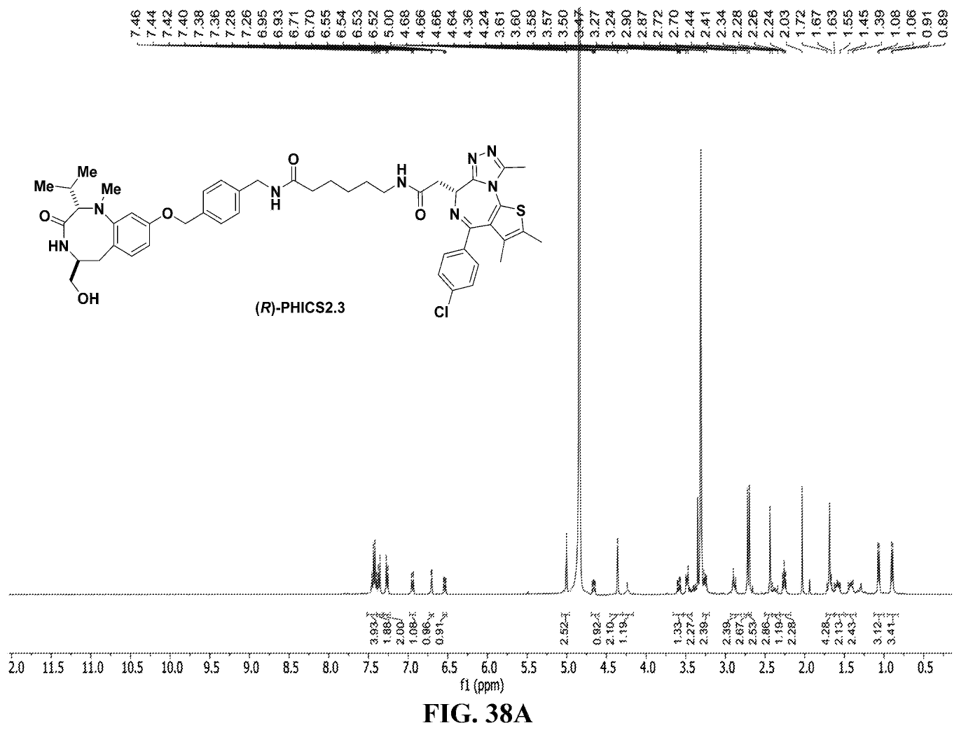

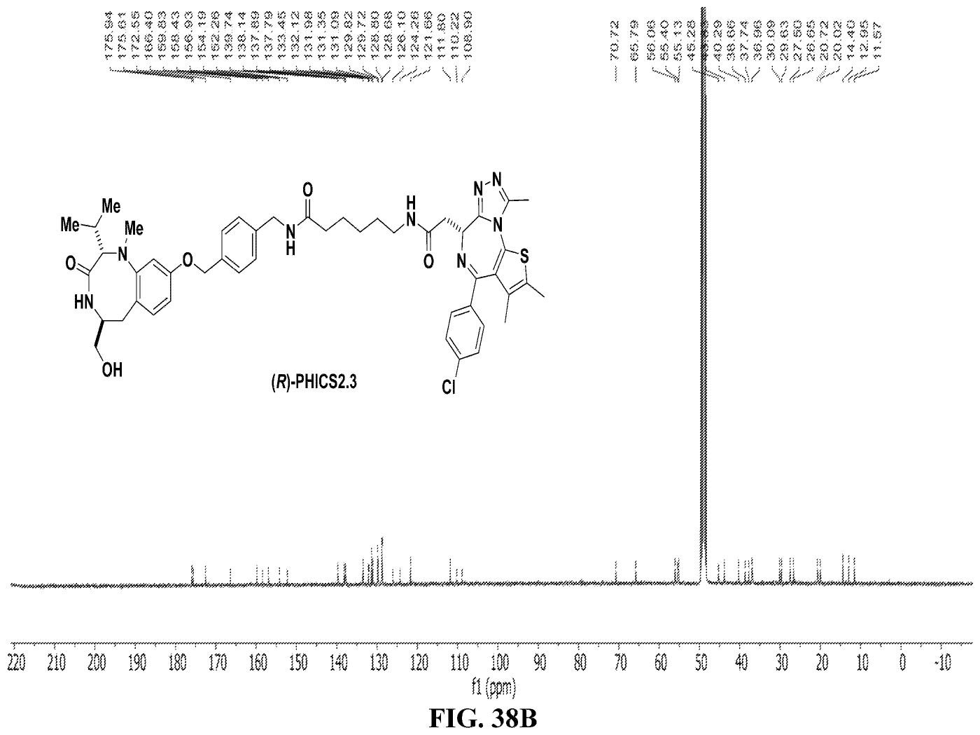

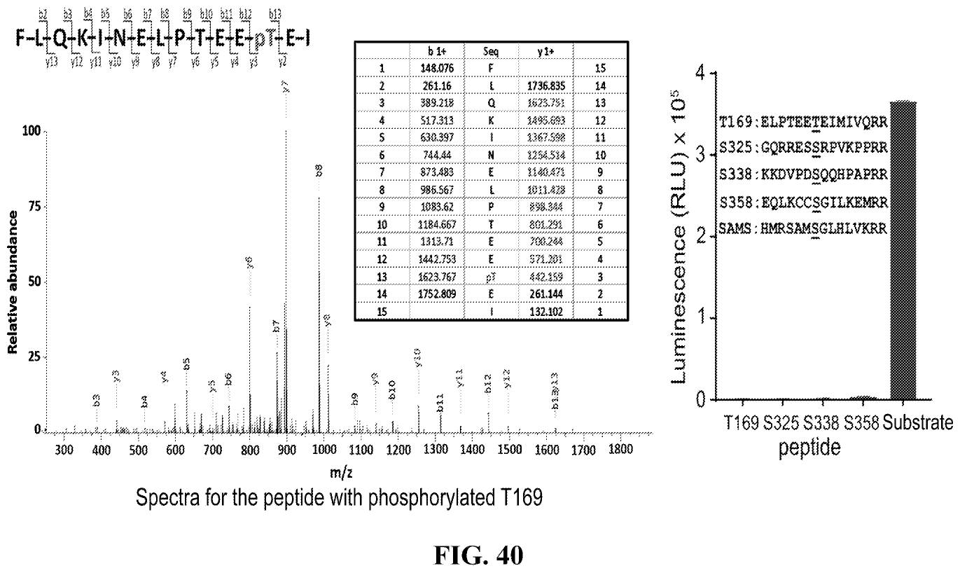

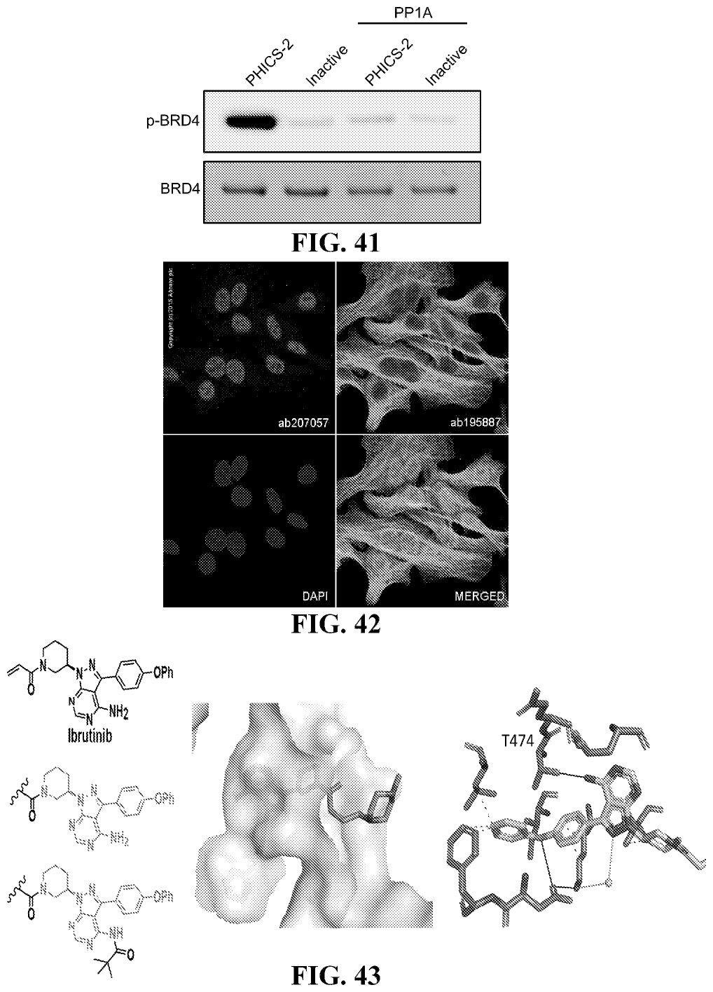

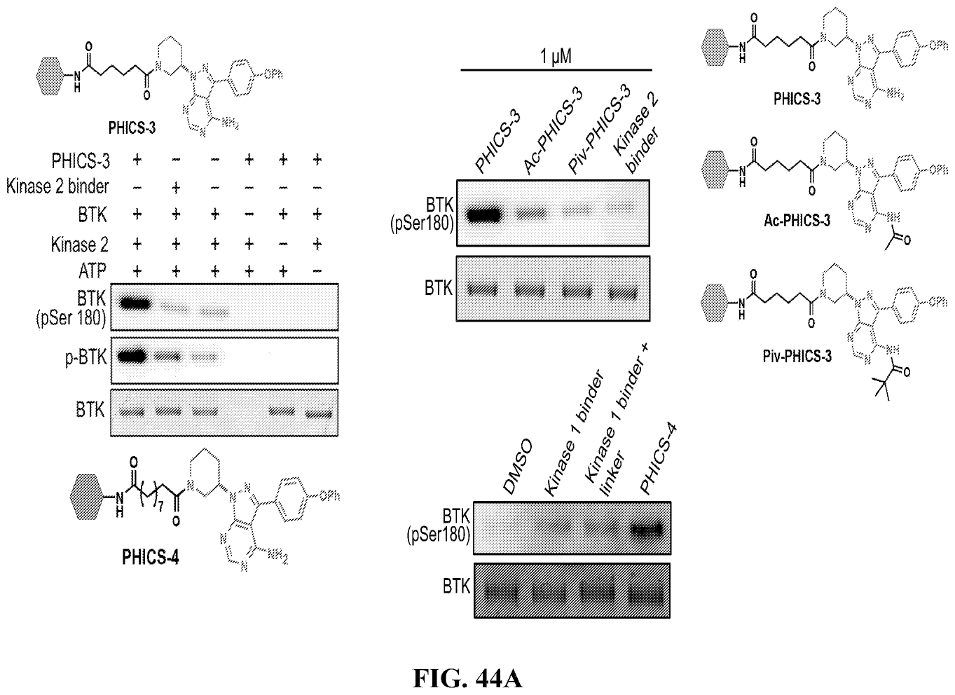

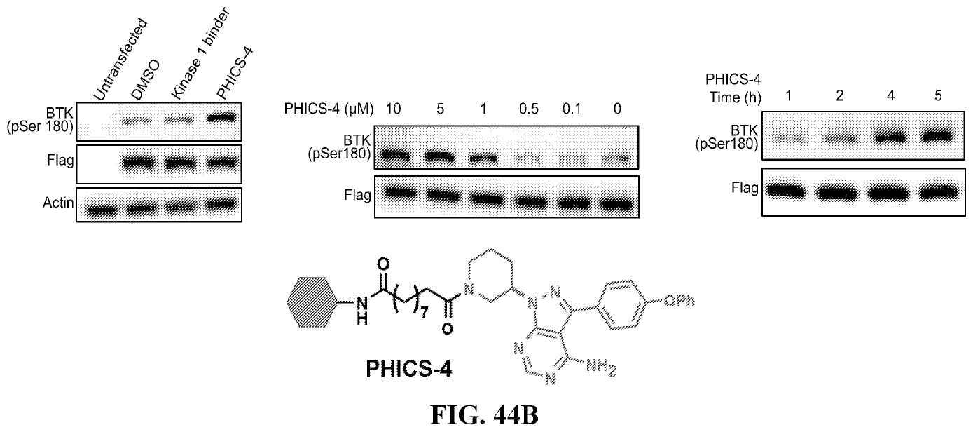

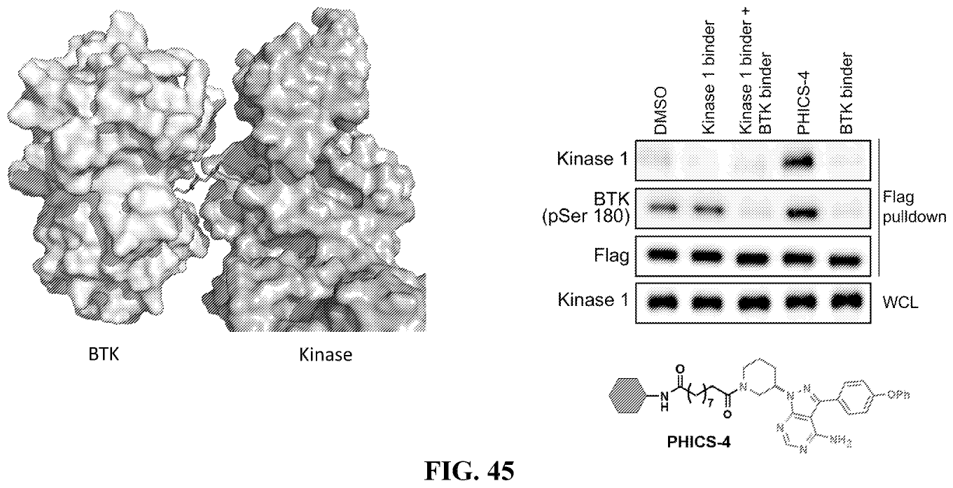

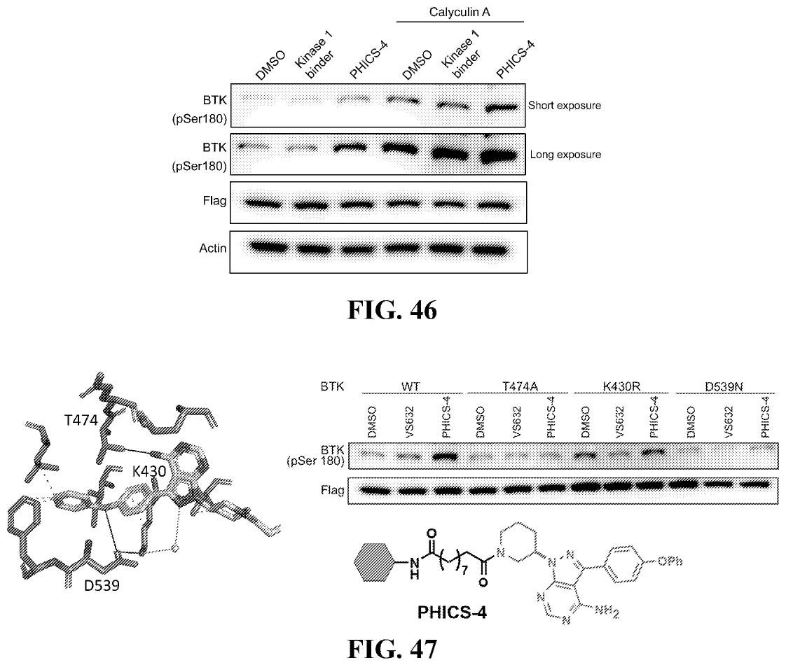

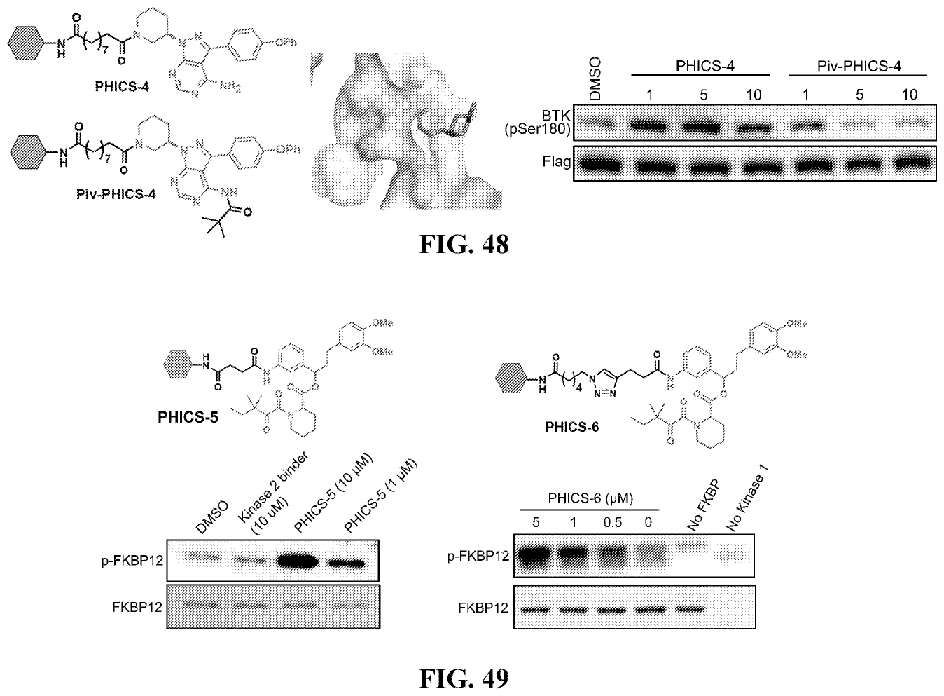

In certain example embodiments, the localizing moiety is a target polypeptide binding moiety used to bind a polypeptide substrate. The target polypeptide binding moiety may be chosen for a specific protein of interest, which may be located in different localization sites of the cell, e.g. nucleus, cytoplasm, mitochondria, cell surface. Example target polypeptide binding moieties are disclosed for example in, Sun et al., Signal Transduction and Targeted Therapy, 4:64 (2019), which provides exemplary proteins and corresponding ligands (i.e. target polypeptide binding moieties, see in particular FIGS. 5-48, which is incorporated herein by reference. The target polypeptide binding moiety may bind to proteins which undergo conformation change upon binding, for example, an androgen receptor (AR).

The target polypeptide binding moiety may bind to, for example, the Bromodomain and Extra Terminal Domain (BRD) Family proteins (e.g., BRD2, BRD3, BRD4). Bromodomains are a family of (−110 amino acid) structurally and evolutionary conserved protein interaction modules that specifically recognize acetylated lysines present in substrate proteins, notably histones. Bromodomains exist as components of large multidomain nuclear proteins that are associated with chromatin remodeling, cell signaling and transcriptional control. Examples of bromodomain-containing proteins with known functions include: (i) histone acetyltransferases (HATs), including CREBBP, GCN5, PCAF and TAFII250; (ii) methyltransferases such as ASH1L and MLL; (iii) components of chromatin-remodeling complexes such as Swi2/Snf2; and (iv) a number of transcriptional regulators (Florence et al. Front. Biosci. 2001, 6, D1008-1018, hereby incorporated by reference in its entirety).

In other example embodiments the target polypeptide binding moiety is a small molecule target polypeptide binding moiety. In one example embodiment, the target polypeptide binding moiety is a JQ1 derived moiety. For example, the target polypeptide binding moiety may be

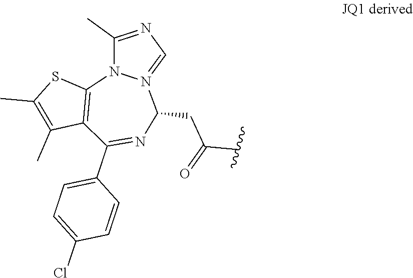

In some embodiments, the JQ1 derived moiety is (+)-JQ1 derived or (−)-JQ1-derived moiety:

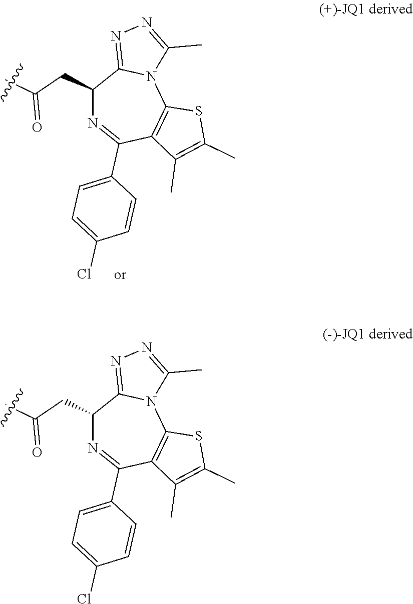

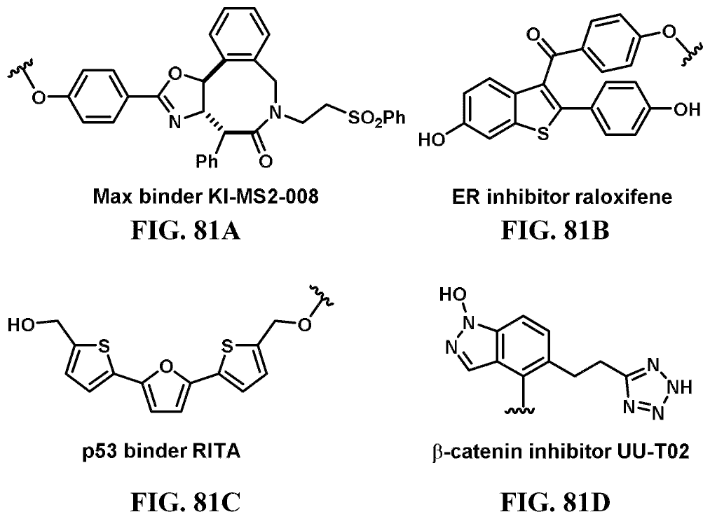

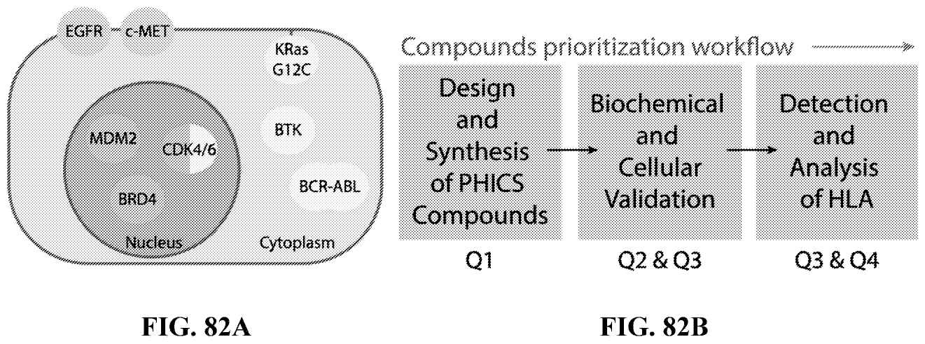

Additional target polypeptide binding molecules. For example, the target polypeptide binding molecule may be selected from a p53 binder (for example, 2,5-bis(5-hydroxymethyl-2-thienyl)furan or RITA), a Max binder KI-MS2-008 (FIG. 81A), ER inhibitor raloxifene, or a beta-catenin inhibitor (UU-T02).

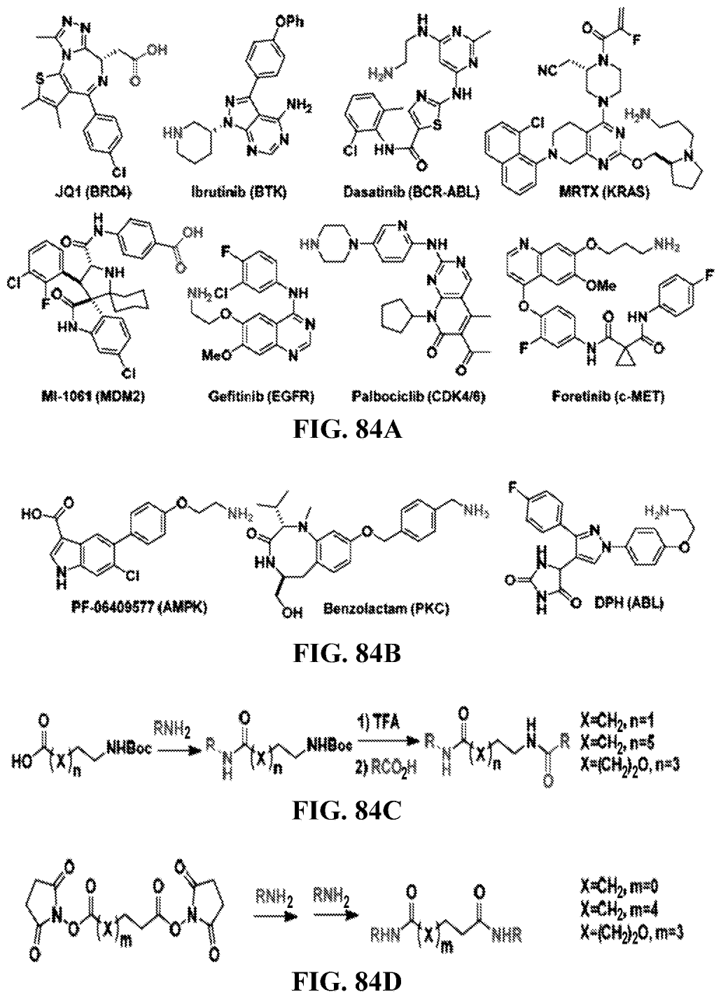

Localizing moieties may also include molecules such as Ibrutinib (BTK), Dsatinib (BCR-ABL), MRTX (KRAS), MI-1061 (MDM2), Gelfitinib (EGFR), Palbociclib (CDK4/6) and Foretinib (C-MET) as described in FIG. 84A.

In one embodiment, the localizing moiety is an antibody or binding portion thereof. The localizing moiety may be a nanobody, comprising single domain antibody fragments that comprise structural and functional properties of naturally occurring heavy chain only antibodies, see, e.g. Bannas et al, Front. Immunol., doi:10.3389/fimmu.2017.01603. The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, VHH, single-chain antibodies, e.g., scFv, and single domain antibodies.

In certain embodiments, “humanized” forms of non-human antibodies contain amino acid residues in frame regions that resemble human antibody frame regions. In certain embodiments, frame regions of camelid antibodies or heavy chain antibodies are modified. In certain embodiments, “humanized” forms of non-human antibodies are chimeric antibodies that contain minimal sequence derived from non-human immunoglobulin (e.g., camelid). For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)2 fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (VL) in the same polypeptide chain (see, e.g., EP 404,097; WO 93/11161; Hollinger et al., 90 PNAS 6444 (1993)); (xi) “linear antibodies” comprising a pair of tandem Fd segments (VH-Ch1-VH-Ch1) which, together with complementary light chain polypeptides, form a pair of antigen binding regions (Zapata et al., Protein Eng. 8(10):1057-62 (1995); and U.S. Pat. No. 5,641,870).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 μM or less, 100 μM or less, 50 μM or less or 25 μM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

### Localizing Moieties for Binding Polynucleotide Substrates

Polynucleotide binding proteins have been identified as factors that exacerbate inflammation. Polynucleotide binding proteins can be identified from nucleotide-binding folds in the proteins, such as the Rossmann-type (see, e.g. Kleiger et al., J. of Mol. Biol. 323: 69-76) and the P-loop containing nucleotide hydrolase folds (see, e.g., Saraste et al., Trends in Bio Sci, 15: 430-434). Chauhan et al. has developed methods for the identification of ATP and GTP binding residues and Ansari et al. has designed a method specifically for NAD. Parca et al. (2012), identified nucleotide-binding sites in protein structures, and include nucleotides bound by the protein, protein name and name of organism in Table S1 of DOI: 10.1371/journal.pone.0050240, incorporated herein by reference. Accordingly, nucleotide binding localizing moieties are known in the art and can be identified by one of skill in the art for use as a localizing moiety in the present compositions.

### Localizing Moieties for Binding Oligosaccharide Substrates

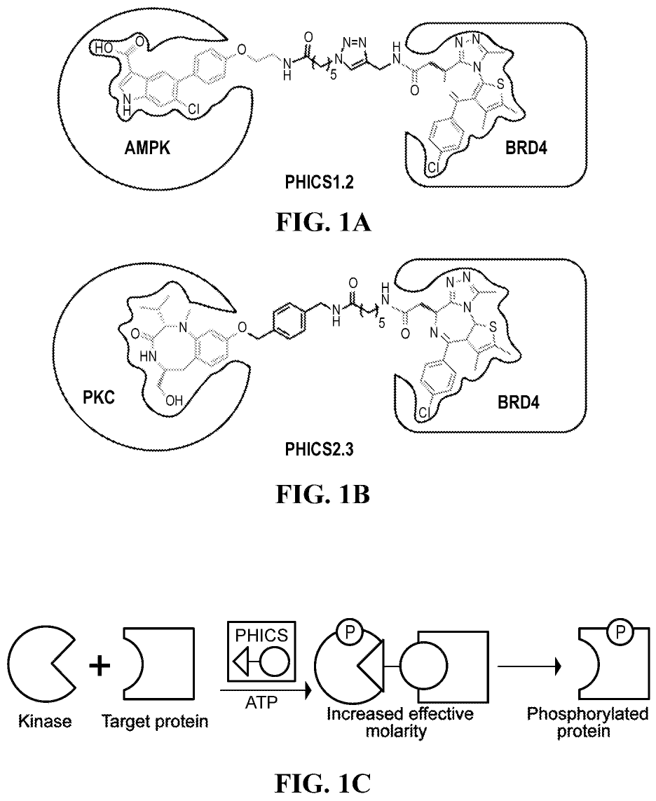

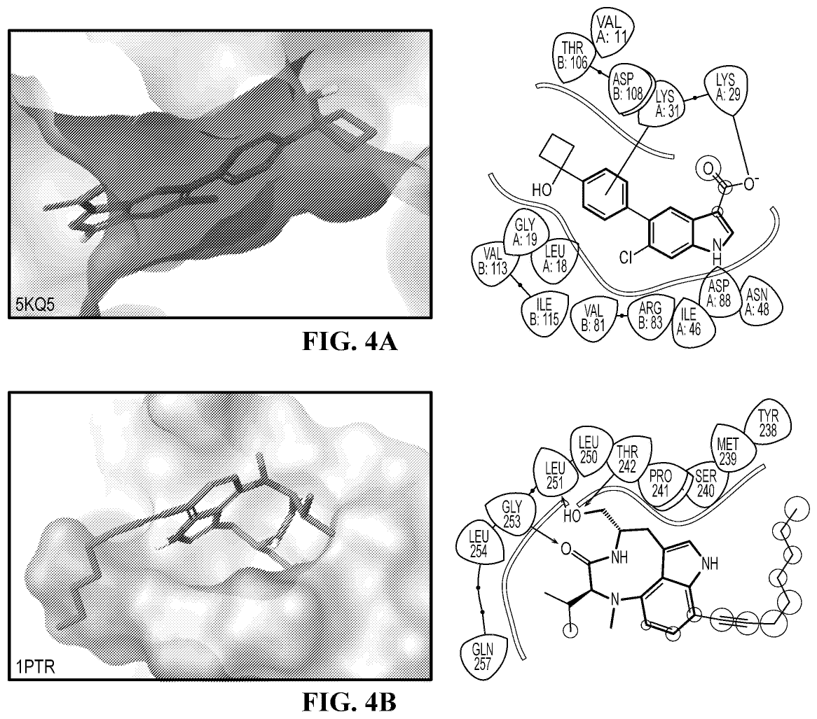

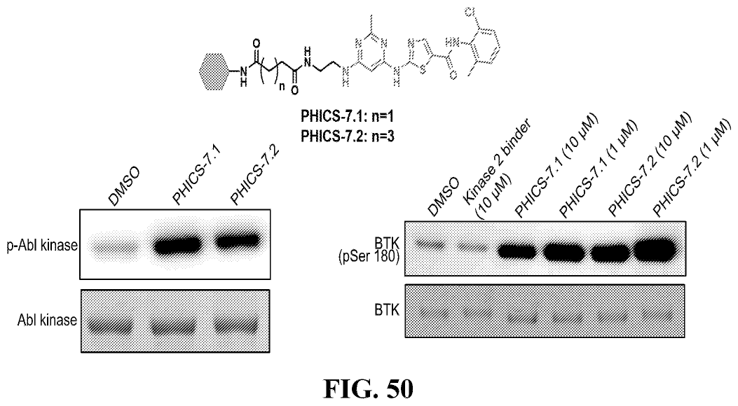

Oligosaccharide binding moieties include carbohydrate binding proteins, are important targets when considering antiviral and anticancer drugs. The localizing moiety can be, for example, a lectin, facilitating interaction sites for carbohydrates. Exemplary molecules include small molecule boronolectins, nucleic acid-based boronolectins, and peptidoboronolectins. See, e.g. Jin et al., Med. Res Rev. 2010 March; 30(2): 171-257; doi: 10.1002/med.20155, incorporated herein by reference, specifically FIGS. 1-50 for binding molecules and the complexes formed. Publicly available computations methods are available using developed bioinformatics to select small molecules capable of binding carbohydrates, see, e.g., Zhao et al., Current Protocols in Protein Science 94: 1 10.1002/cpps.75; Shionyu-Mitsuyama C, Shirai T, Ishida H, Yamane T (2003) Protein Eng 16: 467-478; and Kulharia M, Bridgett S J, Goody R S, Jackson R M (2009) InCa-SiteFinder: a method for structure-based prediction of inositol and carbohydrate binding sites on proteins. J Mol Graph Model 28: 297-303.

Analysis of binding site residues along with stabilizing residues in protein-carbohydrate complexes can allow for identification of folding and binding of the complexes to understand interactions in addition to non-covalent interactions of hydrogen bonding and non-polar interactions. See, e.g., Shanmugam et al., doi.:10.2174/0929866525666180221122529. Utilizing publicly available tools, carbohydrate binding moieties, including binding sites and predicted folding can be used for the design of multifunctional molecules comprising such a carbohydrate binding localizing moiety.

### Localizing Moieties for Binding Lipid Substrates

Lipid binding moieties can be utilized as localizing moieties in the multifunctional molecules disclosed herein. As regulators of cellular stabilization and signaling, modifications in their composition, distribution or trafficking would be useful in treatment, regulation and/or modification of pathways, processes and conditions. Lipids include charged lipids, e.g. phosphatidylserine (PS), phosphatidic acid (PA), phosphatidylinositol (PI), and the PI-phosphate, -bisphosphate, and -trisphosphate (PIPs—a family of seven anionic charged lipids), and ganglioside (GM). Zwitterionic lipids, e.g., phosphatidylcholine (PC), phosphatidylethanolamine (PE), and sphingomyelin (SM) lipids, Ceramides (CER), diacylglycerol (DAG), and lysophosphatidylcholine (LPC) lipids, sphingolipids, glycerophospholipids, cholesterol, phosphatidylglycerols.

Lipid binding moieties such as proteins can either bind lipids specifically, where a clear binding site for a given lipid can be identified, or nonspecifically, where lipids act as a medium, and physical properties like thickness, fluidity, or curvature regulate the protein function. Phosphoinositide binding domains such as FYVE or PX, or the FRRG motif in the β-propeller of PROPPINs are more common domains that can be used to identify lipid binding proteins. The FYVE domain, named after the first four proteins to contain the motif (Fab1, YOTB, Vac1 EEA1) contains several conserved regions, which can also be utilized to identify related domains. See, e.g., A. H. Lystad, A. Simonsen Phosphoinositide-binding proteins in autophagy, FEBS Lett., 590 (2016), pp. 2454-2468, 10.1002/1873-3468.12286. Additional FYVE domain-containing proteins include SARA, FRABIN, DFCP1 FGD1, ANKFY1, EEA1 FGD1, FGD2, FGD3, FGD4, FGD5, FGD6, FYCO1, HGS MTMR3, MTMR4, PIKFYVE, PLEKHF1, PLEKHF2, RUFY1, RUFY2, WDF3, WDFY1, WDFY2, WDFY3, ZFYVE1, ZFYVE16, ZFYVE19, ZFYVE20, ZFYVE21, ZFYVE26, ZFYVE27, ZFYVE28, ZFYVE9.

Eukaryotic cells can degrade intracellular components through a lysosomal degradation pathway called macroautophagy, with pathway malfunction linked to several diseases. Dikic et al., Mechanism and medical implications of mammalian autophagy. Nat. Rev. Mol. Cell Biol., 19 (2018), pp. 349-364, doi: 10.1038/s41580-018-0003-4. Accordingly, autophagy related (ATG) proteins may be utilized as lipid binding moiety in the present invention, including LC3A, LC3B, LC3C, GABARAP, GABARAPL1 and GABARAPL2. De la Ballina (2019), doi.org/10.1016/j.jmb.2019.05.051. Lipid-binding proteins include protein HCLS1 binding protein 3 (HSTBP3) that is able to negatively regulate the activity of phospholipase D1 (PLD1).

### Linker Moiety

A linker or linking moiety is a bifunctional or multifunctional moiety that can be used to link one or more activator moieties to a localizing moiety. In some embodiments, the linker has a functionality capable of reacting with the moieties for covalent attachment. The linker moiety is preferably a chemical linker moiety and is represented in Formula I-A and I-B as L. The linkers may be cleavable or non-cleavable. In embodiments, the linker is cleavable, and can be chosen for efficacy, safety, and rate of degradation in vivo. Linkers may be rigid, flexible, or cleavable in vivo, and can be rationally designed based on the properties of the moieties of the multifunctional molecule, and the additional design considerations detailed herein. See, e.g. Chen et al., Adv Drug Deliv Rev. 2013 Oct. 15; 65(10): 1357-1369 for discussion of rational design of linkers in pharmaceutical preparations. The length of the linker can be varied and studied for degree of ternary complex formation, and varying linker lengths to determine level of modification by the activator moiety. The linker can be bifunctional or multifunctional in nature, and multiple activator moieties can be appended to the linker at one or more functional groups. In particular instances, a multifunctional linker allows for the attachment of a vector and activator molecule at multiple sites on the linker. Branched linkers tuned in length and functionality for each of the activator molecules is within the scope of this invention

Linkers that can be utilized include PEG molecules, alkyl, heterocycloalkyl, cycloalkyl, aryl, alkylene, alkenyl, heteroaryl, amide, amine, thiol or derivatives thereof. The linker may be a multifunctional linker, in embodiments, a multifunctional PEG linker.

In certain embodiments, the linker is a product of azide/alkyne [3+2]cycloaddition, or is selected from amides, carbamates, esters, ureas, thioureas and PEG molecules. In preferred embodiments, the linker is a PEG molecule, alkyl or click chemistry linker such as trans-cyclooctene, cyclooctyne, or terminal alkyne, [e.g. reagent N-(1R,8S,9s)-Bicyclo[6.1.0]non-4-yn-9-ylmethyloxycarbonyl-1,8-diamino-3,6-dioxaoctane (“BCN amine”).

In an aspect, the linking moiety is according to the formula

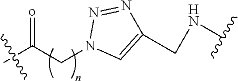

wherein n is between 1 and 15, preferably n=3, 5, 7, 9, or 11.

In embodiments, the linking moiety is according to the formula

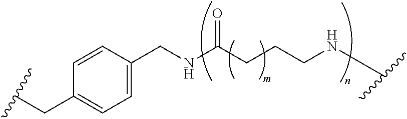

wherein n is between 0 and 10, 0 and 5, or 0 and 2 and m is between 0 and 10, 0 and 5, or 0 and 3.

In embodiments, the linker moiety is according to the formula:

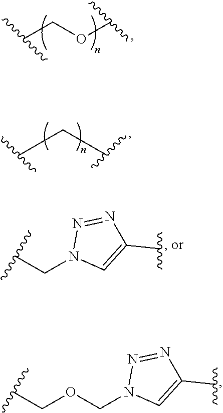

wherein n is between 1 and 50.

The linker can be according to

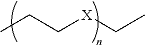

wherein X is O or CH2 and n is 0 to 10. In particular embodiments, when X is O, n=1 or 2 and when X=CH2, n=1.

In embodiments, the linker moiety can be:

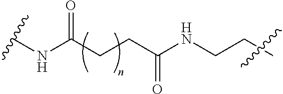

wherein n=0 to 5; or

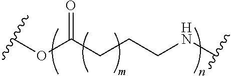

wherein n=0 to 5 and m=0 to 5.

### Activator Moiety

The activator moiety can be chosen based on the types of modification desired. As used herein, the activator moiety may be make a modification to a substrate that inhibits or activates the substrate. In one embodiment, the activator moiety is capable of finding and regulating, e.g. activating or inhibiting an enzyme. In certain embodiments, the activator moiety is an inhibitor molecule such as an allosteric inhibitor. In embodiments, the enzyme is a kinase, a phosphatase, transferase, ligase, histone acetylases (HATs), or histone deacetylases (HDACs), hydroxylase, a Glutamine Synthetase Adenyl Transferases (GSATase), enzymes catalyzing hydroxylation of protein residues, oxygenase, or sulfotransferase.

Activator moieties may be chosen based on the type of modification, for example, post-translational modification. In particular instances, the activator moiety is capable of finding and activating an enzyme, thus the type of enzyme activated may be chosen for the desired modification of a target substrate. Post-translational modification (PTM) one type of modification performed. This may include, cleaving peptide bonds, formation of disulfide bonds, acylation, prenylation, lipoylation, acetylation, deacetylation, formylation, alkylation, carbonylation, phosphorylation, glycosylation or lipidation, hydroxylation, S-nitrosylation, S-sulfenylation, dulfinylation, sulfonylation, succinylation, sulfation, malonylation (Taherzadeh et al. 2018). Accordingly, posttranslational modification enzymes are one set of activators envisaged for use in the present invention.

The activator may be selected based on the desired substrate modification. In embodiments, the activator provides a modification to an amino acid, see, e.g. for example Table 1 of Karve et al., Journal of Amino Acids Volume 2011, Article ID 207691, 13 pages, DOI:10.4061/2011/207691, incorporated herein by reference.

In an embodiment, the activator is a kinase activator moiety. The kinase activator moiety can be a small molecule or compound that activates a kinase. As used herein, a kinase is an enzyme that adds a phosphate group to another molecule, typically an amino acid of a protein substrate. An activator of a kinase enhances phosphorylation. In particular embodiments, the kinase activator moiety promotes an active conformation of an enzyme, in one aspect, trough binding interactions with regulatory subunits. See, e.g. Zorn et al., Nat. Chem. Biol. (2010), doi:10.1038/NCHEMBIO.318. In embodiments, the kinase acts on the amino acid serine, threonine, tyrosine, or a combination thereof.

In embodiments, the activator is an activator of a protein kinase C isozyme. In embodiments, the activator in activator of AMPK. In embodiments, the activator is an activator of an Src kinase, or shares sequence homology with the Src kinase family. In embodiments the kinase is c-Abl, a nonreceptor tyrosine kinase. In embodiments the kinase is Bruton's Tyrosine Kinase (BTK). The activator can be an activator of Insulin Receptor tyrosine kinase. In an aspect the activator moiety is an inhibitor of RAC-alpha serine/threonine-protein kinase (AKT) or mitogen-activated protein kinase (MEK).

Activator moieties can be identified from activators known in the art. The activators may be a derivative of activators known in the art, and may comprise fewer or additional functional groups that still permit their use as an activator, but may enhance or facilitate the desired formation, conformation or attachment sites for the multifunctional molecules described herein. Exemplary modifications may include derivatives for increase solubility, charge, functionality for use with an orienting adaptor or linker, detailed elsewhere in the specification.

### FKBP Activator Moiety

In particular embodiments, the activator can be designed as an activator of an FK506-binding protein (FKBP). In an aspect, the FKBP is FKBP12, which binds to intracellular calcium release channels and TGF-β type I receptor. In an aspect, the FKBP activator moiety is

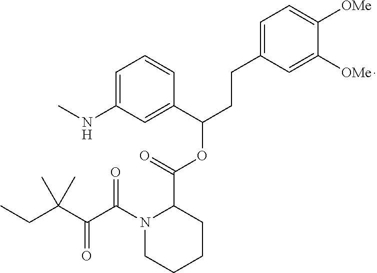 [SMILES: CCC(C)(C)C(=O)C(=O)N1CCCCC1C(=O)OC(CCc1ccc(OC)c(OC)c1)c1cccc(NC)c1]

### PKC Activator Moiety

In particular embodiments, the activator can be designed as an activator of a diacylglycerol (DAG) responsive C1 domain-containing protein, such as Protein Kinase C. Protein Kinase C (PKC) is comprised of multiple isozymes and plays a role in signal transduction pathways, exhibiting a tissue-specific expression and playing a variety of biological roles. Activators of PKC can be utilized in the small chimeric molecules disclosed herein, the activating moiety is selective for a PKC isoform.

The activator of a DAG responsive protein may comprise a DAG-indolactone as described in L. C. Garcia et al., Bioorg. Med. Chem., 22 (2014) 3123-3140. Exemplary DAG-indolactones may be according to the formula

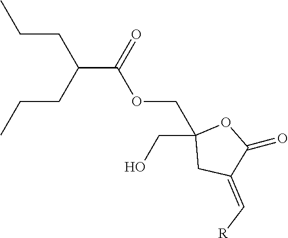

wherein R is an indole. R can be, for example, 1-methyl, 1H-indole5-yl, 1-methyl, 1H-indole6-yl, 1-methyl, 1H-indole4-yl, or 1-methyl, 1H-indole7-yl. In embodiments, the compounds are selective for PKCα or PKCε.

DAG lactones, such as AJH-836, as described in Cooke, et al., J. Biol. Chem. (2018) 293(22) 8330-8341. In embodiments, the DAG lactone can be according to the formula

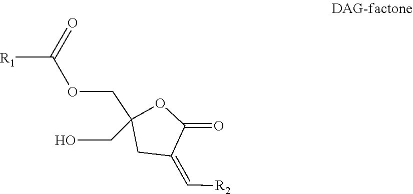

As provided in Cooke, AJH-836 formula is

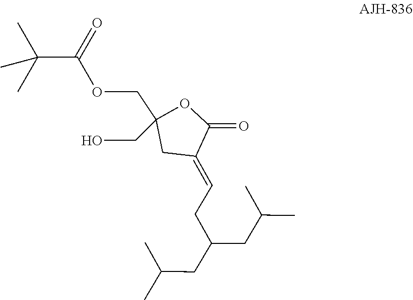 [SMILES: CC(C)CC(C/C=C1\CC(CO)(COC(=O)C(C)(C)C)OC1=O)CC(C)C]

and is selective for PKCδ and PKC.

Teleocidins, such as (−)-indolactam-V (ILV), and benzolactam-V8s, for example, 7-substituted Benzolactam-V8s, can be utilized as PKC activators. The PKC activator can be as described in Ma, et al., Org. Lett. 4:14 (2002) DOI:10.1021/ol026125l.

In embodiments, the PKC activator is according to the formula

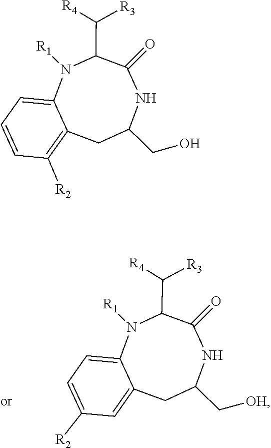

wherein R1, R3, and R4 are each independently alkyl, alkenyl, alkynyl, and R2 can be selected from divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₃-C₂₂ heterocycloalkylene, C₁-C₁₀ branched heteroalkylelene, C₁-C₁₀ linear heteroalkylene, C₃-C₁₀ heterocycloalkylene, C₁-C₈ branched heteroalkylelene, C₁-C₈ linear heteroalkylene, C₃-C₈ heterocycloalkylene), arylene (e.g., C₅-C₂₂ arylene), heteroarylene (e.g., C₅-C₂₂ heteroarylene), or combinations thereof; wherein each of the foregoing may have one or more (e.g., two, three, four, five) points of substitution, substituted amides, including those selected from those as described in Table 1 of Kozikowski et al. J. Med. Chem, 2003, 46:3, 364-373, Table 1 at page 366, incorporated specifically herein by reference. R2 can be selected from one or more of —(C(R^{a})(R^{a}))₁₋₈—, —(OC(R^{a})(R^{a}))₁₋₈—, —(OC(R^{a})(R^{a})—C(R^{a})(R^{a}))₁₋₈—, —N(R^{a})—, —O—, —C(O)—, optionally substituted C₆ arylene, optionally substituted C₅₋₁₂ heteroarylene, C₃₋₆ cycloalkylene substituted with hydroxy, or C₄ heterocycloalkylene substituted with hydroxy; wherein each of the foregoing may have one or more (e.g., two, three, four, five) points of substitution; and R^{a} is independently selected at each occurrence from hydrogen, or alkyl (e.g., C₁-C₇ alkyl, C₁-C₃ alkyl).

In particular embodiments, the formula is according to

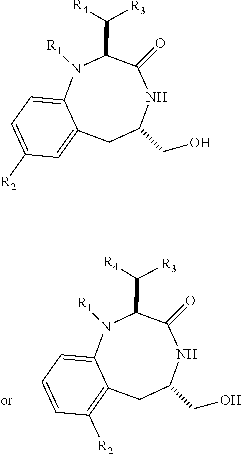

wherein R1, R3, and R4 are each independently alkyl, alkenyl, alkynyl, and R2 can be selected from divalent hydrocarbon selected from saturated or unsaturated alkylene (e.g., branched alkylelene, linear alkylene, cycloalkylene, C₁-C₂₂ branched alkylelene, C₁-C₂₂ linear alkylene, C₃-C₂₂ cycloalkylene, C₁-C₁₀ branched alkylelene, C₁-C₁₀ linear alkylene, C₃-C₁₀ cycloalkylene, C₁-C₈ branched alkylelene, C₁-C₈ linear alkylene, C₃-C₈ cycloalkylene), C₁-C₂₂ saturated or unsaturated heteroalkylene (e.g., branched heteroalkylelene, linear heteroalkylene, heterocycloalkylene, C₁-C₂₂ branched heteroalkylelene, C₁-C₂₂ linear heteroalkylene, C₃-C₂₂ heterocycloalkylene, C₁-C₁₀ branched heteroalkylelene, C₁-C₁₀ linear heteroalkylene, C₃-C₁₀ heterocycloalkylene, C₁-C₈ branched heteroalkylelene, C₁-C₈ linear heteroalkylene, C₃-C₈ heterocycloalkylene), arylene (e.g., C₅-C₂₂ arylene), heteroarylene (e.g., C₅-C₂₂ heteroarylene), or combinations thereof; wherein each of the foregoing may have one or more (e.g., two, three, four, five) points of substitution, substituted amides, including those selected from those as described in Table 1 of Kozikowski et al. J Med. Chem, 2003, 46:3, 364-373, Table 1 at page 366, incorporated specifically herein by reference.

R2 can be selected from one or more of —(C(R^{a})(R^{a}))₁₋₈—, —(OC(R^{a})(R^{a}))₁₋₈—, —(OC(R^{a})(R^{a})—C(R^{a})(R^{a}))₁₋₈—, —N(R^{a})—, —O—, —C(O)—, optionally substituted C₆ arylene, optionally substituted C₅₋₁₂ heteroarylene, C₃₋₆ cycloalkylene substituted with hydroxy, or C₄ heterocycloalkylene substituted with hydroxy; wherein each of the foregoing may have one or more (e.g., two, three, four, five) points of substitution; and R^{a} is independently selected at each occurrence from hydrogen, or alkyl (e.g., C₁-C₇ alkyl, C₁-C₃ alkyl).

In particular embodiments, the formula is according to

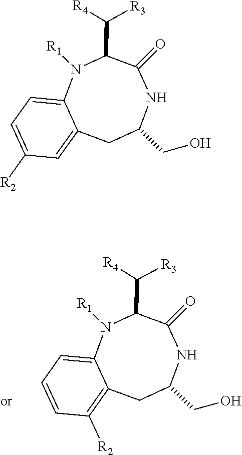

wherein R1, R3 and R4 is independently alkyl, alkenyl, alkylnyl, and R2 can be selected from. In embodiments, the PKC activator is a benzolactam analogue of ILV, with R can be CC(CH₂)₇CH₃ or (CH₂)₉CH₃, as described in Kozikowski et al., J. Med. Chem., 1997, 40:9 1316-1326.

In embodiments, R1, R3 and R4 are alkyl, in some embodiments, R1, R3 and R4 are methyl. In certain embodiments, the formula is according to:

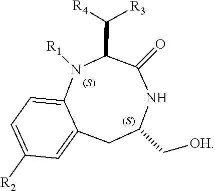

In embodiments, the PKC activator is a natural product activator, for example, DPP, prostratin, mezerein, octahydromexerein, thymeleatoxin, (−)-ocytlindolactam V, OAG, or resiniferatoxin, as described in Kazanietz. et al., Mol. Pharma. 44:296-307 (1993).

In embodiments, the PKC activator is selective for PKCδ. In particular embodiments, the PKC activator is 7α-acetoxy-6β-benzoyloxy-12-Obenzoylroyleanone (Roy-Bz) as described in Bessa et al., Cell Death and Disease (2018) 9:23.

The PKC activator may be an ILV derivative, such as n-hexyl ILV, or a 10 membered ring 1-Hexylindolactam-V10, or a derivative thereof, as described in Yanagita, et al., J. Med. Chem., 2008, 51:1, 46-56, incorporated herein by reference. The PKC activator may be

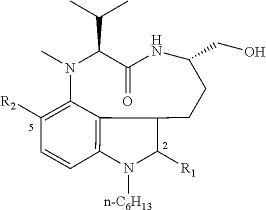

wherein R1 and R2=H, R1=H and R2=Cl, or R1=Br and R2=H, and may, in some instance be PKCδ, PKCε or PKCη.

In embodiments, the activator moiety is 6-Chloro-5-[4-(1-hydroxycyclobutyl)phenyl]-1H-indole-3-carboxylic Acid (PF-06409577), a benzolactam, DPP, Prostratin, Mezerein, Octahydromezerein, Thymeleatoxin, (−)-Indolactam V, (−)-Octylindolactam V, OAG, or derivatives thereof.

In embodiments, the activator moiety is a thieno [2,3-b]pyridine, a thienopyridone, a quinoxalinedione, a imidazo [4,5-b]pyridine, a [2,3-d]pyridine, a benizimidazole, a pyrrolo [2,3-d]pyrimidine, a spirocyclic indolinone, a tetrahydroquinoline, a thieno [2,3-b]pyridinedione, and derivatives thereof. See Expert Opin Ther. Patents (2012) 22(12), incorporated herein by reference.

TABLE 1
Exemplary PKC Activators
PKC Activators

| | | | Crystal | | |
|---|---|---|---|---|---|
| | PKC | Kd (nM) | structure | Docking | Cell tests |
| 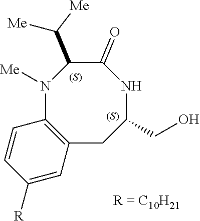 | alpha beta gamma delta epsilon | 46.6 58.2 140 185 187 | − − − − − | − − − − − | − − − − − |
| 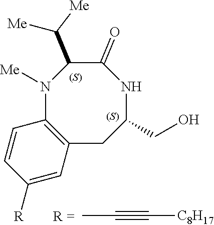 | alpha beta gamma delta epsilon | 14.7 17.4 40.7 122 142 | − − − − − | truncated − − − − | antiproliferative activity against breast carcinoma cell lines MCF-7 and MDA-MB- 231 preferentially down-regulates PKCalpha |
| 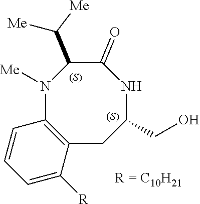 | alpha beta gamma delta epsilon | 513 309 409 169 60 | − − − − − | − − − − − | − − − − − |
| 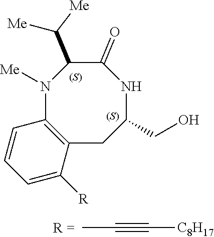 | alpha beta gamma delta epsilon | 4533 1691 >10 000 2098 2562 | − − − − − | − − − − − | − − − − − |
| 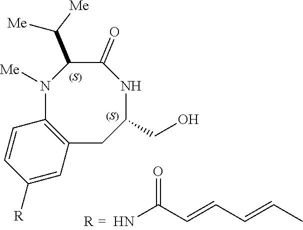 | alpha | 1970 | − | truncated | Enhancement of sAPPR (amyloid precursor protein) secretion was achieved at 1 uM in cell lines derived from patients with Alzheimer's disease |
| 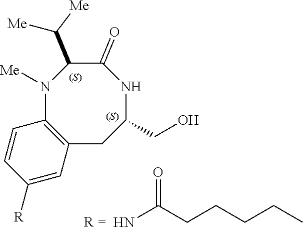 | alpha | 225 | − | − | same |
| 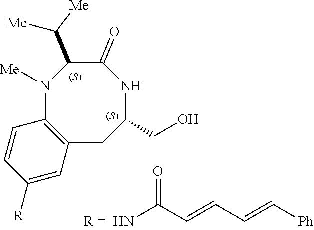 | alpha | 15.8 | − | − | same |
| 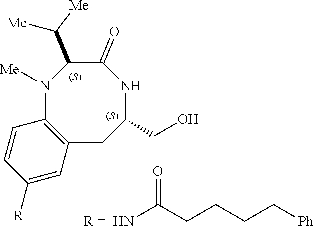 | alpha | 34.2 | − | − | same |
| 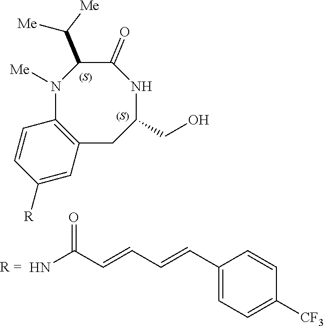 | alpha | 11.9 | − | − | Enhancement of sAPPR (amyloid precursor protein) secretion was achieved at 0.1 uM in cell lines derived from patients with Alzheimer's disease |
| 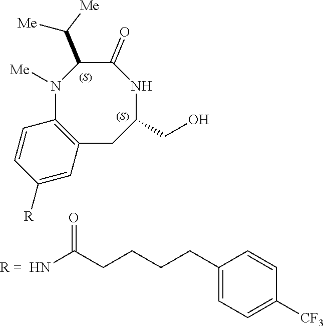 | alpha | 12.5 | − | − | same |
| 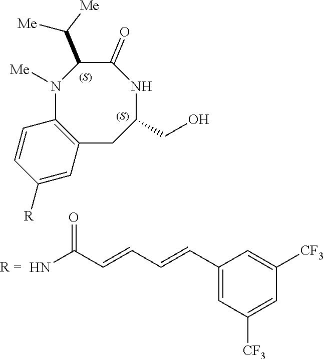 | alpha | 5.6 | − | − | Enhancement of sAPPR (amyloid precursor protein) secretion was achieved at 0.1 uM in cell lines derived from patients with Alzheimer's disease commercially available, patented |
| 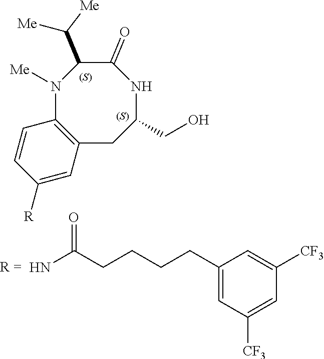 | alpha | 7.8 | − | − | same |
| 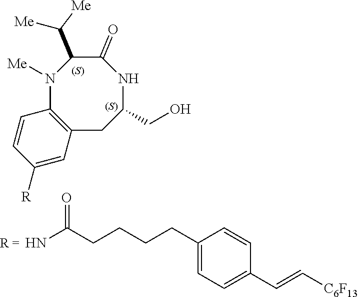 | alpha | 11.2 | − | − | same |
| 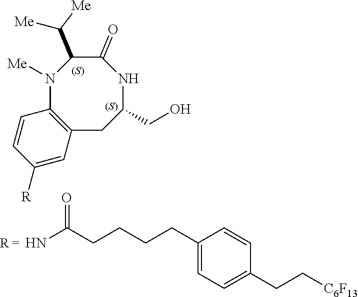 | alpha | 9.2 | − | − | same |
| 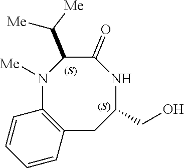 | alpha | 334 | − | − | |
| 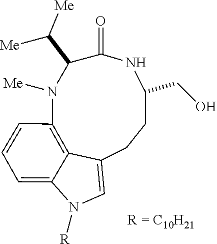 | alpha beta gamma delta epsilon | 6700 9200 420 15 29 | | | |
| 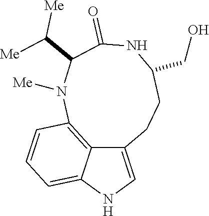 [SMILES: CC(C)[C@H]1C(=O)N[C@H](CO)CCc2c[nH]c3cccc(c23)N1C] | alpha beta gamma delta epsilon | 11 6.1 19.4 8.2 21.9 | − − − − − | − − − − − | inactive against breast carcinoma |
| 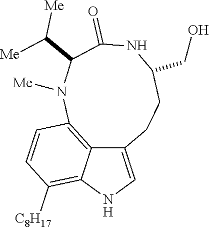 | alpha beta gamma delta epsilon | 0.5 0.4 1.2 0.8 1 | − − − − | − − − − | |
| 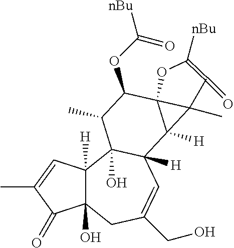 | alpha beta gamma delta epsilon eta zeta | 0.2 0.2 0.33 0.94 0.81 0.87 no binding | | | |
| 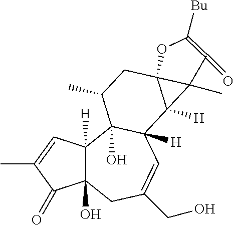 | alpha beta gamma delta epsilon eta | 0.14 0.11 0.28 0.56 0.62 0.61 | | | |
| 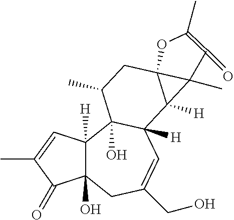 | alpha beta gamma delta epsilon eta | 4.8 3.1 9.8 24.6 27.5 20.9 | | | |
| 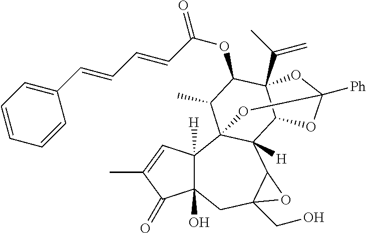 [SMILES: C=C(C)[C@@]12OC3(c4ccccc4)O[C@@H]1[C@@H]1C4OC4(CO)C[C@]4(O)C(=O)C(C)=C[C@H]4[C@@]1(O3)[C@H](C)[C@H]2OC(=O)/C=C/C=C/c1ccccc1] | alpha beta gamma delta epsilon eta | 0.27 0.16 0.21 0.55 1.2 1.27 | | | |
| 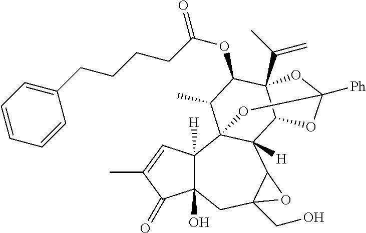 [SMILES: C=C(C)[C@@]12OC3(c4ccccc4)O[C@@H]1[C@@H]1C4OC4(CO)C[C@]4(O)C(=O)C(C)=C[C@H]4[C@@]1(O3)[C@H](C)[C@H]2OC(=O)CCCCc1ccccc1] | alpha beta gamma delta epsilon eta | 0.82 0.51 0.8 2.17 3.13 2.48 | | | |
| 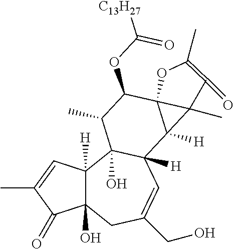 | alpha beta gamma delta epsilon eta | 0.29 0.16 0.26 1.31 3.11 2.63 | | | |
| 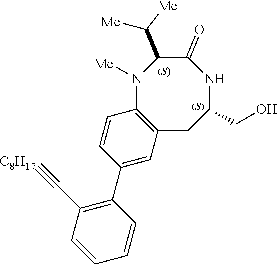 | alpha | 65.9 | | | |
| 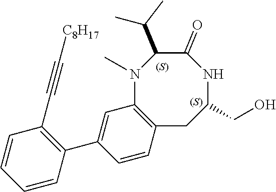 | alpha | 3.6 | | | |
| 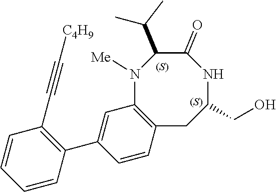 | alpha | 3.1 | | | |
| 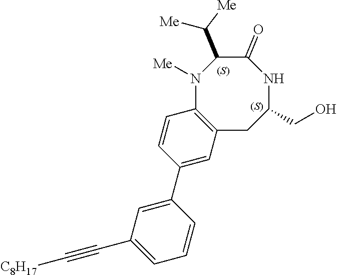 | alpha | 309 | | | |
| 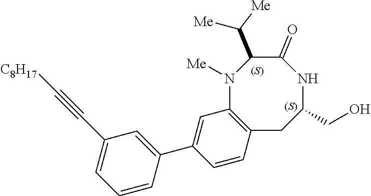 | alpha | 5.9 | | | |
| 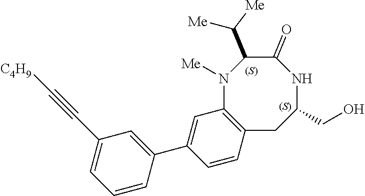 | alpha | 3 | | | |
| 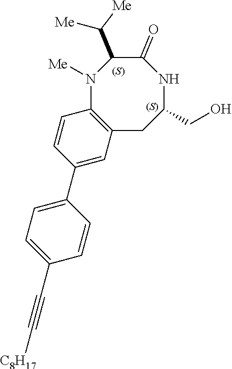 | alpha | 703 | | | |
| 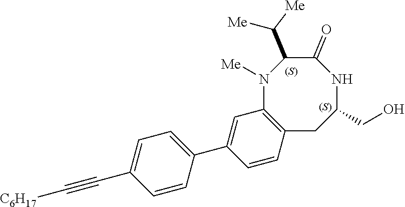 | alpha | 6 | | | |
| 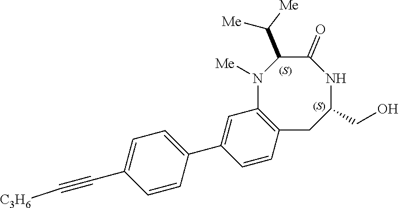 | alpha | 1.2 | | | |
| 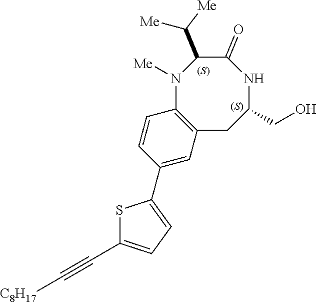 | alpha | 396 | | | |
| 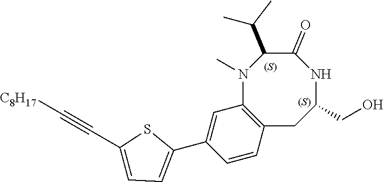 | alpha | 25 | | | |
| 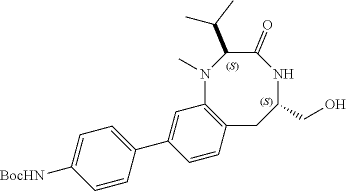 | alpha | 31.5 | | | |
| 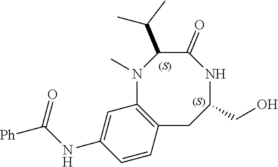 | alpha | 173.9 | | | |
| 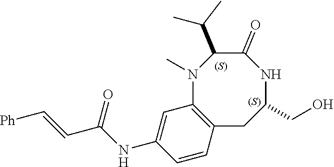 | alpha | 114.4 | | | |
| 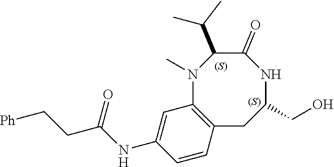 | alpha | 115.7 | | | |
| 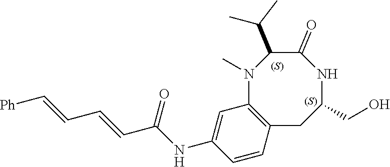 | alpha | 49.8 | | | |
| 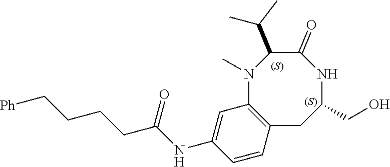 | alpha | 18.3 | | | |
| 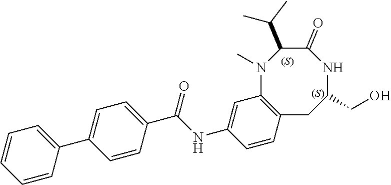 | alpha | 25.9 | | | |
| 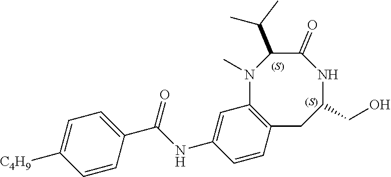 | alpha | 9.5 | | | |
| 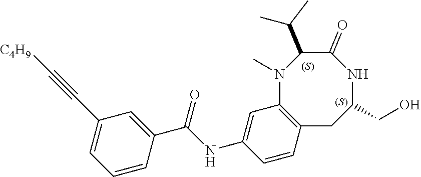 | alpha | 4.1 | | | |
| 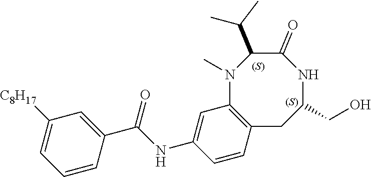 | alpha | 3.2 | | | |
| 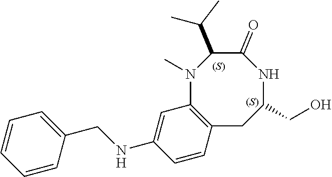 | alpha | 3.8 | − | + | |
| 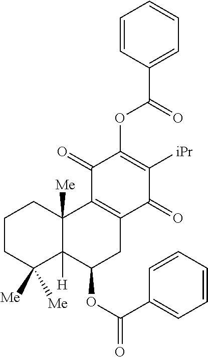 | delta | EC50 = 58.8 | − | + | tested in HCT116, HT-29 and SW- 837 cells inhibited proliferation of colon cancer cells |
| 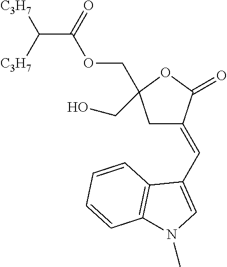 | alpha beta RasGRP3 | 16.2 21.1 0.33 | | | CHO-K1 cells were transiently transfected with GFP-PKCa (A, B) LNCaP cells were transiently transfected with YFP-PKCe |
| 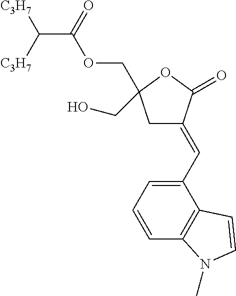 | alpha beta RasGRP3 | 17.8 14.6 1.63 | | | |
| 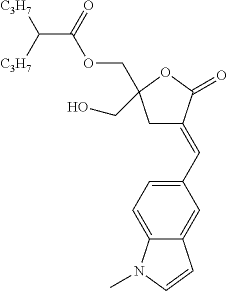 | alpha beta RasGRP3 | 8.3 2.2 0.34 | | | |
| 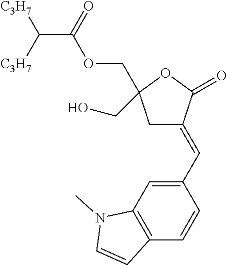 | alpha beta RasGRP3 | 7.5 8.1 1.2 | | | |
| 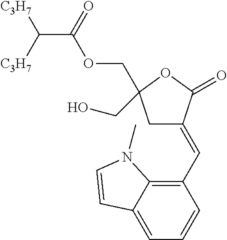 | alpha beta RasGRP3 | 12.9 11.7 1.12 | | | |
| 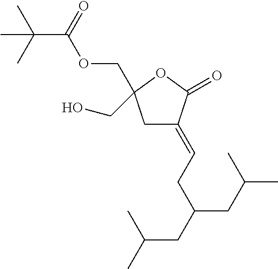 [SMILES: CC(C)CC(C/C=C1\CC(CO)(COC(=O)C(C)(C)C)OC1=O)CC(C)C] | alpha beta delta epsilon | 23.6 19.7 1.89 1.89 | | | AJH-836 induced major changes in cytoskeletal reorganization in lung cancer and HeLa cervical adenocarcinoma cells as determined by the formation of membrane ruffles |

NASA chemistry can be used to functionalize an activator moiety that can be further appended with a localizing moiety. NASA chemistry is generally described in Nat Commun 9, 1870 (2018), incorporated herein by reference. In certain embodiments, the PKC activator moiety can be attached to a localizing moiety utilizing N-acyl N-alkyl sulfonamide (NASA) warhead. In an aspect, the NASA warhead comprises

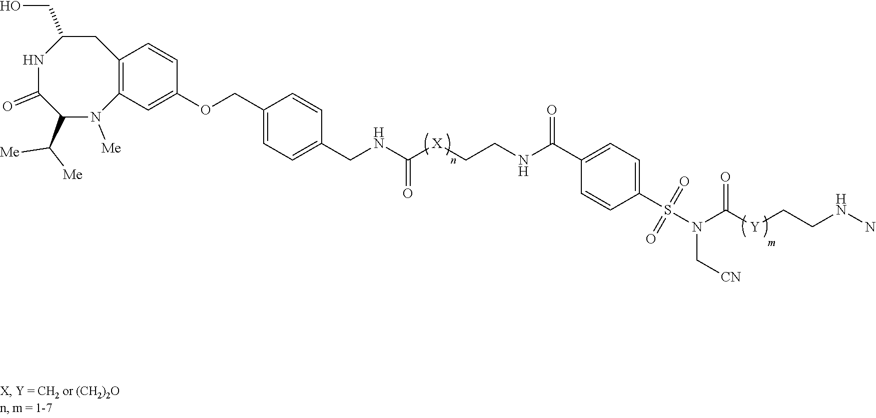

wherein R comprises a fluorescent dye, BRD4 binder, FKBP binder, MDM2 binder, ER binder, or a binder of any other protein of interest.

In another approach, NASA chemistry is used to label PKC activator moiety with tetra-cyclooctene (TCO), allowing the use of tetrazine click chemistry to click to binder of any protein of interest. Accordingly, in an aspect, the PKC activator is prepared according to the formula:

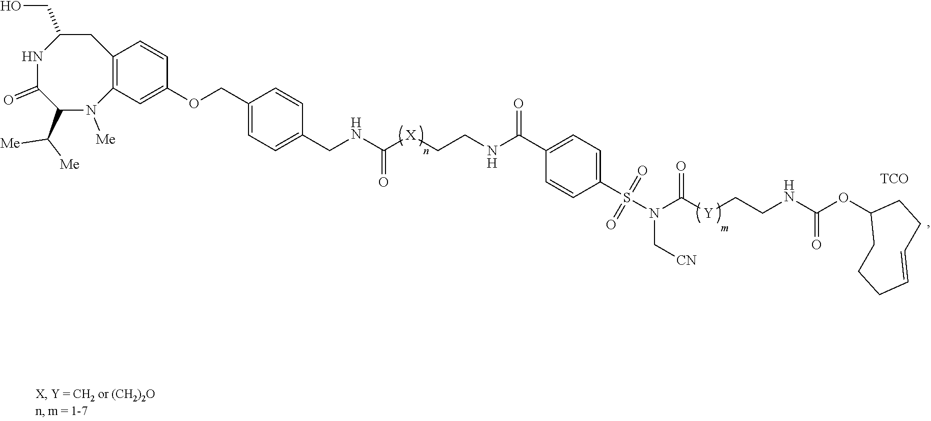

which can then be reacted with a functionalized tetrazine according to

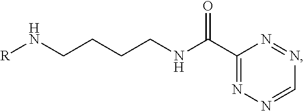

wherein R is a binder of any protein of interest, e.g. localizing moiety. Accordingly, an embodiment comprises methods of making compositions disclosed herein using NASA chemistry, and as further described in the examples.

### AMPK Activator Moiety

AMPK is a serine/threonine kinase that assembles into a heterotrimeric complex composed of a catalytic α-subunit and two regulatory β- and γ-subunits. See, e.g. Wells et al. (2012). It is believed that small molecules that mimic AMP binding to the γ-subunit could directly activate AMPK. AMPK activators may be selected from the AMPK activators, as disclosed herein.

In embodiments, the AMPK activator is selected from

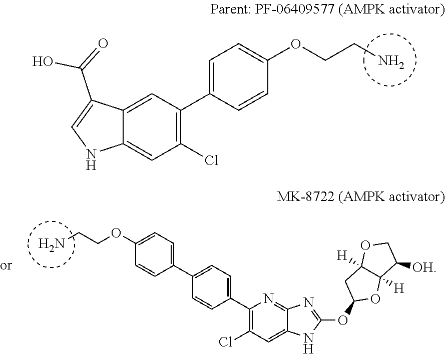 [SMILES: NCCOc1ccc(-c2cc3c(C(=O)O)c[nH]c3cc2Cl)cc1 ; NCCOc1ccc(-c2ccc(-c3nc4nc(O[C@H]5C[C@H]6OC[C@@H](O)[C@H]6O5)[nH]c4cc3Cl)cc2)cc1]

Other AMPK activators include A769662 (Cool et al., Cell Metab. 3, 403-416 (2006)) and PT1 (Pang et al., J. Biol. Chem. 283, 16051-16060 (2008).

AMPK activators can be as described for example in U.S. Patent Publication 20050038068, incorporated herein by reference, and can be according to

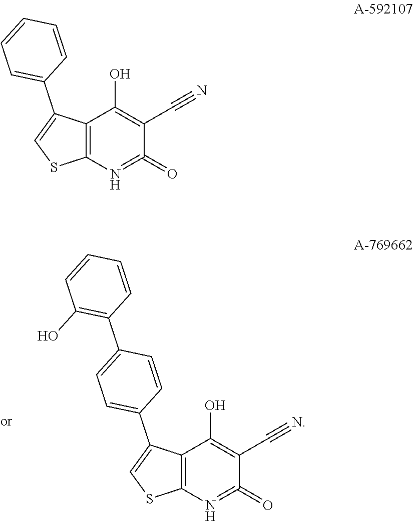

AMPK activators can be as described in International Patent Publications WO2007019914, WO2009124636, WO2009135580, WO2008006432, or WO2009152909, incorporated herein by reference. In embodiments, the activator can be according to

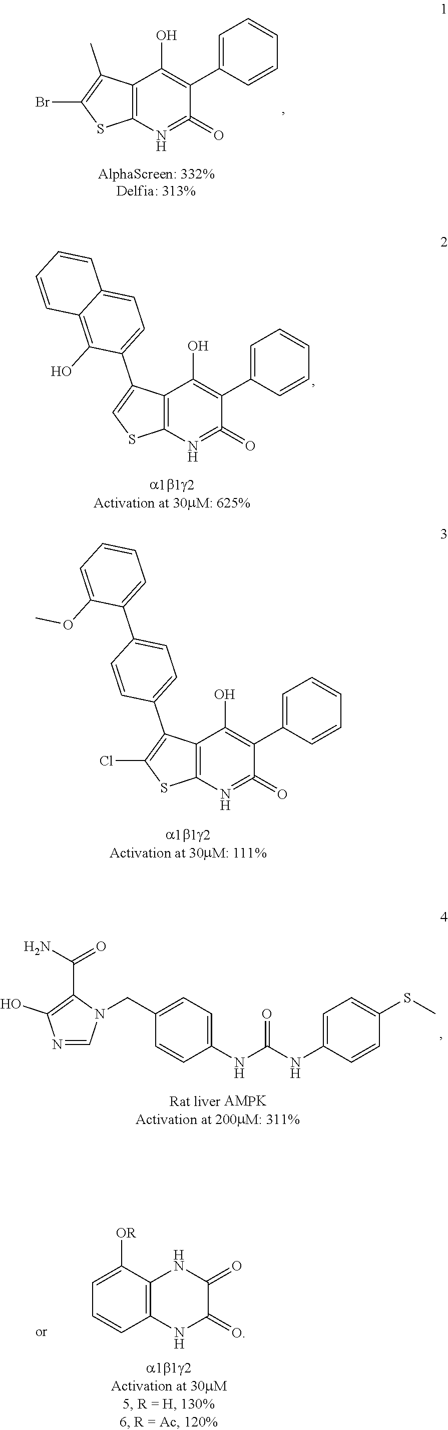

The AMPK activator can be as described in International Patent Publication WO2009100130, incorporated herein by reference. In one aspect, the AMPK activator is according to

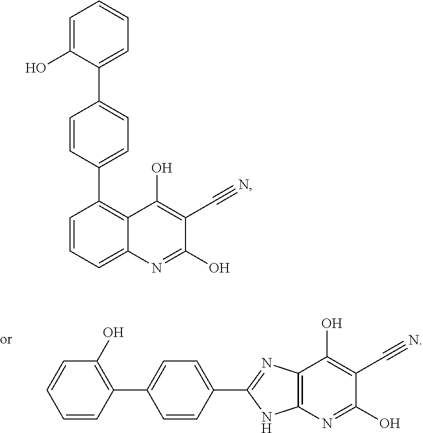 [SMILES: N#Cc1c(O)nc2cccc(-c3ccc(-c4ccccc4O)cc3)c2c1O ; N#Cc1c(O)nc2[nH]c(-c3ccc(-c4ccccc4O)cc3)nc2c1O]

The AMPK activator can be as described in International Patent Publications WO2010036613, WO2010047982, WO2010051176, WO2010051206, WO2011106273, or WO2012116145. In embodiments, the AMPK activator is according to

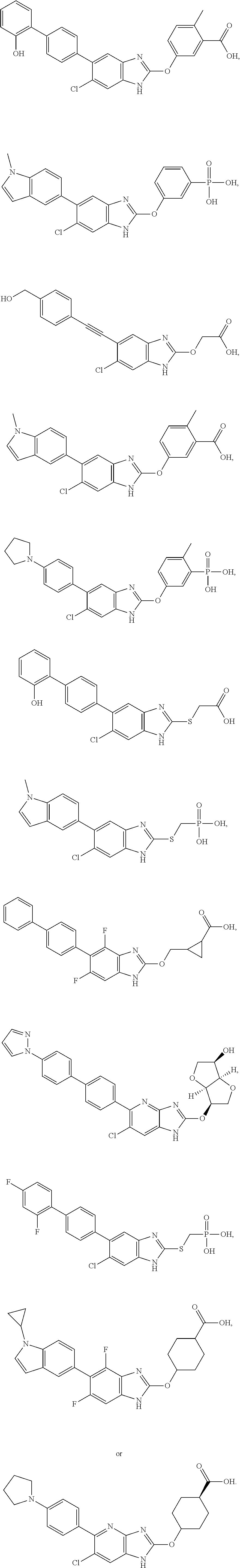

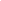

In certain example embodiments, the AMPK activator can be as described in International Patent Publications WO2011029855, WO2011138307, WO2012119979, WO2012119978, incorporated herein by reference. In one aspect the AMPK activator can be selected from

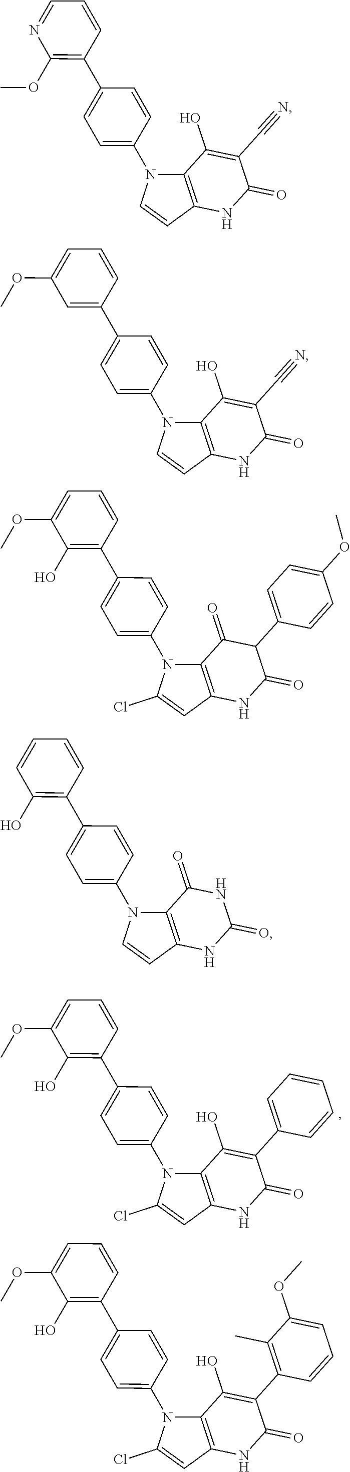

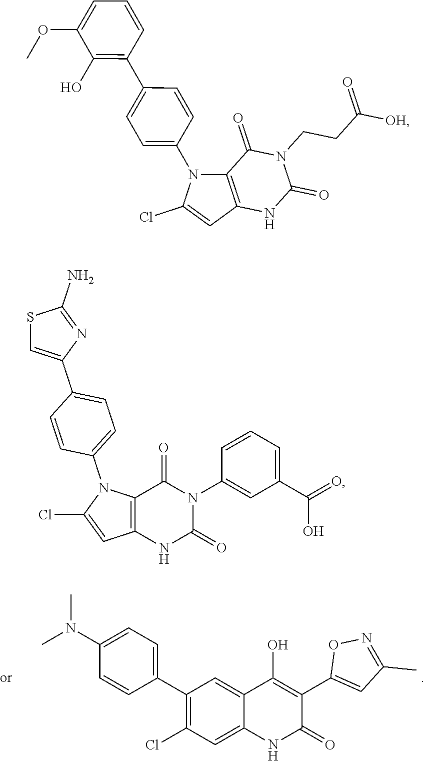

In certain example embodiments, the AMPK activator can be as described in International Patent Publications WO2011032320, WO2011033099, WO2011069298, WO2011070039, WO2011128251, WO2012001020, incorporated herein by reference. In one aspect the AMPK activator can be selected from

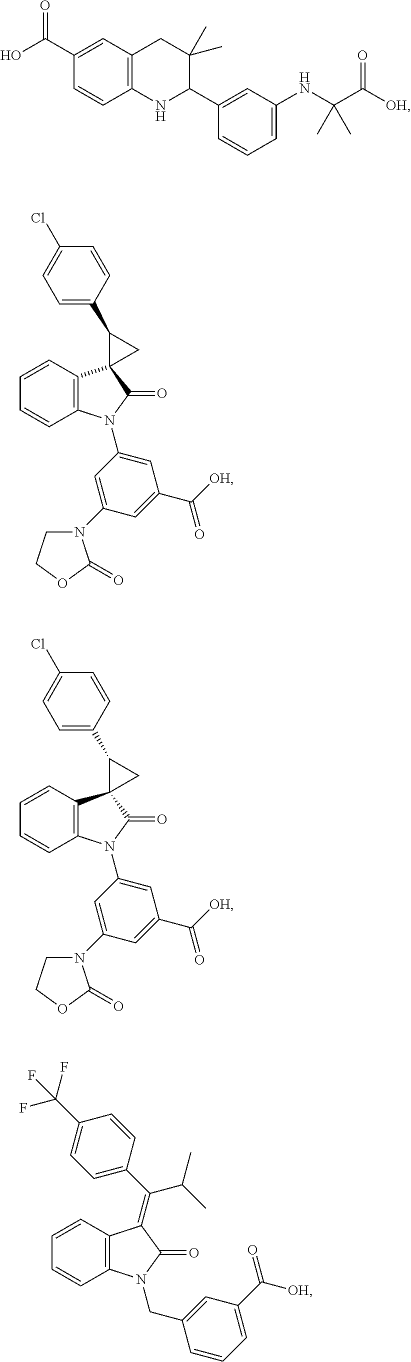 [SMILES: CC(C)(Nc1cccc(C2Nc3ccc(C(=O)O)cc3CC2(C)C)c1)C(=O)O ; O=C(O)c1cc(N2CCOC2=O)cc(N2C(=O)[C@@]3(C[C@@H]3c3ccc(Cl)cc3)c3ccccc32)c1 ; O=C(O)c1cc(N2CCOC2=O)cc(N2C(=O)[C@]3(C[C@H]3c3ccc(Cl)cc3)c3ccccc32)c1 ; CC(C)/C(=C1\C(=O)N(Cc2cccc(C(=O)O)c2)c2ccccc21)c1ccc(C(F)(F)F)cc1]

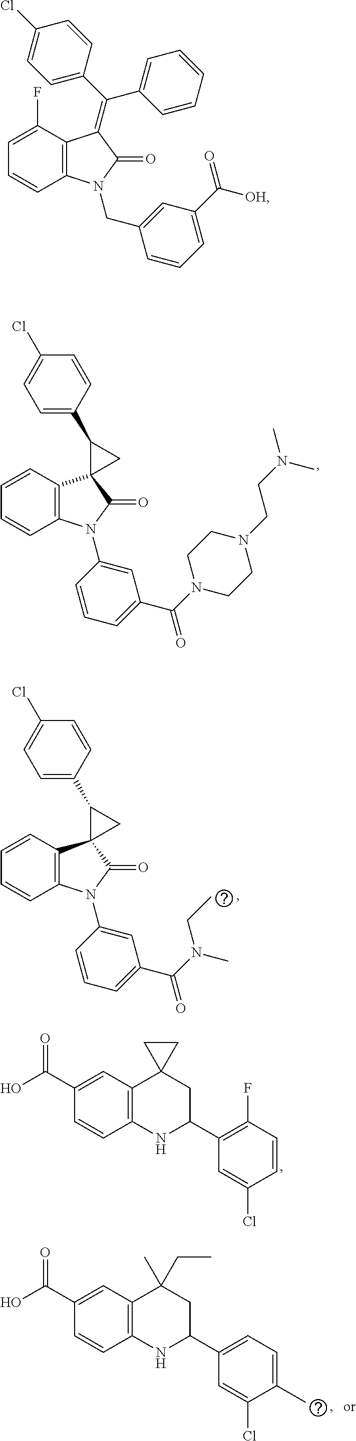

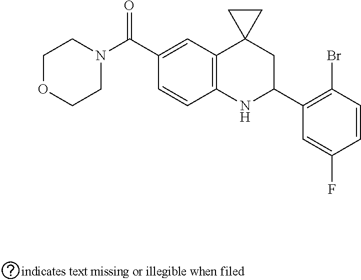 [SMILES: O=C(c1ccc2c(c1)C1(CC1)CC(c1cc(F)ccc1Br)N2)N1CCOCC1]

In certain example embodiments, the AMPK activator can be as described in International Patent Publication WO2011080277, incorporated herein by reference. In one aspect the AMPK activator can be

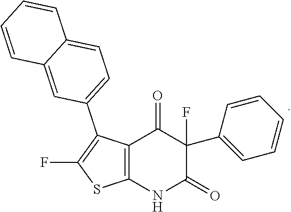 [SMILES: O=C1Nc2sc(F)c(-c3ccc4ccccc4c3)c2C(=O)C1(F)c1ccccc1]

In certain example embodiments, the AMPK activator can be as described in International Patent Publication WO2012033149, incorporated herein by reference. In one aspect the AMPK activator can be selected from

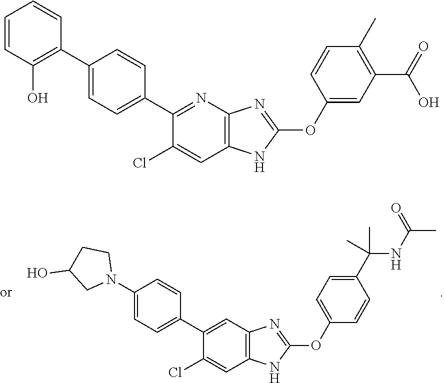 [SMILES: Cc1ccc(Oc2nc3nc(-c4ccc(-c5ccccc5O)cc4)c(Cl)cc3[nH]2)cc1C(=O)O ; CC(=O)NC(C)(C)c1ccc(Oc2nc3cc(-c4ccc(N5CCC(O)C5)cc4)c(Cl)cc3[nH]2)cc1]

### Bruton's Tyrosine Kinase Activator Moiety

Bruton's tyrosine kinase (Btk) is involved in multiple signaling cascades, and plays a role in B-cell development and oncogenic signaling. See, e.g. Singh et al., 2018; Pal et al., 2018. In embodiments, the Btk activator is ibrutinib or a derivative thereof.

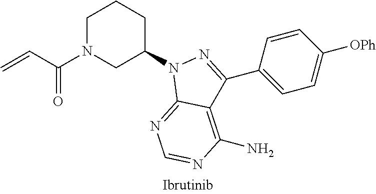 [SMILES: C=CC(=O)N1CCC[C@@H](n2nc(-c3ccc(Oc4ccccc4)cc3)c3c(N)ncnc32)C1]

In certain example embodiments, the Btk activator is selected from

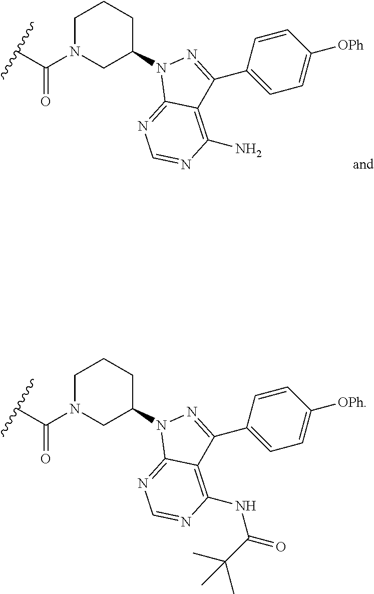 [SMILES: *C(=O)N1CCC[C@@H](n2nc(-c3ccc(Oc4ccccc4)cc3)c3c(N)ncnc32)C1 ; *C(=O)N1CCC[C@@H](n2nc(-c3ccc(Oc4ccccc4)cc3)c3c(NC(=O)C(C)(C)C)ncnc32)C1]

In certain example embodiments, the Btk activator moiety is provided with a targeting moiety of

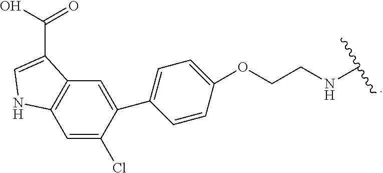 [SMILES: *NCCOc1ccc(-c2cc3c(C(=O)O)c[nH]c3cc2Cl)cc1]

The linker, where present may comprise an alkyl chain comprising between 5 and 10, more preferably between 6 and 8 carbon chain. In certain example embodiments, the molecule comprising a Btk activator moiety is

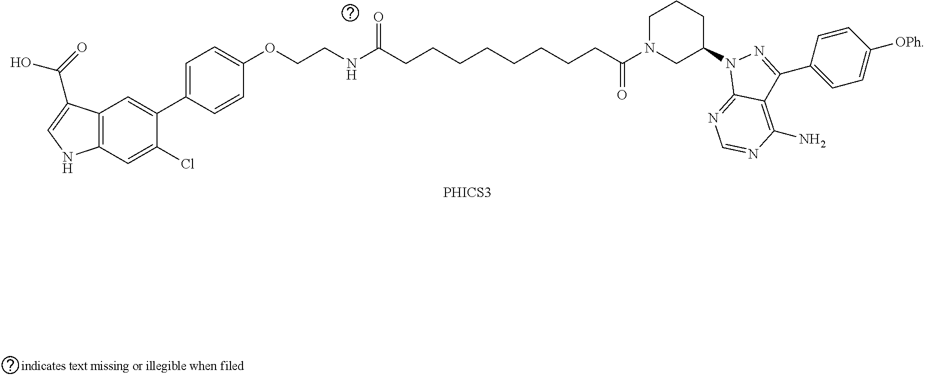 [SMILES: Nc1ncnc2c1c(-c1ccc(Oc3ccccc3)cc1)nn2[C@@H]1CCCN(C(=O)CCCCCCCCC(=O)NCCOc2ccc(-c3cc4c(C(=O)O)c[nH]c4cc3Cl)cc2)C1 ; "PHICS3"]

### ABL Kinase Activator Moiety

Abelson kinase (c-Abl) is a ubiquitously expressed, nonreceptor tyrosine kinase which plays a key role in cell differentiation and survival. Simpson, et al., J. Med. Chem. 2019 62, 2154-2171. ABL tyrosine kinase can be found in the nucleus, cytoplasm, and mitochondria. In embodiments, the c-Abl Kinase activator is (5-[3-(4-fluorophenyl)-1-phenyl-1H-pyrazol-4-yl]-2,4-imidazolidinedione or 5-(1,3-diaryl-1H-pyrazol-4-yl)hydantoin):

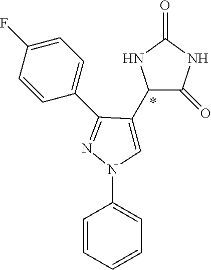 [SMILES: O=C1NC(=O)C(c2cn(-c3ccccc3)nc2-c2ccc(F)cc2)N1]

(DPH) as described in Yang et al., “Discovery and Characterization of a Cell-Permeable, Small-Molecule c-Abl Kinase Activator that Binds to the Myristoyl Binding Site, Chem. & Biol., 18, 177-186, Feb. 25, 2011; DOI: 10.1016/j.chembiol.2010.12.013. In certain embodiments, the c-Abl kinase activator can be selected from

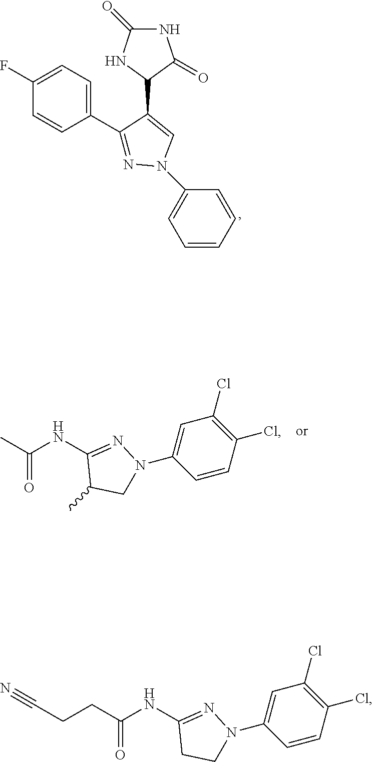

which showed in vivo activation of c-Abl in Simpson et al. 2019. The novel aminopyrazoline small molecule activators described in Simpson et al. at Table 6, are specifically incorporated herein by reference.

In certain example embodiments, the c-Abl kinase activator moiety is

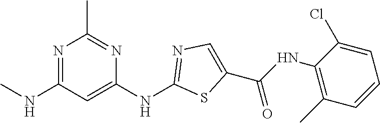 [SMILES: CNc1cc(Nc2ncc(C(=O)Nc3c(C)cccc3Cl)s2)nc(C)n1]

In certain example embodiments, the compound is according to the formula

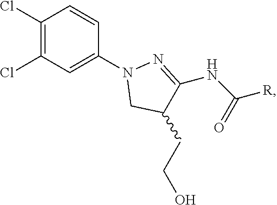

wherein R is

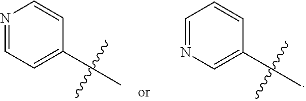 [SMILES: *c1ccncc1 ; *c1cccnc1]

In certain example embodiments, the DPH is functionalized:

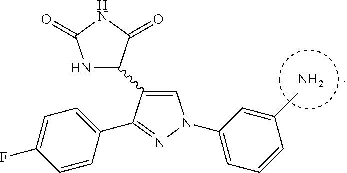

In certain example embodiments, the ABL kinase activator is

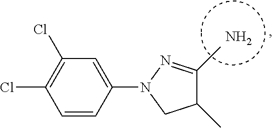

wherein the dashed circle identifies the attachment for the orienting adaptor and/or linker. The functional groups depicted in the dashed circle of the ABL kinase activator can be utilized in methods for attaching a linker and orienting adaptor prior to attachment to the localizing moiety.

Activator moieties may be functionalized for methods of attaching orienting adaptor and linker. ABL kinase activator parent molecule DPH can be functionalized for methods of attaching orienting adaptor and linker. Exemplary molecules may be:

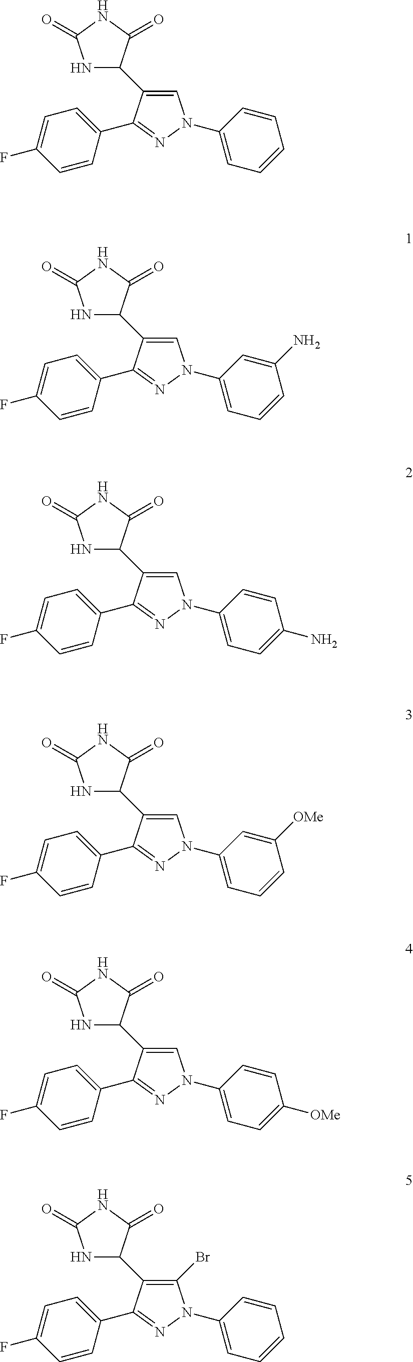 [SMILES: O=C1NC(=O)C(c2cn(-c3ccccc3)nc2-c2ccc(F)cc2)N1 ; Nc1cccc(-n2cc(C3NC(=O)NC3=O)c(-c3ccc(F)cc3)n2)c1 ; Nc1ccc(-n2cc(C3NC(=O)NC3=O)c(-c3ccc(F)cc3)n2)cc1 ; COc1cccc(-n2cc(C3NC(=O)NC3=O)c(-c3ccc(F)cc3)n2)c1 ; COc1ccc(-n2cc(C3NC(=O)NC3=O)c(-c3ccc(F)cc3)n2)cc1 ; O=C1NC(=O)C(c2c(-c3ccc(F)cc3)nn(-c3ccccc3)c2Br)N1]

Once functionalized, the orienting adaptor and linker can be added, either sequentially, or at once, with the orienting adaptor and linker added as one molecule. Exemplary molecules are provided below, with the R group representing the localizing moiety.

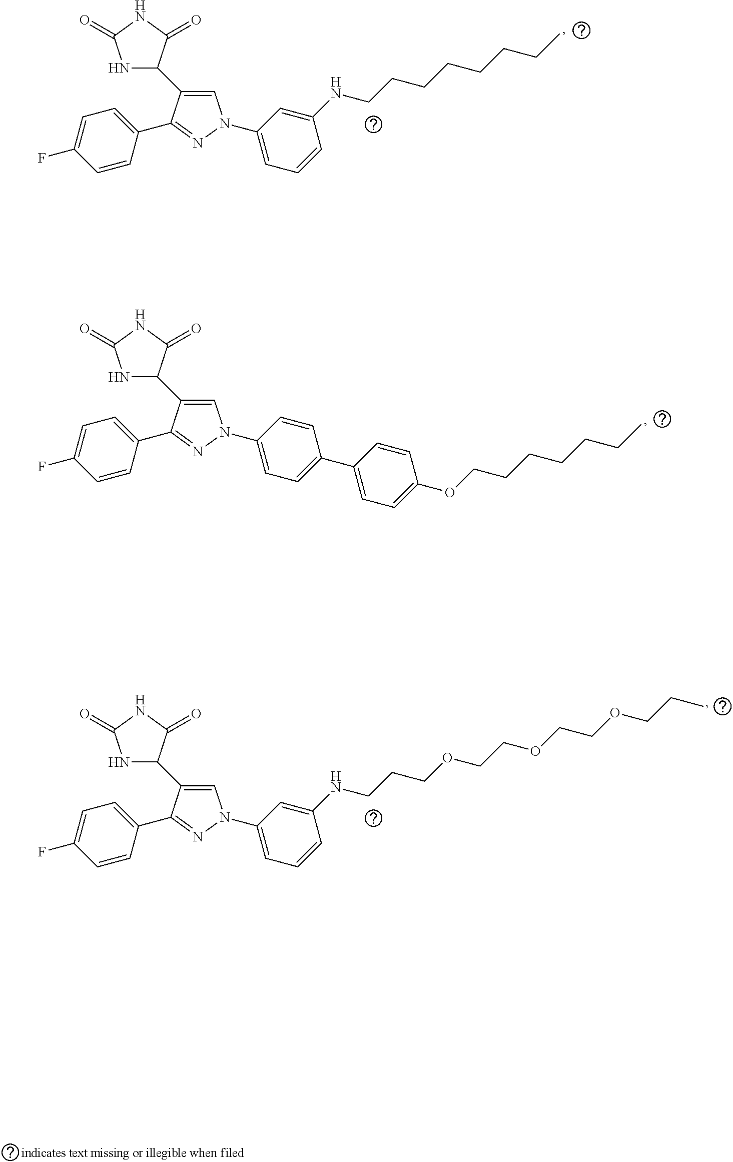 [SMILES: CCCCCCCCNc1cccc(-n2cc(C3NC(=O)NC3=O)c(-c3ccc(F)cc3)n2)c1 ; CCCCCCCOc1ccc(-c2ccc(-n3cc(C4NC(=O)NC4=O)c(-c4ccc(F)cc4)n3)cc2)cc1 ; CCCOCCOCCOCCCNc1cccc(-n2cc(C3NC(=O)NC3=O)c(-c3ccc(F)cc3)n2)c1]

Optionally, more than one activator moiety can be attached to the localizing moiety. In each instance, the activator moiety identified can be functionalized as described herein for methods of attaching a linker and orienting adaptor prior to attachment to the localizing moiety, for example, utilizing the functional groups depicted in a dashed circle.

In an aspect, the Abl kinase activator is DPH or dihyropyrazol activator. An exemplary molecule may comprise

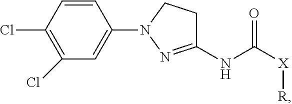

wherein X is (CH2)n, which may be substituted, for example with one or more of amide, acetal, aminal, amine, alkyl, ether, hydrocarbyl, and derivatives thereof, or other groups as described elsewhere herein. In certain embodiments n is 0 to 20, more preferably n is 1 to 10, or 2 to 7, and R is

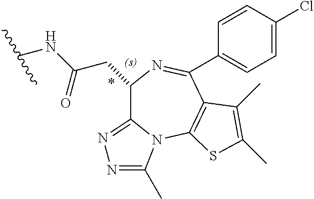 [SMILES: *NC(=O)C[C@@H]1N=C(c2ccc(Cl)cc2)c2c(sc(C)c2C)-n2c(C)nnc21]

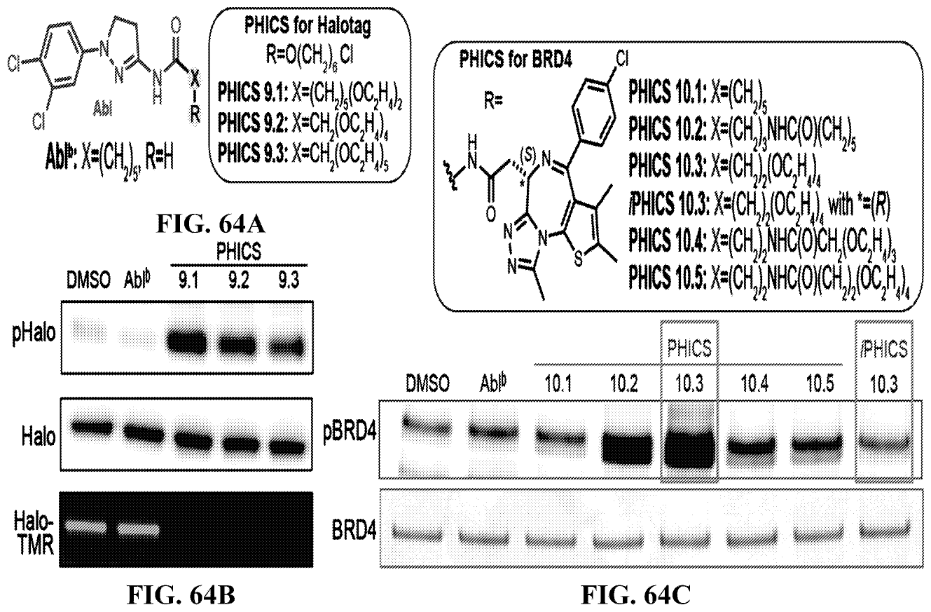

In an aspect, the attachment to the ABL kinase activator dihydropyrazol is via various types of linkers, see, e.g. (PHICS 10.1-10.5, FIG. 64A). In an aspect, when the localizing moiety is for BRD4, an exemplary molecule of

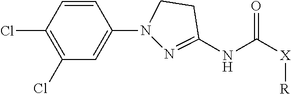

may comprise:

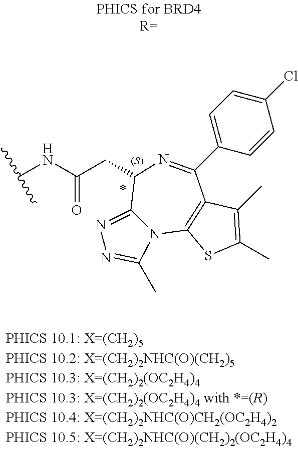

### Insulin Receptor (IRTK) Activator

In certain example embodiments, the kinase activator moiety is a membrane-bound insulin receptor kinase. In an aspect, the activator for ITRK is kojic acid, or a derivative thereof.

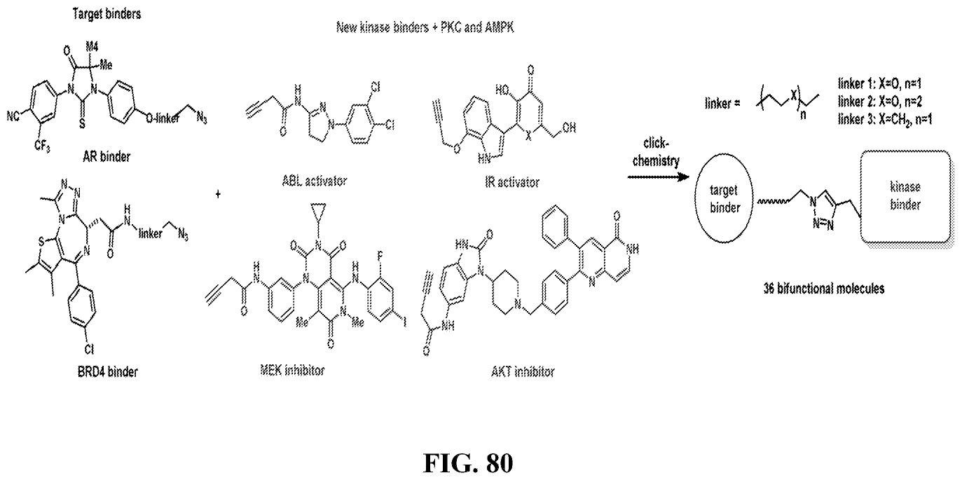

In embodiments, the target is AR. In an aspect, the localizing moiety may comprise enzalutamide. In embodiments, the enzalutamide is attached via an ether bond to a linker comprising an azide end. Thus, in certain embodiments, the addition of alkyne functionality on the activator moiety will enable connection via bioorthogonal click-chemistry. See, e.g. FIG. 80. In certain embodiments, the Insulin Receptor is according to the formula:

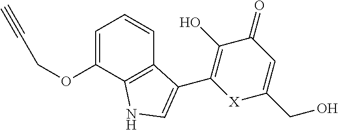

wherein X is C, N, O, S or P.

### CARM-1 Activators

In certain example embodiments, the kinase activator moiety is a CARM1 Activator.

In certain example embodiments, the CARM1 Activator is selected from

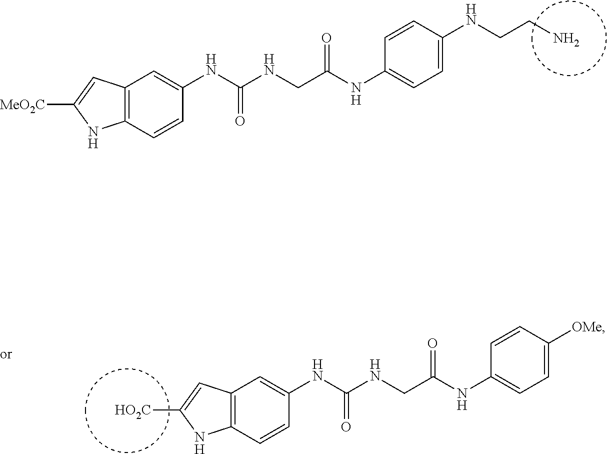 [SMILES: COC(=O)c1cc2cc(NC(=O)NCC(=O)Nc3ccc(NCCN)cc3)ccc2[nH]1 ; COc1ccc(NC(=O)CNC(=O)Nc2ccc3[nH]c(C(=O)O)cc3c2)cc1]

wherein the dashed circle identifies the attachment for the orienting adaptor and linker. The functional groups depicted in the dashed circle of the CARM1 activator can be utilized in methods for attaching a linker and orienting adaptor prior to attachment to the localizing moiety.

### Abl1 Inhibitor

Abl1 is a tyrosine-protein kinase that is implicated in processes of cell differentiation, cell division, cell adhesion, and stress response. Structures of exemplary inhibitors Dasatinib, Imatinib, Ponatinib and Nilotinib are provided in Example 7. In an aspect, the inhibitors are selected for their kinetics and residence times to tune the pharmacological effects based on dose, reduce off-target effects, and optimize residence time. Approaches as described in Example 7 can be used to optimize the effects of multifunctional chimeric molecules of the present invention.

### MEK Inhibitor

In an aspect the moiety is a mitogen-activated protein kinase inhibitor. In an aspect, the MEK inhibitor is trametinib functionalized with alkyne for use in biorthogonal click chemistry reactions with azide functionalized localizing moieties. MEK moieties can be synthesized according to the guidance and design provided herein in view of MEK binding moieties as disclosed, for example, in Sweeney et al. Ann Rheum Dis. 65(3):iii83-iii88 (2006); Wu et al. Pharm Ther 156:59-68 (2015); Suplatov et al. J Biomol Struct Dyn 37(8):2049-2060 (2018); Heald et al. J Medicin Chem 55(10):4594-4604 (2012); Force et al. Circulation 109:1196-1205 (2004).

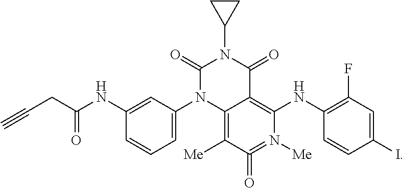

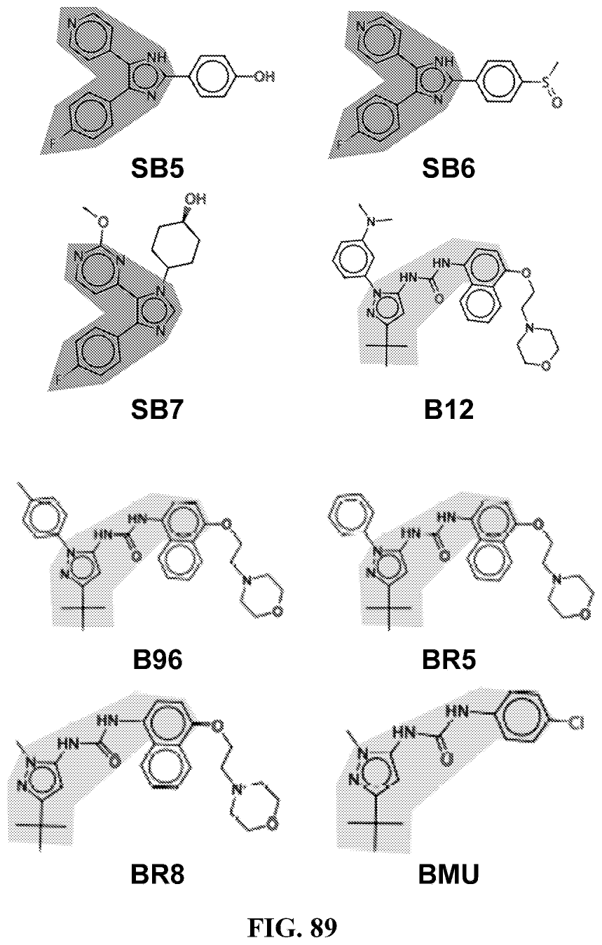

Exemplary p38a mitogen-activated protein kinases inhibitors, SB5, SB6, SB7, B12, B96, BR5, BR8 and BMU as shown in FIG. 89, and derivatives thereof, can be utilized as activating moieties in the multifunctional chimeric molecules of the invention.

### AKT Inhibitor

In an aspect the moiety is a RAC-alpha serine/threonine-protein kinase (AKT) inhibitor. In an aspect, the ATK inhibitor is Borussertib functionalized with alkyne for use in biorthogonal click chemistry reactions with azide functionalized localizing moieties, as described elsewhere herein. AKT moieties can be synthesized according to the guidance and design provided herein in view of AKT binding moieties as disclosed, for example, in Panicker et al. Adv Exp Med Biol 1163:253-278 (2019); Botello-Smith et al. PLoS Comp Biol 13(8):e1005711 (2017); Mou et al. Chem Biol Drug Des 89(5):723-731 (2017); Ruiz-Carillo et al. Sci Rep 8:7365 (2018), and Budas et al. Biochem Soc Trans 35:1021-1026 (2007).

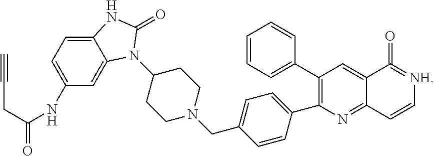

### Orienting Adaptor

The multifunctional molecule may comprise one or more orienting adaptors. In embodiments, an orienting adaptor can be utilized at each instance of a localizing moiety or an activator moiety. In an aspect, an orienting adaptor is appended on different ends of a linker molecule, with an orienting adaptor attached to each activator moiety of the multifunctional molecule, and optionally, provides an orienting adaptor interconnecting the chemical linker molecule on a different end to a localizing moiety.

In embodiments, the orienting adaptor is a small molecule group that aids in the orienting of the localizing moiety and the activator. In embodiments, the orienting adaptors are chosen so that the small molecule compounds bind in one of their preferred, low-energy conformations. By way of example, when a protein is the substrate, a protein dissipates strain energy through small changes across its degrees of freedom more easily than for the small molecule to adopt an unfavorable conformation by straining its few rotatable bonds. Accordingly, ‘soft’ or low-energy torsion barriers are helpful when designing the small molecule compounds. The preferences can be considered when designing orienting molecules between aryl rings. Anisoles (ArOCH₂R) and anilines (ArNHR) prefer coplanar conformations, alkylaryls (ArCH₂R), arylsulfonamides and arylsulfones prefer a perpendicular conformation. Orienting adaptor atoms control both distance and direction. See, e.g. Brameld et al. J. Chem. Inf. Model. 2008, 48, 1-24.

The orienting adaptors can be referred to in embodiments as exit vectors. Exit vector parameters can be identified in part based on average orientation of a substituent attached to a variation point which can be generated using chemoinformatics software. An exit vector may comprise outgoing bonds from a chemical moiety. In certain embodiments, the bond is chosen to be energetically favorable, preferably increasing binding affinity. The orienting adaptor may be represented in certain embodiments with the linker, and may be adjusted depending on the linker utilized in the multifunctional molecules. In embodiments, the orienting adaptor is a chemical moiety or bond that facilitates stereochemical protrusion that may further facilitate subsequent coupling, bonding and/or accessibility. In embodiments, the first and second orienting adaptors are provided as bonds on the linker, providing conformation of attachment between the linker and the activator moiety and/or the localizing moiety.

In embodiments, the first and second orienting adaptor, when present, are independently selected from Table 2 (Orienting Adaptor Table).

TABLE 2

| | |
|---|---|
| 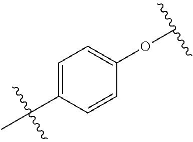 [SMILES: *Oc1ccc(*)cc1] | 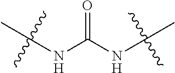 [SMILES: *NC(=O)N*] |

| | |
|---|---|
| 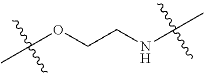 [SMILES: *NCCO*] | 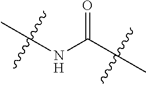 [SMILES: *NC(*)=O] |

| | |
|---|---|
| 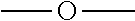 | 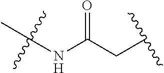 [SMILES: *CC(=O)N*] |

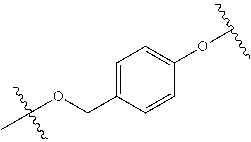 [SMILES: *OCc1ccc(O*)cc1]

| | |
|---|---|
| 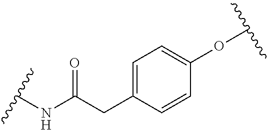 [SMILES: *NC(=O)Cc1ccc(O*)cc1] | 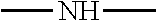 |

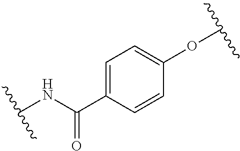 [SMILES: *NC(=O)c1ccc(O*)cc1]

| | |
|---|---|
| 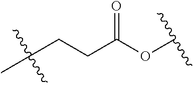 [SMILES: *CCC(=O)O*] | 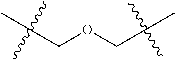 [SMILES: *COC*] |

| | |
|---|---|
| 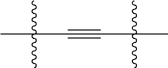 [SMILES: *C#C*] | 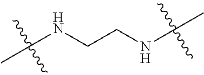 [SMILES: *NCCN*] |

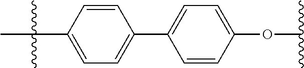 [SMILES: *Oc1ccc(-c2ccc(*)cc2)cc1]

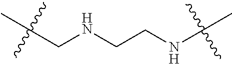 [SMILES: *CNCCN*]

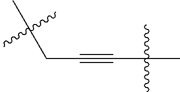 [SMILES: *C#CC*]

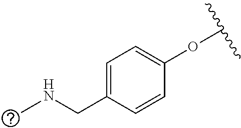 [SMILES: *Oc1ccc(CNC)cc1]

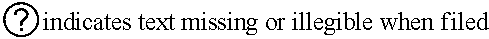

### Methods of Modifying a Target Substrate

The multifunctional molecules disclosed herein can be utilized in methods of modifying a target substrate. Methods of modifying the target substrate can include contacting the target substrate with the multifunctional molecule of the present invention. Contacting can allow for bonding to, or association with the target substrate, or to a molecule in proximity to a target substrate. In an aspect, the target substrate is not a natural substrate of the enzyme, or wherein activation of the enzyme by the activator molecule results in modification of the target substrate by the enzyme at one or more new modification sites that would otherwise remain unmodified by the enzyme when not activated by binding to the activator moiety. Modifying can include the post-translational modification as disclosed herein, including, for example, phosphorylation, hydroxylation, acetylation, methylation, glycosylation, prenylation, amidation, eliminylation, lipidation, acylation, lipoylation, deacetylation, formylation, S-nitrosylation, S-sulfenylation, sulfonylation, sulfinylation, succinylation, sulfation, carbonylation, or alkylation. In an aspect the methods comprise inducing phosphorylation of a protein in the cell. In an aspect, insulin phosphorylation cascade can be replicated by use of the small molecule compounds. The methods may comprise contacting a target substrate with the multifunctional molecule. In particular embodiments, the target substrate is in proximity to a kinase specific to the activator moiety of the molecule. Multifunctional molecules that induce phosphorylation can be optionally provided with adenosine monophosphate (AMP) or another molecule providing an additional phosphate group. Without being bound by theory, the addition of the AMP or other phosphate providing molecule can enhance phosphorylation.

### Target Substrate

Activator moieties of the present invention bind, associate, and/or activate an enzyme that modifies a target substrate associated with the localizing moiety. In an embodiment, the target substrate is not a natural substrate of the enzyme, or the substrate to which the activator moiety associates. In one embodiment, activation of the enzyme by the activator molecule results in modification of the target substrate by the enzyme at one or more new modification sites that would otherwise remain unmodified by the enzyme when not activated by binding to the activator moiety. The target substrate may be bound by the localizing moiety or otherwise associated with the localizing moiety, or may be a substrate known to be in proximity of the localizing moiety, such that the activator moiety is within distance, e.g. proximity, to modify the target substrate. The target substrate is not required to be a natural substrate of the enzyme. The target substrate may be a protein, and discussion herein of genes includes the products of the gene expression. Further applications may be modification of protein:DNA interactions, for example Myc, or protein:protein interactions. Utilization may comprise, for example, phosphorylation to change the charge of a nucleic acid or protein molecule.

By way of non-limiting example, proteins associated with a secretase disorder include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), BACE1 (beta-site APP-cleaving enzyme 1), ITM2B (integral membrane protein 2B), CTSD (cathepsin D), NOTCH1 (Notch homolog 1, translocation-associated (Drosophila)), TNF (tumor necrosis factor (TNF superfamily, member 2)), INS (insulin), DYT10 (dystonia 10), ADAM17 (ADAM metallopeptidase domain 17), APOE (apolipoprotein E), ACE (angiotensin I converting enzyme (peptidyl-dipeptidase A) 1), STN (statin), TP53 (tumor protein p53), IL6 (interleukin 6 (interferon, beta 2)), NGFR (nerve growth factor receptor (TNFR superfamily, member 16)), IL1B (interleukin 1, beta), ACHE (acetylcholinesterase (Yt blood group)), CTNNB1 (catenin (cadherin-associated protein), beta 1, 88 kDa), IGF1 (insulin-like growth factor 1 (somatomedin C)), IFNG (interferon, gamma), NRG1 (neuregulin 1), CASP3 (caspase 3, apoptosis-related cysteine peptidase), MAPK1 (mitogen-activated protein kinase 1), CDH1 (cadherin 1, type 1, E-cadherin (epithelial)), APBB1 (amyloid beta (A4) precursor protein-binding, family B, member 1 (Fe65)), HMGCR (3-hydroxy-3-methylglutaryl-Coenzyme A reductase), CREB1 (cAMP responsive element binding protein 1), PTGS2 (prostaglandin-endoperoxide synthase 2 (prostaglandin G/H synthase and cyclooxygenase)), HES1 (hairy and enhancer of split 1, (Drosophila)), CAT (catalase), TGFB1 (transforming growth factor, beta 1), ENO2 (enolase 2 (gamma, neuronal)), ERBB4 (v-erb-a erythroblastic leukemia viral oncogene homolog 4 (avian)), TRAPPC10 (trafficking protein particle complex 10), MAOB (monoamine oxidase B), NGF (nerve growth factor (beta polypeptide)), MMP12 (matrix metallopeptidase 12 (macrophage elastase)), JAG1 (jagged 1 (Alagille syndrome)), CD40LG (CD40 ligand), PPARG (peroxisome proliferator-activated receptor gamma), FGF2 (fibroblast growth factor 2 (basic)), IL3 (interleukin 3 (colony-stimulating factor, multiple)), LRP1 (low density lipoprotein receptor-related protein 1), NOTCH4 (Notch homolog 4 (Drosophila)), MAPK8 (mitogen-activated protein kinase 8), PREP (prolyl endopeptidase), NOTCH3 (Notch homolog 3 (Drosophila)), PRNP (prion protein), CTSG (cathepsin G), EGF (epidermal growth factor (beta-urogastrone)), REN (renin), CD44 (CD44 molecule (Indian blood group)), SELP (selectin P (granule membrane protein 140 kDa, antigen CD62)), GHR (growth hormone receptor), ADCYAP1 (adenylate cyclase activating polypeptide 1 (pituitary)), INSR (insulin receptor), GFAP (glial fibrillary acidic protein), MMP3 (matrix metallopeptidase 3 (stromelysin 1, progelatinase)), MAPK10 (mitogen-activated protein kinase 10), SP1 (Sp1 transcription factor), MYC (v-myc myelocytomatosis viral oncogene homolog (avian)), CTSE (cathepsin E), PPARA (peroxisome proliferator-activated receptor alpha), JUN (jun oncogene), TIMP1 (TIMP metallopeptidase inhibitor 1), IL5 (interleukin 5 (colony-stimulating factor, eosinophil)), IL1A (interleukin 1, alpha), MMP9 (matrix metallopeptidase 9 (gelatinase B, 92 kDa gelatinase, 92 kDa type IV collagenase)), HTR4 (5-hydroxytryptamine (serotonin) receptor 4), HSPG2 (heparan sulfate proteoglycan 2), KRAS (v-Ki-ras2 Kirsten rat sarcoma viral oncogene homolog), CYCS (cytochrome c, somatic), SMG1 (SMG1 homolog, phosphatidylinositol 3-kinase-related kinase (C. elegans)), IL1R1 (interleukin 1 receptor, type I), PROK1 (prokineticin 1), MAPK3 (mitogen-activated protein kinase 3), NTRK1 (neurotrophic tyrosine kinase, receptor, type 1), IL13 (interleukin 13), MME (membrane metallo-endopeptidase), TKT (transketolase), CXCR2 (chemokine (C—X—C motif) receptor 2), IGF1R (insulin-like growth factor 1 receptor), RARA (retinoic acid receptor, alpha), CREBBP (CREB binding protein), PTGS1 (prostaglandin-endoperoxide synthase 1 (prostaglandin G/H synthase and cyclooxygenase)), GALT (galactose-1-phosphate uridylyltransferase), CHRM1 (cholinergic receptor, muscarinic 1), ATXN1 (ataxin 1), PAWR (PRKC, apoptosis, WT1, regulator), NOTCH2 (Notch homolog 2 (Drosophila)), M6PR (mannose-6-phosphate receptor (cation dependent)), CYP46A1 (cytochrome P450, family 46, subfamily A, polypeptide 1), CSNK1 D (casein kinase 1, delta), MAPK14 (mitogen-activated protein kinase 14, also called p38-α), PRG2 (proteoglycan 2, bone marrow (natural killer cell activator, eosinophil granule major basic protein)), PRKCA (protein kinase C, alpha), L1 CAM (L1 cell adhesion molecule), CD40 (CD40 molecule, TNF receptor superfamily member 5), NR1I2 (nuclear receptor subfamily 1, group I, member 2), JAG2 (jagged 2), CTNND1 (catenin (cadherin-associated protein), delta 1), CDH2 (cadherin 2, type 1, N-cadherin (neuronal)), CMA1 (chymase 1, mast cell), SORT1 (sortilin 1), DLK1 (delta-like 1 homolog (Drosophila)), THEM4 (thioesterase superfamily member 4), JUP (junction plakoglobin), CD46 (CD46 molecule, complement regulatory protein), CCL11 (chemokine (C—C motif) ligand 11), CAV3 (caveolin 3), RNASE3 (ribonuclease, RNase A family, 3 (eosinophil cationic protein)), HSPA8 (heat shock 70 kDa protein 8), CASP9 (caspase 9, apoptosis-related cysteine peptidase), CYP3A4 (cytochrome P450, family 3, subfamily A, polypeptide 4), CCR3 (chemokine (C—C motif) receptor 3), TFAP2A (transcription factor AP-2 alpha (activating enhancer binding protein 2 alpha)), SCP2 (sterol carrier protein 2), CDK4 (cyclin-dependent kinase 4), HIF1A (hypoxia inducible factor 1, alpha subunit (basic helix-loop-helix transcription factor)), TCF7L2 (transcription factor 7-like 2 (T-cell specific, HMG-box)), IL1R2 (interleukin 1 receptor, type II), B3GALTL (beta 1,3-galactosyltransferase-like), MDM2 (Mdm2 p53 binding protein homolog (mouse)), RELA (v-rel reticuloendotheliosis viral oncogene homolog A (avian)), CASP7 (caspase 7, apoptosis-related cysteine peptidase), IDE (insulin-degrading enzyme), FABP4 (fatty acid binding protein 4, adipocyte), CASK (calcium/calmodulin-dependent serine protein kinase (MAGUK family)), ADCYAP1R1 (adenylate cyclase activating polypeptide 1 (pituitary) receptor type I), ATF4 (activating transcription factor 4 (tax-responsive enhancer element B67)), PDGFA (platelet-derived growth factor alpha polypeptide), C21 or f33 (chromosome 21 open reading frame 33), SCG5 (secretogranin V (7B2 protein)), RNF123 (ring finger protein 123), NFKB1 (nuclear factor of kappa light polypeptide gene enhancer in B-cells 1), ERBB2 (v-erb-b2 erythroblastic leukemia viral oncogene homolog 2, neuro/glioblastoma derived oncogene homolog (avian)), CAV1 (caveolin 1, caveolae protein, 22 kDa), MMP7 (matrix metallopeptidase 7 (matrilysin, uterine)), TGFA (transforming growth factor, alpha), RXRA (retinoid X receptor, alpha), STX1A (syntaxin 1A (brain)), PSMC4 (proteasome (prosome, macropain) 26S subunit, ATPase, 4), P2RY2 (purinergic receptor P2Y, G-protein coupled, 2), TNFRSF21 (tumor necrosis factor receptor superfamily, member 21), DLG1 (discs, large homolog 1 (Drosophila)), NUMBL (numb homolog (Drosophila)-like), SPN (sialophorin), PLSCR1 (phospholipid scramblase 1), UBQLN2 (ubiquilin 2), UBQLN1 (ubiquilin 1), PCSK7 (proprotein convertase subtilisin/kexin type 7), SPON1 (spondin 1, extracellular matrix protein), SILV (silver homolog (mouse)), QPCT (glutaminyl-peptide cyclotransferase), HESS (hairy and enhancer of split 5 (Drosophila)), GCC1 (GRIP and coiled-coil domain containing 1), and any combination thereof.

Additional targets can include targets implicated in fatty acid disorders. In certain embodiments, the target is one or more of ACADM, HADHA, ACADVL. In embodiments, the targeted edit is the activity of a gene in a cell selected from the acyl-coenzyme A dehydrogenase for medium chain fatty acids (ACADM) gene, the long-chain 3-hydroxyl-coenzyme A dehydrogenase for long chain fatty acids (HADHA) gene, and the acyl-coenzyme A dehydrogenase for very long-chain fatty acids (ACADVL) gene. In one aspect, the disease is medium chain acyl-coenzyme A dehydrogenase deficiency (MCADD), long-chain 3-hydroxyl-coenzyme A dehydrogenase deficiency (LCHADD), and/or very long-chain acyl-coenzyme A dehydrogenase deficiency (VLCADD). An additional target can be Angiopoietin-like 4 (ANGPTL4). Diseases or disorders associated with ANGPTL4 that can be treated include ANGPTL4 is associated with dyslipidemias, low plasma triglyceride levels, regulator of angiogenesis and modulate tumorigenesis, and severe diabetic retinopathy.

In certain embodiments, the disease or disorder is associated with Apolipoprotein C3 (APOCIII), which can be targeted for modification. In some embodiments, the target can comprise Recombination Activating Gene 1 (RAG1), BCL11 A, PCSK9, laminin, alpha 2 (lama2), ATXN3, alanine-glyoxylate aminotransferase (AGXT), collagen type vii alpha 1 chain (COL7a1), spinocerebellar ataxia type 1 protein (ATXN1), Angiopoietin-like 3 (ANGPTL3), Frataxin (FXN), Superoxidase Dismutase 1, soluble (SOD1), Synuclein, Alpha (SNCA), Sodium Channel, Voltage Gated, Type X Alpha Subunit (SCN10A), Spinocerebellar Ataxia Type 2 Protein (ATXN2), Dystrophia Myotonica-Protein Kinase (DMPK), beta globin locus on chromosome 11, acyl-coenzyme A dehydrogenase for medium chain fatty acids (ACADM), long-chain 3-hydroxyl-coenzyme A dehydrogenase for long chain fatty acids (HADHA), acyl-coenzyme A dehydrogenase for very long-chain fatty acids (ACADVL), Apolipoprotein C3 (APOCIII), Transthyretin (TTR), Angiopoietin-like 4 (ANGPTL4), Sodium Voltage-Gated Channel Alpha Subunit 9 (SCN9A), Interleukin-7 receptor (IL7R), glucose-6-phosphatase, catalytic (G6PC), haemochromatosis (HFE), SERPINA1, C90RF72, β-globin, dystrophin, γ-globin.

In certain embodiments, the target is associated with particular genes. The target may be an AAVS1 (PPPIR12C), an ALB gene, an Angptl3 gene, an ApoC3 gene, an ASGR2 gene, a CCR5 gene, a FIX (F9) gene, a G6PC gene, a Gys2 gene, an HGD gene, a Lp(a) gene, a Pcsk9 gene, a Serpinal gene, a TF gene, and a TTR gene). Assessment of efficiency of HDR/NHEJ mediated knock-in of cDNA into the first exon can utilize cDNA knock-in into “safe harbor” sites such as: single-stranded or double-stranded DNA having homologous arms to one of the following regions, for example: ApoC3 (chr11:116829908-116833071), Angptl3 (chr1:62,597,487-62,606,305), Serpinal (chr14:94376747-94390692), Lp(a) (chr6:160531483-160664259), Pcsk9 (chr1:55,039,475-55,064,852), FIX (chrX:139,530,736-139,563,458), ALB (chr4:73,404,254-73,421,411), TTR (chr1 8:31,591,766-31,599,023), TF (chr3:133,661,997-133,779,005), G6PC (chr17:42,900,796-42,914,432), Gys2 (chr12:21,536,188-21,604,857), AAVS1 (PPP1R12C) (chr19:55,090,912-55,117,599), HGD (chr3:120,628,167-120,682,570), CCR5 (chr3:46,370,854-46,376,206), or ASGR2 (chr17:7,101,322-7,114,310). In one aspect, the target is superoxide dismutase 1, soluble (SOD1).

In an aspect, the disease is associated with cancer. In an aspect, neophosphorylation on oncogenic targets may elicit immune reaction, or phosphorylation is used as an autoantigen. In an aspect, the phosphorylation is used in multiple sclerosis, e.g. uB-crystallin, or in SLE with multiple targets (see, e.g. Doyle and Mamula, Curr Opin Immunol. 2012). In some embodiments, the disease is associated with expression of a tumor antigen, e.g., a proliferative disease, a precancerous condition, a cancer, or a non-cancer related indication associated with expression of the tumor antigen, which may in some embodiments comprise a target selected from B2M, CD247, CD3D, CD3E, CD3G, TRAC, TRBC1, TRBC2, HLA-A, HLA-B, HLA-C, DCK, CD52, FKBPlA, CIITA, NLRC5, RFXANK, RFX5, RFXAP, or NR3C1, HAVCR2, LAG3, PDCD1, PD-L2, CTLA4, CEACAM (CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, or PTPN11 DCK, CD52, NR3C1, LILRB1, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); n kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1), CD19, BCMA, CD70, G6PC, Dystrophin, including modification of exon 51 by deletion or excision, DMPK, CFTR (cystic fibrosis transmembrane conductance regulator). In embodiments, the targets comprise CD70, or a Knock-in of CD33 and Knockout of B2M. In embodiments, the targets comprise a knockout of TRAC and B2M, or TRAC B2M and PD1, with or without additional target genes. In certain embodiments, the disease is cystic fibrosis with targeting of the SCNN1A gene. In one application, the small molecules disclosed herein are utilized in human leukocyte antigen (HLA) display and immune response.

In particular embodiments it is envisaged to specifically modify genes that are involved in the modification of the quantity of lipids and/or the quality of the lipids produced by the algal cell. Examples of genes encoding enzymes involved in the pathways of fatty acid synthesis can encode proteins having for instance acetyl-CoA carboxylase, fatty acid synthase, 3-ketoacyl_acyl-carrier protein synthase III, glycerol-3-phospate dehydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid:diacylglycerol acyltransferase, phoshatidate phosphatase, fatty acid thioesterase such as palmitoyi protein thioesterase, or malic enzyme activities. In further embodiments it is envisaged to generate diatoms that have increased lipid accumulation. This can be achieved by targeting genes that decrease lipid catabolisation. Of particular interest for use in the methods of the present invention are genes involved in the activation of both triacylglycerol and free fatty acids, as well as genes directly involved in β-oxidation of fatty acids, such as acyl-CoA synthetase, 3-ketoacyl-CoA thiolase, acyl-CoA oxidase activity and phosphoglucomutase. The system and methods described herein can be used to specifically activate such genes in diatoms as to increase their lipid content.

In some embodiments, the disease is Metachromatic Leukodystrophy, and the target is Arylsulfatase A, the disease is Wiskott-Aldrich Syndrome and the target is Wiskott-Aldrich Syndrome protein, the disease is Adreno leukodystrophy and the target is ATP-binding cassette DI, the disease is Human Immunodeficiency Virus and the target is receptor type 5-C—C chemokine or CXCR4 gene, the disease is Beta-thalassemia and the target is Hemoglobin beta subunit, the disease is X-linked Severe Combined ID receptor subunit gamma and the target is interelukin-2 receptor subunit gamma, the disease is Multisystemic Lysosomal Storage Disorder cystinosis and the target is cystinosin, the disease is Diamon-Blackfan anemia and the target is Ribosomal protein S19, the disease is Fanconi Anemia and the target is Fanconi anemia complementation groups (e.g. FNACA, FNACB, FANCC, FANCD1, FANCD2, FANCE, FANCF, RAD51C), the disease is Shwachman-Bodian-Diamond Bodian-Diamond syndrome and the target is Shwachman syndrome gene, the disease is Gaucher's disease and the target is Glucocerebrosidase, the disease is Hemophilia A and the target is Anti-hemophiliac factor OR Factor VIII, Christmas factor, Serine protease, Factor Hemophilia B IX, the disease is Adenosine deaminase deficiency (ADA-SCID) and the target is Adenosine deaminase, the disease is GM1 gangliosidoses and the target is beta-galactosidase, the disease is Glycogen storage disease type II, Pompe disease, the disease is acid maltase deficiency acid and the target is alpha-glucosidase, the disease is Niemann-Pick disease, SMPD1-associated (Types Sphingomyelin phosphodiesterase 1 OR A and B) acid and the target is sphingomyelinase, the disease is Krabbe disease, globoid cell leukodystrophy and the target is Galactosylceramidase or galactosylceramide lipidosis and the target is galactercerebrosidease, Human leukocyte antigens DR-15, DQ-6, the disease is Multiple Sclerosis (MS) DRB1, the disease is Herpes Simplex Virus 1 or 2 and the target is knocking down of one, two or three of RS1, RL2 and/or LAT genes. In embodiments, the disease is an HPV associated cancer with treatment including edited cells comprising binding molecules, such as TCRs or antigen binding fragments thereof and antibodies and antigen-binding fragments thereof, such as those that recognize or bind human papilloma virus. The disease can be Hepatitis B with a target of one or more of PreC, C, X, PreS1, PreS2, S, P and/or SP gene(s).

Chromosomal Sequence Encoded Protein ALAS2 Delta-aminolevulinate synthase 2 (ALAS2) ABCA1 ATP-binding cassette transporter (ABCA1) ACE Angiotensin I-converting enzyme (ACE) APOE Apolipoprotein E precursor (APOE) APP amyloid precursor protein (APP) AQP1 aquaporin 1 protein (AQP1) BIN1 Myc box-dependent-interacting protein 1 or bridging integrator 1 protein (BIN1) BDNF brain-derived neurotrophic factor (BDNF) BTNL8 Butyrophilin-like protein 8 (BTNL8) C10RF49 chromosome 1 open reading frame 49 CDH4 Cadherin-4 CHRNB2 Neuronal acetylcholine receptor subunit beta-2 CKLFSF2 CKLF-like MARVEL transmembrane domain-containing protein 2 (CKLFSF2) CLEC4E C-type lectin domain family 4, member e (CLEC4E) CLU clusterin protein (also known as apoplipoprotein J) CR1 Erythrocyte complement receptor 1 (CR1, also known as CD35, C3b/C4b receptor and immune adherence receptor) CR1L Erythrocyte complement receptor 1 (CR1L) CSF3R granulocyte colony-stimulating factor 3 receptor (CSF3R) CST3 Cystatin C or cystatin 3 CYP2C Cytochrome P450 2C DAPK1 Death-associated protein kinase 1 (DAPK1) ESR1 Estrogen receptor 1 FCAR Fc fragment of IgA receptor (FCAR, also known as CD89) FCGR3B Fc fragment of IgG, low affinity IIb, receptor (FCGR3B or CD16b) FFA2 Free fatty acid receptor 2 (FFA2) FGA Fibrinogen (Factor I) GAB2 GRB2-associated-binding protein 2 (GAB2) GAB2 GRB2-associated-binding protein 2 (GAB2) GALP Galanin-like peptide GAPDHS Glyceraldehyde-3-phosphate dehydrogenase, spermatogenic (GAPDHS) GMPB GMBP HP Haptoglobin (HP) HTR7 5-hydroxytryptamine (serotonin) receptor 7 (adenylate cyclase-coupled) IDE Insulin degrading enzyme IF127 IF127 IF16 Interferon, alpha-inducible protein 6 (IF16) IFIT2 Interferon-induced protein with tetratricopeptide repeats 2 (IFIT2) IL1RN interleukin-1 receptor antagonist (IL-iRA) IL8RA Interleukin 8 receptor, alpha (IL8RA or CD181) IL8RB Interleukin 8 receptor, beta (IL8RB) JAG1 Jagged 1 (JAG1) KCNJ15 Potassium inwardly-rectifying channel, subfamily J, member 15 (KCNJ15) LRP6 Low-density lipoprotein receptor-related protein 6 (LRP6) MAPT microtubule-associated protein tau (MAPT) MARK4 MAP/microtubule affinity-regulating kinase 4 (MARK4) MPHOSPH1 M-phase phosphoprotein 1 MTHFR 5,10-methylenetetrahydrofolate reductase MX2 Interferon-induced GTP-binding protein Mx2 NBN Nibrin, also known as NBN NCSTN Nicastrin NIACR2 Niacin receptor 2 (NIACR2, also known as GPR109B) NMNAT3 nicotinamide nucleotide adenylyltransferase 3 NTM Neurotrimin (or HNT) ORM1 Orosmucoid 1 (ORM1) or Alpha-1-acid glycoprotein 1 P2RY13 P2Y purinoceptor 13 (P2RY13) PBEF1 Nicotinamide phosphoribosyltransferase (NAmPRTase or Nampt) also known as pre-B-cell colony-enhancing factor 1 (PBEF1) or visfatin PCK1 Phosphoenolpyruvate carboxykinase PICALM phosphatidylinositol binding clathrin assembly protein (PICALM) PLAU Urokinase-type plasminogen activator (PLAU) PLXNC1 Plexin C1 (PLXNC1) PRNP Prion protein PSEN1 presenilin 1 protein (PSEN1) PSEN2 presenilin 2 protein (PSEN2) PTPRA protein tyrosine phosphatase receptor type A protein (PTPRA) RALGPS2 Ral GEF with PH domain and SH3 binding motif 2 (RALGPS2) RGSL2 regulator of G-protein signaling like 2 (RGSL2) SELENBP1 Selenium binding protein 1 (SELNBP1) SLC25A37 Mitoferrin-1 SORL1 sortilin-related receptor L (DLR class) A repeats-containing protein (SORL1) TF Transferrin TFAM Mitochondrial transcription factor A TNF Tumor necrosis factor TNFRSF10C Tumor necrosis factor receptor superfamily member 10C (TNFRSF10C) TNFSF10 Tumor necrosis factor receptor superfamily, (TRAIL) member 10a (TNFSF10) UBA1 ubiquitin-like modifier activating enzyme 1 (UBA1) UBA3 NEDD8-activating enzyme E1 catalytic subunit protein (UBE1C) UBB ubiquitin B protein (UBB) UBQLN1 Ubiquilin-1 UCHL1 ubiquitin carboxyl-terminal esterase L1 protein (UCHL1) UCHL3 ubiquitin carboxyl-terminal hydrolase isozyme L3 protein (UCHL3) VLDLR very low density lipoprotein receptor protein (VLDLR).

Targets can include very low density lipoprotein receptor protein (VLDLR) encoded by the VLDLR gene, the ubiquitin-like modifier activating enzyme 1 (UBA1) encoded by the UBA1 gene, the NEDD8-activating enzyme E1 catalytic subunit protein (UBE1C) encoded by the UBA3 gene, the aquaporin 1 protein (AQP1) encoded by the AQP1 gene, the ubiquitin carboxyl-terminal esterase L1 protein (UCHL1) encoded by the UCHL1 gene, the ubiquitin carboxyl-terminal hydrolase isozyme L3 protein (UCHL3) encoded by the UCHL3 gene, the ubiquitin B protein (UBB) encoded by the UBB gene, the microtubule-associated protein tau (MAPT) encoded by the MAPT gene, the protein tyrosine phosphatase receptor type A protein (PTPRA) encoded by the PTPRA gene, the phosphatidylinositol binding clathrin assembly protein (PICALM) encoded by the PICALM gene, the clusterin protein (also known as apoplipoprotein J) encoded by the CLU gene, the presenilin 1 protein encoded by the PSEN1 gene, the presenilin 2 protein encoded by the PSEN2 gene, the sortilin-related receptor L (DLR class) A repeats-containing protein (SORL1) protein encoded by the SORL1 gene, the amyloid precursor protein (APP) encoded by the APP gene, the Apolipoprotein E precursor (APOE) encoded by the APOE gene, or the brain-derived neurotrophic factor (BDNF) encoded by the BDNF gene. In an exemplary embodiment, the genetically modified animal is a rat, and the edited chromosomal sequence encoding the protein associated with AD is as follows: APP amyloid precursor protein (APP) NM_019288 AQP1 aquaporin 1 protein (AQP1) NM_012778 BDNF Brain-derived neurotrophic factor NM_012513 CLU clusterin protein (also known as NM_053021 apoplipoprotein J) MAPT microtubule-associated protein NM_017212 tau (MAPT) PICALM phosphatidylinositol binding NM_053554 clathrin assembly protein (PICALM) PSEN1 presenilin 1 protein (PSEN1) NM_019163 PSEN2 presenilin 2 protein (PSEN2) NM_031087 PTPRA protein tyrosine phosphatase NM_012763 receptor type A protein (PTPRA) SORL1 sortilin-related receptor L (DLR NM_053519, class) A repeats-containing XM_001065506, protein (SORL1) XM_217115 UBA1 ubiquitin-like modifier activating NM 001014080 enzyme 1 (UBA1) UBA3 NEDD8-activating enzyme E1 NM_057205 catalytic subunit protein (UBE1C) UBB ubiquitin B protein (UBB) NM_138895 UCHL1 ubiquitin carboxyl-terminal NM_017237 esterase L1 protein (UCHL1) UCHL3 ubiquitin carboxyl-terminal NM_001110165 hydrolase isozyme L3 protein (UCHL3) VLDLR very low density lipoprotein NM_013155 receptor protein (VLDLR)

### Delivery of Compounds

Methods for modifying a target of interest comprises administering or delivering or otherwise contacting a cell via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the composition is introduced into an embryo by microinjection. The compositions may be microinjected into the nucleus or the cytoplasm of the embryo.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these aspects are within the ambit of the skilled artisan. In the field of active targeting, there are a number of cell-, e.g., tumor-, specific targeting ligands.

Also as to active targeting, with regard to targeting cell surface receptors such as cancer cell surface receptors, targeting ligands on liposomes can provide attachment of liposomes to cells, e.g., vascular cells, via a noninternalizing epitope; and, this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR is can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm. With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation. Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be been utilized for targeted delivery in the invention, e.g., whereby the targeting moiety can be an RGD peptide such as a cyclic RGD. Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer). The targeting moiety can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)). Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes. The invention also comprehends redox-triggered delivery: The difference in redox potential between normal and inflamed or tumor tissues, and between the intra- and extra-cellular environments has been exploited for delivery; e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment. Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 1) can be incorporated into a linker, and can have antibody targeting, e.g., antibody 2C5. The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe2O3, e.g., those that are less than 10 nm in size. Targeted delivery can be then by exposure to a magnetic field.

Also as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

Also as to active targeting, cell-penetrating peptides (CPPs) facilitate uptake of macromolecules through cellular membranes and, thus, enhance the delivery of CPP-modified molecules inside the cell. CPPs can be split into two classes: amphipathic helical peptides, such as transportan and MAP, where lysine residues are major contributors to the positive charge; and Arg-rich peptides, such as TATp, Antennapedia or penetratin. TATp is a transcription-activating factor with 86 amino acids that contains a highly basic (two Lys and six Arg among nine residues) protein transduction domain, which brings about nuclear localization and RNA binding. Other CPPs that have been used for the modification of liposomes include the following: the minimal protein transduction domain of Antennapedia, a Drosophila homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, multipara, a wasp venom peptide; VP22, a major structural component of HSV-1 facilitating intracellular transport and transportan (18-mer) amphipathic model peptide that translocates plasma membranes of mast cells and endothelial cells by both energy-dependent and -independent mechanisms. The invention comprehends a lipid entity of the invention modified with CPP(s), for intracellular delivery that may proceed via energy dependent micropinocytosis followed by endosomal escape. The invention further comprehends organelle-specific targeting. A lipid entity of the invention surface-functionalized with the triphenyl phosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

An embodiment of the system may comprise an actively targeting lipid particle or nanoparticle or liposome or lipid bilayer delivery system; or a lipid particle or nanoparticle or liposome or lipid bilayer comprising a targeting moiety whereby there is active targeting or wherein the targeting moiety is an actively targeting moiety. A targeting moiety can be one or more targeting moieties, and a targeting moiety can be for any desired type of targeting such as, e.g., to target a cell such as any herein-mentioned; or to target an organelle such as any herein-mentioned; or for targeting a response such as to a physical condition such as heat, energy, ultrasound, light, pH, chemical such as enzymatic, or magnetic stimuli; or to target to achieve a particular outcome such as delivery of payload to a particular location, such as by cell penetration.

It should be understood that as to each possible targeting or active targeting moiety herein-discussed, there is an aspect of the invention wherein the delivery system comprises such a targeting or active targeting moiety.

### Pharmaceutical Compositions

The methods described herein include the manufacture and use of pharmaceutical compositions, which include an agent described herein as active ingredient(s). Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, and combinations of two or more thereof, compatible with pharmaceutical administration. Supplementary active compounds can also be incorporated into the compositions.

Pharmaceutical compositions are typically formulated to be compatible with the intended route of administration. Examples of routes of administration that are especially useful in the present methods include parenteral (e.g., intravenous), intrathecal, oral, and nasal or intranasal (e.g., by administration as drops or inhalation) administration. In some embodiments, such as for compounds that don't cross the blood brain barrier, delivery directly into the CNS or CSF can be used, e.g., using implanted intrathecal pumps (see, e.g., Borrini et al., Archives of Physical Medicine and Rehabilitation 2014; 95:1032-8; Penn et al., N. Eng. J. Med. 320:1517-21 (1989); and Rezai et al., Pain Physician 2013; 16:415-417) or nanoparticles, e.g., gold nanoparticles (e.g., glucose-coated gold nanoparticles, see, e.g., Gromnicova et al. (2013) PLoS ONE 8(12): e81043). Methods of formulating and delivering suitable pharmaceutical compositions are known in the art, see, e.g., the books in the series Drugs and the Pharmaceutical Sciences: a Series of Textbooks and Monographs (Dekker, N.Y.); and Allen et al., Ansel's Pharmaceutical Dosage Forms and Drug Delivery Systems, Lippincott Williams & Wilkins; 8th edition (2004).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

For oral administration, the compositions can be formulated with an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998).

Liposomes (e.g., as described in U.S. Pat. No. 6,472,375) and microencapsulation can also be used to deliver a compound described herein. Biodegradable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser, e.g., single-dose dispenser together with instructions for administration. The container, pack, or dispenser can also be included as part of a kit that can include, for example, sufficient single-dose dispensers for one day, one week, or one month of treatment.

### Dosage

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect, which may be tied to the degree of modifications made. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

### Methods of Treatment

The present invention also contemplates use of the systems described herein, for treatment in a variety of diseases and disorders. A localizing moiety can bind to a target of interest, or localize in the region of a target of interest, allowing the activating moiety to modify a target. The present invention also contemplates use of the multifunctional molecules described herein, for treatment in a variety of diseases and disorders. Exemplary applications include use as small-molecule analogs of insulin and rewiring of cellular signaling. See, Lim et al., Nat Rev Mol Cell Biol 2010, 11(6), 393-403. Requiring cell signaling can be addressed by appending phosphoryl groups to specific signaling protein of interest with dose and temporal control to allow rewiring of kinase signaling pathways in disease or health. The multifunctional systems herein may enable targeted degradation of the protein where phosphorylation sites are targets that recruit ubiquitin ligase and signal degradation. See, Toure et al., Angewandte Chemie (Inter'l ed. In English) 2016, 55(6), 1966-73. Similarly, preventing protein aggregation can aid in treatment in cancer treatment approaches. As described herein, addition of negatively charged phosphoryl groups using the multifunctional molecules on a protein prone to aggregation may increase solubility and reduce self-aggregation. Guo et al., FEBS Letters, 2005, 579 (17), 3574-3578; Zhang et al., Protein Expression and Purification 2004 36(2) 207-216. Finally, neo-phosphorylation to elicit an immune response can find use an cancer immunotherapy approaches. Treatment of kinasopathies are also contemplated, see, generally, Lahiry et al., Nature Reviews Genetics, 2011, with Table 1 disclosure of inherited kinasopathies incorporated herein by reference. Methods of modifying a target substrate in a subject in need thereof is provided, the method comprising administering a molecule as disclosed herein to the subject. Delivery can be as described elsewhere herein. In embodiments, the invention described herein relates to a method for therapy in which cells are modified ex vivo by the multifunctional molecules to modify at least one target substrate, with subsequent administration of the edited cells to a patient in need thereof.

In embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

Particular diseases/disorders include chondroplasia, achromatopsia, acid maltase deficiency, adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefelter syndrome, Krabbes Disease, Langer-Giedion Syndrome, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, and Wiskott-Aldrich syndrome.

In embodiments, the disease is associated with expression of a tumor antigen, e.g., a proliferative disease, a precancerous condition, a cancer, or a non-cancer related indication associated with expression of the tumor antigen, which may in some embodiments comprise a target selected from B2M, CD247, CD3D, CD3E, CD3G, TRAC, TRBC1, TRBC2, HLA-A, HLA-B, HLA-C, DCK, CD52, FKBP1A, CIITA, NLRC5, RFXANK, RFX5, RFXAP, or NR3C1, HAVCR2, LAG3, PDCD1, PD-L2, CTLA4, CEACAM (CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, or PTPN11 DCK, CD52, NR3C1, LILRB1, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11R^{a}); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); n kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1), CD19, BCMA, CD70, G6PC, Dystrophin, DMPK, CFTR (cystic fibrosis transmembrane conductance regulator).

In embodiments, the disease is Metachromatic Leukodystrophy, and the target is Arylsulfatase A, the disease is Wiskott-Aldrich Syndrome and the target is Wiskott-Aldrich Syndrome protein, the disease is Adreno leukodystrophy and the target is ATP-binding cassette DI, the disease is Human Immunodeficiency Virus and the target is receptor type 5-C—C chemokine or CXCR4 gene, the disease is Beta-thalassemia and the target is Hemoglobin beta subunit, the disease is X-linked Severe Combined ID receptor subunit gamma and the target is interelukin-2 receptor subunit gamma, the disease is Multisystemic Lysosomal Storage Disorder cystinosis and the target is cystinosin, the disease is Diamon-Blackfan anemia and the target is Ribosomal protein S19, the disease is Fanconi Anemia and the target is Fanconi anemia complementation groups (e.g. FNACA, FNACB, FANCC, FANCD1, FANCD2, FANCE, FANCF, RAD51C), the disease is Shwachman-Bodian-Diamond Bodian-Diamond syndrome and the target is Shwachman syndrome gene, the disease is Gaucher's disease and the target is Glucocerebrosidase, the disease is Hemophilia A and the target is Anti-hemophiliac factor OR Factor VIII, Christmas factor, Serine protease, Factor Hemophilia B IX, the disease is Adenosine deaminase deficiency (ADA-SCID) and the target is Adenosine deaminase, the disease is GM1 gangliosidoses and the target is beta-galactosidase, the disease is Glycogen storage disease type II, Pompe disease, the disease is acid maltase deficiency acid and the target is alpha-glucosidase, the disease is Niemann-Pick disease, SMPDl-associated (Types Sphingomyelin phosphodiesterase 1 OR A and B) acid and the target is sphingomyelinase, the disease is Krabbe disease, globoid cell leukodystrophy and the target is Galactosylceramidase or galactosylceramide lipidosis and the target is galactercerebrosidease, Human leukocyte antigens DR-15, DQ-6, the disease is Multiple Sclerosis (MS) DRB1, the disease is Herpes Simplex Virus 1 or 2. The disease can be Hepatitis B with a target of one or more of PreC, C, X, PreS1, PreS2, S, P and/or SP gene(s).

In embodiments, the immune disease is severe combined immunodeficiency (SCID), Omenn syndrome, and in one aspect the target is Recombination Activating Gene 1 (RAG1) or an interleukin-7 receptor (IL7R). In particular embodiments, the disease is Transthyretin Amyloidosis (ATTR), Familial amyloid cardiomyopathy, and in one aspect, the target is the TTR gene, including one or more mutations in the TTR gene. In embodiments, the disease is Alpha-1 Antitrypsin Deficiency (AATD) or another disease in which Alpha-1 Antitrypsin is implicated, for example GvHD, Organ transplant rejection, diabetes, liver disease, COPD, Emphysema and Cystic Fibrosis, in particular embodiments, the target is SERPINA1.

In embodiments, the disease is primary hyperoxaluria, which, in certain embodiments, the target comprises one or more of Lactate dehydrogenase A (LDHA) and hydroxy Acid Oxidase 1 (HAO 1). In embodiments, the disease is primary hyperoxaluria type 1 (ph1) and other alanine-glyoxylate aminotransferase (agxt) gene related conditions or disorders, such as Adenocarcinoma, Chronic Alcoholic Intoxication, Alzheimer's Disease, Cooley's anemia, Aneurysm, Anxiety Disorders, Asthma, Malignant neoplasm of breast, Malignant neoplasm of skin, Renal Cell Carcinoma, Cardiovascular Diseases, Malignant tumor of cervix, Coronary Arteriosclerosis, Coronary heart disease, Diabetes, Diabetes Mellitus, Diabetes Mellitus Non-Insulin-Dependent, Diabetic Nephropathy, Eclampsia, Eczema, Subacute Bacterial Endocarditis, Glioblastoma, Glycogen storage disease type II, Sensorineural Hearing Loss (disorder), Hepatitis, Hepatitis A, Hepatitis B, Homocystinuria, Hereditary Sensory Autonomic Neuropathy Type 1, Hyperaldosteronism, Hypercholesterolemia, Hyperoxaluria, Primary Hyperoxaluria, Hypertensive disease, Inflammatory Bowel Diseases, Kidney Calculi, Kidney Diseases, Chronic Kidney Failure, leiomyosarcoma, Metabolic Diseases, Inborn Errors of Metabolism, Mitral Valve Prolapse Syndrome, Myocardial Infarction, Neoplasm Metastasis, Nephrotic Syndrome, Obesity, Ovarian Diseases, Periodontitis, Polycystic Ovary Syndrome, Kidney Failure, Adult Respiratory Distress Syndrome, Retinal Diseases, Cerebrovascular accident, Turner Syndrome, Viral hepatitis, Tooth Loss, Premature Ovarian Failure, Essential Hypertension, Left Ventricular Hypertrophy, Migraine Disorders, Cutaneous Melanoma, Hypertensive heart disease, Chronic glomerulonephritis, Migraine with Aura, Secondary hypertension, Acute myocardial infarction, Atherosclerosis of aorta, Allergic asthma, pineoblastoma, Malignant neoplasm of lung, Primary hyperoxaluria type I, Primary hyperoxaluria type 2, Inflammatory Breast Carcinoma, Cervix carcinoma, Restenosis, Bleeding ulcer, Generalized glycogen storage disease of infants, Nephrolithiasis, Chronic rejection of renal transplant, Urolithiasis, pricking of skin, Metabolic Syndrome X, Maternal hypertension, Carotid Atherosclerosis, Carcinogenesis, Breast Carcinoma, Carcinoma of lung, Nephronophthisis, Microalbuminuria, Familial Retinoblastoma, Systolic Heart Failure Ischemic stroke, Left ventricular systolic dysfunction, Cauda Equina Paraganglioma, Hepatocarcinogenesis, Chronic Kidney Diseases, Glioblastoma Multiforme, Non-Neoplastic Disorder, Calcium Oxalate Nephrolithiasis, Ablepharon-Macrostomia Syndrome, Coronary Artery Disease, Liver carcinoma, Chronic kidney disease stage 5, Allergic rhinitis (disorder), Crigler Najjar syndrome type 2, and Ischemic Cerebrovascular Accident. In certain embodiments, treatment is targeted to the liver. In embodiments, the gene is AGXT, with a cytogenetic location of 2q37.3 and the genomic coordinate are on Chromosome 2 on the forward strand at position 240,868,479-240,880,502.

Treatment can also target collagen type vii alpha 1 chain (col7a1) gene related conditions or disorders, such as Malignant neoplasm of skin, Squamous cell carcinoma, Colorectal Neoplasms, Crohn Disease, Epidermolysis Bullosa, Indirect Inguinal Hernia, Pruritus, Schizophrenia, Dermatologic disorders, Genetic Skin Diseases, Teratoma, Cockayne-Touraine Disease, Epidermolysis Bullosa Acquisita, Epidermolysis Bullosa Dystrophica, Junctional Epidermolysis Bullosa, Hallopeau-Siemens Disease, Bullous Skin Diseases, Agenesis of corpus callosum, Dystrophia unguium, Vesicular Stomatitis, Epidermolysis Bullosa With Congenital Localized Absence Of Skin And Deformity Of Nails, Juvenile Myoclonic Epilepsy, Squamous cell carcinoma of esophagus, Poikiloderma of Kindler, pretibial Epidermolysis bullosa, Dominant dystrophic epidermolysis bullosa albopapular type (disorder), Localized recessive dystrophic epidermolysis bullosa, Generalized dystrophic epidermolysis bullosa, Squamous cell carcinoma of skin, Epidermolysis Bullosa Pruriginosa, Mammary Neoplasms, Epidermolysis Bullosa Simplex Superficialis, Isolated Toenail Dystrophy, Transient bullous dermolysis of the newborn, Autosomal Recessive Epidermolysis Bullosa Dystrophica Localisata Variant, and Autosomal Recessive Epidermolysis Bullosa Dystrophica Inversa.

In embodiments, the disease is acute myeloid leukemia (AML), targeting Wilms Tumor I (WTI) and HLA expressing cells. In embodiments, the therapy is T cell therapy, as described elsewhere herein, comprising engineered T cells with WTI specific TCRs. In certain embodiments, the target is CD157 in AML.

In embodiments, the disease is a blood disease. In certain embodiments, the disease is hemophilia, in one aspect the target is Factor XI. In other embodiments, the disease is a hemoglobinopathy, such as sickle cell disease, sickle cell trait, hemoglobin C disease, hemoglobin C trait, hemoglobin S/C disease, hemoglobin D disease, hemoglobin E disease, a thalassemia, a condition associated with hemoglobin with increased oxygen affinity, a condition associated with hemoglobin with decreased oxygen affinity, unstable hemoglobin disease, methemoglobinemia. Hemostasis and Factor X and XII deficiencies can also be treated. In embodiments, the target is BCL11A gene (e.g., a human BCL11a gene), a BCL11a enhancer (e.g., a human BCL11a enhancer), or a HFPH region (e.g., a human HPFH region), beta globulin, fetal hemoglobin, γ-globin genes (e.g., HBG1, HBG2, or HBG1 and HBG2), the erythroid specific enhancer of the BCL11A gene (BCL11Ae), or a combination thereof.

In embodiments, the target locus can be one or more of RAC, TRBC1, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, HCF2, PAI, TFPI, PLAT, PLAU, PLG, RPOZ, F7, F8, F9, F2, F5, F7, F10, F11, F12, F13A1, F13B, STAT1, FOXP3, IL2RG, DCLRE1C, ICOS, MHC2TA, GALNS, HGSNAT, ARSB, RFXAP, CD20, CD81, TNFRSF13B, SEC23B, PKLR, IFNG, SPTB, SPTA, SLC4A1, EPO, EPB42, CSF2 CSF3, VFW, SERPINCA1, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, PTPN11, and combinations thereof. In embodiments, the target sequence within the genomic nucleic acid sequence at Chrl 1:5,250,094-5,250,237, − strand, hg38; Chrl 1:5,255,022-5,255,164, − strand, hg38; nondeletional HFPH region; Chrl 1:5,249,833 to Chrl 1:5,250,237, − strand, hg38; Chrl 1:5,254,738 to Chrl 1:5,255, 164, − strand, hg38; Chrl 1: 5,249,833-5,249,927, − strand, hg3; Chrl 1: 5,254,738-5,254,851, − strand, hg38; Chrl 1:5,250, 139-5,250,237, − strand, hg38.

In embodiments, the disease is associated with high cholesterol, and regulation of cholesterol is provided, in some embodiments, regulation is effected by modification in the target PCSK9. Other diseases in which PCSK9 can be implicated, and thus would be a target for the systems and methods described herein include Abetaiipoproteinemia, Adenoma, Arteriosclerosis, Atherosclerosis, Cardiovascular Diseases, Cholelithiasis, Coronary Arteriosclerosis, Coronary heart disease, Non-Insulin-Dependent Diabetes Meliitus, Hypercholesterolemia, Familial Hypercholesterolemia, Hyperinsuiinism, Hyperlipidemia, Familial Combined Hyperlipidemia, Hypobetalipoproteinemias, Chronic Kidney Failure, Liver diseases, Liver neoplasms, melanoma, Myocardial Infarction, Narcolepsy, Neoplasm Metastasis, Nephroblastoma, Obesity, Peritonitis, Pseudoxanthoma Elasticum, Cerebrovascular accident, Vascular Diseases, Xanthomatosis, Peripheral Vascular Diseases, Myocardial Ischemia, Dyslipidemias, Impaired glucose tolerance, Xanthoma, Polygenic hypercholesterolemia, Secondary malignant neoplasm of liver, Dementia, Overweight, Hepatitis C, Chronic, Carotid Atherosclerosis, Hyperlipoproteinemia Type Ha, Intracranial Atherosclerosis, Ischemic stroke, Acute Coronary Syndrome, Aortic calcification, Cardiovascular morbidity, Hyperlipoproteinemia Type lib, Peripheral Arterial Diseases, Familial Hyperaldosteronism Type II, Familial hypobetalipoproteinemia, Autosomal Recessive Hypercholesterolemia, Autosomal Dominant Hypercholesterolemia 3, Coronary Artery Disease, Liver carcinoma, Ischemic Cerebrovascular Accident, and Arteriosclerotic cardiovascular disease NOS. In embodiments, the treatment can be targeted to the liver, the primary location of activity of PCSK9.

In embodiments, the disease or disorder is Hyper IGM syndrome or a disorder characterized by defective CD40 signaling. In certain embodiments, the insertion of CD40L exons are used to restore proper CD40 signaling and B cell class switch recombination. In particular embodiments, the target is CD40 ligand (CD40L)-edited at one or more of exons 2-5 of the CD40L gene, in cells, e.g., T cells or hematopoietic stem cells (HSCs).

In embodiments, the disease is merosin-deficient congenital muscular dystrophy (mdcmd) and other laminin, alpha 2 (lama2) gene related conditions or disorders. The therapy can be targeted to the muscle, for example, skeletal muscle, smooth muscle, and/or cardiac muscle. In certain embodiments, the target is Laminin, Alpha 2 (LAMA2) which may also be referred to as Laminin-12 Subunit Alpha, Laminin-2 Subunit Alpha, Laminin-4 Subunit Alpha 3, Merosin Heavy Chain, Laminin M Chain, LAMM, Congenital Muscular Dystrophy and Merosin. LAMA2 has a cytogenetic location of 6q22.33 and the genomic coordinate are on Chromosome 6 on the forward strand at position 128,883, 141-129,516,563. In embodiments, the disease treated can be Merosin-Deficient Congenital Muscular Dystrophy (MDCMD), Amyotrophic Lateral Sclerosis, Bladder Neoplasm, Charcot-Marie-Tooth Disease, Colorectal Carcinoma, Contracture, Cyst, Duchenne Muscular Dystrophy, Fatigue, Hyperopia, Renovascular Hypertension, melanoma, Mental Retardation, Myopathy, Muscular Dystrophy, Myopia, Myositis, Neuromuscular Diseases, Peripheral Neuropathy, Refractive Errors, Schizophrenia, Severe mental retardation (I.Q. 20-34), Thyroid Neoplasm, Tobacco Use Disorder, Severe Combined Immunodeficiency, Synovial Cyst, Adenocarcinoma of lung (disorder), Tumor Progression, Strawberry nevus of skin, Muscle degeneration, Microdontia (disorder), Walker-Warburg congenital muscular dystrophy, Chronic Periodontitis, Leukoencephalopathies, Impaired cognition, Fukuyama Type Congenital Muscular Dystrophy, Scleroatonic muscular dystrophy, Eichsfeld type congenital muscular dystrophy, Neuropathy, Muscle eye brain disease, Limb-Muscular Dystrophies, Girdle, Congenital muscular dystrophy (disorder), Muscle fibrosis, cancer recurrence, Drug Resistant Epilepsy, Respiratory Failure, Myxoid cyst, Abnormal breathing, Muscular dystrophy congenital merosin negative, Colorectal Cancer, Congenital Muscular Dystrophy due to Partial LAMA2 Deficiency, and Autosomal Dominant Craniometaphyseal Dysplasia.

In one aspect, the target is superoxide dismutase 1, soluble (SOD1), which can aid in treatment of a disease or disorder associated with the gene. In particular embodiments, the disease or disorder is associated with SOD1, and can be, for example, Adenocarcinoma, Albuminuria, Chronic Alcoholic Intoxication, Alzheimer's Disease, Amnesia, Amyloidosis, Amyotrophic Lateral Sclerosis, Anemia, Autoimmune hemolytic anemia, Sickle Cell Anemia, Anoxia, Anxiety Disorders, Aortic Diseases, Arteriosclerosis, Rheumatoid Arthritis, Asphyxia Neonatorum, Asthma, Atherosclerosis, Autistic Disorder, Autoimmune Diseases, Barrett Esophagus, Behcet Syndrome, Malignant neoplasm of urinary bladder, Brain Neoplasms, Malignant neoplasm of breast, Oral candidiasis, Malignant tumor of colon, Bronchogenic Carcinoma, Non-Small Cell Lung Carcinoma, Squamous cell carcinoma, Transitional Cell Carcinoma, Cardiovascular Diseases, Carotid Artery Thrombosis, Neoplastic Cell Transformation, Cerebral Infarction, Brain Ischemia, Transient Ischemic Attack, Charcot-Marie-Tooth Disease, Cholera, Colitis, Colorectal Carcinoma, Coronary Arteriosclerosis, Coronary heart disease, Infection by Cryptococcus neoformans, Deafness, Cessation of life, Deglutition Disorders, Presenile dementia, Depressive disorder, Contact Dermatitis, Diabetes, Diabetes Mellitus, Experimental Diabetes Mellitus, Insulin-Dependent Diabetes Mellitus, Non-Insulin-Dependent Diabetes Mellitus, Diabetic Angiopathies, Diabetic Nephropathy, Diabetic Retinopathy, Down Syndrome, Dwarfism, Edema, Japanese Encephalitis, Toxic Epidermal Necrolysis, Temporal Lobe Epilepsy, Exanthema, Muscular fasciculation, Alcoholic Fatty Liver, Fetal Growth Retardation, Fibromyalgia, Fibrosarcoma, Fragile X Syndrome, Giardiasis, Glioblastoma, Glioma, Headache, Partial Hearing Loss, Cardiac Arrest, Heart failure, Atrial Septal Defects, Helminthiasis, Hemochromatosis, Hemolysis (disorder), Chronic Hepatitis, HIV Infections, Huntington Disease, Hypercholesterolemia, Hyperglycemia, Hyperplasia, Hypertensive disease, Hyperthyroidism, Hypopituitarism, Hypoproteinemia, Hypotension, natural Hypothermia, Hypothyroidism, Immunologic Deficiency Syndromes, Immune System Diseases, Inflammation, Inflammatory Bowel Diseases, Influenza, Intestinal Diseases, Ischemia, Kearns-Sayre syndrome, Keratoconus, Kidney Calculi, Kidney Diseases, Acute Kidney Failure, Chronic Kidney Failure, Polycystic Kidney Diseases, leukemia, Myeloid Leukemia, Acute Promyelocytic Leukemia, Liver Cirrhosis, Liver diseases, Liver neoplasms, Locked-In Syndrome, Chronic Obstructive Airway Disease, Lung Neoplasms, Systemic Lupus Erythematosus, Non-Hodgkin Lymphoma, Machado-Joseph Disease, Malaria, Malignant neoplasm of stomach, Animal Mammary Neoplasms, Marfan Syndrome, Meningomyelocele, Mental Retardation, Mitral Valve Stenosis, Acquired Dental Fluorosis, Movement Disorders, Multiple Sclerosis, Muscle Rigidity, Muscle Spasticity, Muscular Atrophy, Spinal Muscular Atrophy, Myopathy, Mycoses, Myocardial Infarction, Myocardial Reperfusion Injury, Necrosis, Nephrosis, Nephrotic Syndrome, Nerve Degeneration, nervous system disorder, Neuralgia, Neuroblastoma, Neuroma, Neuromuscular Diseases, Obesity, Occupational Diseases, Ocular Hypertension, Oligospermia, Degenerative polyarthritis, Osteoporosis, Ovarian Carcinoma, Pain, Pancreatitis, Papillon-Lefevre Disease, Paresis, Parkinson Disease, Phenylketonurias, Pituitary Diseases, Pre-Eclampsia, Prostatic Neoplasms, Protein Deficiency, Proteinuria, Psoriasis, Pulmonary Fibrosis, Renal Artery Obstruction, Reperfusion Injury, Retinal Degeneration, Retinal Diseases, Retinoblastoma, Schistosomiasis, Schistosomiasis mansoni, Schizophrenia, Scrapie, Seizures, Age-related cataract, Compression of spinal cord, Cerebrovascular accident, Subarachnoid Hemorrhage, Progressive supranuclear palsy, Tetanus, Trisomy, Turner Syndrome, Unipolar Depression, Urticaria, Vitiligo, Vocal Cord Paralysis, Intestinal Volvulus, Weight Gain, HMN (Hereditary Motor Neuropathy) Proximal Type I, Holoprosencephaly, Motor Neuron Disease, Neurofibrillary degeneration (morphologic abnormality), Burning sensation, Apathy, Mood swings, Synovial Cyst, Cataract, Migraine Disorders, Sciatic Neuropathy, Sensory neuropathy, Atrophic condition of skin, Muscle Weakness, Esophageal carcinoma, Lingual-Facial-Buccal Dyskinesia, Idiopathic pulmonary hypertension, Lateral Sclerosis, Migraine with Aura, Mixed Conductive-Sensorineural Hearing Loss, Iron deficiency anemia, Malnutrition, Prion Diseases, Mitochondrial Myopathies, MELAS Syndrome, Chronic progressive external ophthalmoplegia, General Paralysis, Premature aging syndrome, Fibrillation, Psychiatric symptom, Memory impairment, Muscle degeneration, Neurologic Symptoms, Gastric hemorrhage, Pancreatic carcinoma, Pick Disease of the Brain, Liver Fibrosis, Malignant neoplasm of lung, Age related macular degeneration, Parkinsonian Disorders, Disease Progression, Hypocupremia, Cytochrome-c Oxidase Deficiency, Essential Tremor, Familial Motor Neuron Disease, Lower Motor Neuron Disease, Degenerative myelopathy, Diabetic Polyneuropathies, Liver and Intrahepatic Biliary Tract Carcinoma, Persian Gulf Syndrome, Senile Plaques, Atrophic, Frontotemporal dementia, Semantic Dementia, Common Migraine, Impaired cognition, Malignant neoplasm of liver, Malignant neoplasm of pancreas, Malignant neoplasm of prostate, Pure Autonomic Failure, Motor symptoms, Spastic, Dementia, Neurodegenerative Disorders, Chronic Hepatitis C, Guam Form Amyotrophic Lateral Sclerosis, Stiff limbs, Multisystem disorder, Loss of scalp hair, Prostate carcinoma, Hepatopulmonary Syndrome, Hashimoto Disease, Progressive Neoplastic Disease, Breast Carcinoma, Terminal illness, Carcinoma of lung, Tardive Dyskinesia, Secondary malignant neoplasm of lymph node, Colon Carcinoma, Stomach Carcinoma, Central neuroblastoma, Dissecting aneurysm of the thoracic aorta, Diabetic macular edema, Microalbuminuria, Middle Cerebral Artery Occlusion, Middle Cerebral Artery Infarction, Upper motor neuron signs, Frontotemporal Lobar Degeneration, Memory Loss, Classical phenylketonuria, CADASIL Syndrome, Neurologic Gait Disorders, Spinocerebellar Ataxia Type 2, Spinal Cord Ischemia, Lewy Body Disease, Muscular Atrophy, Spinobulbar, Chromosome 21 monosomy, Thrombocytosis, Spots on skin, Drug-Induced Liver Injury, Hereditary Leber Optic Atrophy, Cerebral Ischemia, ovarian neoplasm, Tauopathies, Macroangiopathy, Persistent pulmonary hypertension, Malignant neoplasm of ovary, Myxoid cyst, Drusen, Sarcoma, Weight decreased, Major Depressive Disorder, Mild cognitive disorder, Degenerative disorder, Partial Trisomy, Cardiovascular morbidity, hearing impairment, Cognitive changes, Ureteral Calculi, Mammary Neoplasms, Colorectal Cancer, Chronic Kidney Diseases, Minimal Change Nephrotic Syndrome, Non-Neoplastic Disorder, X-Linked Bulbo-Spinal Atrophy, Mammographic Density, Normal Tension Glaucoma Susceptibility To Finding), Vitiligo-Associated Multiple Autoimmune Disease Susceptibility 1 (Finding), Amyotrophic Lateral Sclerosis And/Or Frontotemporal Dementia 1, Amyotrophic Lateral Sclerosis 1, Sporadic Amyotrophic Lateral Sclerosis, monomelic Amyotrophy, Coronary Artery Disease, Transformed migraine, Regurgitation, Urothelial Carcinoma, Motor disturbances, Liver carcinoma, Protein Misfolding Disorders, TDP-43 Proteinopathies, Promyelocytic leukemia, Weight Gain Adverse Event, Mitochondrial cytopathy, Idiopathic pulmonary arterial hypertension, Progressive cGVHD, Infection, GRN-related frontotemporal dementia, Mitochondrial pathology, and Hearing Loss.

In particular embodiments, the disease is associated with the gene ATXN1, ATXN2, or ATXN3, which may be targeted for treatment. In some embodiments, the CAG repeat region located in exon 8 of ATXN1, exon 1 of ATXN2, or exon 10 of the ATXN3 is targeted. In embodiments, the disease is spinocerebellar ataxia 3 (sca3), sca1, or sca2 and other related disorders, such as Congenital Abnormality, Alzheimer's Disease, Amyotrophic Lateral Sclerosis, Ataxia, Ataxia Telangiectasia, Cerebellar Ataxia, Cerebellar Diseases, Chorea, Cleft Palate, Cystic Fibrosis, Mental Depression, Depressive disorder, Dystonia, Esophageal Neoplasms, Exotropia, Cardiac Arrest, Huntington Disease, Machado-Joseph Disease, Movement Disorders, Muscular Dystrophy, Myotonic Dystrophy, Narcolepsy, Nerve Degeneration, Neuroblastoma, Parkinson Disease, Peripheral Neuropathy, Restless Legs Syndrome, Retinal Degeneration, Retinitis Pigmentosa, Schizophrenia, Shy-Drager Syndrome, Sleep disturbances, Hereditary Spastic Paraplegia, Thromboembolism, Stiff-Person Syndrome, Spinocerebellar Ataxia, Esophageal carcinoma, Polyneuropathy, Effects of heat, Muscle twitch, Extrapyramidal sign, Ataxic, Neurologic Symptoms, Cerebral atrophy, Parkinsonian Disorders, Protein S Deficiency, Cerebellar degeneration, Familial Amyloid Neuropathy Portuguese Type, Spastic syndrome, Vertical Nystagmus, Nystagmus End-Position, Antithrombin III Deficiency, Atrophic, Complicated hereditary spastic paraplegia, Multiple System Atrophy, Pallidoluysian degeneration, Dystonia Disorders, Pure Autonomic Failure, Thrombophilia, Protein C, Deficiency, Congenital Myotonic Dystrophy, Motor symptoms, Neuropathy, Neurodegenerative Disorders, Malignant neoplasm of esophagus, Visual disturbance, Activated Protein C Resistance, Terminal illness, Myokymia, Central neuroblastoma, Dyssomnias, Appendicular Ataxia, Narcolepsy-Cataplexy Syndrome, Machado-Joseph Disease Type I, Machado-Joseph Disease Type II, Machado-Joseph Disease Type III, Dentatorubral-Pallidoluysian Atrophy, Gait Ataxia, Spinocerebellar Ataxia Type 1, Spinocerebellar Ataxia Type 2, Spinocerebellar Ataxia Type 6 (disorder), Spinocerebellar Ataxia Type 7, Muscular Spinobulbar Atrophy, Genomic Instability, Episodic ataxia type 2 (disorder), Bulbo-Spinal Atrophy X-Linked, Fragile X Tremor/Ataxia Syndrome, Thrombophilia Due to Activated Protein C Resistance (Disorder), Amyotrophic Lateral Sclerosis 1, Neuronal Intranuclear Inclusion Disease, Hereditary Antithrombin Iii Deficiency, and Late-Onset Parkinson Disease.

In embodiments, the disease is associated with expression of a tumor antigen-cancer or non-cancer related indication, for example acute lymphoid leukemia, diffuse large B cell lymphoma, follicular lymphoma, chronic lymphocytic leukemia, Hodgkin lymphoma, non-Hodgkin lymphoma. In embodiments, the target can be TET2 intron, a TET2 intron-exon junction, a sequence within a genomic region of chr4.

In embodiments, neurodegenerative diseases can be treated. In particular embodiments, the target is Synuclein, Alpha (SNCA). In certain embodiments, the disorder treated is a pain related disorder, including congenital pain insensitivity, Compressive Neuropathies, Paroxysmal Extreme Pain Disorder, High grade atrioventricular block, Small Fiber Neuropathy, and Familial Episodic Pain Syndrome 2. In certain embodiments, the target is Sodium Channel, Voltage Gated, Type X Alpha Subunit (SCNIOA).

In certain embodiments, hematopoetic stem cells and progenitor stem cells are modified, including for treatment of lysosomal storage diseases, glycogen storage diseases, mucopolysaccharoidoses, or any disease in which the secretion of a protein will ameliorate the disease. In one embodiment, the disease is sickle cell disease (SCD). In another embodiment, the disease is β-thalessemia.

Methods and systems can target Dystrophia Myotonica-Protein Kinase (DMPK). Disorders or diseases associated with DMPK include Atherosclerosis, Azoospermia, Hypertrophic Cardiomyopathy, Celiac Disease, Congenital chromosomal disease, Diabetes Mellitus, Focal glomerulosclerosis, Huntington Disease, Hypogonadism, Muscular Atrophy, Myopathy, Muscular Dystrophy, Myotonia, Myotonic Dystrophy, Neuromuscular Diseases, Optic Atrophy, Paresis, Schizophrenia, Cataract, Spinocerebellar Ataxia, Muscle Weakness, Adrenoleukodystrophy, Centronuclear myopathy, Interstitial fibrosis, myotonic muscular dystrophy, Abnormal mental state, X-linked Charcot-Marie-Tooth disease 1, Congenital Myotonic Dystrophy, Bilateral cataracts (disorder), Congenital Fiber Type Disproportion, Myotonic Disorders, Multisystem disorder, 3-Methylglutaconic aciduria type 3, cardiac event, Cardiogenic Syncope, Congenital Structural Myopathy, Mental handicap, Adrenomyeloneuropathy, Dystrophia myotonica 2, and Intellectual Disability.

In embodiments, the disease is an inborn error of metabolism. The disease may be selected from Disorders of Carbohydrate Metabolism (glycogen storage disease, G6PD deficiency), Disorders of Amino Acid Metabolism (phenylketonuria, maple syrup urine disease, glutaric acidemia type 1), Urea Cycle Disorder or Urea Cycle Defects (carbamoyl phosphate synthease I deficiency), Disorders of Organic Acid Metabolism (alkaptonuria, 2-hydroxyglutaric acidurias), Disorders of Fatty Acid Oxidation/Mitochondrial Metabolism (Medium-chain acyl-coenzyme A dehydrogenase deficiency), Disorders of Porphyrin metabolism (acute intermittent porphyria), Disorders of Purine/Pyrimidine Metabolism (Lesch-Nynan syndrome), Disorders of Steroid Metabolism (lipoid congenital adrenal hyperplasia, congenital adrenal hyperplasia), Disorders of Mitochondrial Function (Kearns-Sayre syndrome), Disorders of Peroxisomal function (Zellweger syndrome), or Lysosomal Storage Disorders (Gaucher's disease, Niemann-Pick disease).

In embodiments, the target can comprise Recombination Activating Gene 1 (RAG1), BCL11 A, PCSK9, laminin, alpha 2 (lama2), ATXN3, alanine-glyoxylate aminotransferase (AGXT), collagen type vii alpha 1 chain (COL7a1), spinocerebellar ataxia type 1 protein (ATXN1), Angiopoietin-like 3 (ANGPTL3), Frataxin (FXN), Superoxidase Dismutase 1, soluble (SOD1), Synuclein, Alpha (SNCA), Sodium Channel, Voltage Gated, Type X Alpha Subunit (SCN10A), Spinocerebellar Ataxia Type 2 Protein (ATXN2), Dystrophia Myotonica-Protein Kinase (DMPK), beta globin locus on chromosome 11, acyl-coenzyme A dehydrogenase for medium chain fatty acids (ACADM), long-chain 3-hydroxyl-coenzyme A dehydrogenase for long chain fatty acids (HADHA), acyl-coenzyme A dehydrogenase for very long-chain fatty acids (ACADVL), Apolipoprotein C3 (APOCIII), Transthyretin (TTR), Angiopoietin-like 4 (ANGPTL4), Sodium Voltage-Gated Channel Alpha Subunit 9 (SCN9A), Interleukin-7 receptor (IL7R), glucose-6-phosphatase, catalytic (G6PC), haemochromatosis (HFE), SERPINA1, C90RF72, β-globin, dystrophin, γ-globin.

In certain embodiments, the disease or disorder is associated with Apolipoprotein C3 (APOCIII), which can be targeted for editing. In embodiments, the disease or disorder may be Dyslipidemias, Hyperalphalipoproteinemia Type 2, Lupus Nephritis, Wilms Tumor 5, Morbid obesity and spermatogenic, Glaucoma, Diabetic Retinopathy, Arthrogryposis renal dysfunction cholestasis syndrome, Cognition Disorders, Altered response to myocardial infarction, Glucose Intolerance, Positive regulation of triglyceride biosynthetic process, Renal Insufficiency, Chronic, Hyperlipidemias, Chronic Kidney Failure, Apolipoprotein C-III Deficiency, Coronary Disease, Neonatal Diabetes Mellitus, Neonatal, with Congenital Hypothyroidism, Hypercholesterolemia Autosomal Dominant 3, Hyperlipoproteinemia Type III, Hyperthyroidism, Coronary Artery Disease, Renal Artery Obstruction, Metabolic Syndrome X, Hyperlipidemia, Familial Combined, Insulin Resistance, Transient infantile hypertriglyceridemia, Diabetic Nephropathies, Diabetes Mellitus (Type 1), Nephrotic Syndrome Type 5 with or without ocular abnormalities, and Hemorrhagic Fever with renal syndrome.

In certain embodiments, the target is Angiopoietin-like 4 (ANGPTL4). Diseases or disorders associated with ANGPTL4 that can be treated include ANGPTL4 is associated with dyslipidemias, low plasma triglyceride levels, regulator of angiogenesis and modulate tumorigenesis, and severe diabetic retinopathy. both proliferative diabetic retinopathy and non-proliferative diabetic retinopathy.

The protein binding binds to the protein of interest in order to induce phosphorylation from kinases, even if the protein of interest is not a substrate for the kinase. One such protein is in the bromodomain family of proteins. Bromodomains are a family of (−110 amino acid) structurally and evolutionary conserved protein interaction modules that specifically recognize acetylated lysines present in substrate proteins, notably histones. Bromodomains exist as components of large multidomain nuclear proteins that are associated with chromatin remodeling, cell signaling and transcriptional control. Examples of bromodomain-containing proteins with known functions include: (i) histone acetyltransferases (HATs), including CREBBP, GCN5, PCAF and TAFII250; (ii) methyltransferases such as ASH1L and MLL; (iii) components of chromatin-remodeling complexes such as Swi2/Snf2; and (iv) a number of transcriptional regulators (Florence et al. Front. Biosci. 2001, 6, D1008-1018, hereby incorporated by reference in its entirety).

Bromodomain mediated or BET-mediated such as BRD2-mediated, BRD3-mediated, BRD4-mediated, and/or BRDT-mediated disorders or conditions may be any disease or other deleterious condition in which one or more of the bromodomain-containing proteins, such as BET proteins including BRD2, BRD3, BRD4 and/or BRDT, or a mutant thereof, are known to play a role. Accordingly, another embodiment of the present disclosure relates to treating or lessening the severity of one or more diseases in which one or more of the bromodomain-containing proteins, such as BET proteins, such as BRD2, BRD3, BRD4, and/or BRDT, or a mutant thereof, are known to play a role. For example, a disease or condition in which the biological function of bromodomain affects the development and/or course of the disease or condition, and/or in which modulation of bromodomain alters the development, course, and/or symptoms. Bromodomain mediated disease or condition includes a disease or condition for which bromodomain inhibition provides a therapeutic benefit, e.g. wherein treatment with bromodomain inhibitors, including compounds described herein, provides a therapeutic benefit to the subject suffering from or at risk of the disease or condition. Compounds for inhibiting bromodomains or bromodomain inhibitors are typically compounds which inhibit the binding of a bromodomain with its cognate acetylated proteins, for example, the bromodomain inhibitor is a compound which inhibits the binding of a bromodomain to acetylated lysine residues.

Methods for modifying a protein of interest are also provided, the method comprising contacting the protein of interest with a compound disclosed herein in an environment comprising one or more activators. Methods for the treatment of a disease, disorder, or condition in a subject in need thereof can comprise administering a molecule disclosed herein to a subject.

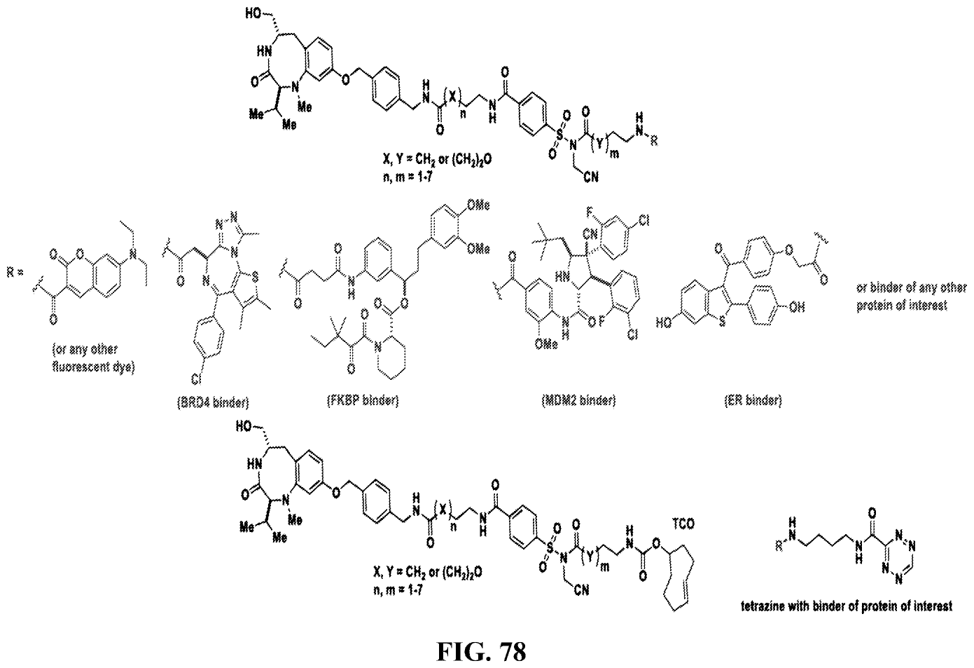

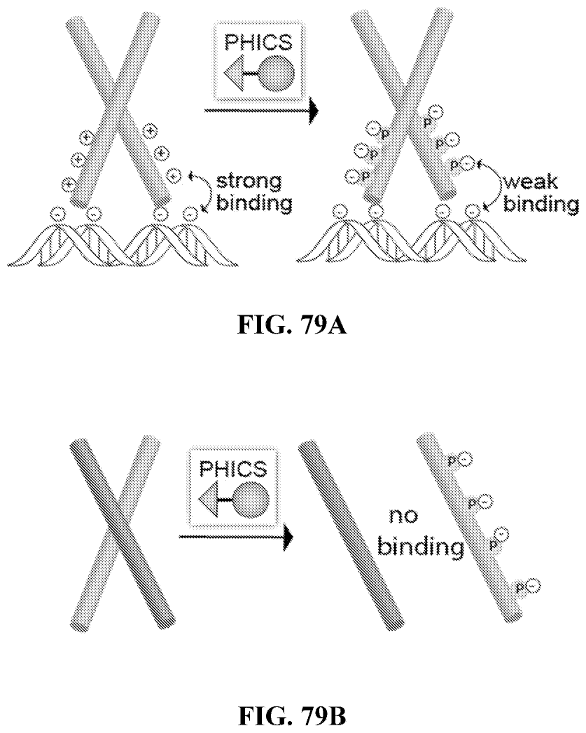

Methods of making a multifunctional conjugation molecules are also provided, comprising binding a localizing moiety and an activator moiety to different ends of a linker molecule, the localizing moiety and activator moiety optionally bound to the linker molecule via orienting adaptors wherein the linker molecule links the activator molecule such that both the activator molecule and localizing moiety is active in a cell. Exemplary methods include those described in the examples herein, and as depicted, for example in FIGS. 5, 57, 58, 78, 80, 84C and 84D.

The invention is further described in the following examples, which do not limit the scope of the invention described in the claims.

### EXAMPLES

### Example 1

Small molecules have been classically used to inhibit enzyme function (i.e., loss-of-function) but several new classes of small molecules are emerging that endow new functions to enzymes via proximity-mediated effects. Herein, Applicants describe a new class of molecules that Applicants term as phosphorylation-inducing chimeric small molecules (PHICS) that enables two kinases (AMPK and PKC) to phosphorylate the target protein (BRD4) that is not substrate for these kinases. PHICS was formed by joining small-molecule activators of these kinases with BRD4 binder, (+)-JQ1, using a linker and exhibited several features of a bifunctional molecule, including “hook-effect,” turnover, isoform specificity, and dependence of activity on proximity (i.e. linker length). The studies provide yet another example of expansion of the scope of chemical inducers of dimerization to induce a post-translational modification on a protein by rewiring the specificity of the enzyme. It is envisioned that the PHICS-mediated site-specific and biologically-relevant phosphorylation, as well as neo-phosphorylations, will find utility in basic research and medicine.

The appendage of a phosphoryl group to many proteins profoundly influences their structure and function. Unsurprisingly, small molecules that block protein phosphorylation via kinase inhibition have had a transformative impact in basic science and medicine. Applicants hypothesize that small molecules that induce phosphorylation of any given protein-of-interest on-demand will also be useful in myriad scenarios. For example, inducing specific and biologically-relevant phosphorylation can trigger a signaling event while neophosphorylation of a protein may impact its structure, evoke an immune response, induce phase separation in cells, or affect protein's interaction with other biomolecules, particularly with RNA/DNA that have negatively-charged phosphodiester backbone. To design such phosphorylation-inducing molecules, Applicants drew inspiration from chemical inducers of dimerization⁵⁻⁶ and ubiquitination-inducing small molecules (e.g., PROTACs)⁷ that increase the effective molarity of the ubiquitin ligase around the target protein, triggering ubiquitination even when the target protein is not ligase's substrate. Herein is described a new class of bifunctional molecules termed phosphorylation-inducing chimeric small molecules (PHICS) formed by conjoining a kinase activator with the small-molecule binder of the target protein. Specifically, demonstrated herein is the PHICS-induced rewiring of specificity of two kinases, AMP-activated protein kinase (AMPK) and protein kinase C (PKC), to induce phosphorylation of bromodomain-containing protein 4 (BRD4), which is not a substrate of AMPK or PKC (FIG. 1A). While kinase specificity has been rewired using adaptor proteins,⁸⁻⁹ it is believed these studies provide first examples kinase specificity rewiring using small molecules.

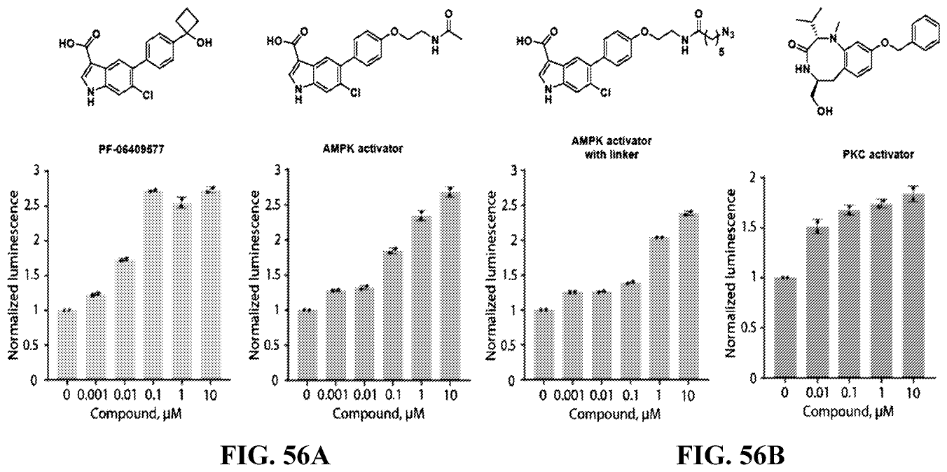

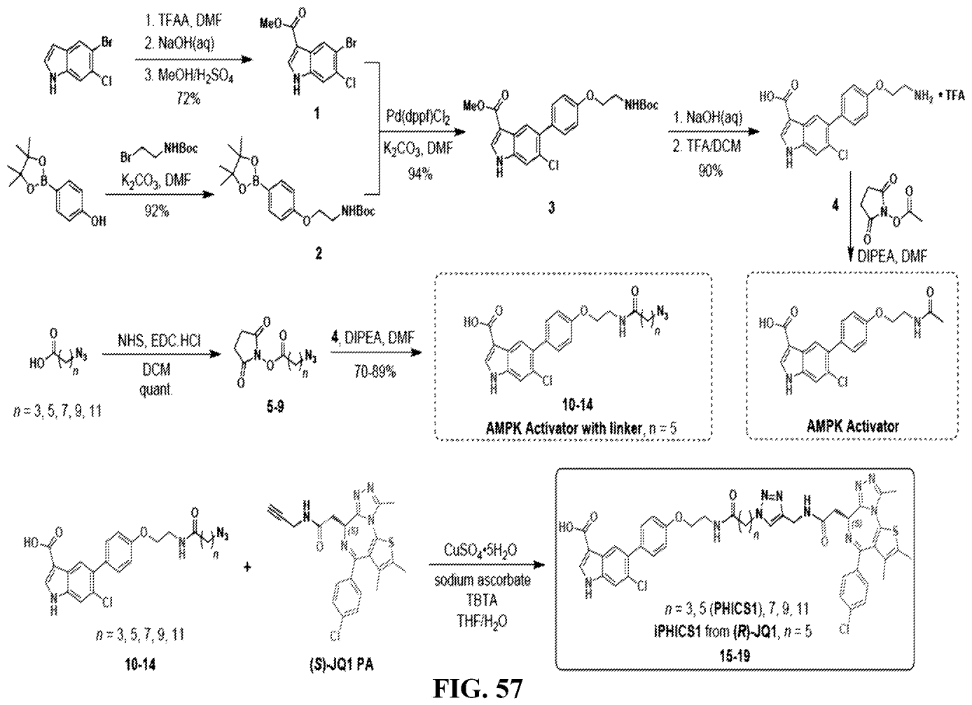

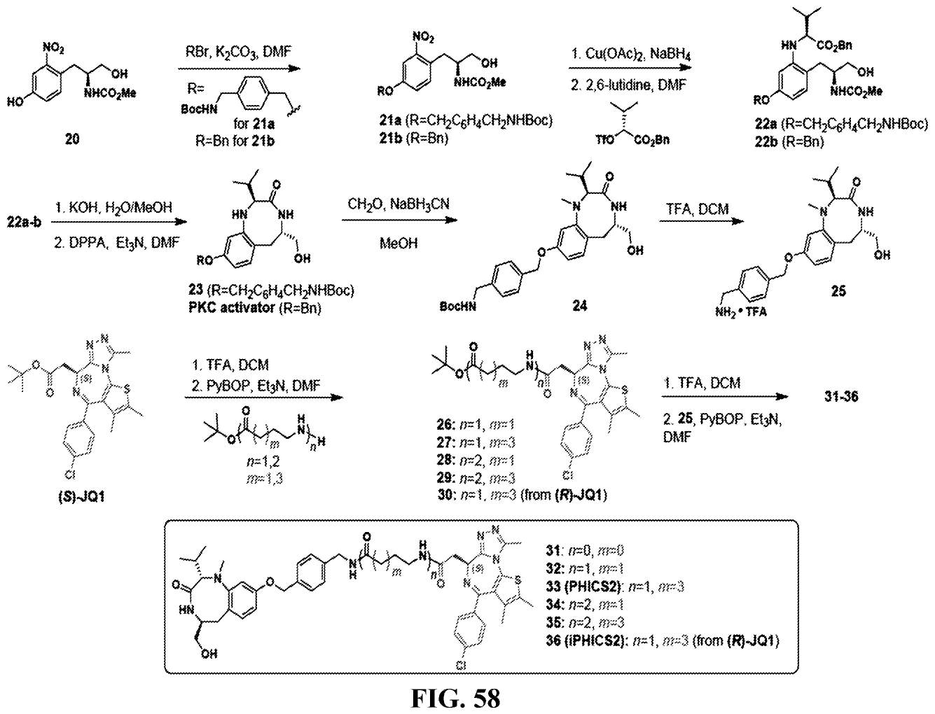

To generate PHICS for AMPK and PKC, Applicants designed small-molecule kinase binders with functional groups for linker attachment. For AMPK, Applicants modified its allosteric activator (14) PF-06409577, by replacing the cyclobutyl ring with the more synthetically amenable aminoethyl handle (FIG. 55A), while for PKC 9-(4-aminomethylbenzyloxy)-substituted benzolactam activator was used (15). These chemical modifications did not perturb the ability of the binders to activate AMPK or PKC as assessed by the ADP-Glo™ assay (16), which measures the amount of ADP produced from the kinase reaction (FIG. 56). Next, Applicants generated PHICS by conjugating these binders to (+)-JQ1, a BRD4 binder, with linkers of varying length (FIGS. 57 and 58). The AMPK PHICS were synthesized using a modular, click-chemistry-based convergent synthesis (17) (FIG. 57), while the PKC PHICS were constructed using two consecutive amidation steps (FIG. 58).

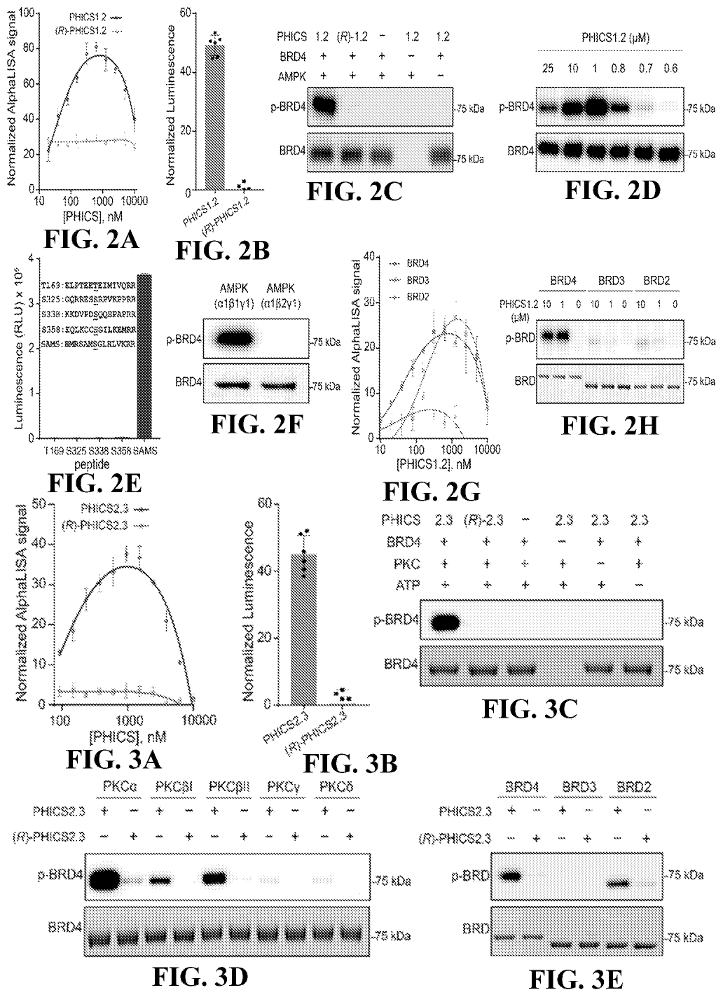

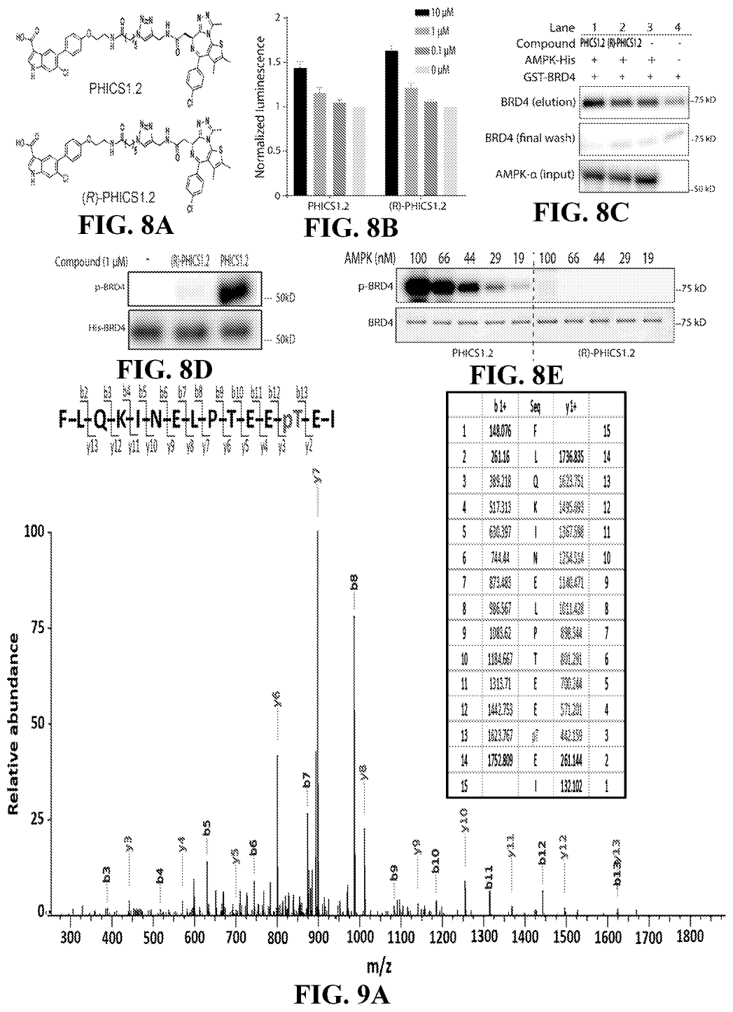

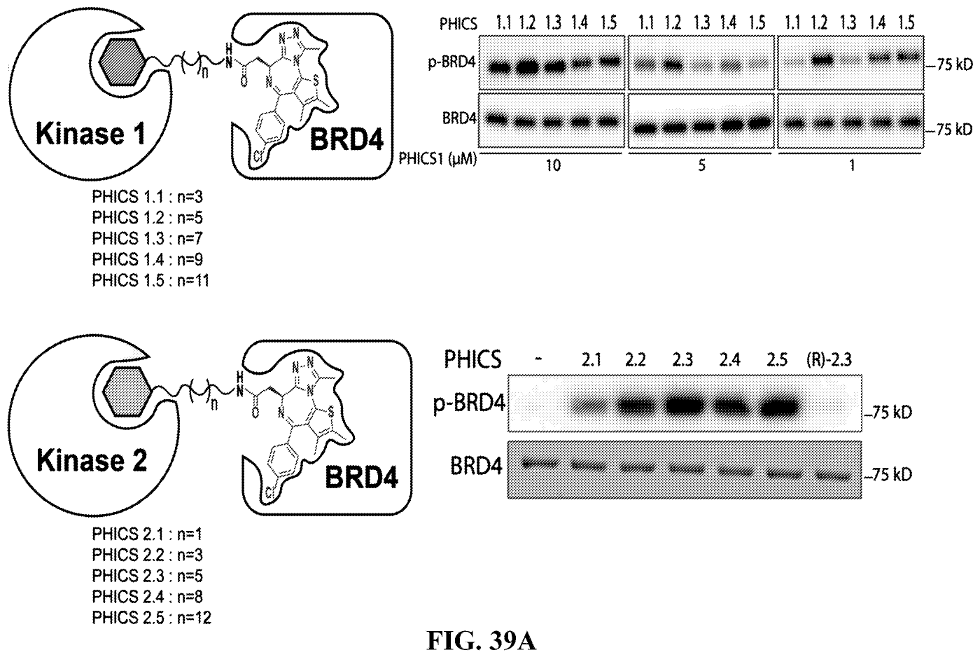

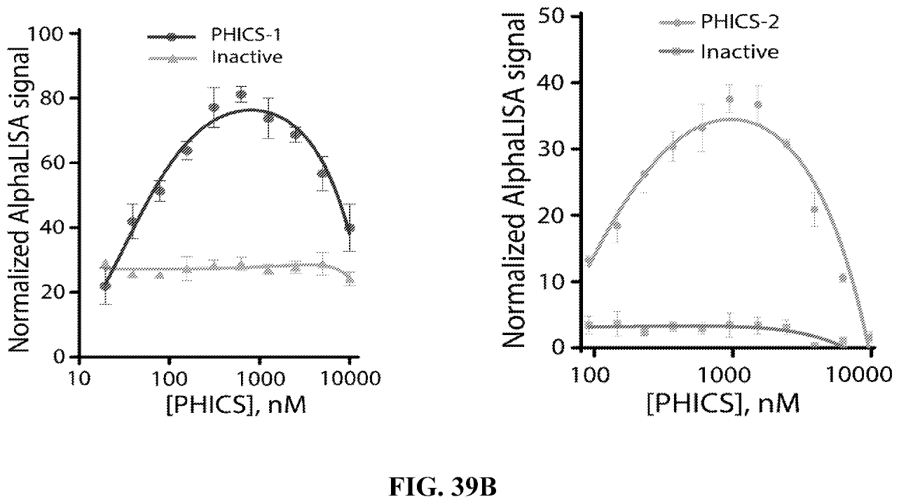

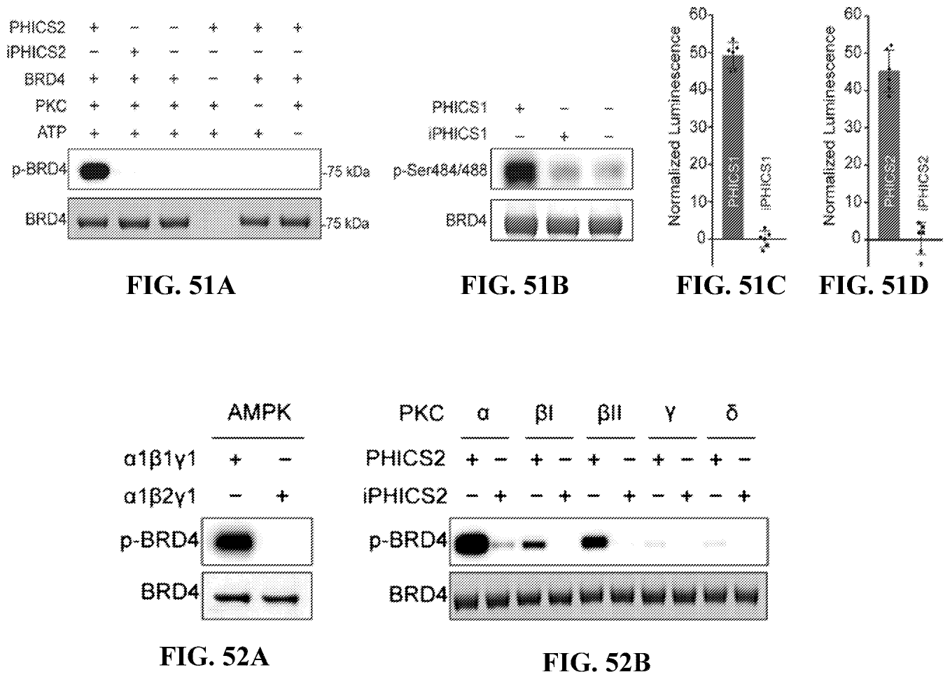

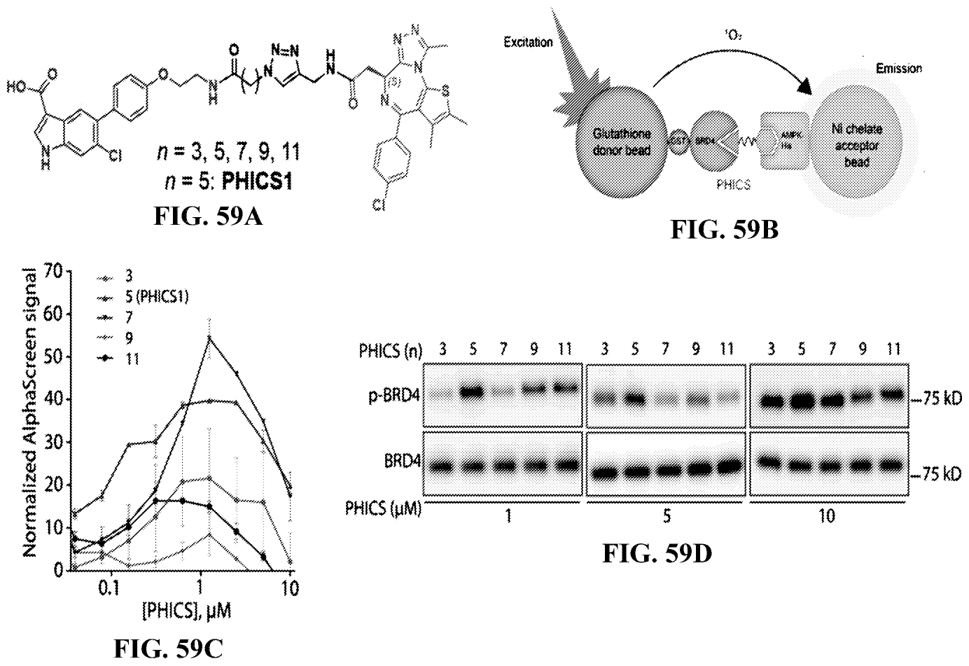

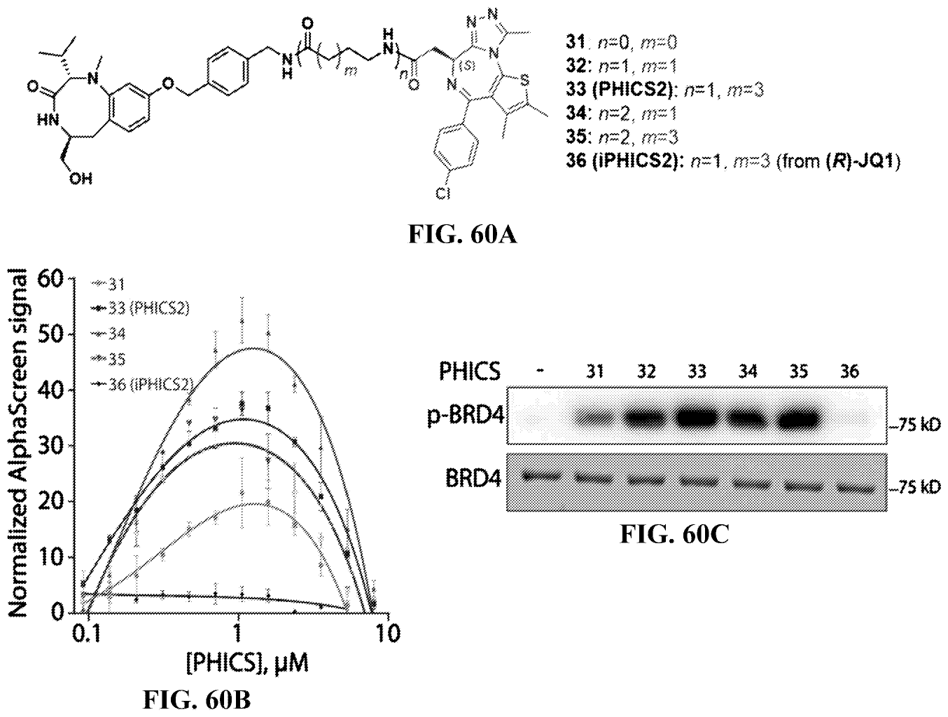

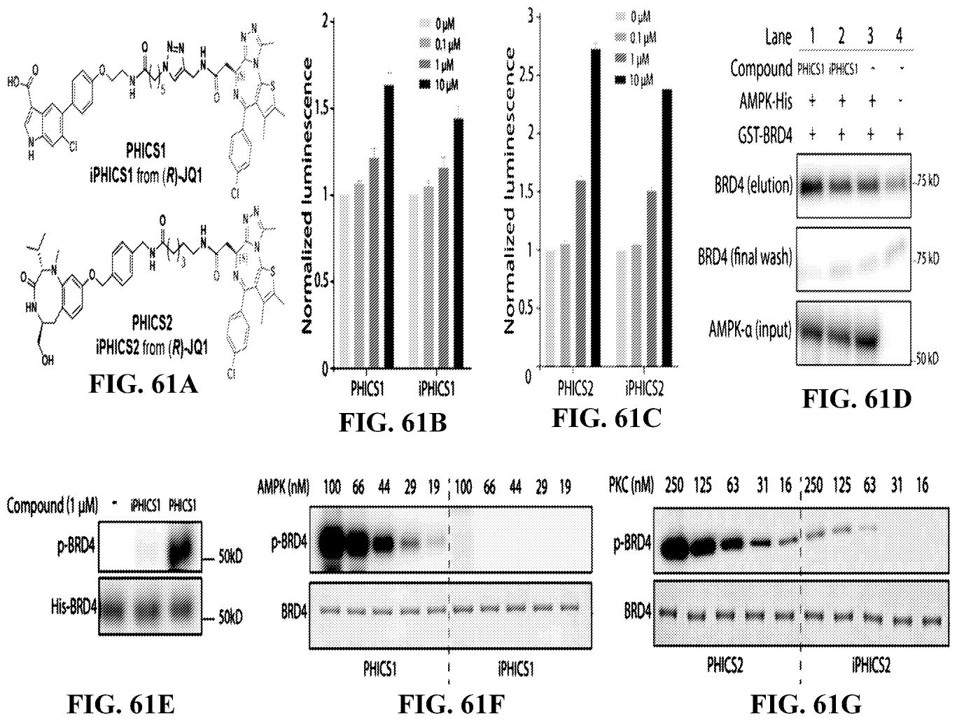

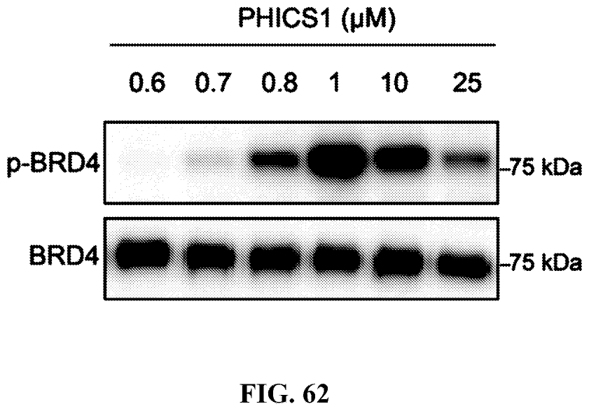

Applicants assessed both ternary complex formation and levels of BRD4 phosphorylation (FIGS. 59 and 60) induced by these PHICS to identify PHICS1 and PHICS2 as the most optimal for AMPK and PKC, respectively (FIGS. 8A and 11). Applicants used (−)-JQ1, the inactive enantiomer of the BRD4 binder (18), to synthesize iPHICS1 and iPHICS2 to serve as negative controls. Using the ADP-Glo™ assay, Applicants confirmed that iPHICS1 and iPHICS2 still activated AMPK and PKC (FIG. 61A-61C), as the kinase-binding moiety remains unaltered. The ternary complex formation was assessed by an AlphaScreen assay (Amplified Luminescent Proximity Homogenous assay) (19-20) using BRD4 and AMPK (α1β1γ1 isoform) or PKC (α-isoform). PHICS1 and PHICS2, but not iPHICS1 and iPHICS2, displayed the bell-shaped curve consistent with ternary-complex equilibria (FIGS. 39B and 39C) (21), where at high concentrations of the bifunctional molecule, the kinase-PHICS and BRD4-PHICS species dominate the equilibrium eliciting the “hook effect” (22). Using phospho-AMPK- or PKC-substrate motif antibodies, Applicants observed BRD4 phosphorylation only when PHICS, BRD4, and kinase were present (FIGS. 2C and 51A) (23-24). BRD4 phosphorylation was observed irrespective of the nature of the tag (i.e., GST vs. His tag) (FIGS. 61D-61E). The levels of BRD4 phosphorylation also increased in an AMPK-/PKC-dependent manner with PHICS but not with iPHICS (FIGS. 61F-61G). Applicants also observed the hook effect in BRD4 phosphorylation with an increasing PHICS concentration (FIG. 62) with the highest levels of phosphorylation achieved at 1 μM of PHICS1, corresponding to the concentration of maximum signal in the AlphaScreen assay for ternary-complex formation (FIG. 39B). Using pSer484/488 antibody, Applicants observed PHICS1 induced phosphorylation of those sites on BRD4 by AMPK (FIG. 51B), which is phosphorylated in the native environment by casein kinase II (CK2) (25).

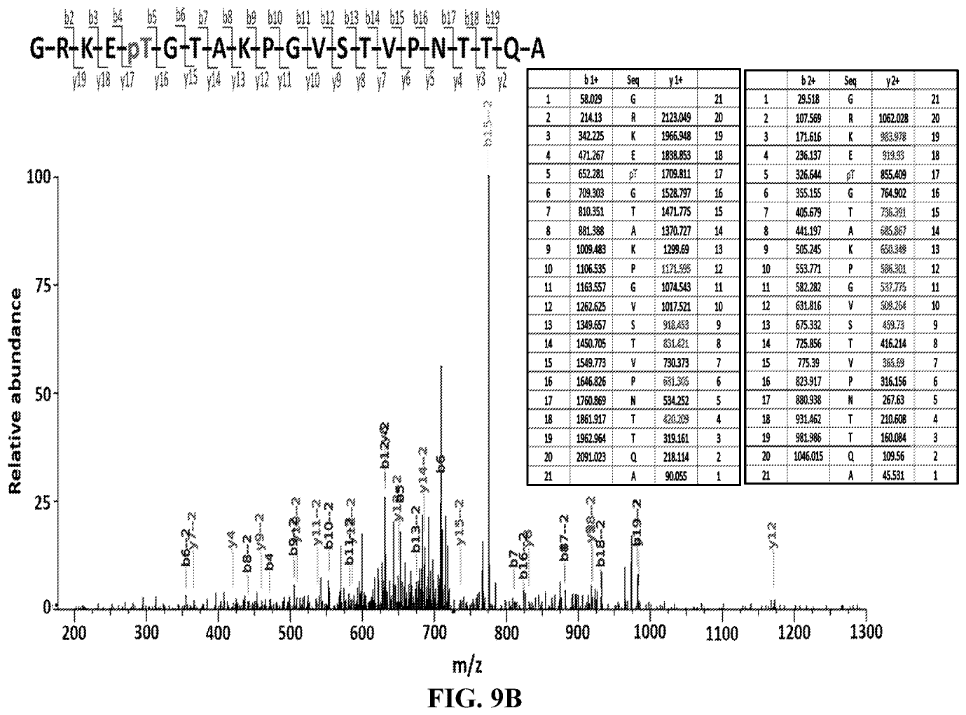

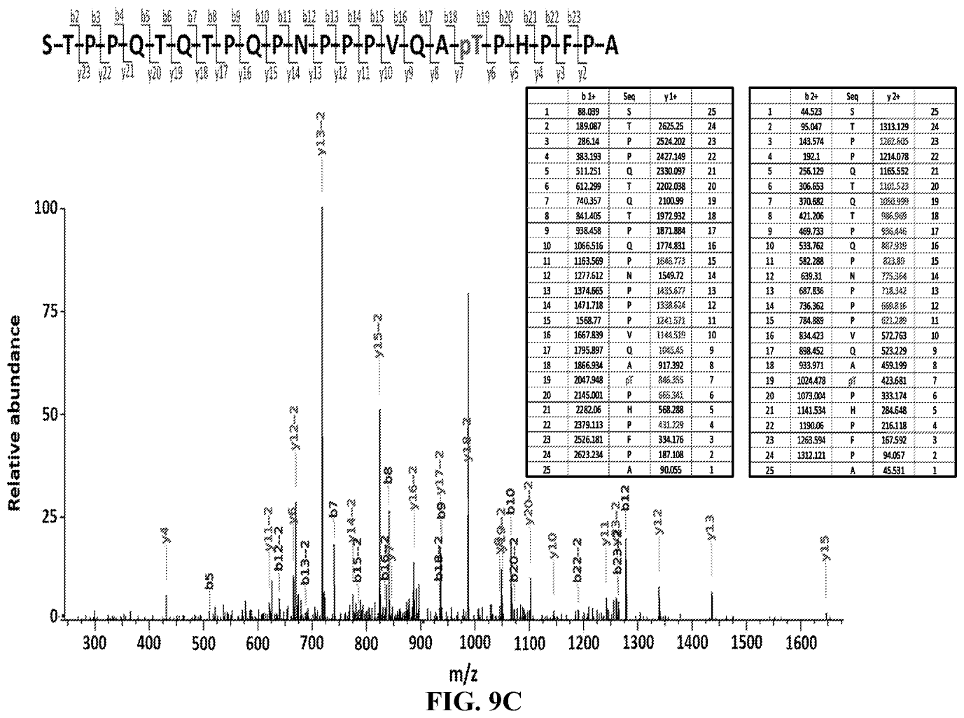

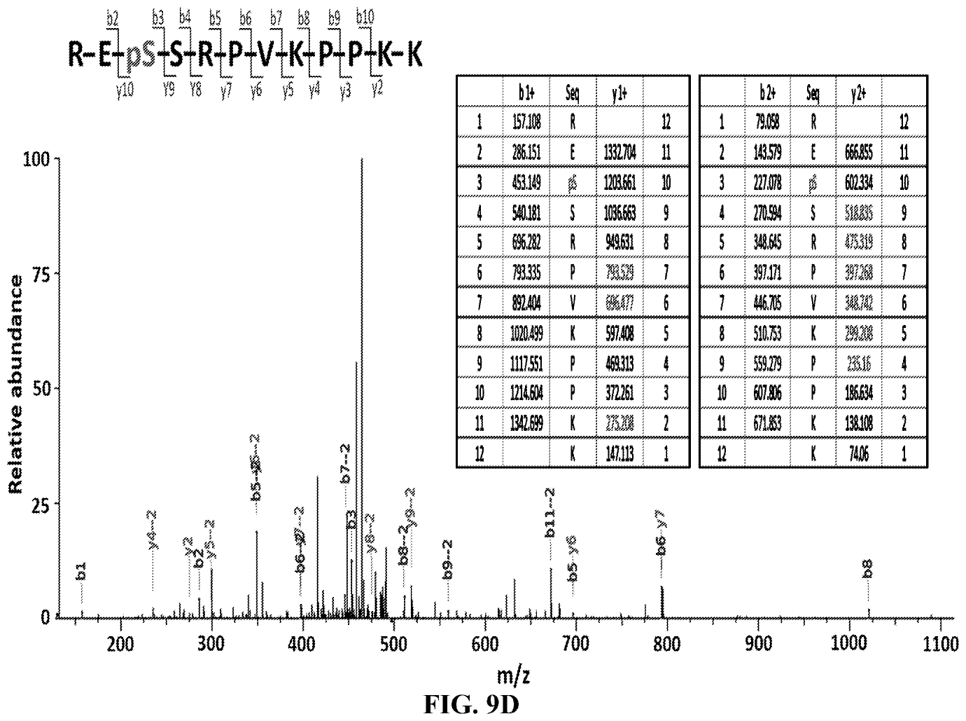

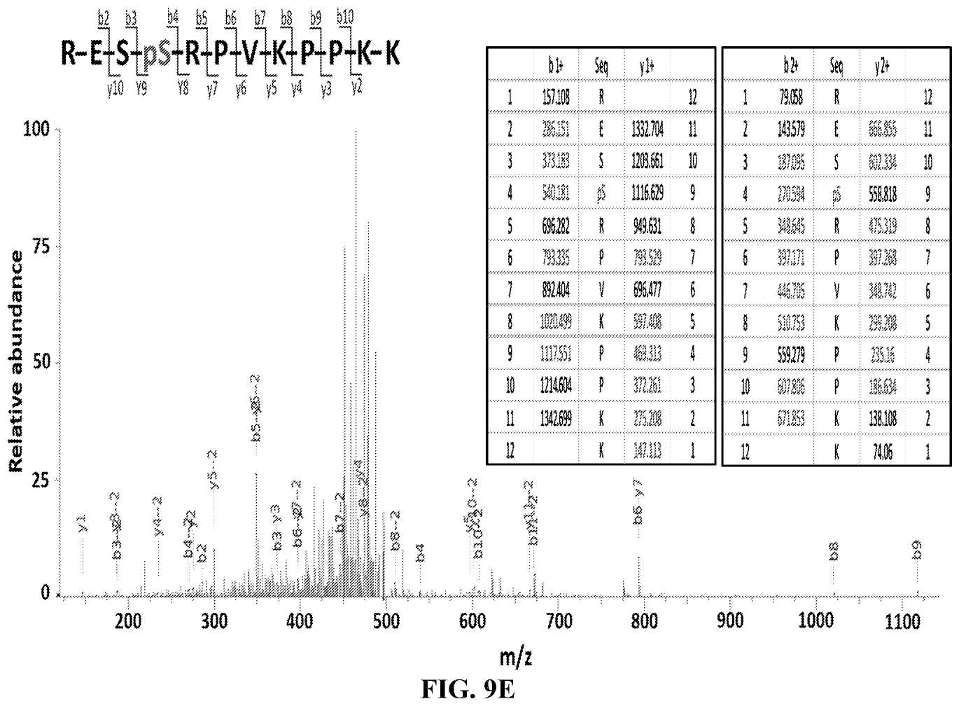

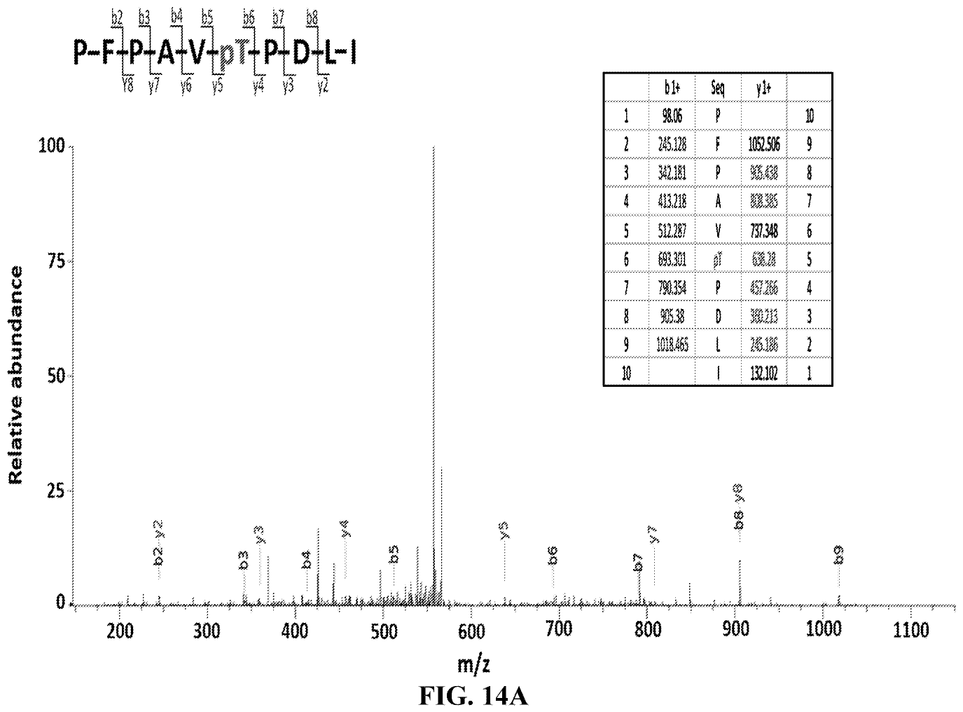

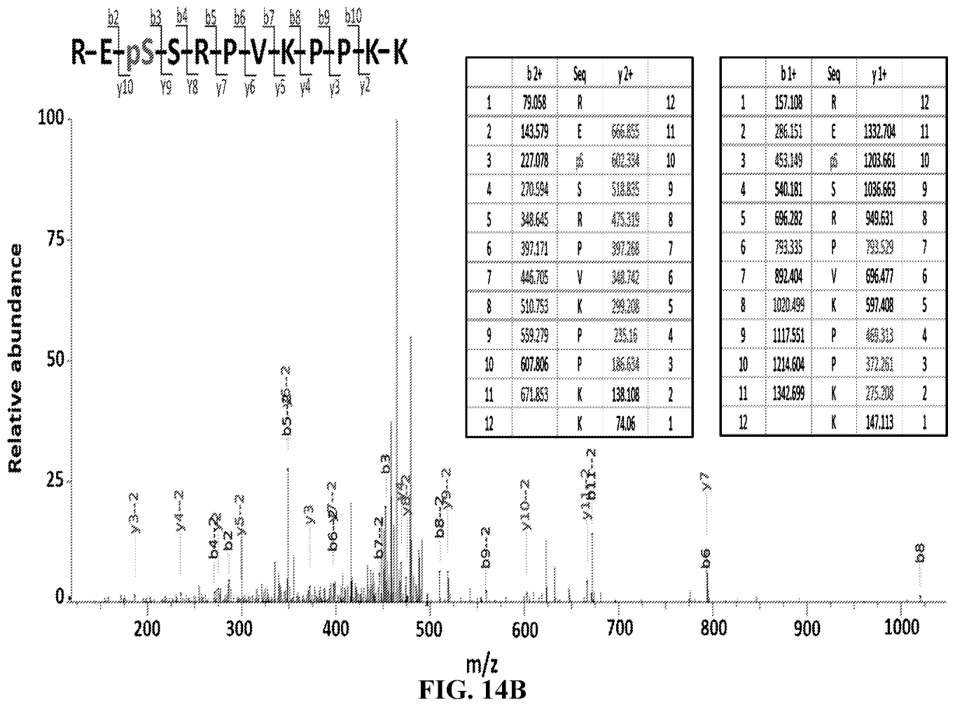

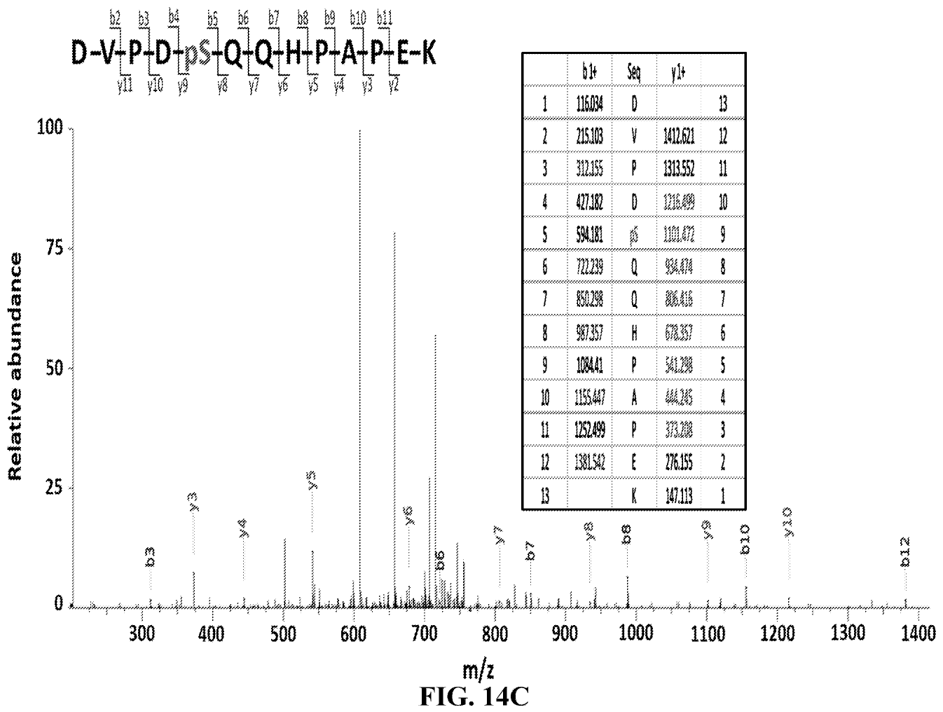

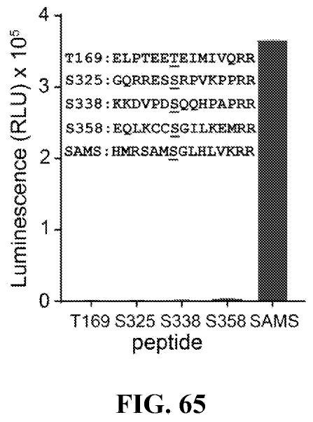

Next, Applicants confirmed that PHICS can induce neo-phosphorylation on the truncated BRD4 (49-460 aa) using mass spectrometry. For AMPK PHICS, the statistically significant phosphorylation sites were T169, T186, T221, S324, and S325, whereas those for PKC were T229, S324, and S338 (FIGS. 9 and 14). Phosphorylation at sites T169, T186, T221, and T229 are neo-phosphorylations as they have not been reported before (26). To confirm that AMPK does not have an intrinsic preference for phosphorylation of these BRD4 sites and that they are not part of unknown substrate motif, Applicants tested peptides derived from the BRD4 sequence bearing those residues. In an ADP-Glo assay, these peptides showed a 250-fold lower preference for phosphorylation than AMPK's natural ACC substrate—SAMS peptide (FIG. 65) (27), further confirming that the PHICS-induced proximity of AMPK and BRD4 is essential for rewiring the AMPK specificity. Finally, Applicants note that the sites T186, S324, and S325 but not T169 and T221 are in the most preferred AMPK consensus substrate recognition motif, wherein a basic residue is preferred in the −3 position (RXXpS/pT), but AMPK can also phosphorylate proteins lacking this motif (28-30).

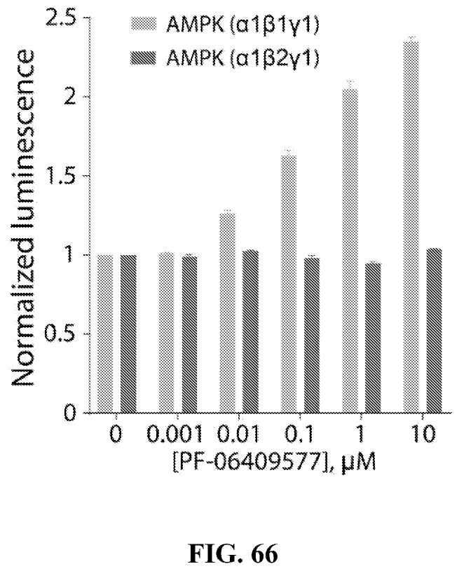

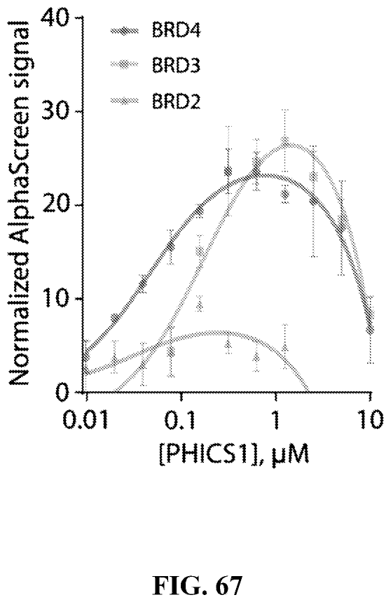

Since PHICS were designed based on reversible binders, Applicants hypothesized that they will exhibit turnover with each PHICS molecule phosphorylating multiple BRD4 molecules. Using the ADP-Glo™ assay and iPHICS1 and iPHICS2 as negative controls (FIGS. 51C and 51D, the calculated ADP production in the presence of the PHICS1 (324±22 nM) and PHICS2 (740±31 nM) was higher than the limiting AMPK (20 nM) or PKC (50 nM) concentrations, respectively, suggesting that PHICS exhibit turnover. Another hallmark of bifunctional molecules is the isoform specificity that arises from not only a differential binding affinity of PHICS to various isoforms but also from intrinsically different interactions between the enzyme and the target isoform upon ternary complex formation (31). With an AMPK isoform (α1β2γ1) that is not activated by PF-06409577 (FIG. 66) (14) Applicants did not observe the induction of BRD4 phosphorylation by PHICS1 (FIG. 52A). Additionally, PHICS2 also exhibited isoform specificity, with the highest BRD4 phosphorylation occurring with PKCα, modest phosphorylation with PKCβI and II, and only minor phosphorylation with the PKCγ and δ isoforms (FIG. 52B) (32). Isoform specificity was also observed for the target proteins BRD (2/3/4), with BRD4 showing the highest level of phosphorylation (FIGS. 2H, 3E and 67).

Applicants were unable to observe PHICS-mediated BRD4 phosphorylation in cells perhaps owing to different localization of the kinases and BRD4—while the former is mostly cytosolic, the latter primarily resides in the nucleus. Applicants chose Bruton's Tyrosine Kinase (BTK), a protein widely expressed in B cells (33-34) for several reasons. First, BTK is a cytoplasmic protein and thus available for interactions with cytoplasmic AMPK. Second, while BTK can interact with PKC, it is not known to interact with AMPK (35). Third, high-quality chemical probes of BTK and their co-crystal structures are available, allowing rational design of PHICS and inactive controls by engineering BTK or PHICS (36). Fourth, BTK possess a phosphorylation site (S180) that not only lies in substrate-like motif of AMPK but plays an important role in negative regulation of BTK (35). Finally, BTK is undetectable in HEK293T cells allowing us to assess the ability of PHICS to induce BTK phosphorylation in a non-native cellular environment.

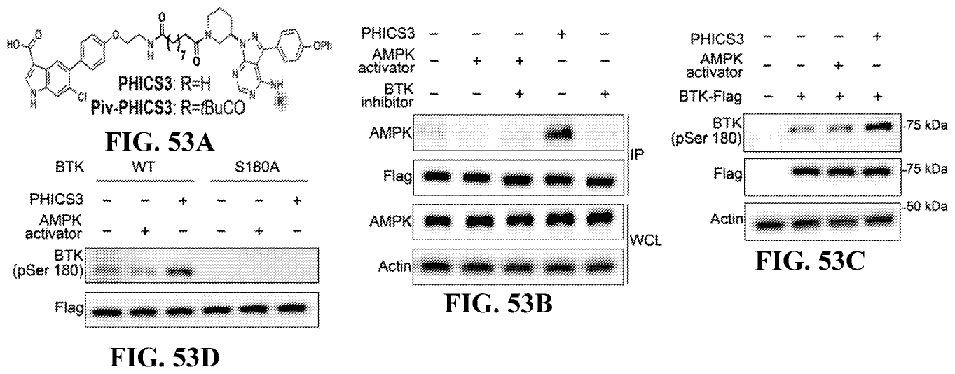

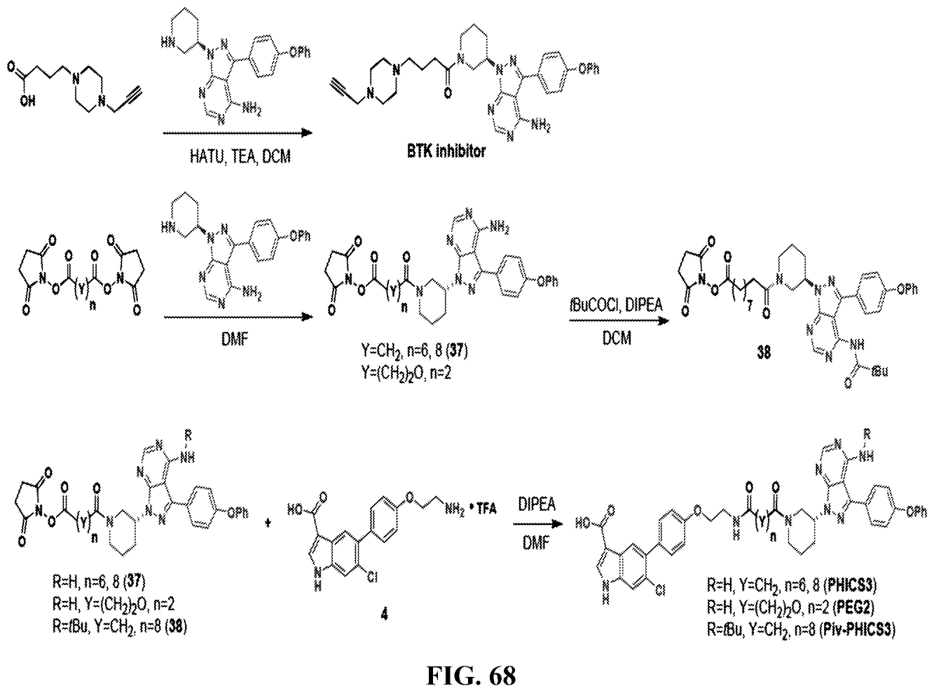

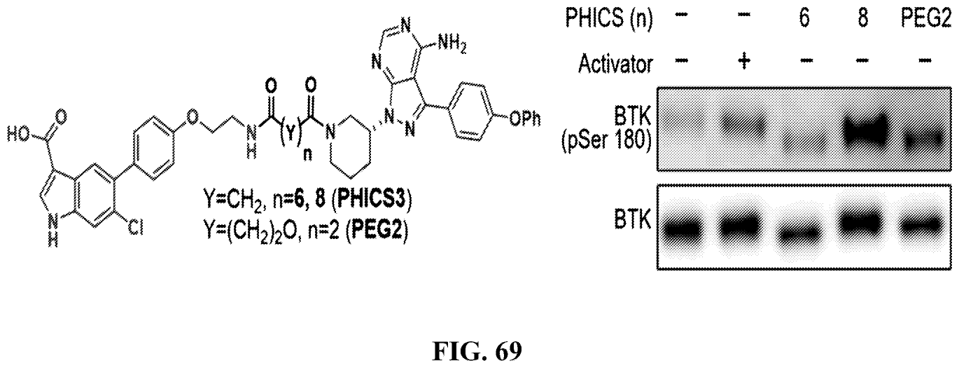

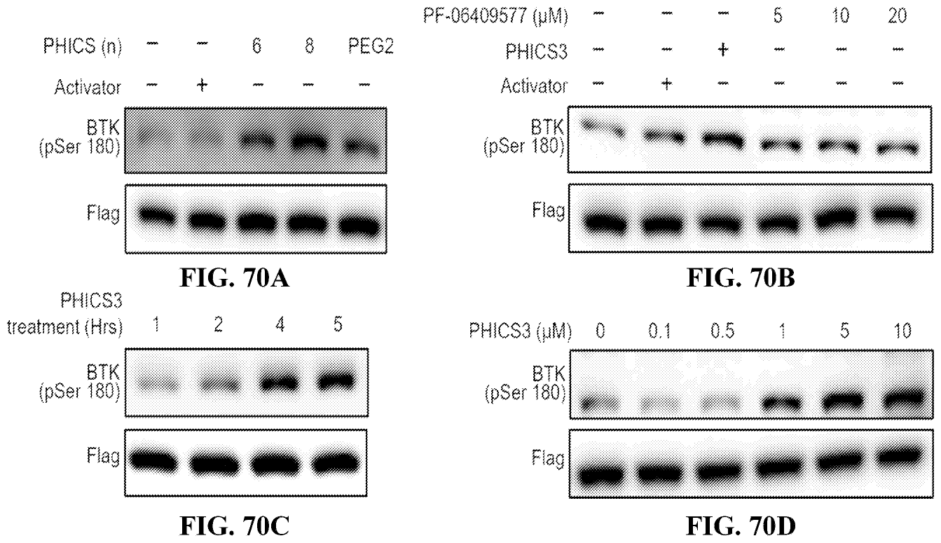

Applicants linked AMPK binder with a non-covalent analog of ibrutinib via various linkers (37) (FIG. 68) and found eight carbon alkyl linker (PHICS3, FIG. 53A) to be optimum for the in vitro phosphorylation (FIG. 69), which was monitored using phospho BTK (Ser180) antibody. To demonstrate the ternary complex formation inside the cells, Applicants transfected HEK293T with BTK-Flag and performed co-immunoprecipitation of AMPK with BTK in the presence of PHICS3 (FIG. 53B). Furthermore, Applicants were able to detect PHICS-mediated BTK phosphorylation at Ser180 (FIGS. 53C and 70A) and Applicants validated this site of phosphorylation using S180A mutant, where the phosphorylation was not detected (FIG. 53D). It is noted that the potent AMPK activator, PF-06409577, alone did not induce the same levels of BTK phosphorylation as PHICS3, even at 4-fold higher concentration (FIG. 70B). Finally, Applicants were able to dosably control PHICS-induced S180 phosphorylation with fast kinetics (FIGS. 70C and 70D).

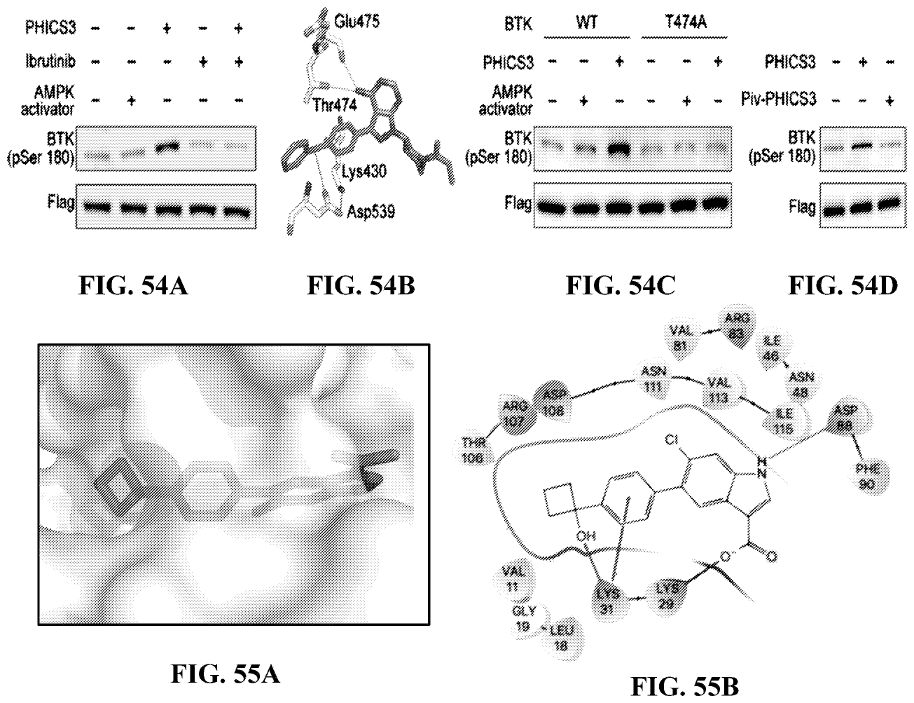

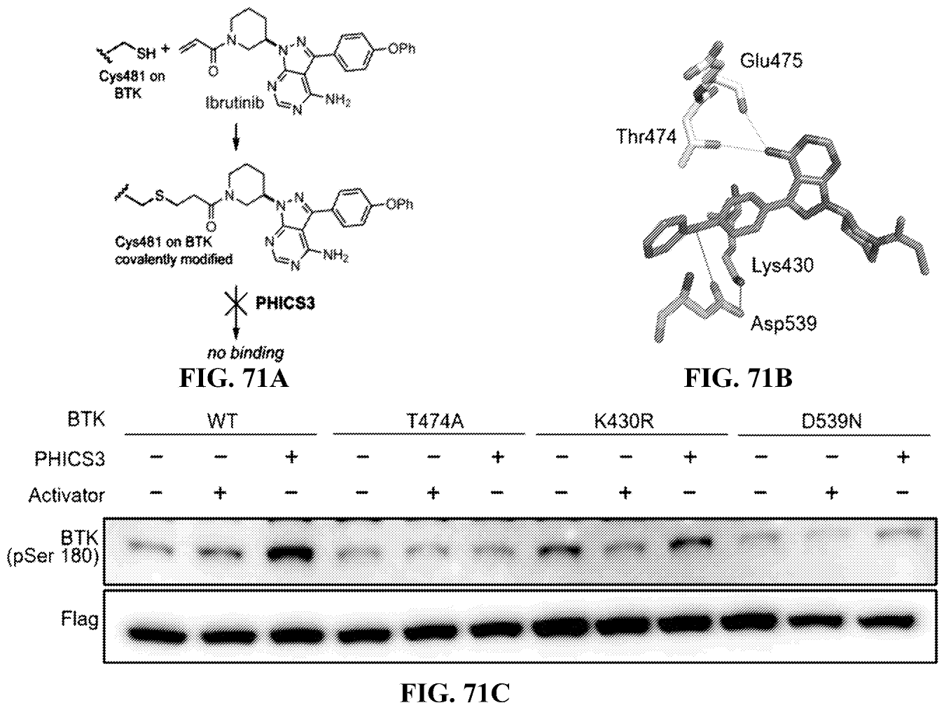

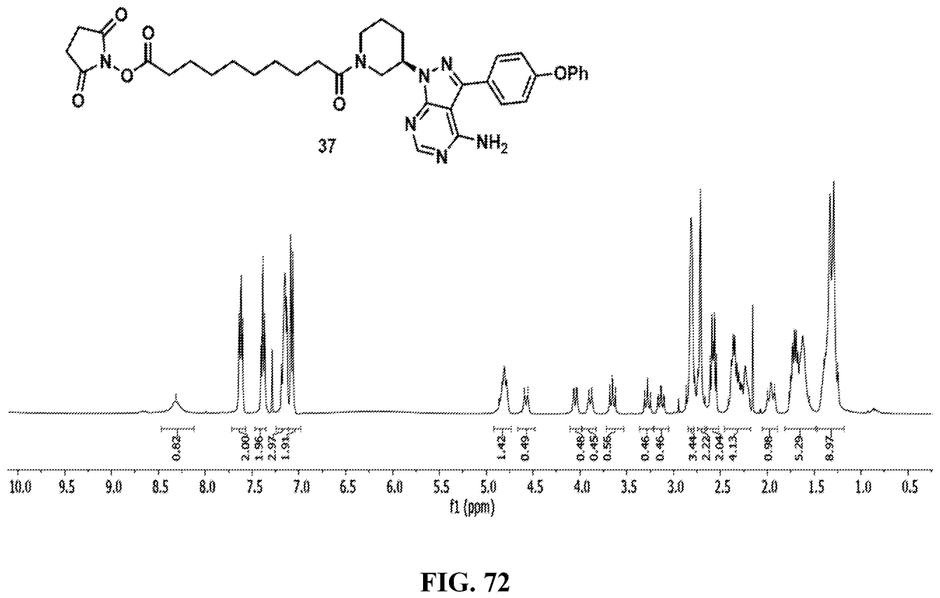

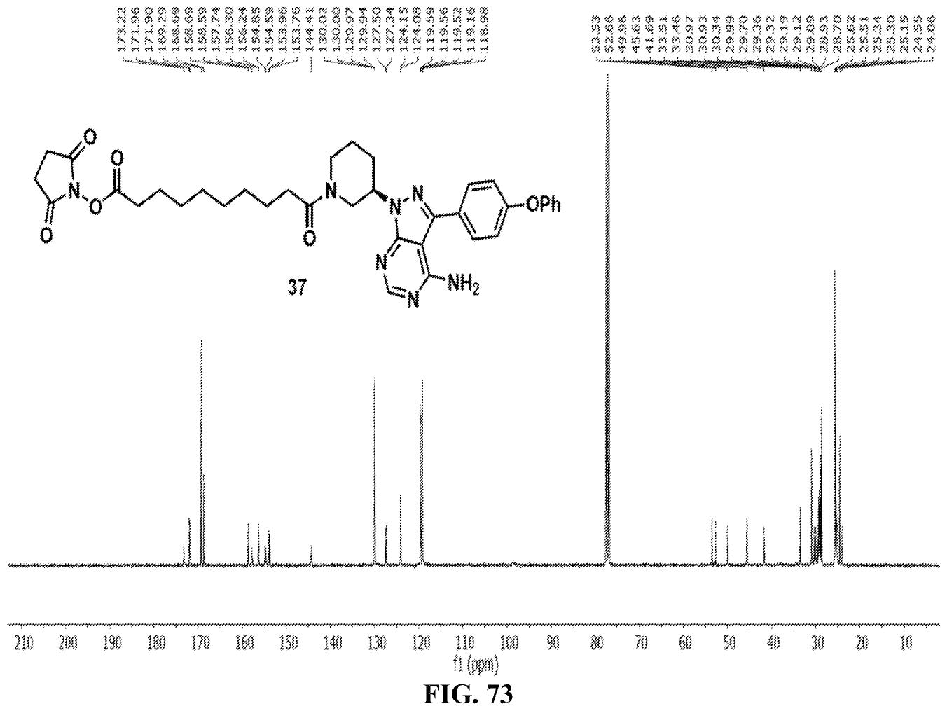

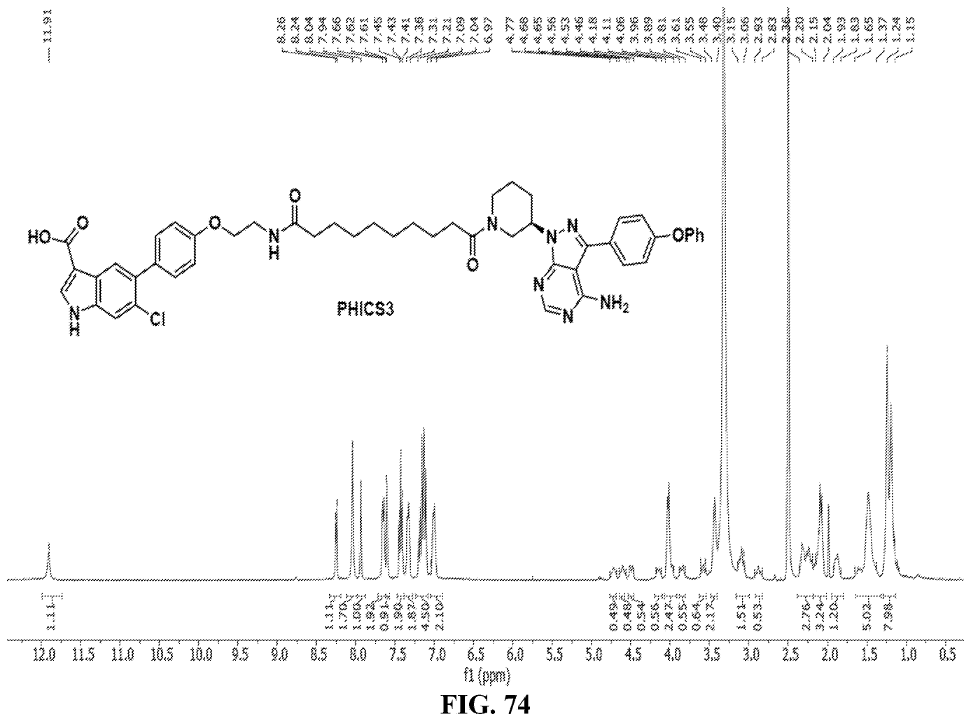

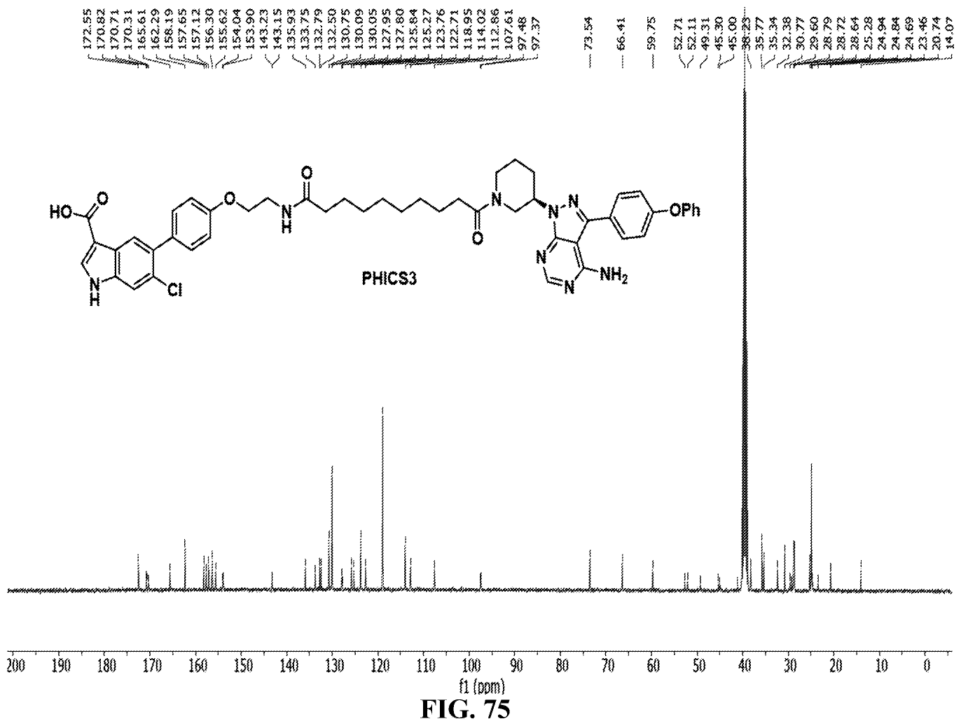

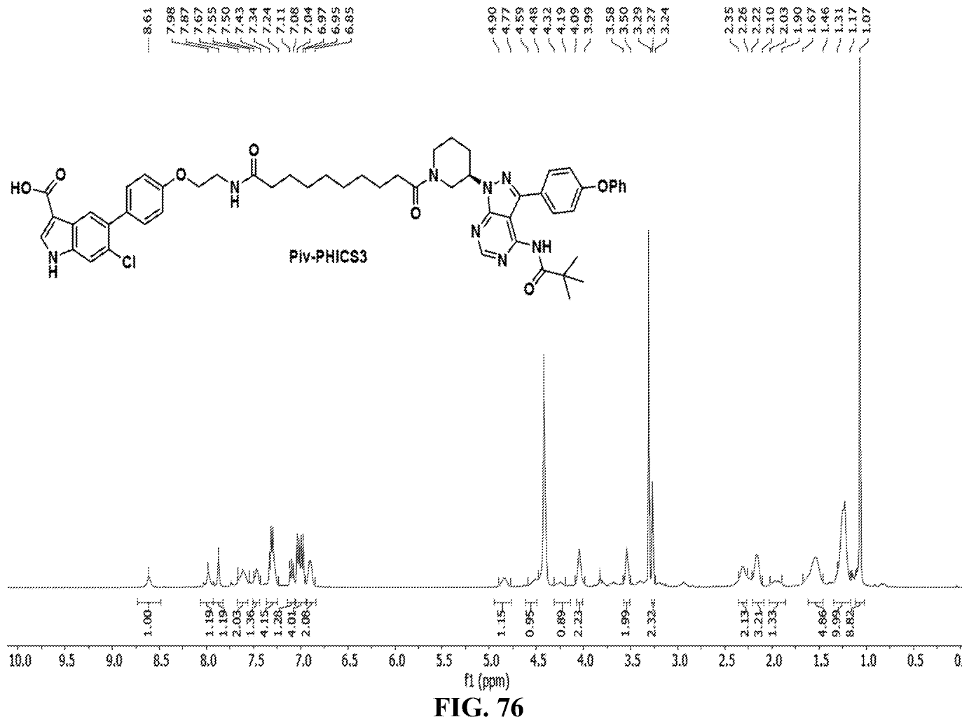

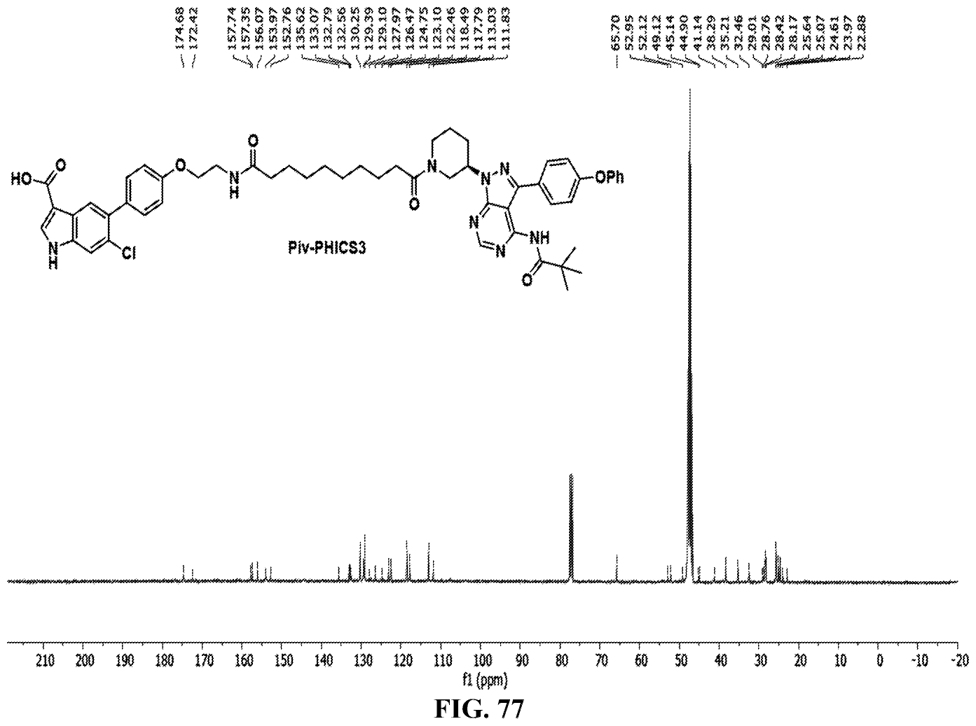

To confirm that the observed BTK phosphorylation in the non-native environment was mediated by PHICS, Applicants leveraged several chemical genetics approaches. First, the pre-treatment of cells with covalent BTK binder, ibrutinib, demolished the ability of PHICS3 to induce BTK phosphorylation (FIGS. 54A and 71A). Second, Applicants mutated the residues purported to be involved in binding of PHICS to BTK (Thr474, Lys430, and Asp539). Notably, Thr474 forms hydrogen bond with the 4-amino group of Ibrutinib (FIG. 54B) (38) and Applicants observed drastically lowered BTK phosphorylation for the T474A mutant (FIG. 54C). Alterations in the ATP binding pocket by D539N and K430R mutations also markedly reduced the PHICS3 induced phosphorylation (FIGS. 71B and 71C). Finally, Applicants designed an inactive analog of PHICS3, by placing a bulky pivaloyl (Piv) group on the aza-purine nitrogen that should sterically prevent the binding to BTK. Indeed, the pivaloyl-bearing PHICS (Piv-PHICS3, FIG. 53A) was unable to induce a significant BTK phosphorylation (FIG. 54D). Taken together, these studies confirm that the Ser180 phosphorylation of BTK arises owing to proximity-effects induced by PHICS.

Herein, Applicants successfully rewired both AMPK and PKC kinases using chimeric small molecules to induce novel phosphorylation events, including neo-phosphorylation of BRD4 and a signaling-relevant phosphorylation of BTK. PHICS exhibited the hallmarks of a typical bifunctional molecule, including the hook effect, turnover, dependence on proximity (linker length), isoform specificity, and dose- and temporal-control of phosphorylation. The turnover likely arises from the reversible binding of the PHICS to the kinase and target protein, which is also observed for PROTACs. Kinase specificity has been previously rewired using adaptor proteins (12-13), but Applicants focused on small molecules since they are cell-permeable and non-immunogenic. Furthermore, they afford facile dose and temporal controls, exhibit fast kinetics and turnover, and possess modular design allowing rapid assembly. While these studies have focused on serine/threonine-kinases, Applicants are investigating the PHICS-mediated rewiring of tyrosine kinases. Future studies will deploy PHICS to induce signaling-relevant phosphorylation as well as neophosphorylation of oncogenic proteins that could evoke an immune response against a tumor or the PHICS-mediated deposition of a negative charge on the DNA-binding domains of transcription factors (which are often deemed chemically undruggable) (39) that could adversely impact their ability to bind to DNA. Overall, PHICS expands the toolkit of chimeric small molecules that can be used to induce various post-translational modifications.

### Materials and Methods

### 2.1 Materials and General Procedures

AMPK α1β1γ1-His tag (P47-10H), AMPK α1β2γ1-His tag (P50-10H), PKCα-GST (P61-18G), PKCβI-GST (P62-18G), PKCβII-GST (P63-18G), PKCγ-GST (P66-18G), PKCδ-GST (P64-18G), kinase assay buffer III 5× (K03-09), SAMStide peptide, HMRSAMSGLHLVKRR (S07-58) and CREBtide peptide, KRREILSRRPSYR (C50-58) were all purchased from SignalChem. BRD4-GST ((BD1 and BD2 (49-460))(31044) and BRD4-His tag ((BD1 and BD2 (49-460))(31045), BRD3-GST ((BD1 and BD2 (29-417))(31035), BRD2-GST ((BD1 and BD2 (65-459))(31024) were purchased from BPS Bioscience. ADP-Glo™ Kinase Assay kit (V6930) was from Promega Corporation. OptiPlate-384 (White Opaque 384-well Microplate, 6007290), Nickel Chelate AlphaLISA Acceptor Beads (AL108C) and Alpha Glutathione Donor beads (6765300) were all purchased from Perkin Elmer. NuPAGE 4-12% Bis-Tris Protein Gels (NP0336 or NP0335) were from ThermoFisher and Ni-NTA agarose beads were purchased from Qiagen.

For chemical synthesis, all reagents were purchased and used as received from commercial sources without further purification. Reactions were performed in round-bottom flasks stirred with Teflon®-coated magnetic stir bars. Moisture and air-sensitive reactions were performed under a dry nitrogen/argon atmosphere. Moisture and air-sensitive liquids or solutions were transferred via nitrogen-flushed syringes. As necessary, organic solvents were degassed by bubbling nitrogen/argon through the liquid. The reaction progress was monitored by thin-layer chromatography (TLC) and ultra-performance liquid chromatography mass spectrometry (UPLC-MS). Flash column chromatography was performed using silica gel (60 Å mesh, 20-40 μm) on a Teledyne Isco CombiFlash Rf system. Analytical TLC was performed using Merck Silica gel 60 F254 pre-coated plates (0.25 mm); illumination at 254 nm allowed the visualization of UV-active material, and a phosphomolybdic acid (PMA) stain was used to visualize UV-inactive material. UPLC-MS was performed on a Waters ACQUITY UPLC I-Class PLUS System with an ACQUITY SQ Detector 2. Nuclear magnetic resonance (NMR) spectra were collected on a Bruker AVANCE III HD 400 MHz spectrometer at room temperature (¹H NMR, 400 MHz; ¹³C, 101 MHz) at the Broad Institute of MIT and Harvard. ¹H and ¹³C chemical shifts are indicated in parts per million (ppm) and internally referenced to residual solvent signals. NMR solvents were purchased from Cambridge Isotope Laboratories, Inc., and NMR data were obtained in CDCl₃ and DMSO-d₆. Data for ¹H NMR are reported as follows: chemical shift value in ppm, multiplicity (s=singlet, br s=broad singlet, d=doublet, t=triplet, dd=doublet of doublets, and m=multiplet), integration value, and coupling constant value in Hz. Tandem liquid chromatography-mass spectrometry (LCMS) was performed on a Waters 2795 separations module with a 3100 mass detector.

### 2.2 Molecular Docking

Docking was performed with the standard precision protocol using Schrödinger Maestro v11.6. The ligands were prepared by generating possible states at pH 7.0±2.0 using Epik, desalted, and subjected to OPLS3e force field. 2D ligand interaction maps were generated from the docking results to predict linker attachment sites.

### 2.3 ADP-Glo Kinase Assay to Validate the Kinase Activation by PHICS Molecule

ADP-Glo assay¹ was performed in the presence of PHICS molecules with different linkers to validate their potential to activate the kinase (AMPKα1β1γ1 or PKCα) and to confirm their binding to the kinase. In a 96 well plate (white, flat bottom), concentration series of PHICS (1.1, 1.2, 1.3, 1.4 and 1.5) molecules were prepared using kinase assay buffer (40 mM Tris-HCl pH 7.5, 20 mM MgCl2, 0.1 mg/ml BSA, 50 μM DTT, 1% DMSO) and incubated with 5 ng of AMPK and 0.2 μg/μL SAMStide peptide in the presence of 150 μM ATP. After 2 hrs of incubation at room temperature, ADP-Glo reagent was added to the kinase reaction mixture in 1:1 ratio and maintained the reaction mixture for another 40 min at room temperature. Finally, kinase detection reagent was added to the mixture in 1:2 ratio and incubated for another 30 min at room temperature before recording luminescence by Envision 2104 plate reader (PerkinElmer). Kinase Activation by PHICS molecule was calculated after removing the background signal coming from the no AMPK control and data was plotted using GraphPad PRISM version 8.1.1 normalized to DMSO control.

ADP-Glo assay was performed with PHICS (PKC) in a similar manner with some modifications as follows. In a 96 well plate (white, flat bottom), similar concentration series of PHICS (2.1, 2.2, 2.3, 2.4 and 2.5) were prepared using same kinase assay buffer and incubated with 2 ng of PKC, 0.2 μg/μL CREBtide peptide and 50 μM ATP for 1 hr at room temperature before incubation with ADP-Glo assay reagents.

### 2.4 ADP-Glo Kinase Assay to Evaluate the Catalytic Nature Between Kinase and PHICS Molecules

ADP-Glo assay was performed with a mixture of Kinase, BRD4-GST (BD1 and BD2) and PHICS molecule to determine the enzyme turnover efficiency with PHICS (1.2 or 2.3, facilitator of ternary complex formation) compared to the PHICS ((R)-1.2 or (R)-2.3. obstructer of ternary complex formation). Initially, 1 μM of PHICS1.2 and (R)-PHICS1.2 were prepared in a 96 well plate (white, flat bottom) using kinase assay buffer (40 mM Tris-HCl pH 7.5, 20 mM MgCl2, 0.1 mg/ml BSA, 50 μM DTT, 1% DMSO) and incubated with 20 nM AMPK and 700 nM BRD4-GST (BD1 and BD2) for 2 hrs at room temperature in the presence of 150 μM ATP. Then ADP-Glo assay reagents were added in a similar manner and luminescence was recorded using Envision 2104 plate reader (PerkinElmer). The actual signal coming from the PHICS1.2 mediated kinase reaction (20 nM AMPK, 1 μM PHICS1.2, 700 nM BRD4-GST and 150 μM ATP) was calculated by subtracting the luminescence signal of inactive control (20 nM AMPK, 1 μM (R)-PHICS1.2, 700 nM BRD4-GST and 150 μM ATP). ADP generation during the kinase reaction was determined through a standard curve (luminescence (RLU) versus % ATP to ADP conversion) which was plotted according to the Promega specifications.

The same protocol was followed for PHICS2.3 as well. Initially, 1 μM of PHICS2.3 and (R)-PHICS2.3 were prepared in a 96 well plate (white, flat bottom) using kinase assay buffer and incubated with 50 nM PKC and 700 nM BRD4-GST (BD1 and BD2) for 1 hr at room temperature in the presence of 50 μM ATP before adding ADP-Glo reagents.

### 2.5 ADP-Glo Kinase Assay to Assess the BRD4 Peptide Phosphorylation

ADP-Glo assay was performed with several synthesized peptides from BRD4 protein to evaluate the preference of AMPK towards phosphorylating these peptide substrates. Peptide regions shown below were selected based on the mass spectrometry analysis and purchased from GenScript.

S325=GQRRESSRPVKPPRR (SEQ ID NO: 2) (Confirmed by the Mass spec as a target of phosphorylation)

T169=ELPTEETEIMIVQRR (SEQ ID NO: 3) (Confirmed by the Mass spec as a target of phosphorylation)

S338=KKDVPDSQQHPAPRR (SEQ ID NO: 4) (Potential phosphorylation site on BRD4)

S358=EQLKCCSGILKEMRR (SEQ ID NO: 5) (Potential phosphorylation site on BRD4)

Kinase reaction was performed in a similar manner by incubating 0.2 μg/μL of each peptide with 20 nM AMPK for 2 hrs at room temperature in the presence of 150 μM ATP before adding the ADP-Glo reagents. ADP-Glo kinase assay with 0.2 μg/μL SAMStide peptide and 20 nM AMPK was used as a positive control and luminescence signal generated from each BRD4 peptide was compared with this positive control to determine the preference of AMPK on phosphorylating these BRD4 peptides.

### 2.6 AlphaScreen Assay to Determine the PHICS Induced Protein Dimerization

AlphaScreen assay² was performed to validate the PHICS induced ternary complex formation between kinase (AMPK or PKC): PHICS molecule: BRD4 (BD1 and BD2). Initially, concentration series of PHICS (1.1, 1.2, 1.3, 1.4 and 1.5) with different linkers or (R)-PHICS1.2 with DMSO control were prepared in a white opaque 384-well microplate using a dilution assay buffer (50 mM HEPES pH 7.4, 150 mM NaCl, 0.1% w/v BSA, 0.01% v/v Tween 20) with 1% DMSO in the final mixture. Then 7 nM AMPK (6×His tag) and 67 nM BRD4-GST were added to the mixture and incubated at room temperature for 1 hr. Then, Nickel Chelate AlphaLISA acceptor beads (PerkinElmer) and Glutathione donor beads (PerkinElmer) were added to the mixture in a final concentration of 20 μg/mL. Following 1 hr incubation at room temperature, luminescence was recorded using Envision 2104 plate reader (PerkinElmer). Normalized luminescence value was calculated after removing background signals from DMSO control or no protein wells. Data were analyzed and plotted using GraphPad PRISM version 8.1.1. AlphaScreen assay was performed in a similar manner to determine the ternary complex formation between AMPK: PHICS1.2: BRD3 (BD1 and BD2) or BRD2 (BD1 and BD2) as well. Initially, concentration series of PHICS1.2 was prepared using dilution buffer and incubated with 7 nM AMPK and 67 nM BRD3-GST or BRD2-GST for 1 hr at room temperature before addition of acceptor and donor beads.

A similar approach was followed to determine the PHICS2 induced ternary complex formation between PKC: PHICS2: BRD4-GST (BD1 and BD2). Initially, concentration series of PHICS (2.1, 2.2, 2.3, 2.4 and 2.5) with different linkers and (R)-PHICS2.3 with DMSO control were prepared in a white opaque 384-well microplate using a dilution assay buffer with 1% DMSO in the final mixture. Then 10 nM PKC-GST and 100 nM BRD4 (6×His tag) were added to the mixture and incubated at room temperature. Following 1 hr incubation, acceptor beads and donor beads were added to the system.

### 2.7 Pull-Down Assay to Determine the Ternary Complex Formation

The interaction between AMPK and BRD4 via PHICS1.2 was determined by in vitro pull-down assay. His-tagged AMPK (70 nM) was immobilized on Ni-NTA agarose beads by incubating at 4° C. in binding buffer (50 mM HEPES pH 7.4, 150 mM NaCl, 0.1% w/v BSA, 0.01% v/v Tween 20, 10 mM imidazole). After 1 hr, beads were washed twice and incubated with BRD4-GST (300 nM) and PHICS1.2 or (R)-PHICS1.2 (1 μM) for 4 hrs at 4° C. Next the beads were extensively washed, mixed with SDS loading buffer and heated for 5 min at 95° C. Western blot analysis was performed with antibodies against AMPK-α (Cell Signaling, Cat #5832) and BRD4 (Biovision, Cat #6644).

### 2.8 Immunoblotting Analysis to Confirm the BRD4 Phosphorylation

Proximity driven phosphorylation at Ser/Thr on BRD4 induced by PHICS was confirmed through western blotting using phospho-(Ser/Thr) kinase substrate antibodies. Initially, 10 μM, 5 μM, and 1 μM concentrations of PHICS1 with different linkers (1.1, 1.2, 1.3, 1.4 and 1.5), (R)-PHICS1.2 and DMSO control were prepared using 1× kinase assay buffer with 1% DMSO. Then kinase reaction was performed in the presence 20 nM AMPK, 700 nM BRD4-GST and 150 μM ATP for 2 hrs at room temperature. In another experiment, kinase reaction was performed with diluting concentrations of PHICS1.2 in a similar manner. In addition to BRD4-GST, same kinase reaction was performed with 700 nM BRD3-GST (BD1 and BD2) and 700 nM BRD3-GST (BD1 and BD2) in the presence or absence of PHICS1.2. Importance of GST tag on BRD4 for PHICS induced phosphorylation was validated with BRD4-(6×His tag) (BD1 and BD2) (700 nM) version following same kinase assay. Following 2 hrs of incubation, kinase reaction was quenched by adding SDS loading buffer. Proteins were resolved by NuPAGE 4-12% Bis-Tris protein gels and transferred to a PVDF membrane. Then membrane was incubated at room temperature for 1 hr in a blocking buffer (TBS with 0.1% tween 20 and 5% BSA). After that membrane was incubated with phospho-AMPK substrate motif [LXRXX(pS/pT) (Cell Signaling, Cat #5759) (1:1000) primary antibody to detect the phosphorylation at Ser/Thr on BRD4 and anti-BRD4 primary antibody (Biovision, Cat #6644) (1:1000) to detect loading levels. Following overnight incubation at 4° C., membrane was washed three times with TBST buffer (TBS with 0.1% tween 20). Protein bands were visualized by either chemiluminescence (Azure Biosystems C600 imager) with appropriate HRP-conjugated secondary antibodies (Rabbit/Mouse, Cell Signaling, Cat #7074/7076) or NIR fluorescence (LI-COR Odyssey Imager) with IRDye 800CW/IRDye 680RD secondary antibodies (Rabbit/Mouse, LI-COR, Cat #926-32211/926-68070) after washing membrane three times with TBST buffer.

PHICS induced phosphorylation on BRD4 by PKC was also validated using similar conditions. Kinase reaction was performed using 1 μM PHICS (2.1, 2.2, 2.3, 2.4, 2.5 and (R)-2.3), 50 nM PKC, 700 nM BRD4-GST and 50 μM ATP for 1 hr at room temperature before quenching with SDS loading buffer. Proteins were resolved in NuPAGE 4-12% Bis-Tris protein gels and transferred to a PVDF membrane. After incubation of the membrane with the blocking buffer, phospho-PKC substrate motif [(R/K)XpSX(R/K)] (Cell Signaling, Cat #6967) (1:1000) primary antibody was added to the membrane and further incubated at 4° C. for overnight. HRP-conjugated secondary antibodies or IRDye 800CW/IRDye 680RD secondary antibodies were used to visualize the protein bands.

Similar approaches were followed to determine the phosphorylation of BRD4 with different AMPK and PKC isoforms as well as phosphorylation of different BRD proteins.

### 2.9 Mass Spectrometry Analysis to Identify the Phosphorylated Sites in BRD4

Kinase reaction was performed in the presence of AMPK, BRD4, and PHICS and mass spectrometry study was performed to determine the proximity driven phosphorylation sites on BRD4. Initially, kinase reaction was performed in a similar manner using 20 nM AMPK with 700 nM BRD4-GST (BD1 and BD2) in the presence of 1 μM PHICS1.2 or (R)-PHICS1.2. The reaction was quenched after 2 hrs incubation by adding 6× loading dye. Proteins in each reaction mixture with PHICS1.2, (R)-PHICS1.2 and DMSO control were resolved by NuPAGE 4-12% Bis-Tris protein gels and the gel slice with BRD4-GST was submitted to the Taplin Mass Spectrometry Facility in Harvard Medical School to determine the phosphorylation sites after digestion with trypsin or elastase.

### 2.10 Synthesis of PHICS1 Analogs

(+)-JQ1 PA was purchased from MedChemExpress and (−)-JQ1 PA, the inactive analog, was synthesized according to literature³. Compounds 5-9 were either purchased or synthesized via amide coupling with the precursor azido acid and N-hydroxysuccinimide, washed with water, then used without further purification for the next step⁴.

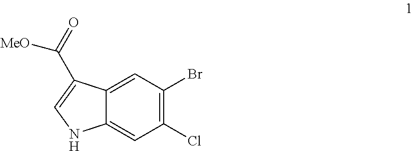 [SMILES: COC(=O)c1c[nH]c2cc(Cl)c(Br)cc12]

Methyl 5-bromo-6-chloro-1H-indole-3-carboxylate (1)

Following a literature procedure, 5-bromo-6-chloro-1H-indole (2.22 g, 0.63 mmol) was dissolved in 50 mL of DMF and cooled to 0° C. in an ice bath. Trifluoroacetic acid anhydride (5.4 mL, 38.5 mmol) was added slowly to this solution while stirring. After 1 hour, the reaction was quenched with saturated Na2CO3(aq) and a tan precipitate was filtered and collected. This crude precipitate was directly treated with 3M NaOH(aq) and refluxed overnight to form the carboxylic acid. Then, the reaction was acidified to a pH of 1-2, extracted with EtOAc (100 mL×3), dried over Na2SO4, and evaporated to dryness under reduced pressure. The crude solid was refluxed in 50 mL MeOH and 1 mL of conc. H2SO4 to afford the methyl ester as a reddish solid (2.00 g, 72% yield).

The data match literature reports.

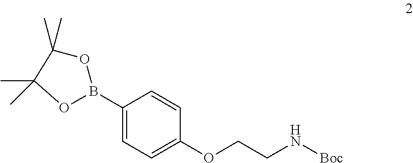 [SMILES: CC(C)(C)OC(=O)NCCOc1ccc(B2OC(C)(C)C(C)(C)O2)cc1]

### N-Boc-((4-aminoethoxy)phenyl)boronic acid pinacol ester (2)

Following a literature procedure, 4-hydroxyphenyl boronic acid (2.00 g, 9.09 mmol), 2-(Boc-amino)ethylbromide (2.04 g, 9.09 mmol), and oven-dried K₂CO₃ (3.77 g, 27.26 mmol) was added to 40 mL DMF and the resulting suspension was heated at 60° C. overnight. Then, water was added to the reaction mixture and the aqueous layer extracted with EtOAc (100 mL×3). After drying over Na₂SO₄ and concentrating under reduced pressure, the crude oil was purified by silica gel column chromatography (4:1 hexanes:EtOAc) to afford a colorless oil (3.04 g, 92% yield). The data matches literature reports⁵.

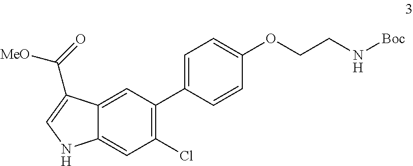 [SMILES: COC(=O)c1c[nH]c2cc(Cl)c(-c3ccc(OCCNC(=O)OC(C)(C)C)cc3)cc12]

### Methyl 5-(4-(2-((N-Boc)amino)ethoxy)phenyl)-6-chloro-1H-indole-3-carboxylate (3)

Methyl 5-bromo-6-chloro-1H-indole-3-carboxylate (0.97 g, 3.36 mmol) and N-Boc-((4-aminoethoxy)phenyl)boronic acid pinacol ester (1.22 g, 3.36 mmol) was suspended in 67 mL toluene. Potassium carbonate (3.02 g, 21.85 mmol) was dissolved in 33 mL deionized water and added to the toluene mixture, then Pd(dppf)Cl₂ DCM (0.247 g, 0.303 mmol) was added. The reaction mixture was quickly degassed then refluxed for 2 hours. Then, the reaction was concentrated under reduced pressure, redissolved in ethyl acetate, filtered through a pad of celite, washed with water and brine, then dried with Na₂SO₄ and concentrated. The residue was purified by silica gel column chromatography (1:1 hexanes:EtOAc) to afford a white solid (1.40 g, 94%). ¹H NMR (400 MHz, DMSO-d₆) δ 12.05 (s, 1H), 8.17 (s, 1H), 7.91 (s, 1H), 7.64 (s, 1H), 7.35 (d, J=8.6 Hz, 2H), 7.02 (d, J=8.7 Hz, 2H), 4.03 (t, J=5.9 Hz, 2H), 3.80 (s, 3H), 3.34 (t, J=5.8 Hz, 2H) 1.40 (s, 9H).

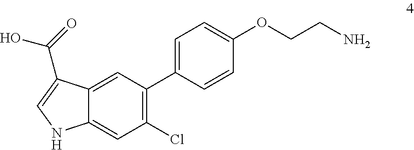 [SMILES: NCCOc1ccc(-c2cc3c(C(=O)O)c[nH]c3cc2Cl)cc1]

### 5-(4-(2-aminoethoxy)phenyl)-6-chloro-1H-indole-3-carboxylic acid (4)

Methyl 5-(4-(2-((N-Boc)amino)ethoxy)phenyl)-6-chloro-1H-indole-3-carboxylate (1.40 g, 3.17 mmol) was first hydrolyzed into the corresponding carboxylic acid by treatment with 3 eq 1M NaOH and microwaved for 2 hours at 100° C. as a 1:1:1 THF:MeOH:water solution. Then, the solution was concentrated under reduced pressure, the aqueous layer acidified to pH=3-4, and extracted 3× with EtOAc. The combined organic layers were dried with Na₂SO₄, filtered, and concentrated to a white residue that is taken to the next step without further purification. TFA (8 mL) as a 50% solution in DCM was slowly added to the white solid dissolved in 25 mL DCM at 0° C. The reaction was stirred at room temperature for 2 hours then concentrated under reduced pressure to afford a red-purple oil. The oil was treated with multiple 20 mL aliquots of ether and evaporated and dried under high vacuum to afford a reddish tan solid (0.94 g, 90%). ¹H NMR (400 MHz, DMSO-d₆) δ 11.99 (s, 1H), 8.08 (s, 1H), 7.93 (s, 1H), 7.63 (s, 1H), 7.39 (d, J=8.7 Hz, 2H), 7.07 (d, J=8.7 Hz, 2H), 4.22 (t, J=5.1 Hz, 2H), 3.26 (t, J=5.1 Hz, 2H). ¹³C NMR (101 MHz, DMSO-d₆) δ 165.6, 157.1, 136.0, 133.9, 133.1, 132.7, 130.9, 125.8, 125.3, 122.7, 114.2, 112.9, 107.6, 64.5.

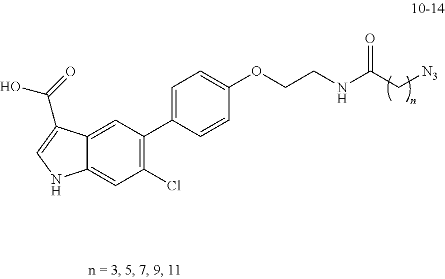

### 5-(4-(2-aminoethoxy)phenyl)-6-chloro-1H-indole-3-acyl azides (10-14)

The following general method was used to prepare the azides of varying linker lengths: Compound 5-9 (0.22 mmol, 1 eq) was added to a solution of 4 (0.20 mmol, 0.9 mmol) in DMF (0.1 M). DIPEA was added under nitrogen (2 eq) and the solution was stirred for 2 hours at room temperature under nitrogen then concentrated under reduced pressure. The residue was purified by silica gel column chromatography (1:10 MeOH:DCM) to afford 10-14 as white powder.

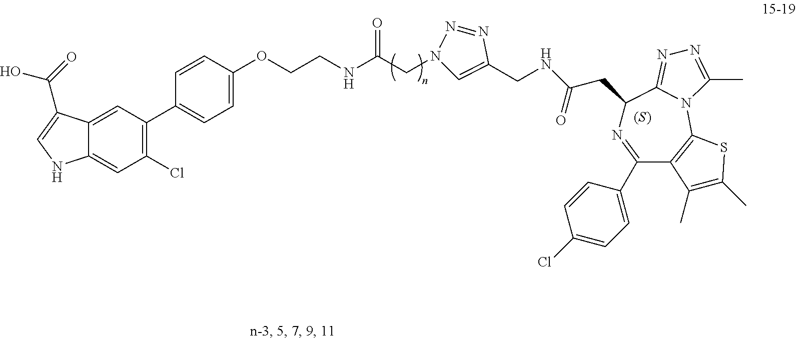

### PHICS1.1-1.5

The following general method was used to prepare the AMPK PHICS problems of varying linker lengths: compound 10-14 (0.037 mmol, 1 eq), (+) or (−)-JQ1-PA (0.041 mmol, 1.1 eq) was dissolved in THF (0.05 M). To this solution was added 0.5 M CuSO₄ (0.2 eq), 0.5 M sodium ascorbate (0.2 eq), and 0.5 M TBTA (0.2 eq). Upon completion (5 minutes to an hour), the solution turns from brown to blue and directly purified by silica gel column chromatography (1:10 MeOH:DCM) or HPLC to afford PHICS1.1 (n=3), PHICS1.2 (n=5), PHICS1.3 (n=7), PHICS1.4 (n=9), PHICS1.5 (n=11), and (R)-PHICS1.2 (n=5, with the inactive (−)-JQ1) as light beige solids.

PHICS1.1: ¹H NMR (400 MHz, DMSO-d₆) δ 11.93 (br), 11.42 (s), 8.71 (t, J=5.7 Hz, 1H), 8.17 (t, J=5.6 Hz, 1H), 8.07 (s, 1H), 7.94 (d, J=10.7 Hz, 2H), 7.62 (s, 1H), 7.47 (d, J=8.4 Hz, 2H), 7.39 (d, J=8.4 Hz, 2H), 7.34 (d, J=8.5 Hz, 2H), 7.02 (d, J=8.7 Hz, 2H), 4.53 (m, J=7.1 Hz, 1H), 4.38-4.32 (m, 4H), 4.05 (t, J=5.8 Hz, 2H), 3.47 (q, J=5.7 Hz, 2H), 2.60 (s, 3H), 2.40 (s, 3H), 2.15 (t, J=7.3 Hz, 2H), 2.05 (q, J=7.1 Hz, 2H), 1.62 (s, 3H). ¹³C NMR (101 MHz, DMSO) δ 171.3, 169.5, 165.6, 163.0, 157.6, 155.0, 149.8, 144.9, 136.7, 135.9, 135.2, 133.8, 133.7, 132.8, 132.5, 132.2, 130.8, 130.6, 130.2, 129.9, 129.5, 128.4, 125.8, 125.2, 122.8, 122.7, 114.0, 112.8, 66.3, 53.9, 48.9, 38.3, 37.5, 31.9, 25.9, 14.0, 12.7, 11.3.

PHICS1.2: ¹H NMR (400 MHz, DMSO-d₆) δ 12.19-12.00 (br, 1H), 11.92 (s, 1H), 8.71 (t, J=5.7 Hz, 1H), 8.05 (s, 2H), 7.93 (s, 2H), 7.61 (s, 1H), 7.45 (d, J=8.6 Hz, 2H), 7.38 (d, J=8.6 Hz, 2H), 7.34 (d, J=8.6 Hz, 2H), 7.01 (d, J=8.7 Hz, 2H), 4.52 (m, J=7.1 Hz, 1H), 4.35 (d, J=5.6 Hz, 2H), 4.28 (t, J=7.2 Hz, 2H), 4.06-4.00 (m, 2H), 3.44 (q, J=5.6 Hz, 2H), 3.27 (dd, 2H), 2.59 (s, 3H), 2.39 (s, 3H), 2.09 (t, J=7.4 Hz, 2H), 1.78 (p, J=7.3 Hz, 2H), 1.61 (s, 3H), 1.52 (p, J=7.5 Hz, 2H), 1.29-1.18 (m, 2H). ¹³C NMR (101 MHz, DMSO-d₆) δ 172.3, 169.5, 165.6, 163.0, 157.6, 155.1, 149.8, 144.9, 136.7, 135.9, 135.2, 133.7, 133.7, 132.7, 132.5, 132.3, 130.8, 130.7, 130.2, 129.8, 129.5, 128.4, 125.8, 125.3, 122.7, 122.7, 114.0, 112.8, 66.4, 53.9, 49.1, 38.2, 37.5, 35.0, 34.3, 29.5, 25.5, 24.6, 14.0, 12.7, 11.3.

(R)-PHICS1.2: The NMR data matches PHICS1.2.

PHICS1.3: ¹H NMR (400 MHz, DMSO-d₆) δ 11.93 (s, 1H), 8.72 (t, J=5.7 Hz, 1H), 8.06 (s, 2H), 7.93 (d, J=6.8 Hz, 2H), 7.61 (s, 1H), 7.45 (dd, J=8.6, 1.6 Hz, 2H), 7.41-7.31 (m, 4H), 7.01 (d, J=7.8 Hz, 2H), 4.52 (m, J=7.2 Hz, 1H), 4.35 (d, J=5.6 Hz, 2H), 4.27 (t, J=7.2 Hz, 2H), 4.03 (s, 2H), 3.44 (d, J=5.7 Hz, 2H), 3.29-3.22 (m, 2H), 2.59 (s, 3H), 2.40 (s, 3H), 2.09 (t, J=7.1 Hz, 2H), 1.61 (s, 3H), 1.49 (q, J=7.5 Hz, 2H), 1.25-1.18 (br, 8H). ¹³C NMR (101 MHz, DMSO) δ 172.5, 169.6, 165.6, 163.0, 157.6, 155.1, 149.8, 144.9, 136.7, 135.9, 135.2, 133.8, 133.8, 132.8, 132.5, 132.3, 130.8, 130.7, 130.2, 129.8, 129.5, 128.4, 125.8, 125.3, 122.7, 122.7, 114.0, 112.9, 66.4, 53.9, 50.6, 49.2, 38.2, 37.5, 35.2, 34.3, 29.7, 28.4, 28.1, 25.7, 14.1, 12.7, 11.3.

PHICS1.4: ¹H NMR (400 MHz, DMSO-d₆) δ 11.92 (s, 1H), 8.72 (t, J=5.7 Hz, 1H), 8.05 (d, J=7.6 Hz, 2H), 7.92 (d, J=8.4 Hz, 2H), 7.61 (s, 1H), 7.45 (d, J=8.3 Hz, 2H), 7.35 (dd, J=11.0, 8.4 Hz, 4H), 7.01 (d, J=8.5 Hz, 2H), 4.52 (m, J=8.1, 6.2 Hz, 1H), 4.35 (t, J=5.8 Hz, 2H), 4.26 (t, J=7.2 Hz, 2H), 4.03 (t, J=5.6 Hz, 2H), 3.44 (q, J=5.6 Hz, 2H), 3.28-3.20 (m, 2H), 2.59 (s, 3H), 2.40 (s, 3H), 2.10-2.06 (m, 2H), 1.61 (s, 3H), 1.51-1.46 (m, 2H), 1.21 (s, 12H). ¹³C NMR (101 MHz, DMSO) δ 172.7, 169.6, 165.6, 163.0, 157.6, 155.1, 149.8, 144.9, 136.7, 135.9, 135.2, 133.8, 133.7, 132.8, 132.5, 132.3, 130.7, 130.7, 130.2, 129.8, 129.5, 128.4, 125.8, 125.2, 122.7, 114.0, 112.8, 107.6, 66.4, 53.9, 49.3, 38.2, 37.5, 35.3, 34.3, 29.7, 28.7, 28.6, 28.4, 25.8, 25.2, 25.1, 14.1, 12.7, 11.3.

PHICS1.5: ¹H NMR (400 MHz, DMSO-d₆) δ 11.92 (s, 1H), 8.72 (t, J=5.7 Hz, 1H), 8.05 (d, J=7.6 Hz, 2H), 7.92 (d, J=8.4 Hz, 2H), 7.61 (s, 1H), 7.45 (d, J=8.3 Hz, 2H), 7.35 (dd, J=11.0, 8.4 Hz, 4H), 7.01 (d, J=8.5 Hz, 2H), 4.52 (m, J=8.1, 6.2 Hz, 1H), 4.35 (t, J=5.8 Hz, 2H), 4.26 (t, J=7.2 Hz, 2H), 4.03 (t, J=5.6 Hz, 2H), 3.44 (q, J=5.6 Hz, 2H), 3.28-3.20 (m, 2H), 2.59 (s, 3H), 2.40 (s, 3H), 2.10-2.06 (m, 2H), 1.61 (s, 3H), 1.51-1.46 (m, 2H), 1.21 (s, 12H). ¹³C NMR (101 MHz, DMSO) δ 172.95, 170.03, 163.48, 158.12, 137.17, 136.38, 135.66, 134.32, 133.24, 132.94, 132.77, 131.21, 131.14, 130.65, 130.28, 129.99, 128.87, 126.27, 125.70, 123.14, 114.50, 113.33, 66.90, 54.38, 49.73, 38.68, 38.01, 35.78, 34.75, 30.21, 29.39, 29.36, 29.30, 29.27, 29.10, 28.86, 26.34, 25.73, 14.52, 13.13, 11.78.

### 2.11 Synthesis of PHICS2 Analogs

Methyl (S)-(1-hydroxy-3-(4-hydroxy-2-nitrophenyl)propan-2-yl)carbamate (20) was synthesized according to reported procedure.⁶

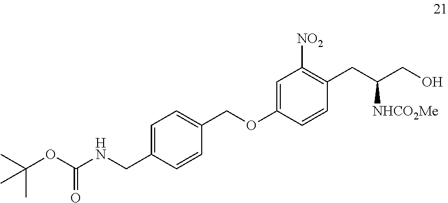

### Synthesis of methyl (S)-(1-(4-((4-(((tert-butoxycarbonyl)amino)methyl)benzyl)oxy)-2-nitrophenyl)-3-hydroxypropan-2-yl)carbamate (21)

To a solution of 20 (1.2 g, 4.4 mmol) and K₂CO₃ (1.2 g, 8.7 mmol) in 33 mL of DMF was added tert-butyl (4-(bromomethyl)benzyl)carbamate (1.4 g, 4.7 mmol) in 3 mL DMF in a dropwise manner. The reaction mixture was heated at 50° C. overnight and progress of the reaction was monitored by LCMS. When signal of starting material VS-3 disappeared, solvent was removed under reduced pressure. The solid residue was partitioned between 220 mL of ethyl acetate and 40 mL of water. The organic layer was washed with brine, dried over Na₂SO₄, and concentrated under reduced pressure. The crude product was purified by column chromatography (gradient from 20:80 ethyl acetate/petroleum ether as eluent up to 50:50/petroleum ether) to provide 1.5 g (70%) of desired product as a yellow solid was obtained. ¹H NMR (400 MHz, CDCl₃): δ 7.50 (s, 1H), 7.38-7.36 (m, 2H), 7.31-7.29 (m, 2H), 7.15-7.12 (m, 1H), 5.32 (br s, 1H), 5.06 (s, 2H), 4.92 (br s, 1H), 4.31 (s, 2H), 3.96 (s, 1H), 3.73-3.62 (m, 2H), 3.57 (s, 3H), 3.14-3.11 (m, 1H), 2.99-2.95 (m, 1H), 2.39 (br s, 1H), 1.45 ppm (s, 9H); ¹³C NMR (101 MHz, CDCl₃) δ 157.8, 157.2, 156.1, 150.2, 139.4, 134.9, 133.7, 128.0, 127.9, 125.5, 120.7, 110.6, 79.81, 70.43, 64.58, 54.32, 52.31, 33.46, 28.52 ppm. LS-MS [M+Na]=512.

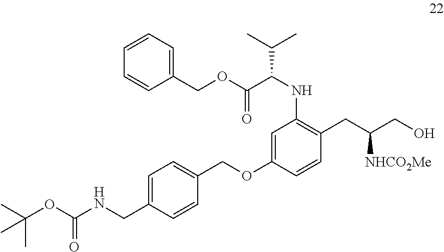

### Synthesis of benzyl (5-((4-(((tert-butoxycarbonyl)amino)methyl)benzyl)oxy)-2-((S)-3-hydroxy-2-((methoxycarbonyl)amino)propyl)phenyl)-D-valinate (22)

Step 1. To the solution of 21 (1.5 g, 3.1 mmol) in MeOH (52 mL) saturated solution of Cu (II) acetate (17 mL) was added. The resulting solution was cooled with ice-water while NaBH₄ (1.8 g, 47.4 mmol) was added in portion over 1 hour. The progress of the reaction was monitored via LCMS (if the reaction didn't reach completion more NaBH4 should be added). When nitro group was completely reduced, reaction mixture was passed over a short column of silica gel. The filtrate was concentrated under reduced pressure and solid residue was partitioned between 180 mL EtOAc and 35 mL of water. The organic layer was washed with brine, dried over Na₂SO₄, and concentrated to provide the crude aniline, which was used in the next step without additional purification.

Step 2: To the product from previous step 1.1 g of triflate obtained from benzyl (R)-3-methyl-2-hydroxybutanoate was added. Mixture was degassed, dissolved in 35 mL of dichloromethane added under nitrogen atmosphere, treated by 600 μL of 2,6-luthidine and stirred at 70° C. Reaction progress was monitored via LCMS and when starting materials were completely consumed (˜72 hours), solvent was removed under reduced pressure and crude mixture purified via LCMS (gradient from 20:80 ethyl acetate/petroleum ether as eluent up to 70:30 ethyl acetate/petroleum ether). 970 mg (48%) of desired product as a white solid was obtained. ¹H NMR (400 MHz, CDCl₃): δ 7.32 (d, J=8.0 Hz, 2H), 7.26-7.23 (m, 7H), 6.97 (d, J=8.4 Hz, 1H), 6.30 (d, J=8.4 Hz, 1H), 6.22 (s, 1H), 5.34 (d, J=8.0 Hz, 1H), 5.10 (s, 2H), 4.90-4.83 (m, 3H), 4.29 (d, J=5.6 Hz, 2H), 3.85 (d, J=5.6 Hz, 1H), 3.74 (br s, 1H), 3.65 (s, 3H), 3.52 (d, J=12.8 Hz, 1H), 3.44 (d, J=14.4 Hz, 1H), 2.82-2.78 (m, 1H), 2.72-2.66 (m, 1H), 2.15-2.10 (m, 1H), 1.44 (s, 9H), 1.03 (d, J=6.4 Hz, 3H), 0.98 ppm (d, J=6.8 Hz, 3H); ¹³C NMR (101 MHz, CDCl₃) δ 174.7, 159.0, 157.0, 156.0, 146.4, 138.7, 136.5, 135.4, 132.1, 128.7, 128.6, 127.9, 127.8, 116.2, 103.9, 99.2, 79.7, 69.7, 67.1, 62.6, 61.8, 53.4, 52.2, 44.6, 31.7, 28.5, 19.4, 19.2 ppm. LS-MS [M+H]=651.

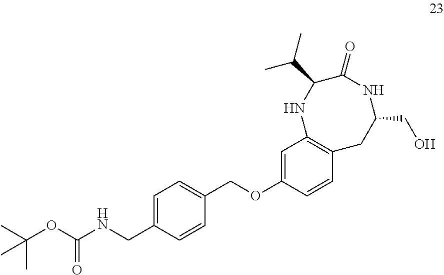 [SMILES: CC(C)[C@@H]1Nc2cc(OCc3ccc(CNC(=O)OC(C)(C)C)cc3)ccc2C[C@@H](CO)NC1=O]

### Synthesis of tert-butyl (4-((((2S,5S)-5-(hydroxymethyl)-2-isopropyl-3-oxo-1,2,3,4,5,6-hexahydrobenzo[e][1,4]diazocin-9-yl)oxy)methyl)benzyl)carbamate (23)

A mixture 22 (220 mg, 0.34 mmol), 5.8 mL of methanol, and 2.9 mL of 2 N KOH (aqueous) was heated at 70° C. overnight. Reaction progress was monitored via LCMS. Once reaction was completed, mixture was cooled to room temperature, neutralized with 2 N HCl to pH 7 and then concentrated under reduced pressure. The residual solid was dried in vacuo for 1 day before it was dissolved in 34 mL of DMF. To this solution were added triethylamine (102 μL) and DPPA (90 μL) at 0° C. After the stirring was continued for 1 h at 0° C. and 17 h at room temperature, DMF was evaporated under reduced pressure. The residue was partitioned between 200 mL of ethyl acetate and 35 mL of water. The organic layer was washed with brine, dried over Na₂SO₄, concentrated and purified via flash column chromatography (gradient from 20:80 ethyl acetate/petroleum ether as eluent up to 70:30 ethyl acetate/petroleum ether). 120 mg (74%) of desired product as a yellow oil was obtained. ¹H NMR (400 MHz, CDCl₃): δ 7.34 (d, J=8.0 Hz, 2H), 7.29-7.26 (m, 3H), 6.84 (d, J=8.0 Hz, 1H), 6.75-6.72 (br s, 1H), 6.46 (d, J=8.0 Hz, 1H), 6.42 (s, 1H), 5.01 (t, J=6.0 Hz, 1H), 4.93 (s, 2H), 4.30 (d, J=5.6 Hz, 1H), 3.82 (d, J=8.4 Hz, 1H), 3.70-3.66 (m, 1H), 3.62-3.59 (m, 1H), 3.14-3.07 (m, 1H), 2.80-2.76 (m, 1H), 2.14-2.02 (m, 1H), 1.47 (s, 9H), 1.09 (d, J=6.8 Hz, 3H), 0.96 ppm (d, J=6.8 Hz, 3H); 6 ¹³C NMR (101 MHz, CDCl₃) 175.3, 158.4, 156.1, 147.7, 138.8, 136.2, 132.8, 127.8, 127.7, 118.4, 108.1, 106.7, 79.6, 69.6, 66.8, 66.0, 55.46, 44.4, 35.4, 30.5, 28.5, 20.3 and 19.2 ppm. LS-MS [M+H]=485.

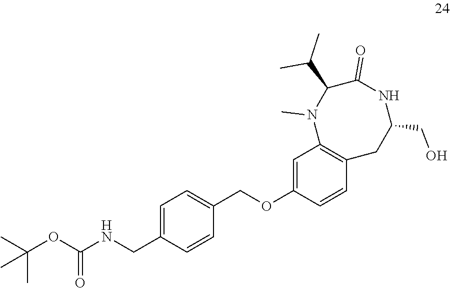 [SMILES: CC(C)[C@H]1C(=O)N[C@H](CO)Cc2ccc(OCc3ccc(CNC(=O)OC(C)(C)C)cc3)cc2N1C]

### Synthesis of tert-butyl (4-((((2S,5S)-5-(hydroxymethyl)-2-isopropyl-1-methyl-3-oxo-1,2,3,4,5,6-hexahydrobenzo[e][1,4]diazocin-9-yl)oxy)methyl)benzyl)carbamate (24)

To a solution of 23 (120 mg, 0.25 mmol) in 5 mL of CH₃CN were added formaldehyde (0.2 mL, 37% wt in water), NaCNBH₃ (47 mg), and HOAc (25 μM) at 0° C., sequentially. Reaction progress was monitored via LCMS and when signal of starting material disappeared, reaction was partitioned between 50 mL of ethyl acetate and 10 mL of water. The organic layer was washed with brine, dried over Na₂SO₄, and concentrated under reduced pressure. The residue was purified via flash column chromatography (gradient from 10:90 ethyl acetate/petroleum ether as eluent up to 40:60 ethyl acetate/petroleum ether) to afford 100 mg (80%) of desired product. ¹H NMR (400 MHz, CDCl₃): δ 7.39 (d, J=8.4 Hz, 2H), 7.30 (d, J=8.4 Hz, 2H), 6.93 (d, J=8.4 Hz, 1H), 6.59 (d, J=2.8 Hz, 1H), 6.55 (br s, 1H), 6.48 (dd, J=8.4 & 2.8 Hz, 1H), 5.00 (s, 2H), 4.88 (br s, 1H), 4.31 (s, 2H), 3.91 (br s, 1H), 3.69 (d, J=3.8 Hz & 7.0 Hz, 1H), 3.52-3.46 (m, 2H), 3.03 (dd, J=16.8 & 8.0 Hz, 1H), 2.75 (s, 3H), 2.45-2.36 (m, 1H), 1.46 (s, 9H), 1.04 (d, J=6.4 Hz, 3H), 0.85 ppm (d, J=6.8 Hz, 3H); ¹³C NMR (101 MHz, CDCl₃) δ 174.0, 158.7, 156.18, 156.15, 152.9, 139.0, 136.4, 132.6, 128.1, 127.9, 123.7, 107.3, 107.1, 79.9, 70.1, 66.1, 54.6, 54.5, 36.9, 35.3, 28.7, 28.5, 20.7 and 20.1 ppm. LS-MS [M+Na]=521.

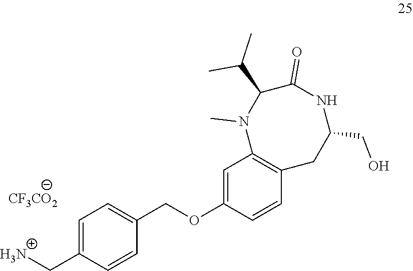 [SMILES: "CF3CO2" ; CC(C)[C@H]1C(=O)N[C@H](CO)Cc2ccc(OCc3ccc(C[NH3+])cc3)cc2N1C]

### Synthesis of (4-((((2S,5S)-5-(hydroxymethyl)-2-isopropyl-1-methyl-3-oxo-1,2,3,4,5,6-hexahydrobenzo[e][1,4]diazocin-9-yl)oxy)methyl)phenyl)methanaminium trifluoroacetate (25)

To an ice-cold solution of VS-43 (12 mg, 24.0 μmol) in DCM (300 μL) was added 300 μL trifluroacetic acid and reaction was allowed to warm up to room temperature and stirred for 30 min. Reaction mixture was concentrated to afford 12.2 mg (99%) of crude VS-35 which was used in the next step immediately. ¹H NMR (400 MHz, CD₃OD): δ 7.52 (d, J=8.0 Hz, 2H), 7.46 (d, J=8.0 Hz, 2H), 6.96 (d, J=8.0 Hz, 1H), 6.75 (d, J=2.6 Hz, 1H), 6.59 (dd, J=8.4 & 2.6 Hz, 1H), 5.09 (s, 2H), 4.11 (s, 2H), 3.59 (d, J=4.8 Hz & 11.0 Hz, 1H), 3.51-3.46 (m, 2H), 2.97-2.83 (m, 2H), 2.74, (s, 3H), 2.44-2.35 (m, 1H), 1.09 (d, J=6.6 Hz, 3H), 0.92 ppm (d, J=6.8 Hz, 3H); ¹³C NMR (101 MHz, CD₃OD) δ 175.6, 159.7, 154.3, 140.2, 133.9, 133.5, 130.3, 130.1, 129.34, 129.31, 129.27, 126.5, 110.5, 109.1, 98.2, 70.4, 65.7, 55.3, 40.1, 37.7, 29.7, 20.8, 19.9 ppm. LS-MS [M-CF₃CO₂]=398.

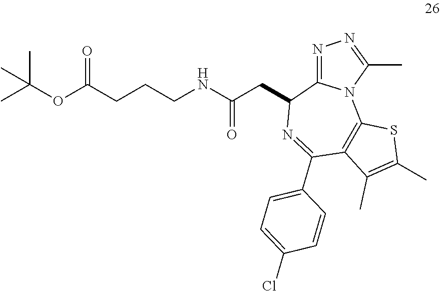

### Synthesis of tert-butyl (S)-4-(2-(4-(4-chlorophenyl)-2,3,9-trimethyl-6H-thieno[3,2-f][1,2,4]triazolo[4,3-a][1,4]diazepin-6-yl)acetamido)butanoate (26)

Freshly prepared free acid of (+)-JQ-1 (20 mg, 50 μmol)⁷ was dissolved in 1.0 mL DMF and treated with tert-butyl 4-aminobutanoate (9.6 mg, 60 μmol), PyBOP (32 mg, 30 μmol) and Et₃N (40 μL). Reaction mixture was stirred overnight and purified via HPLC to afford 19.5 mg (72%) of VS-327. ¹H NMR (400 MHz, CD₃OD): δ 7.48 (d, J=8.4 Hz, 2H), 7.43 (d, J=8.4 Hz, 2H), 4.71 (dd, J=5.2 & 9.2 Hz, 1H), 3.46-3.41 (m, 1H), 3.35-3.24 (m, 3H), 2.75 (s, 3H), 2.46 (s, 3H), 2.32 (t, J=7.4 Hz, 2H), 1.82 (t, J=7.2 Hz, 2H), 1.71 (s, 3H), 1.44 ppm (s, 9H); ¹³C NMR (101 MHz, CD₃OD) δ 174.3, 172.5, 166.7, 156.8, 152.6, 138.4, 134.0, 133.3, 132.3, 132.1, 131.5, 129.9, 81.6, 54.9, 39.8, 38.4, 33.7, 28.4, 26.1, 14.4, 13.0, 11.5 ppm. LS-MS [M+H]=542.

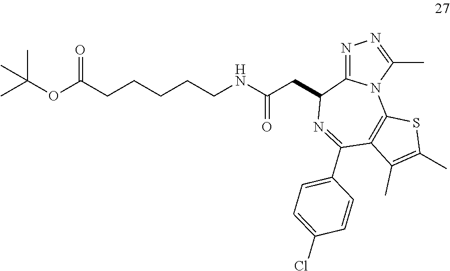

### Synthesis of tert-butyl (S)-6-(2-(4-(4-chlorophenyl)-2,3,9-trimethyl-6H-thieno[3,2-f][1,2,4]triazolo[4,3-a][1,4]diazepin-6-yl)acetamido)hexanoate (27)

Freshly prepared free acid of (+)-JQ-1 (10 mg, 25 μmol)⁷ was dissolved in 0.5 mL DMF and treated with tert-butyl 6-aminohexanoate (5.6 mg, 30 μmol), PyBOP (16 mg, 30 μmol) and Et₃N (20 μL). Reaction mixture was stirred overnight and purified via HPLC to afford 12 mg (85%) of VS-359. ¹H NMR (400 MHz, CD₃OD): δ 7.47 (d, J=8.6 Hz, 2H), 7.42 (d, J=8.6 Hz, 2H), 4.71 (dd, J=5.4 & 9.0 Hz, 1H), 3.43 (dd, J=9.2 and 15.2 Hz, 1H), 3.32-3.19 (m, 3H), 2.75 (s, 3H), 2.45 (s, 3H), 2.24-2.20 (m, 2H). 1.70 (s, 3H), 1.64-1.54 (m, 4H), 1.49-1.37 ppm (s and m, 13H); ¹³C NMR (101 MHz, CD₃OD) δ 174.8, 172.3, 166.7, 156.8, 152.6, 138.4, 137.45, 134.01, 133.3, 132.3, 132.06, 131.5, 129.9, 81.4, 54.9, 40.3, 38.3, 36.3, 30.2, 28.4, 27.4, 25.9, 14.4, 13.0, 11.5 ppm. LS-MS [M+H]=571.

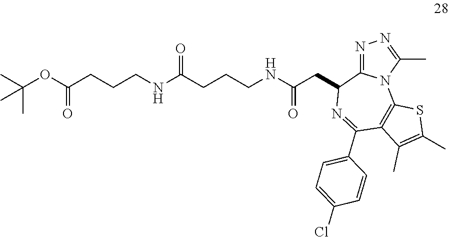

### Synthesis of tert-butyl (S)-4-(4-(2-(4-(4-chlorophenyl)-2,3,9-trimethyl-6H-thieno[3,2-f][1,2,4]triazolo[4,3-a][1,4]diazepin-6-yl)acetamido)butanamido)butanoate (28)

Freshly prepared free acid of (+)-JQ-1 (10 mg, 25 μmol)⁷ was dissolved in 0.5 mL DMF and treated with tert-butyl 4-(4-aminobutanamido)butanoate (7.3 mg, 30 μmol), PyBOP (16 mg, 30 μmol) and Et₃N (20 μL). Reaction mixture was stirred overnight and purified via HPLC to afford 12.4 mg (79%) of VS-329. ¹H NMR (400 MHz, CD₃OD): δ 7.46 (d, J=8.4 Hz, 2H), 7.42 (d, J=8.4 Hz, 2H), 4.69 (dd, J=5.8 & 9.0 Hz, 1H), 3.42 (dd, J=8.4 & 15.2 Hz, 1H), 3.36-3.27 (m, 3H), 3.2 (t, J=7.0 Hz, 2H), 2.76 (s, 3H), 2.45 (s, 3H), 2.29-2.23 (m, 4H), 1.89-1.82 (m, 2H), 1.78-1.72 (m, 2H), 1.70 (s, 3H), 1.44 ppm (s, 9H); ¹³C NMR (101 MHz, CD₃OD) δ 175.4, 174.2, 172.6, 166.6, 156.9, 152.4, 138.2, 137.7, 133.7, 133.5, 132.2, 132.0, 131.4, 129.9, 81.5, 55.0, 39.9, 39.7, 38.5, 34.4, 33.7, 28.3, 26.9, 25.9, 14.4, 13.0, 11.6 ppm. LS-MS [M+H]=628.

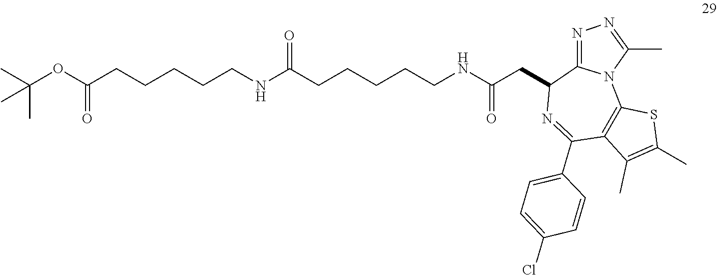

### Synthesis of tert-butyl (S)-6-(6-(2-(4-(4-chlorophenyl)-2,3,9-trimethyl-6H-thieno[3,2-f][1,2,4]triazolo[4,3-a][1,4]diazepin-6-yl)acetamido)hexanamido)hexanoate (29)

Freshly prepared free acid of (+)-JQ-1 (20 mg, 50 μmol)⁷ was dissolved in 1.0 mL DMF and treated with tert-butyl 6-(6-aminohexanamido)hexanoate (18.0 mg, 60 μmol), PyBOP (32 mg, 30 μmol) and Et₃N (40 μL). Reaction mixture was stirred overnight and purified via HPLC to afford 27.3 mg (80%) of VS-355. ¹H NMR (400 MHz, CD₃OD): δ 7.47 (d, J=8.4 Hz, 2H), 7.43 (d, J=8.4 Hz, 2H), 4.69 (dd, J=5.6 & 8.8 Hz, 1H), 3.46-3.40 (m, 1H), 3.35-3.26 (m, 3H), 3.16 (t, J=7.0 Hz, 2H), 2.76 (s, 3H), 2.46 (s, 3H), 2.22-2.18 (m, 4H), 1.71 (s, 3H), 1.67-1.48 (m, 8H), 1.43 (s+m, 11H), 1.37-1.28 ppm (m, 2H); ¹³C NMR (101 MHz, CD₃OD) δ 176.0, 174.9, 172.3, 166.7, 156.8, 152.6, 138.4, 137.5, 134.0, 133.3, 132.3, 132.0, 131.5, 129.91, 129.87, 81.3, 54.9, 40.3, 40.2, 38.3, 37.0, 36.3, 30.09, 30.11, 28.4, 27.6, 27.4, 26.7, 25.8, 14.4, 13.0 and 11.5 ppm. LS-MS [M+H]=684.

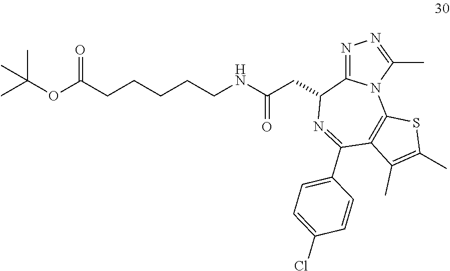

### Synthesis of tert-butyl (R)-6-(2-(4-(4-chlorophenyl)-2,3,9-trimethyl-6H-thieno[3,2-f][1,2,4]triazolo[4,3-a][1,4]diazepin-6-yl)acetamido)hexanoate (30)

Freshly prepared free acid of (−)-JQ-1 (6 mg, 15 μmol) 7 was dissolved in 0.25 mL DMF and treated with tert-butyl 6-aminohexanoate (2.8 mg, 15 μmol), PyBOP (8 mg, 15 μmol) and Et₃N 10 μL). Reaction mixture was stirred overnight and purified via HPLC to afford 5.7 mg (80%) of VS-361. ¹H NMR (400 MHz, CD₃OD): δ 7.47 (d, J=8.4 Hz, 2H), 7.42 (d, J=8.4 Hz, 2H), 4.66 (dd, J=5.2 & 9.1 Hz, 1H), 3.45-3.39 (m, 1H), 3.27-3.21 (m, 3H), 2.72 (s, 3H), 2.45 (s, 3H), 2.23 (t, J=7.4 Hz, 2H), 1.71 (s, 3H), 1.65-1.55 (m, 4H), 1.44-1.29 ppm (s and m, 12H); ¹³C NMR (101 MHz, CD₃OD) δ 174.9, 172.5, 166.4, 156.9, 152.3, 138.3, 137.9, 133.6, 133.4, 132.1, 132.0, 131.4, 129.8, 81.4, 55.1, 40.3, 38.6, 36.3, 30.2, 28.4, 27.4, 25.9, 14.4, 13.0, 11.6 ppm. LS-MS [M+H]=571.

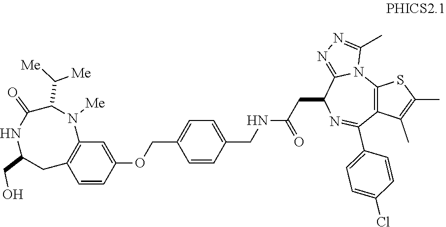 [SMILES: Cc1sc2c(c1C)C(c1ccc(Cl)cc1)=N[C@@H](CC(=O)NCc1ccc(COc3ccc4c(c3)N(C)[C@@H](C(C)C)C(=O)N[C@H](CO)C4)cc1)c1nnc(C)n1-2]

### Synthesis of PHICS2.1 (31)

Freshly prepared free acid of (+)-JQ-1 (5 mg, 12.5 μmol)⁷ was dissolved in 0.35 mL DMF and was treated with freshly prepared 25 (6.4 mg, 12.5 μmol) followed by PyBOP (8.8 mg) and Et₃N (25 μL). Reaction was stirred overnight and purified via HPLC affording 5.5 mg of desired product PHICS2.1 (56% yield) as an off-white solid. ¹H NMR (400 MHz, CD₃OD): 7.49-7.32 (m, 8H), 6.97 (d, J=8.4 Hz, 1H), 6.72 (d, J=2.4 Hz, 1H), 6.58 (dd, J=8.4 & 2.4 Hz, 1H), 5.04 (s, 2H), 4.68 (dd, J=9.6 & 4.2 Hz, 1H), 4.60 (d, J=14.8 Hz, 1H), 4.35 (d, J=14.8 Hz, 1H), 4.24 (s, 1H), 3.59 (dd, J=11.2 & 4.8 Hz, 1H), 3.53-3.45 (m, 3H), 2.95-2.87 (m, 2H), 2.74 (s, 3H), 2.72 (s, 3H), 2.45 (s, 3H), 2.45-2.35 (m, 1H), 1.68 (s, 3H), 1.07 (d, J=6.4 Hz, 3H), 0.91 ppm (d, J=6.4 Hz, 3H); ¹³C NMR (101 MHz, CD₃OD) δ 172.6, 166.5, 159.9, 156.9, 154.2, 139.8, 138.1, 137.9, 137.8, 133.6, 133.5, 133.4, 132.2, 132.1, 131.4, 129.8, 128.9, 128.8, 110.3, 110.9, 70.8, 65.8, 55.4, 55.2, 43.9, 38.7, 37.8, 36.7, 29.6, 24.2, 20.7, 20, 14.4, 12.9 and 11.5 ppm. LS-MS [M+H]=780.

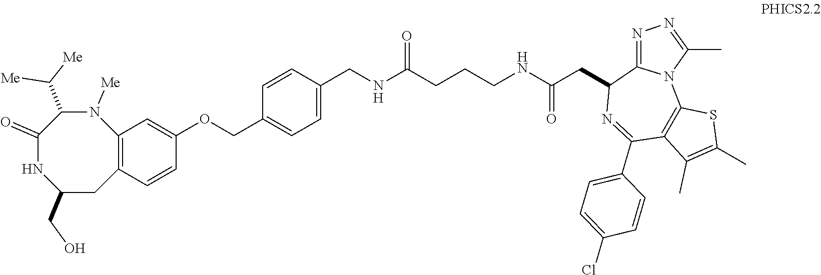

### Synthesis of PHICS2.2 (32)

Solution of 26 (13.5 mg, 25 μmol) in 1.25 mL of DCM was cooled to 0° C. and treated with trifluoroacetic acid (0.35 mL). Reaction mixture was warmed to room temperature and stirred for 3 hours before solvent was concentrated under reduced pressure. Freshly prepared 25 (12.8 mg, 25 μmol) was added in 0.7 mL DMF followed by PyBOP (17.5 mg) and Et₃N (50 μL). Reaction was stirred overnight and purified via HPLC affording 14.7 mg of desired product PHICS2.2 (68% yield) as an off-white solid. ¹H NMR (400 MHz, CD₃OD): 7.45 (d, J=8.6 Hz, 2H), 7.40 (d, J=8.4 Hz, 2H), 7.36 (d, J=8.0 Hz, 2H), 7.27 (d, J=8.0 Hz, 2H), 6.95 (d, J=8.4 Hz, 1H), 6.71 (d, J=2.4 Hz, 1H), 6.55 (dd, J=8.4 & 2.4 Hz, 1H), 5.01 (s, 2H), 4.69 (dd, J=8.4 & 5.6 Hz, 1H), 4.37 (dd, J=14.8 & 6.4 Hz, 1H), 4.24 (s, 1H), 3.59 (dd, J=10.8 & 6.4 Hz, 1H), 3.50-3.39 (m, 3H), 2.94-2.87 (m, 2H), 2.73 (s, 3H), 2.70 (s, 3H), 2.43 (s, 3H), 2.36-2.32 (m, 2H), 1.68 (s, 3H), 1.07 (d, J=6.4 Hz, 3H), 0.91 ppm (d, J=6.4 Hz, 3H); ¹³C NMR (101 MHz, CD₃OD) δ 175.3, 166.6, 159.8, 154.2, 139.6, 138.2, 138.1, 137.8, 137.7, 133.6, 133.5, 132.2, 131.9, 131.4, 129.9, 128.9, 128.8, 128.6, 126.1, 110.2, 108.9, 70.7, 65.8, 55.4, 55.0, 43.9, 39.9, 38.5, 37.7, 36.8, 34.4, 29.6, 26.9, 20.7, 20.0, 14.4, 13.0 and 11.6 ppm. LS-MS [M+H]=866.

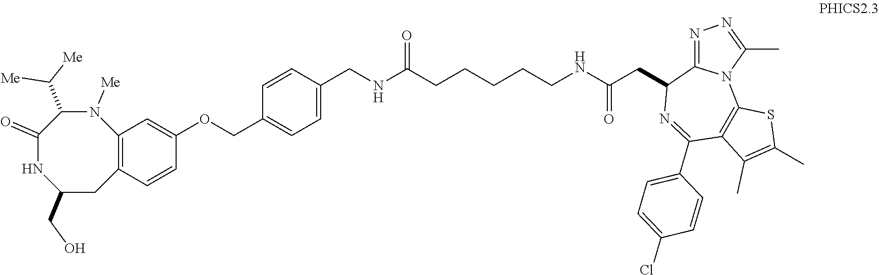

### Synthesis of PHICS2.3 (33)

Solution of 27 (11.4 mg, 20 μmol) in 1 mL of DCM was cooled to 0° C. and treated with trifluoroacetic acid (0.3 mL). Reaction mixture was warmed to room temperature and stirred for 3 hours before solvent was concentrated under reduced pressure. Freshly prepared 25 (10.2 mg, 20 μmol) was added in 0.5 mL DMF followed by PyBOP (14 mg) and Et₃N (40 μL). Reaction was stirred overnight and purified via HPLC affording 9 mg of desired product PHICS2.3 (50% yield) as an off-white solid. ¹H NMR (400 MHz, CD₃OD): 7.45-7.36 (m, 6H), 7.27 (d, J=7.6 Hz, 2H), 6.94 (d, J=8.4 Hz, 1H), 6.71 (d, J=2.4 Hz, 1H), 6.54 (dd, J=8.4 and 2.4 Hz, 1H), 5.00 (s, 2H), 4.63 (dd, J=5.4 & 8.6 Hz, 1H), 4.36 (s, 2H), 4.23 (br s, 1H), 3.61-3.57 (m, 1H), 3.50-3.37 (m, 3H), 3.28-3.23 (m, 2H), 2.91-2.87 (m, 2H), 2.72 (s, 3H), 2.68 (s, 3H), 2.43 (s, 3H), 2.26 (t, J=7.6 Hz, 2H), 1.69 (s and m, 4H), 1.63-1.55 (m, 3H), 1.45-1.39 (m, 2H), 1.33-1.29 (m, 2H), 1.07 (d, J=6.6 Hz, 3H) and 0.9 (d, J=6.6 Hz, 3H); ¹³C NMR (101 MHz, CD₃OD) δ 175.9, 175.6, 167.0, 159.8, 154.1, 139.7, 138.7, 137.8, 137.1, 134.4, 133.5, 132.5, 132.0, 131.6, 129.9, 128.91, 128.87, 128.8, 128.7, 126.1, 110.3, 108.9, 70.7, 65.8, 55.4, 54.8, 44.0, 43.8, 40.3, 38.1, 37.7, 36.9, 36.8, 30.1, 29.7, 27.5, 26.6, 20.7, 20.0, 14.4, 13.0, 11.5 ppm. LS-MS [M+H]=894.

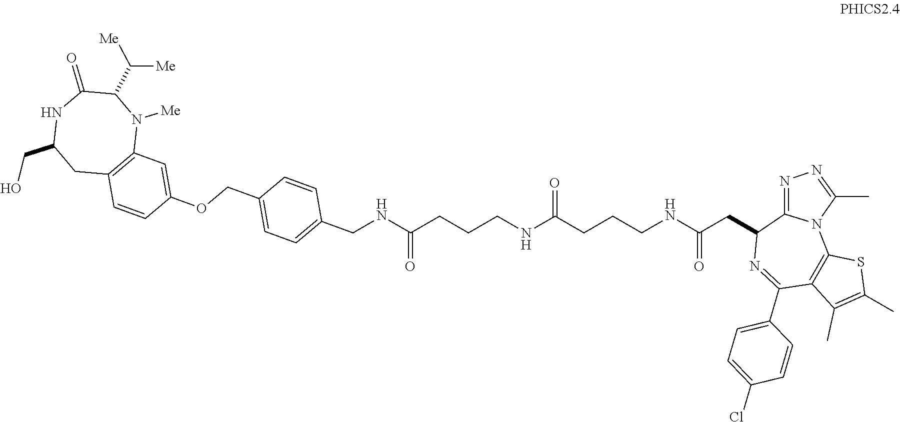

### Synthesis of PHICS2.4 (34)

Solution of 28 (12.4 mg, 19.8 μmol) in 1 mL of DCM was cooled to 0° C. and treated with trifluoroacetic acid (0.3 mL). Reaction mixture was warmed to room temperature and stirred for 3 hours before solvent was concentrated under reduced pressure. Freshly prepared 25 (10.2 mg, 20 μmol) was added in 0.5 mL DMF followed by PyBOP (14 mg) and Et₃N (40 μL). Reaction was stirred overnight and purified via HPLC affording 9.2 mg of desired product PHICS2.4 (49% yield) as an off-white solid. ¹H NMR (400 MHz, CD₃OD): 7.45 (d, J=8.4 Hz, 2H), 7.41 (d, J=8.4 Hz, 2H), 7.35 (d, J=8.0 Hz, 2H), 7.25 (d, J=7.6 Hz, 2H), 6.95 (d, J=8.4 Hz, 1H), 6.71 (d, J=2.4 Hz, 1H), 6.54 (dd, J=8.4 and 2.4 Hz, 1H), 5.00 (s, 2H), 4.63 (dd, J=5.8 & 8.4 Hz, 1H), 4.33 (s, 2H), 4.24 (br s, 1H), 3.59 (dd, J=10.8 & 4.8 Hz, 1H), 3.50-3.45 (m, 2H), 3.42-3.34 (m, 3H), 3.23-3.18 (m, 2H), 2.94-2.87 (m, 2H), 2.73 (s, 3H), 2.69 (s, 3H), 2.44 (s, 3H), 2.41-2.36 (m, 1H), 2.28-2.23 (m, 4H), 1.89-1.77 (m, 4H), 1.68 (s, H), 1.45-1.39 (m, 2H), 1.07 (d, J=6.8 Hz, 3H) and 0.9 (d, J=6.8 Hz, 3H); ¹³C NMR (101 MHz, CD₃OD) δ 175.5, 166.5, 159.8, 154.2, 139.6, 137.8, 133.5, 132.2, 131.4, 129.9, 128.8, 128.7, 110.2, 108.9, 70.7, 65.8, 55.4, 55.1, 43.9, 39.9, 39.8, 38.6, 37.7, 34.4, 34.3, 29.6, 26.8, 20.7, 20.0, 14.4, 12.9 and 11.6 ppm. LS-MS [M+H]=951.

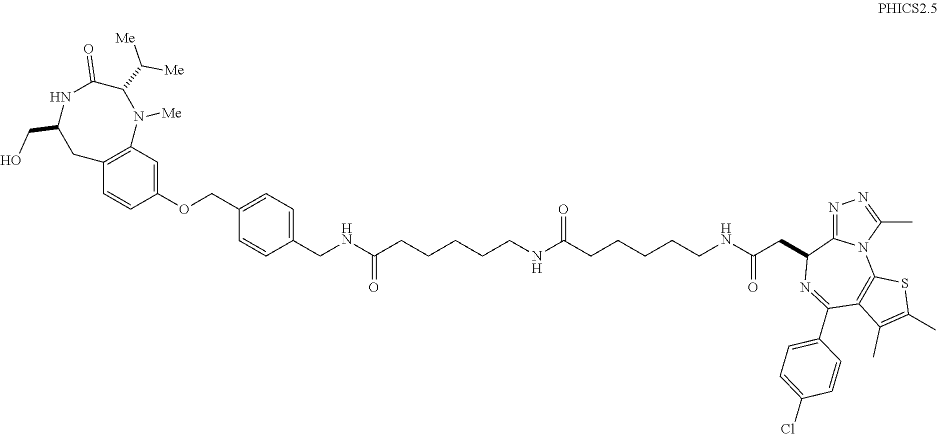

### Synthesis of PHICS2.5 (35)

Solution of 29 (17.1 mg, 25 μmol) in 1.25 mL of DCM was cooled to 0° C. and treated with trifluoroacetic acid (0.35 mL). Reaction mixture was warmed to room temperature and stirred for 3 hours before solvent was concentrated under reduced pressure. Freshly prepared 25 (12.8 mg, 25 μmol) was added in 0.7 mL DMF followed by PyBOP (17.5 mg) and Et₃N (50 μL). Reaction was stirred overnight and purified via HPLC affording 15.0 mg of desired product PHICS2.5 (60% yield) as an off-white solid. ¹H NMR (400 MHz, CD₃OD): ¹H NMR (400 MHz, CD₃OD): 7.46 (d, J=8.4 Hz, 2H), 7.42 (d, J=8.4 Hz, 2H), 7.37 (d, J=8.0 Hz, 2H), 7.26 (d, J=7.6 Hz, 2H), 6.95 (d, J=8.4 Hz, 1H), 6.72 (d, J=2.8 Hz, 1H), 6.56 (dd, J=8.4 and 2.8 Hz, 1H), 5.00 (s, 2H), 4.69 (dd, J=5.6 & 8.4 Hz, 1H), 4.34 (s, 2H), 4.25 (br s, 1H), 3.59 (dd, J=10.8 & 4.8 Hz, 1H), 3.49-3.35 (m, 4H), 3.27-3.23 (m, 3H), 3.17-3.13 (m, 2H), 2.91-2.87 (m, 3H), 2.74 (s, 3H), 2.73 (s, 3H), 2.45 (s, 3H), 2.41-2.34 (m, 1H), 2.25-2.17 (m, 4H), 1.70 (s, H), 1.67-1.31 (m, 15H), 1.07 (d, J=6.8 Hz, 3H) and 0.90 (d, J=6.8 Hz, 3H); ¹³C NMR (101 MHz, CD₃OD) δ 175.5, 166.5, 159.8, 154.2, 139.6, 137.8, 133.5, 132.2, 131.4, 129.9, 128.8, 128.7, 110.2, 108.9, 70.7, 65.8, 55.4, 55.1, 43.9, 39.9, 39.8, 38.6, 37.7, 34.4, 34.3, 29.6, 26.8, 20.7, 20.0, 14.4, 12.9 and 11.6 ppm; 13C NMR (101 MHz, CD₃OD) δ 176.0, 175.9, 172.3, 166.7, 159.8, 154.2, 139.7, 138.4, 137.8, 137.5, 134.0, 133.5, 132.3, 132.0, 131.5, 129.9, 128.8, 128.7, 70.7, 65.8, 55.4, 54.9, 43.8, 40.3, 40.2, 38.3, 37.7, 37.0, 36.9, 30.1, 29.7, 27.5, 26.71, 26.66, 20.7, 20.0, 14.4, 13.0 and 11.6 ppm. LS-MS [M+H]=1007.

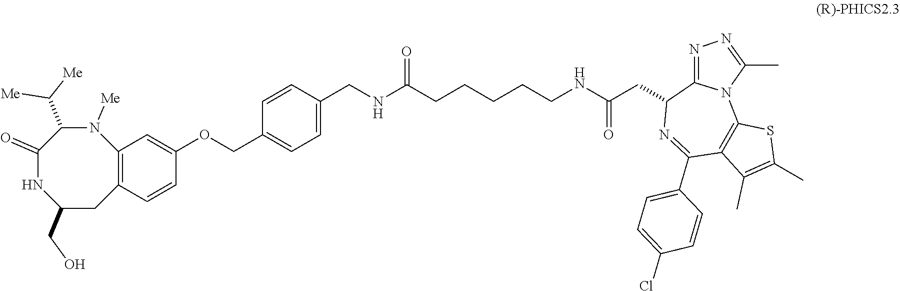

### Synthesis of (R)-PHICS2.3 (36)

Solution of 30 (5.7 mg, 12 μmol) in 0.6 mL of DCM was cooled to OC and treated with trifluoroacetic acid (0.18 mL). Reaction mixture was warmed to room temperature and stirred for 3 hours before solvent was concentrated under reduced pressure. Freshly prepared 25 (6.1 mg, 12 μmol) was added in 0.3 mL DMF followed by PyBOP (8.4 mg) and Et₃N (24 μL). Reaction was stirred overnight and purified via HPLC affording 6.8 mg of desired product (R)-PHICS2.3 (63% yield) as an off-white solid. ¹H NMR (400 MHz, CD₃OD): 7.45 (d, J=8.6 Hz, 2H), 7.41 (d, J=8.6 Hz, 2H), 7.37 (d, J=8.0 Hz, 2H) 7.27 (d, J=8.0 Hz, 2H), 6.94 (d, J=8.4 Hz, 1H), 6.71 (d, J=2.5 Hz, 1H), 6.54 (dd, J=8.4 and 2.5 Hz, 1H), 5.00 (s, 2H), 4.66 (dd, J=5.5 & 8.7 Hz, 1H), 4.36 (s, 2H), 4.24 (br s, 1H), 3.61-3.57 (m, 1H), 3.50-3.47 (m, 2H), 3.27-3.24 (m, 2H), 2.91-2.87 (m, 2H), 2.72 (s, 3H), 2.70 (s, 3H), 2.43 (s, 3H), 2.41-2.34 (m, 1H), 2.26 (t, J=7.4 Hz, 2H), 1.72-1.67 (s and m, 4H), 1.63-1.55 (m, 2H), 1.45-1.39 (m, 2H), 1.33-1.29 (m, 2H), 1.07 (d, J=6.5 Hz, 3H) and 0.9 ppm (d, J=6.7 Hz, 3H). 13C NMR (101 MHz, CD3OD) δ 175.9, 175.6, 172.6, 166.4, 159.8, 158.4, 154.2, 152.3, 139.7, 138.1, 137.9, 137.8, 133.4, 132.1, 132.0, 131.4, 131.1, 129.9, 128.8, 128.7, 128.7, 126.1, 124.3, 121.7, 111.8, 110.2, 108.9, 70.7, 65.8, 56.2, 55.4, 55.1, 45.3, 43.8, 40.3, 38.7, 37.7, 37.0, 30.1, 29.7, 27.5, 26.7, 20.7, 20.0, 14.4, 13.0, 11.6 ppm. LS-MS [M+H]=894.

### Synthesis of PHICS3 and Piv-PHICS3

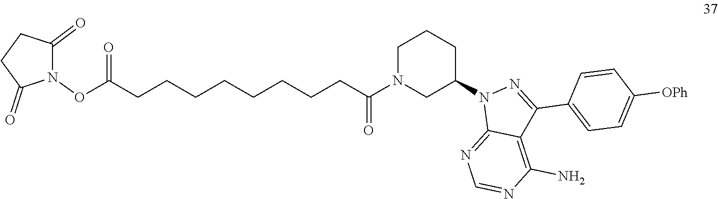 [SMILES: Nc1ncnc2c1c(-c1ccc(Oc3ccccc3)cc1)nn2[C@@H]1CCCN(C(=O)CCCCCCCCC(=O)ON2C(=O)CCC2=O)C1]

### Synthesis of 2,5-dioxopyrrolidin-1-yl (R)-10-(3-(4-amino-3-(4-phenoxyphenyl)-1H-pyrazolo[3,4-d]pyrimidin-1-yl)piperidin-1-yl)-10-oxodecanoate (37)

To the solution of 39.6 mg (0.1 mmol, 1 eq) of sebacic acid bis(N-succinimidyl) ester in 1 mL DMF was added 38.6 mg (0.1 mmol, 1 eq) of (R)-3-(4-phenoxyphenyl)-1-(1-piperidin-3-yl)-1H-pyrazolo[3,4-d]pyrimidin-4-amine. Reaction mixture was stirred at room temperature for 1 hour, concentrated under reduced pressure and purified by silica gel column chromatography (DCM: MeOH gradient from 100:0 to 95:5) affording 20 mg (30%) of desired product 37 as a white solid. 1H NMR (400 MHz, CD3Cl): 8.31 (br s, 1H), 7.62 (t, J=8 Hz, 2H), 7.40-7.37 (m, 2H), 7.19-7.13 (m, 3H), 7.08 (d, 8 Hz, 2H), 4.87-4.76 (m, 1.5H), 4.58 (d, J=12 Hz, 0.5H), 4.05 (dd, J=12 & 4 Hz, 0.5H), 3.89 (d, J=16 Hz, 0.5H), 3.68-3.62 (m, 0.5H), 3.31-3.25 (m, 0.5H), 3.17-3.10 (m, 0.5H), 2.87-2.78 (m, 3H), 2.74-2.67 (m, 2H), 2.61-2.55 (m, 2H), 2.42-2.18 (m, 2H), 2.00-1.92 (m, 1H), 1.77-1.55 (m, 5H), 1.40-1.25 ppm (m, 9H). 13C NMR (101 MHz, CD3Cl) δ 173.22, 171.96, 171.90, 169.29, 168.69, 158.69, 158.59, 157.74, 156.30, 156.24, 154.85, 154.59, 153.96, 153.76, 144.41, 130.02, 130.00, 129.97, 129.94, 127.50, 127.34, 124.15, 124.08, 119.59, 119.56, 119.52, 119.16, 118.98, 53.53, 52.66, 49.96, 45.63, 41.69, 33.51, 33.46, 30.97, 30.93, 30.34, 29.99, 29.70, 29.36, 29.32, 29.19, 29.12, 29.09, 28.93, 28.70, 25.62, 25.51, 25.34, 25.30, 25.15, 24.55, 24.06 ppm. LS-MS [M+H]=668.5.

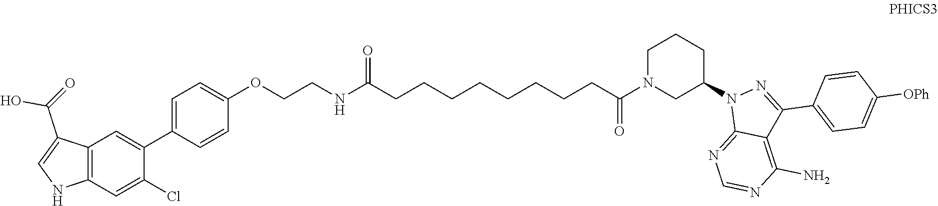 [SMILES: Nc1ncnc2c1c(-c1ccc(Oc3ccccc3)cc1)nn2[C@@H]1CCCN(C(=O)CCCCCCCCC(=O)NCCOc2ccc(-c3cc4c(C(=O)O)c[nH]c4cc3Cl)cc2)C1]

### Synthesis of PHICS3

Solution of 3 (28 mg, 65 μmol) in 1 mL of DCM was cooled to 0° C. and treated with trifluoroacetic acid (0.1 mL). Reaction mixture was warmed to room temperature and stirred for 2 hours before solvent was concentrated under reduced pressure. Freshly prepared 37 (40 mg, 60 μmol) was added in 1 mL DMF followed by DIPEA (40 μL). Reaction was stirred overnight, concentrated under reduced pressure and purified via HPLC affording 30 mg (57%) of desired product PHICS3 as a white solid. 1H NMR (400 MHz, DMSO-d6): 11.91 (br s, 1H), 8.25 (d, J=8 Hz, 1H), 8.04 (br s, 2H), 7.94 (s, 1H), 7.66-7.62 (m, 2H), 7.61 (s, 1H), 7.45-7.41 (m, 2H), 7.36-7.31 (m, 2H), 7.21-7.09 (m, 4H), 7.04-6.97 (m, 2H), 4.77-4.68 (m, 0.5H), 4.65-4.56 (m, 0.5H), 4.53-4.46 (m, 0.5H), 4.18-4.11 (m, 0.5H), 4.06-3.96 (m, 2.5H), 3.89-3.81 (m, 0.5H), 3.61-3.55 (m, 0.5H), 3.48-3.40 (m, 2H), 3.15-3.06 (m, 1.5H), 2.93-2.83 (m, 0.5H), 2.36-2.20 (m, 3H), 2.15-2.04 (m, 3H), 1.93-1.83 (m, 1H), 1.65-1.37 (m, 5H), 1.24-1.15 (m, 8H). 13C NMR (100 MHz, DMSO-d6): 172.55, 170.82, 170.71, 170.31, 165.61, 162.29, 158.19, 157.65, 157.12, 156.30, 155.62, 154.04, 153.90, 143.23, 143.15, 135.93, 133.75, 132.79, 132.50, 130.75, 130.09, 130.05, 127.95, 127.80, 125.84, 125.27, 123.76, 122.71, 118.95, 114.02, 112.86, 107.61, 97.48, 97.37, 73.54, 66.41, 59.75, 52.71, 52.11, 49.31, 45.30, 45.00, 41.08, 38.23, 35.77, 35.34, 32.38, 30.77, 29.60, 29.22, 28.79, 28.72, 28.64, 25.28, 24.94, 24.84, 24.69, 23.46, 20.74, 14.07 ppm. HRMS (ESI-TOF): calculated for C49H52ClN8O6 (M+H): 883.3693, found: 883.3690.

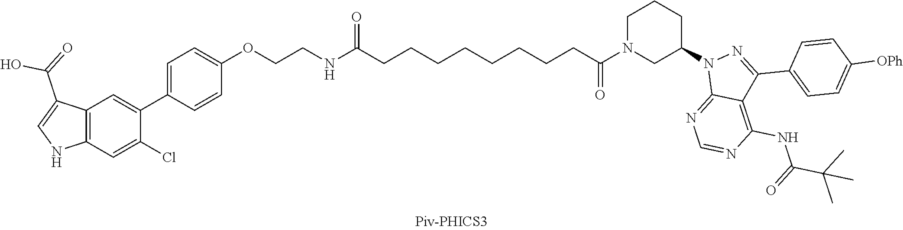 [SMILES: CC(C)(C)C(=O)Nc1ncnc2c1c(-c1ccc(Oc3ccccc3)cc1)nn2[C@@H]1CCCN(C(=O)CCCCCCCCC(=O)NCCOc2ccc(-c3cc4c(C(=O)O)c[nH]c4cc3Cl)cc2)C1]

### Synthesis of Piv-PHICS3

Step 1: Solution of 37 (20 mg, 30 μmol) in DCM (0.5 mL) was cooled to zero and treated with pivaloyl chloride (10 μL, 80 μmol) and DIPEA (20 μL) at zero. Reaction mixture was warmed to room temperature, stirred for 2 hours and concentrated under reduced pressure affording crude 38. Product 38 was used in the next step without further purification.

Step 2: Solution of 3 (13 mg, 30 μmol) in 0.5 mL of DCM was cooled to 0° C. and treated with trifluoroacetic acid (0.1 mL). Reaction mixture was warmed to room temperature and stirred for 2 hours before solvent was concentrated under reduced pressure. Product 38 from step 1 (30 μmol) was added in 1 mL DMF followed by DIPEA (20 μL). Reaction was stirred overnight, concentrated under reduced pressure and purified via HPLC affording 7 mg (24%) of desired product Piv-PHICS3 as an off-white solid. 1H NMR (400 MHz, CDCl3/CD3OH 1:1): 8.61 (br s, 1H), 7.98 (br s 1H), 7.87 (br s, 1H), 7.55-7.67 (m, 2H), 7.43-7.50 (m, 1H), 7.34-7.24 (m, 4H), 7.11-7.08 (m, 1H), 7.04-6.97 (m, 4H), 6.85 (m, 2H), 4.90-4.77 (m, 1H), 4.59-4.48 (m, 1H), 4.32-4.19 (m, 1H), 4.09-3.99 (m, 2H), 3.58-3.50 (m, 2H), 3.29-3.24 (m, 2H), 2.35-2.26 (m, 2H), 2.22-2.10 (m, 3H), 2.03-1.90 (m, 1H), 1.67-1.46 (m, 5H), 1.31-1.17 (m, 10H), 1.07 (s, 9H). 13C NMR (100 MHz, CDCl3/CD3OH 1:1): 174.68, 172.42, 157.74, 157.35, 156.07, 153.97, 152.76, 133.62, 133.07, 132.79, 132.56, 130.25, 129.39, 129.10, 127.97, 126.47, 124.75, 123.10, 122.46, 118.49, 117.79, 113.03, 111.83, 65.70, 52.95, 52.12, 49.12, 45.14, 44.90, 41.14, 38.29, 35.21, 32.46, 29.01, 28.76, 28.42, 28.17, 25.64, 25.07, 24.61, 23.97, 22.88 ppm. HRMS (ESI-TOF): calculated for C54H60ClN8O7 (M+H): 967.4268, found: 967.4265.

### 2.12. Validation of Ternary Complex (AMPK:PHICS3:BTK) Formation in HEK293T Cells

Co-immunoprecipitation was performed to evaluate the ternary complex (AMPK: PHICS3: BTK) formation in HEK293T cells after overexpressing the BTK, which is otherwise absent in these cells. HEK293T cells were cultured in DMEM supplemented with 10% FBS, penicillin (100 units/mL) and streptomycin (100 μg/mL). Cells were maintained at 37° C. in 5% CO2 humidified atmosphere.

After 36 hrs transfection of pcDNA3.1(+) BTK-Flag plasmid using TransIT®-293 Transfection Reagent, HEK 293T cells were maintained in a serum-free media overnight before incubation with compounds. After that, cells were treated with 5 μM concentration of AMPK activator or BTK binder or PHICS3 or DMSO control for 4 hrs in a fresh serum-free media before cells were lysed on ice in a lysis buffer (M-PER™ Mammalian Protein Extraction Reagent, Halt™ Protease and Phosphatase Inhibitor Cocktail (2×) and 50 mM NaF). Next, the protein concentrations of cell lysates were measured using Pierce™ BCA Protein Assay Kit. Equal amounts of cell lysates were incubated with Anti-FLAG® M2 Magnetic Beads overnight at 4° C. Then beads were washed three times with TBS buffer (50 mM Tris HCl, 150 mM NaCl, pH 7.4) and eluted the proteins with SDS-loading buffer by heating 5 min at 95° C. Eluted proteins were analyzed by western blotting using AMPK-α rabbit antibody (Cell Signaling, Cat #5832) or Flag mouse antibody (Cell Signaling, Cat #8146) (1:2000) or Beta-actin mouse antibody (Cell Signaling, Cat #3700) (1:2000).

### 2.13. Immunoblotting Analysis to Confirm the BTK Phosphorylation in HEK293T Cells

Co-immunoprecipitation was performed to evaluate the ternary complex (AMPK: PHICS3: BTK) formation in HEK293T cells after overexpressing the BTK, which is otherwise absent in these cells. HEK293T cells were cultured in DMEM supplemented with 10% FBS, penicillin (100 units/mL) and streptomycin (100 μg/mL). Cells were maintained at 37° C. in 5% CO2 humidified atmosphere.

After 36 hrs transfection of pcDNA3.1(+) BTK-Flag plasmid using TransIT®-293 Transfection Reagent, HEK 293T cells were maintained in a serum-free media overnight before incubation with compounds. After that, cells were treated with 5 μM concentration of AMPK activator or BTK binder or PHICS3 or DMSO control for 4 hrs in a fresh serum-free media before cells were lysed on ice in a lysis buffer (M-PER™ Mammalian Protein Extraction Reagent, Halt™ Protease and Phosphatase Inhibitor Cocktail (2×) and 50 mM NaF). Next, the protein concentrations of cell lysates were measured using Pierce™ BCA Protein Assay Kit. Equal amounts of cell lysates were incubated with Anti-FLAG® M2 Magnetic Beads overnight at 4° C. Then beads were washed three times with TBS buffer (50 mM Tris HCl, 150 mM NaCl, pH 7.4) and eluted the proteins with SDS-loading buffer by heating 5 min at 95° C. Eluted proteins were analyzed by western blotting using AMPK-α rabbit antibody (Cell Signaling, Cat #5832) or Flag mouse antibody (Cell Signaling, Cat #8146) (1:2000) or Beta-actin mouse antibody (Cell Signaling, Cat #3700) (1:2000).

A similar protocol was followed to observe the linker length, concentration, and dose dependency of PHICS3 induced phosphorylation in cells. HEK293T cells were maintained in a serum-free media overnight before incubation with the compounds after overexpressing Flag-BTK. In one experiment, 5 μM concentration of PHICS3 with different linkers (n=6, n=8, and PEG2) was incubated for 4 hrs. In another experiment, different concentrations (0.1, 0.5, 1, 5, and 10 μM) of PHICS3 were incubated for 4 hrs and 5 μM of PHICS3 was incubated with HEK293T cells for different time points (1, 2, 4 and 5 hrs) before lysis. Then western blotting was performed with Phospho-Btk (Ser180) (3D3) antibody (Cell Signaling, Cat #3537) (1:1000) and Flag mouse antibody (Cell Signaling, Cat #8146) (1:2000). To further validate that the AMPK activation alone is not responsible to induce significant Ser180 phosphorylation in cells, different concentrations (5, 10 and 20 μM) of a potent commercially available AMPK activator (PF-06409577) or 5 μM of PHICS3 or 5 μM of AMPK activator or DMSO control was incubated with HEK293T cells for 4 hrs before lysis of the cells and western blotting was performed using phospho-Btk (Ser180) (3D3) antibody and Flag mouse antibody.

Next, Applicants confirmed the target engagement of PHICS3 in cells through a competition experiment in the presence of ibrutinib as well as using a chemically modified PHICS3 (Piv-PHICS3). HEK293T cells were maintained in a serum-free media overnight before incubation of the compounds after overexpressing Flag-BTK. Then, cells were treated with 5 μM concentration of PHICS3 or Piv-PHICS3 for 4 hrs before cell lysis and western blotting was performed using phospho-Btk (Ser180) (3D3) antibody and Flag mouse antibody. In the competition experiment, HEK293T cells were incubated with DMSO or AMPK activator (5 μM) or PHICS3 (5 μM) or PHICS3 (5 μM)+ibrutinib (1 μM) or ibrutinib (1 μM) for 4 hrs in serum-free media. In this experiment, ibrutinib was treated 1 hr before the addition of PHICS3 molecule. After cell lysis, western blotting was performed using phospho-Btk (Ser180) (3D3) antibody and Flag mouse antibody.

### 2.14. Confirmation of the Phosphorylation Site of BTK and Target Engagement of PHICS3 by Mutational Studies.

S180A mutation was performed to confirm the PHICS3 induced phosphorylation site in HEK293T cells. Additionally, since the reversible analog of ibrutinib is an ATP competitive inhibitor of BTK, several mutations were performed in the ATP binding pocket of BTK to validate the target engagement of PHICS3. These mutant constructs were prepared using Q5® Site-Directed Mutagenesis Kit with the following primers: BTK S180A (forward: 5′-ACCTGGGAGTGCTCACCGGA-3′ (SEQ ID NO: 6), reverse: 5′-TTTAAGCTTCCATTCCTGTTCTCC-3′ (SEQ ID NO: 7)) K430R (forward: 5′-CGTGGCCATCAGGATGATCAAAG-3′ (SEQ ID NO: 8), reverse: 5′-TCGTACTGGCCTCTCCAT-3′ (SEQ ID NO: 9)) T474A (forward: 5′-CTTCATCATCGCTGAGTACATGGCCAATG-3′ (SEQ ID NO: 10), reverse: 5′-ATGGGGCGCTGCTTGGTG-3′ (SEQ ID NO: 11)) D539N (forward: 5′-TAAAGTATCTAATTTCGGCCTGTC-3′ (SEQ ID NO: 12), reverse: 5′-ACAACTCCTTGATCGTTTAC-3′ (SEQ ID NO: 13)). The entire open reading frame (ORF) of all prepared plasmids was confirmed by DNA sequencing.

Next, HEK293T cells were transfected with wild-type (WT) or mutant BTK-Flag plasmids using TransIT®-293 Transfection Reagent. After 36 hrs of transfection, cells were maintained in serum-free media overnight before incubation with compounds. After that, cells were treated with 5 μM concentration of AMPK activator or PHICS3 or DMSO control for 4 hrs in a fresh serum-free media before cells were lysed on ice in a lysis buffer (M-PER™ Mammalian Protein Extraction Reagent, Halt™ Protease and Phosphatase Inhibitor Cocktail (2×) and 50 mM NaF). Then, western blotting was performed with Phospho-Btk (Ser180) (3D3) antibody (Cell Signaling, Cat #3537) (1:1000) and Flag mouse antibody (Cell Signaling, Cat #8146) (1:2000).

### References

1. Tarrant, M. K.; Cole, P. A., The chemical biology of protein phosphorylation. Annu. Rev. Biochem. 2009, 78, 797-825.
2. Ferguson, F. M.; Gray, N. S., Kinase inhibitors: the road ahead. Nat. Rev. Drug Discov. 2018, 17, 353.
3. McCormick, J. W.; Pincus, D.; Resnekov, O.; Reynolds, K. A., Strategies for Engineering and Rewiring Kinase Regulation. Trends Biochem Sci 2020, 45 (3), 259-271.
4. Ku, N. O.; Azhar, S.; Omary, M. B., Keratin 8 phosphorylation by p38 kinase regulates cellular keratin filament reorganization: modulation by a keratin 1-like disease causing mutation. J Biol Chem 2002, 277 (13), 10775-82.
5. McLaughlin, R. J.; Spindler, M. P.; van Lummel, M.; Roep, B. O., Where, How, and When: Positioning Posttranslational Modification Within Type 1 Diabetes Pathogenesis. Curr. Diab. Rep. 2016, 16 (7), 63-63.
6. Neugebauer, K. M.; Merrill, J. T.; Wener, M. H.; Lahita, R. G.; Roth, M. B., SR proteins are autoantigens in patients with systemic lupus erythematosus. Importance of phosphoepitopes. Arthritis Rheum 2000, 43 (8), 1768-78.
7. Thapar, R., Structural basis for regulation of RNA-binding proteins by phosphorylation. ACS Chem Biol 2015, 10 (3), 652-66.
8. Garcia-Garcia, T.; Poncet, S.; Derouiche, A.; Shi, L.; Mijakovic, I.; Noirot-Gros, M. F., Role of Protein Phosphorylation in the Regulation of Cell Cycle and DNA-Related Processes in Bacteria. Front Microbiol 2016, 7, 184.
9. Gerry, C. J.; Schreiber, S. L., Unifying principles of bifunctional, proximity-inducing small molecules. Nat Chem Biol 2020, 16 (4), 369-378.
10. Stanton, B. Z.; Chory, E. J.; Crabtree, G. R., Chemically induced proximity in biology and medicine. Science 2018, 359 (6380).
11. Lai, A. C.; Crews, C. M., Induced protein degradation: an emerging drug discovery paradigm. Nat. Rev. Drug Discov. 2017, 16 (2), 101-114.
12. Hobert, E. M.; Schepartz, A., Rewiring kinase specificity with a synthetic adaptor protein. Journal of the American Chemical Society 2012, 134 (9), 3976-8.
13. Bhattacharyya, R. P.; Remenyi, A.; Yeh, B. J.; Lim, W. A., Domains, motifs, and scaffolds: the role of modular interactions in the evolution and wining of cell signaling circuits. Annu. Rev. Biochem. 2006, 75, 655-680.
14. Cameron, K. O.; Kung, D. W.; Kalgutkar, A. S.; Kurumbail, R. G.; Miller, R.; Salatto, C. T.; Ward, J.; Withka, J. M.; Bhattacharya, S. K.; Boehm, M.; Borzilleri, K. A.; Brown, J. A.; Calabrese, M.; Caspers, N. L.; Cokorinos, E.; Conn, E. L.; Dowling, M. S.; Edmonds, D. J.; Eng, H.; Fernando, D. P.; Frisbie, R.; Hepworth, D.; Landro, J.; Mao, Y.; Rajamohan, F.; Reyes, A. R.; Rose, C. R.; Ryder, T.; Shavnya, A.; Smith, A. C.; Tu, M.; Wolford, A. C.; Xiao, J., Discovery and Preclinical Characterization of 6-Chloro-5-[4-(1-hydroxycyclobutyl)phenyl]-1H-indole-3-carboxylic Acid (PF-06409577), a Direct Activator of Adenosine Monophosphate-activated Protein Kinase (AMPK), for the Potential Treatment of Diabetic Nephropathy. J. Med. Chem. 2016, 59 (17), 8068-81.
15. Mach, U. R.; Lewin, N. E.; Blumberg, P. M.; Kozikowski, A. P., Synthesis and pharmacological evaluation of 8- and 9-substituted benzolactam-V8 derivatives as potent ligands for protein kinase C, a therapeutic target for Alzheimer's disease. ChemMedChem 2006, 1 (3), 307-314.
16. Zegzouti, H.; Zdanovskaia, M.; Hsiao, K.; Goueli, S. A., ADP-Glo: A Bioluminescent and homogeneous ADP monitoring assay for kinases. Assay and drug development technologies 2009, 7 (6), 560-72.
17. Wurz, R. P.; Dellamaggiore, K.; Dou, H.; Javier, N.; Lo, M. C.; McCarter, J. D.; Mohl, D.; Sastri, C.; Lipford, J. R.; Cee, V. J., A “Click Chemistry Platform” for the Rapid Synthesis of Bispecific Molecules for Inducing Protein Degradation. Journal of medicinal chemistry 2018, 61 (2), 453-461.
18. Filippakopoulos, P.; Qi, J.; Picaud, S.; Shen, Y.; Smith, W. B.; Fedorov, O.; Morse, E. M.; Keates, T.; Hickman, T. T.; Felletar, I.; Philpott, M.; Munro, S.; McKeown, M. R.; Wang, Y.; Christie, A. L.; West, N.; Cameron, M. J.; Schwartz, B.; Heightman, T. D.; La Thangue, N.; French, C. A.; Wiest, O.; Kung, A. L.; Knapp, S.; Bradner, J. E., Selective inhibition of BET bromodomains. Nature 2010, 468 (7327), 1067-73.
19. Bielefeld-Sevigny, M., AlphaLISA immunoassay platform—the “no-wash” high-throughput alternative to ELISA. Assay Drug Dev Technol 2009, 7 (1), 90-2.
20. Beaudet, L.; Rodriguez-Suarez, R.; Venne, M.-H.; Caron, M.; Bedard, J.; Brechler, V.; Parent, S.; Bielefeld-Sevigny, M., AlphaLISA immunoassays: the no-wash alternative to ELISAs for research and drug discovery. Nature Methods 2008, 5, A10.
21. Douglass, E. F., Jr.; Miller, C. J.; Sparer, G.; Shapiro, H.; Spiegel, D. A., A comprehensive mathematical model for three-body binding equilibria. Journal of the American Chemical Society 2013, 135 (16), 6092-9.
22. Rodbard, D.; Feldman, Y.; Jaffe, M. L.; Miles, L. E., Kinetics of two-site immunoradiometric (‘sandwich’) assays-II. Studies on the nature of the ‘high-dose hook effect’. Immunochemistry 1978, 15 (2), 77-82.
23. Johanns, M.; Lai, Y. C.; Hsu, M. F.; Jacobs, R.; Vertommen, D.; Van Sande, J.; Dumont, J. E.; Woods, A.; Carling, D.; Hue, L.; Viollet, B.; Foretz, M.; Rider, M. H., AMPK antagonizes hepatic glucagon-stimulated cyclic AMP signalling via phosphorylation-induced activation of cyclic nucleotide phosphodiesterase 4B. Nat. Commun. 2016, 7, 10856.
24. Wang, Z.; Ma, J.; Miyoshi, C.; Li, Y.; Sato, M.; Ogawa, Y.; Lou, T.; Ma, C.; Gao, X.; Lee, C.; Fujiyama, T.; Yang, X.; Zhou, S.; Hotta-Hirashima, N.; Klewe-Nebenius, D.; Ikkyu, A.; Kakizaki, M.; Kanno, S.; Cao, L.; Takahashi, S.; Peng, J.; Yu, Y.; Funato, H.; Yanagisawa, M.; Liu, Q., Quantitative phosphoproteomic analysis of the molecular substrates of sleep need. Nature 2018, 558 (7710), 435-439.
25. Wu, S. Y.; Lee, A. Y.; Lai, H. T.; Zhang, H.; Chiang, C. M., Phospho switch triggers Brd4 chromatin binding and activator recruitment for gene-specific targeting. Molecular cell 2013, 49 (5), 843-57.
26. Hornbeck, P. V.; Zhang, B.; Murray, B.; Kornhauser, J. M.; Latham, V.; Skrzypek, E., PhosphoSitePlus, 2014: mutations, PTMs and recalibrations. Nucleic acids research 2015, 43 (Database issue), D512-20.
27. Davies, S. P.; Carling, D.; Hardie, D. G., Tissue distribution of the AMP-activated protein kinase, and lack of activation by cyclic-AMP-dependent protein kinase, studied using a specific and sensitive peptide assay. Europeanjoumal of biochemistry 1989, 186 (1-2), 123-8.
28. Hardie, D. G.; Schaffer, B. E.; Brunet, A., AMPK: An Energy-Sensing Pathway with Multiple Inputs and Outputs. Trends in cell biology 2016, 26 (3), 190-201.
29. Weekes, J.; Ball, K. L.; Caudwell, F. B.; Hardie, D. G., Specificity determinants for the AMP-activated protein kinase and its plant homologue analyzed using synthetic peptides. FEBS letters 1993, 334 (3), 335-9.
30. Schaffer, B. E.; Levin, R. S.; Hertz, N. T.; Maures, T. J.; Schoof, M. L.; Hollstein, P. E.; Benayoun, B. A.; Banko, M. R.; Shaw, R. J.; Shokat, K. M.; Brunet, A., Identification of AMPK Phosphorylation Sites Reveals a Network of Proteins Involved in Cell Invasion and Facilitates Large-Scale Substrate Prediction. Cell metabolism 2015, 22 (5), 907-21.
31. Gadd, M. S.; Testa, A.; Lucas, X.; Chan, K. H.; Chen, W.; Lamont, D. J.; Zengerle, M.; Ciulli, A., Structural basis of PROTAC cooperative recognition for selective protein degradation. Nat Chem Biol 2017, 13 (5), 514-521.
32. Kozikowski, A. P.; Wang, S.; Ma, D.; Yao, J.; Ahmad, S.; Glazer, R. I.; Bogi, K.; Acs, P.; Modarres, S.; Lewin, N. E.; Blumberg, P. M., Modeling, Chemistry, and Biology of the Benzolactam Analogs of Indolactam V (ILV). 2. Identification of the Binding Site of the Benzolactams in the CRD2 Activator-Binding Domain of PKC6 and Discovery of an ILV Analog of Improved Isoenzyme Selectivity. J. Med. Chem. 1997, 40 (9), 1316-1326.
33. Pal Singh, S.; Dammeijer, F.; Hendriks, R. W., Role of Bruton's tyrosine kinase in B cells and malignancies. Mol Cancer 2018, 17 (1), 57.
34. Burger, J. A.; Wiestner, A., Targeting B cell receptor signalling in cancer: preclinical and clinical advances. Nat Rev Cancer 2018, 18 (3), 148-167.
35. Kang, S. W.; Wahl, M. I.; Chu, J.; Kitaura, J.; Kawakami, Y.; Kato, R. M.; Tabuchi, R.; Tarakhovsky, A.; Kawakami, T.; Turck, C. W.; Witte, O. N.; Rawlings, D. J., PKCbeta modulates antigen receptor signaling via regulation of Btk membrane localization. The EMBOjournal 2001, 20 (20), 5692-702.
36. Liang, C.; Tian, D.; Ren, X.; Ding, S.; Jia, M.; Xin, M.; Thareja, S., The development of Bruton's tyrosine kinase (BTK) inhibitors from 2012 to 2017: A mini-review. Eur J Med Chem 2018, 151, 315-326.
37. Johnson, A. R.; Kohli, P. B.; Katewa, A.; Gogol, E.; Belmont, L. D.; Choy, R.; Penuel, E.; Burton, L.; Eigenbrot, C.; Yu, C.; Ortwine, D. F.; Bowman, K.; Franke, Y.; Tam, C.; Estevez, A.; Mortara, K.; Wu, J.; Li, H.; Lin, M.; Bergeron, P.; Crawford, J. J.; Young, W. B., Battling Btk Mutants With Noncovalent Inhibitors That Overcome Cys481 and Thr474 Mutations. ACS Chem Biol 2016, 11 (10), 2897-2907.
38. Bender, A. T.; Gardberg, A.; Pereira, A.; Johnson, T.; Wu, Y.; Grenningloh, R.; Head, J.; Morandi, F.; Haselmayer, P.; Liu-Bujalski, L., Ability of Bruton's Tyrosine Kinase Inhibitors to Sequester Y551 and Prevent Phosphorylation Determines Potency for Inhibition of Fc Receptor but not B-Cell Receptor Signaling. Mol Pharmacol 2017, 91 (3), 208-219.
39. Koehler, A. N., A complex task? Direct modulation of transcription factors with small molecules. Curr. Opin. Chem. Biol. 2010, 14 (3), 331-340.

### Example 2—AMP Enhancement of Phosphorylation In Vitro

In vitro kinase assay was conducted for BRD4 phosphorylation by AMPK (1 uM compound). Compounds MS231, and VS806 tested are depicted below.

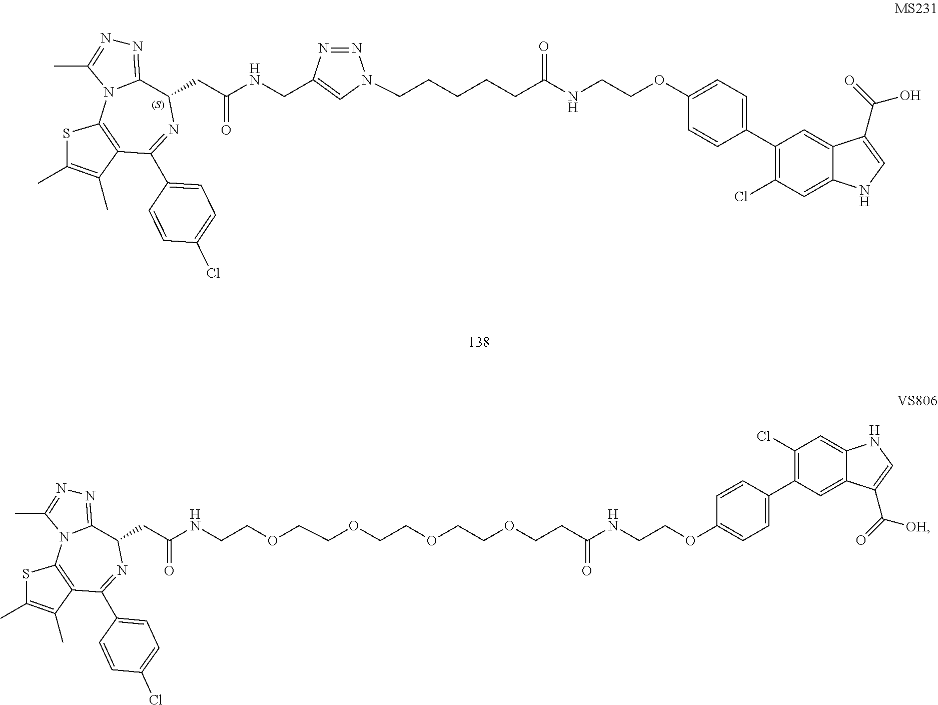

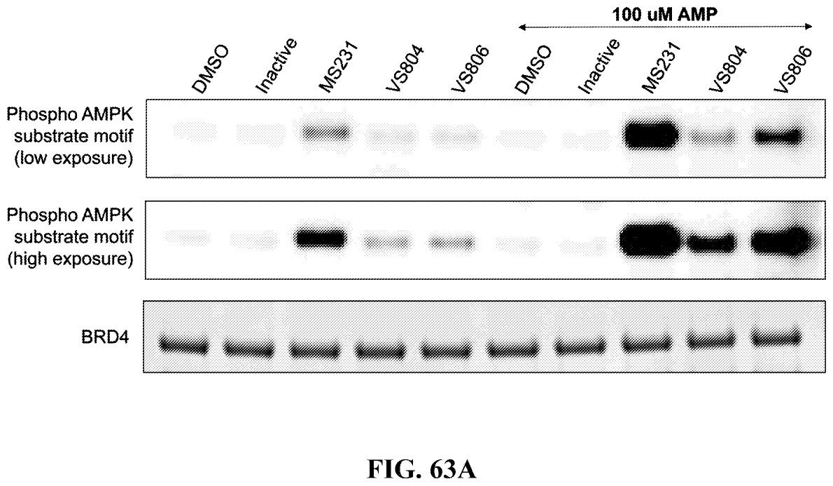

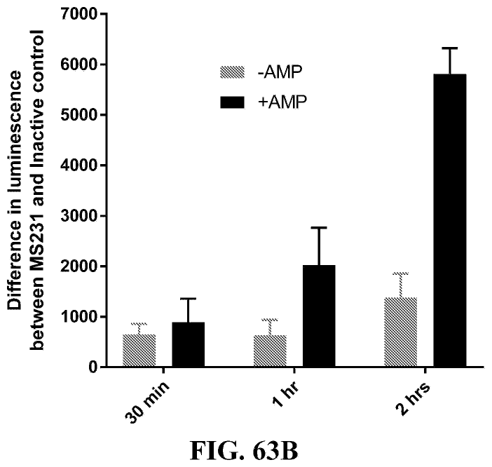

Compounds MS231, VS806 and VS804 were tested with and without 100 uM AMP. Addition of AMP enhances the phosphorylation signal, as shown in FIG. 63A. ADP glo for BRD4 phosphorylation by AMPK with and without AMP was conducted. The addition of AMP enhanced the phosphorylation signal, with ADP production measured at 2 hours, 20 nM AMPK.

TABLE 3
ADP production: 2 hrs, 20 nM AMPK

| | No AMP | With AMP |
|---|---|---|
| Average (nM) | 313.4 | 1319.8 |
| SD (+/−) | 93.5 | 100.4 |
| turnover | 16 | 66 |

### Example 3—Design of Abl-Engaging PHICS Molecules

To design Abl-engaging PHICS molecules Applicants selected two known Abl kinase activators: DPH1 and a dihydropyrazol activator (2). Based on the reported crystal structures of Abl with its activators, solvent-exposed phenyl and amino groups in positions 1 and 3 of DPH and dihydropyrazol rings, respectively, were chosen as linker attachment sites. First, for proof-of-concept experiments, the Halotag binding chloroalkyl chain was attached to both activators via three linkers, which varied in their length and polarity. Next, Applicants evaluated the Abl-PHICS induce phosphorylation on HaloTag protein using Adenosine 5′-[γ-thio]triphosphate (ATP-γ-S) as the donor of phosphate and probe the phosphorylation with anti-thiophosphate ester antibody (3). In vitro phosphorylation revealed that dihydropyrazol-derived bifunctional molecules (PHICS 9.1-9.3, FIG. 64A-64B) significantly increased the level of Halo-tag phosphorylation by Abl kinase when compared to phosphorylation levels with Abl binder alone. Interestingly, PHICS 9.1 containing the most nonpolar linker exhibiting the highest level of phosphorylation (FIG. 64B). At the same time, DPH-derived PHICS with the same linkers did not provide detectable levels of phosphorylation in tested conditions (data not shown). Both DPH and dihydropyrazol derived PHICS efficiently labeled Halotag protein, which was confirmed by the suppression of TMR-labeling4 (FIG. 64B). DPH and dihydropyrazol activators are binding to Abl with similar affinity (100-200 nM), but chosen linker attachment sites provided different exit vectors, which could potentially lead to two different ternary complexes. The orientation of the proteins in the ternary complex with the DPH-based exit vector might be unproductive. This can explain observed differences in phosphorylation levels of Halotag with tested bifunctional molecules. However, additional experiments (e.g., Alpha Screen, design of DPH analogs with by changing exit vector positions similar to dihydropyrazol) will be performed for better understanding of two systems.

After the validation of dihydropyrazol Abl binders in the Halotag targeting experiment, Applicants decided to design PHICS molecules against one of the proposed targets—BRD4. Similarly to Halo-Abl PHICS design, the binder of BRD4 protein—(+)-JQ1—was attached to dihydropyrazol via various types of linkers (PHICS 10.1-10.5, FIG. 64A). Resulting molecules were tested in vitro following the same thio-ATP based protocol and demonstrated different levels of BRD4 phosphorylation when compared to Abl binder alone (FIG. 64C). Interestingly, in the case of BRD4, more polar linker with an average length PHICS 10.3 showed the highest level of phosphorylation. As a control, Applicants have prepared an enantiomer of PHICS 10.3-PHICS i10.3 (derived from (−)-JQ1), which should be unable to bind BRD4. As expected, in the presence of PHICS i10.3 phosphorylation level is the same as with DMSO or Abl binder alone, proving that observed signal in the presence of PHICS 10.3 is a result of productive PHICS-induced ternary complex formation. Further, if any of the three components of the ternary complex or thio-ATP were absent, phosphorylation was not detected. Finally, Applicants observed ternary complex formation in the presence of PHICS 10.3, but not with PHICS i10.3 by Alpha Screen assay (data not shown).

### The Following References Relate to Example 3

1. Yang, J.; Campobasso, N.; Biju, M. P.; Fisher, K.; Pan, X. Q.; Cottom, J.; Galbraith, S.; Ho, T.; Zhang, H.; Hong, X.; Ward, P.; Hofmann, G.; Siegfried, B.; Zappacosta, F.; Washio, Y.; Cao, P.; Qu, J.; Bertrand, S.; Wang, D. Y.; Head, M. S.; Li, H.; Moores, S.; Lai, Z.; Johanson, K.; Burton, G.; Erickson-Miller, C.; Simpson, G.; Tummino, P.; Copeland, R. A.; Oliff, A., Discovery and characterization of a cell-permeable, small-molecule c-Abl kinase activator that binds to the myristoyl binding site. Chem Biol 2011, 18 (2), 177-86.
2. Simpson, G. L.; Bertrand, S. M.; Borthwick, J. A.; Campobasso, N.; Chabanet, J.; Chen, S.; Coggins, J.; Cottom, J.; Christensen, S. B.; Dawson, H. C.; Evans, H. L.; Hobbs, A. N.; Hong, X.; Mangatt, B.; Munoz-Muriedas, J.; Oliff, A.; Qin, D.; Scott-Stevens, P.; Ward, P.; Washio, Y.; Yang, J.; Young, R. J., Identification and Optimization of Novel Small c-Abl Kinase Activators Using Fragment and HTS Methodologies. Journal of Medicinal Chemistry 2019, 62 (4), 2154-2171.
3. Allen, J. J.; Li, M.; Brinkworth, C. S.; Paulson, J. L.; Wang, D.; Hubner, A.; Chou, W. H.; Davis, R. J.; Burlingame, A. L.; Messing, R. O.; Katayama, C. D.; Hedrick, S. M.; Shokat, K. M., A semisynthetic epitope for kinase substrates. Nature methods 2007, 4 (6), 511-6.
4. Los, G. V.; Encell, L. P.; McDougall, M. G.; Hartzell, D. D.; Karassina, N.; Zimprich, C.; Wood, M. G.; Learish, R.; Ohana, R. F.; Urh, M.; Simpson, D.; Mendez, J.; Zimmerman, K.; Otto, P.; Vidugiris, G.; Zhu, J.; Darzins, A.; Klaubert, D. H.; Bulleit, R. F.; Wood, K. V., HaloTag: a novel protein labeling technology for cell imaging and protein analysis. ACS chemical biology 2008, 3 (6), 373-82.

### Example 4—Diversification of the Nature of Kinases and their Binders Used for PHICS Generation

As mentioned above, Applicants have developed two proof-of-concept types of PHICS using AMPK and PKC kinases, which both phosphorylate Ser and Thr residues. However, the scope of cellular phosphorylation is much broader: there are more than 500 kinases, with approximately one-fifth of them belonging to Tyr kinase family, which are responsible for phosphorylation of Tyr residues. Taking into account the fact that abundance and localization of kinases vary significantly in different types of cells, Applicants propose to go beyond AMPK/PKC and expand the scope of PHICS to other kinases.

First, PHICS for Tyr phosphorylation should be established. In addition to this, so far Applicants utilized only validated activators of kinases, the scope of which is extremely limited. It is likely that reversible allosteric inhibitors can also produce functional PHICS. Thalidomide, bestatin, and pomalidomide are inhibitors of E3 ligase; nevertheless, PROTACs containing these compounds lead to efficient degradation of POIs. The primary purpose of bifunctional molecules (PROTACs or PHICS) is to bring appropriate enzyme and protein of interest close to each other. Since binding of the noncovalent inhibitor to the enzyme is reversible, upon dissociation of E3 ligase or kinase from a bifunctional molecule, it changes its conformation to active form and ubiquitination (or phosphorylation in case of PHICS) of proximal protein may take place.

To expand the scope of kinases utilized by PHICS, Applicants will try two relatively abundant tyrosine kinases with different localization sites: membrane-bound Insulin Receptor (IRTK) and Abelson (ABL) Tyr kinase, which can be found in the nucleus, cytoplasm, and mitochondria. To construct PHICS molecules for these kinases, Applicants will use their well-characterized activators DPH and kojic acid (16-18). With regards to allosteric inhibitors, Borussertib and Trametnib are validated chemical matters targeting RAC-alpha serine/threonine-protein kinase (AKT) and mitogen-activated protein kinase (MEK), enzymes with relatively high abundance (8.6×103 and 1.2×105 molecules per U2OS cell respectively) (19) and different cell localization (cytoplasm, membrane, and nucleus) (20).

To evaluate four proposed kinases, Applicants are going to design PHICS molecules for a nuclear target (BRD4) and cytoplasmic target (AR). PROTACs derived from their binders, (+)-JQ1 and enzalutamide, successfully degraded both proteins and one of them (ARV-110) even received fast track designation from the FDA for the treatment of patients with metastatic castration-resistant prostate cancer. With binders of six kinases and two targets in hand, Applicants plan to connect them with three different types of linkers which will result in multiple combinations. To simplify the synthesis of these molecules, a rapid modular approach will be utilized: (+)-JQ1 and enzalutamide will be attached (via amide and ether bonds respectively) to three different linkers containing azide in the end (6 unique molecules), when six kinase activators will be functionalized with alkyne (6 unique molecules, FIG. 80). Obtained building blocks will be connected via biorthogonal click-chemistry. All combinations will be synthesized and evaluated in vitro using assays described above and in cellulo using U2OS and HEK293 cell lines. The most promising kinases will be utilized for targeting of transcription factors in Example 5. Applicants note that if designed PHICS does not induce phosphorylation of BRD4 and AR in biochemical assays, other kinases with be utilized (e.g., Lyn (21), BTK (22), Src (23), GSK (24), and LIMK (25), for which several kinase binders exist.

### Example 5—Design and In Vitro Evaluation of PHICS for Modulation of Various Transcription Factors

Approximately 1600 human transcription factors (TFs) are known which represent 8% of all genes and account for 20% of oncogenes (26). Applicants have selected four TFs with different subclass assignments, modes of targeted interaction and intended mechanisms of cancer suppression (FIGS. 81A-81D). Applicants' first goal is the disruption of protein-protein interaction in oncogenic Myc-Max pair (latent cytoplasmic factor subclass). Recently, using small molecule microarray screening assay, Koehler's lab found compound KI-MS2-008 (FIG. 81A) that was able to disrupt heterodimer Myc-Max and suppress tumor growth in-vivo via stabilization of Max-Max homodimer (27). Applicants plan to leverage on this finding and use KI-MS2-008 for construction of PHICS that can phosphorylate Max. It is expected that the introduction of phosphate groups on the surface of Max will prevent the formation of Max-Myc heterodimer and shift equilibrium towards unbound Myc.

Applicants' second goal is the disruption of protein-DNA interaction for Estrogen Receptor (ER, nuclear resident factor subclass). PHICS molecule will be designed using a known inhibitor of ER—raloxifene (FIG. 81B) (28). Using raloxifene alone, constant ligand saturation should be maintained to keep the ER from interacting with DNA. PHICS strategy relies on catalytic phosphorylation of ER with a small bifunctional molecule, and it has the potential to exhibit increased therapeutic effect with a smaller dose. Applicants' third target p53 protein is even more interesting because depending on the site and valency of phosphorylation, either protein-protein or protein-DNA interaction can be disrupted. It was found that defect in the phosphorylation of p53 contributes to the acquisition of p53 resistance in oral squamous cell carcinomas due to its inability to dissociate from its degrader MDM2 (29). Thus Applicants aim to restore phosphorylation by 2,5-bis(5-hydroxymethyl-2-thienyl)furan or RITA-derived PHICS and disrupt protein-protein p53-MDM2 interaction (30). In the same time, phosphorylation of p53 at the DNA-binding domain will lead to interference with protein-DNA interaction which can be extremely beneficial in types of cancer with p53 overexpression (31-32). For a final target, Applicants plan to improve the stability of protein-protein interaction: O-catenin is known to form stable degradation complexes and prevent downstream signaling upon phosphorylation (33). For this purpose, UU-T02-derived PHICS will be designed (34).

Using kinases identified above, Applicants will design several PHICS molecules for each target and evaluate their ability to induce phosphorylation in vitro. Preliminary data will be generated in U2OS and HEK293 cell lines. Cells will be treated with an active or inactive PHICS, and target phosphorylation will be monitored after immunoprecipitation followed by immunoblotting with antibodies specific for phospho Ser/Thr or Tyr. Co-immunoprecipitation of the kinase and target will be attempted after treating cells with the active or inactive PHICS to further confirm complex formation. Second, to identify the phosphorylation sites and determine if any changes to PHICS design affect the site and level of target's phosphorylation, mass spectrometry studies will also be performed. Third, the effect of designed PHICS on various cancer cells models will be evaluated. More specifically P493-6, ST486 and of Myc-induced T cell acute lymphoblastic leukemia (T-ALL) cell lines will be used for studies involving Myc-Max pair. In the case of ER, PHICS molecules will be evaluated in MCF-7 and T47D ER+ cells. Finally, SW480, HCT116, HT29, MDA-MB-231 Daoy MB, and Rh36 cell lines will be used to study p53 and β-catenin phosphorylation effects.

If direct phosphorylation of proposed targets fails, Applicants will modulate transcription factors via phosphorylation of their binding partners. For example, MDM2, binding of which to p53 labels it for degradation, or HSP90, which is stabilizing HIF-α, can be targeted with MI-1061 or deguelin-derived PHICS respectively (35-37).

### The Following References Relate to Examples 4-5

1. Klaeger, S.; Heinzlmeir, S.; Wilhelm, M.; Polzer, H.; Vick, B.; Koenig, P.-A.; Reinecke, M.; Ruprecht, B.; Petzoldt, S.; Meng, C.; Zecha, J.; Reiter, K.; Qiao, H.; Helm, D.; Koch, H.; Schoof, M.; Canevari, G.; Casale, E.; Depaolini, S. R.; Feuchtinger, A.; Wu, Z.; Schmidt, T.; Rueckert, L.; Becker, W.; Huenges, J.; Garz, A.-K.; Gohlke, B.-O.; Zolg, D. P.; Kayser, G.; Vooder, T.; Preissner, R.; Hahne, H.; Tonisson, N.; Kramer, K.; Goetze, K.; Bassermann, F.; Schlegl, J.; Ehrlich, H.-C.; Aiche, S.; Walch, A.; Greif, P. A.; Schneider, S.; Felder, E. R.; Ruland, J.; Medard, G.; Jeremias, I.; Spiekermann, K.; Kuster, B., The target landscape of clinical kinase drugs. Science (Washington, D.C., U. S.) 2017, 358 (6367), 1148.
2. Wu, P.; Nielsen, T. E.; Clausen, M. H., FDA-approved small-molecule kinase inhibitors. Trends Pharmacol. Sci. 2015, 36 (7), 422-439.
3. Bhullar, K. S.; Lagaron, N. O.; McGowan, E. M.; Parmar, I.; Jha, A.; Hubbard, B. P.; Rupasinghe, H. P. V., Kinase-targeted cancer therapies: progress, challenges and future directions. Mol. Cancer 2018, 17, 48/1-48/20.
4. Koehler, A. N., A complex task? Direct modulation of transcription factors with small molecules. Curr. Opin. Chem. Biol. 2010, 14 (3), 331-340.
5. Hagenbuchner, J.; Ausserlechner, M. J., Targeting transcription factors by small compounds-Current strategies and future implications. Biochem. Pharmacol. (Amsterdam, Neth.) 2016, 107, 1-13.
6. Choudhary, A.; Wu, P.; Ding, E. A. Compositions and methods for inducing protein phosphorylation. Mar. 31, 2016, 2016.
7. Fegan, A.; White, B.; Carlson, J. C. T.; Wagner, C. R., Chemically Controlled Protein Assembly: Techniques and Applications. Chem Rev 2010, 110 (6), 3315-3336.
8. Spencer, D.; Wandless, T.; Schreiber, S.; Crabtree, G., Controlling signal transduction with synthetic ligands. Science 1993, 262 (5136), 1019-1024.
9. Guilinger, J. P.; Pattanayak, V.; Reyon, D.; Tsai, S. Q.; Sander, J. D.; Joung, J. K.; Liu, D. R., Broad Specificity Profiling of TALENs Results in Engineered Nucleases With Improved DNA Cleavage Specificity. Nat Meth 2014, 11 (4), 429-435.
10. Lu, J.; Qian, Y.; Altieri, M.; Dong, H.; Wang, J.; Raina, K.; Hines, J.; Winkler, J. D.; Crew, A. P.; Coleman, K.; Crews, C. M., Hijacking the E3 Ubiquitin Ligase Cereblon to Efficiently Target BRD4. Chem. Biol. (Oxford, U. K.) 2015, 22 (6), 755-763.
11. Zengerle, M.; Chan, K.-H.; Ciulli, A., Selective Small Molecule Induced Degradation of the BET Bromodomain Protein BRD4. ACS Chem. Biol. 2015, 10 (8), 1770-1777.
12. Salami, J.; Alabi, S.; Willard, R. R.; Vitale, N. J.; Wang, J.; Dong, H.; Jin, M.; McDonnell, D. P.; Crew, A. P.; Neklesa, T. K.; Crews, C. M., Androgen receptor degradation by the proteolysis-targeting chimera ARCC-4 outperforms enzalutamide in cellular models of prostate cancer drug resistance. Commun. Biol. 2018, 1 (1), 1-9.
13. Neklesa, T. K.; Winkler, J. D.; Crews, C. M., Targeted protein degradation by PROTACs. Pharmacol. Ther. 2017, 174, 138-144.
14. Mach, U. R.; Lewin, N. E.; Blumberg, P. M.; Kozikowski, A. P., Synthesis and pharmacological evaluation of 8- and 9-substituted benzolactam-V8 derivatives as potent ligands for protein kinase C, a therapeutic target for Alzheimer's disease. ChemMedChem 2006, 1 (3), 307-314.
15. Cameron, K. O.; Kung, D. W.; Kalgutkar, A. S.; Kurumbail, R. G.; Miller, R.; Salatto, C. T.; Ward, J.; Withka, J. M.; Bhattacharya, S. K.; Boehm, M.; Borzilleri, K. A.; Brown, J. A.; Calabrese, M.; Caspers, N. L.; Cokorinos, E.; Conn, E. L.; Dowling, M. S.; Edmonds, D. J.; Eng, H.; Fernando, D. P.; Frisbie, R.; Hepworth, D.; Landro, J.; Mao, Y.; Rajamohan, F.; Reyes, A. R.; Rose, C. R.; Ryder, T.; Shavnya, A.; Smith, A. C.; Tu, M.; Wolford, A. C.; Xiao, J., Discovery and Preclinical Characterization of 6-Chloro-5-[4-(1-hydroxycyclobutyl)phenyl]-1H-indole-3-carboxylic Acid (PF-06409577), a Direct Activator of Adenosine Monophosphate-activated Protein Kinase (AMPK), for the Potential Treatment of Diabetic Nephropathy. J. Med. Chem. 2016, 59 (17), 8068-8081.
16. Yang, J.; Campobasso, N.; Biju, M. P.; Fisher, K.; Pan, X.-Q.; Cottom, J.; Galbraith, S.; Ho, T.; Zhang, H.; Hong, X.; Ward, P.; Hofmann, G.; Siegfried, B.; Zappacosta, F.; Washio, Y.; Cao, P.; Qu, J.; Bertrand, S.; Wang, D.-Y.; Head, M. S.; Li, H.; Moores, S.; Lai, Z.; Johanson, K.; Burton, G.; Erickson-Miller, C.; Simpson, G.; Tummino, P.; Copeland, R. A.; Oliff, A., Discovery and Characterization of a Cell-Permeable, Small-Molecule c-Abl Kinase Activator that Binds to the Myristoyl Binding Site. Chem. Biol. (Cambridge, Mass., U.S.) 2011, 18 (2), 177-186.
17. Simpson, G. L.; Bertrand, S. M.; Borthwick, J. A.; Campobasso, N.; Chabanet, J.; Chen, S.; Coggins, J.; Cottom, J.; Christensen, S. B.; Dawson, H. C.; Evans, H. L.; Hobbs, A. N.; Hong, X.; Mangatt, B.; Munoz-Muriedas, J.; Oliff, A.; Qin, D.; Scott-Stevens, P.; Ward, P.; Washio, Y.; Yang, J.; Young, R. J., Identification and Optimization of Novel Small c-Abl Kinase Activators Using Fragment and HTS Methodologies. J. Med. Chem. 2019, 62 (4), 2154-2171.
18. Pirrung, M. C.; Deng, L.; Lin, B.; Webster, N. J. G., Quinone replacements for small molecule insulin mimics. ChemBioChem 2008, 9 (3), 360-362.
19. Beck, M.; Schmidt, A.; Malmstroem, J.; Claassen, M.; Ori, A.; Szymborska, A.; Herzog, F.; Rinner, O.; Ellenberg, J.; Aebersold, R., The quantitative proteome of a human cell line. Mol Syst Biol 2011, 7, 549.
20. Wu, P.; Clausen, M. H.; Nielsen, T. E., Allosteric small-molecule kinase inhibitors. Pharmacol. Ther. 2015, 156, 59-68.
21. Saporito, M. S.; Ochman, A. R.; Lipinski, C. A.; Handler, J. A.; Reaume, A. G., MLR-1023 is a potent and selective allosteric activator of Lyn kinase in vitro that improves glucose tolerance in vivo. J. Pharmacol. Exp. Ther. 2012, 342 (1), 15-22.
22. Tinworth, C. P.; Lithgow, H.; Dittus, L.; Bassi, Z. I.; Hughes, S. E.; Muelbaier, M.; Dai, H.; Smith, I. E. D.; Kerr, W. J.; Burley, G. A.; Bantscheff, M.; Harling, J. D., PROTAC-Mediated Degradation of Bruton's Tyrosine Kinase Is Inhibited by Covalent Binding. ACS Chem. Biol. 2019, 14 (3), 342-347.
23. Simard, J. R.; Klueter, S.; Gruetter, C.; Getlik, M.; Rabiller, M.; Rode, H. B.; Rauh, D., A new screening assay for allosteric inhibitors of cSrc. Nat. Chem. Biol. 2009, 5 (6), 394-396.
24. Palomo, V.; Perez, D. I.; Roca, C.; Anderson, C.; Rodriguez-Muela, N.; Perez, C.; Morales-Garcia, J. A.; Reyes, J. A.; Campillo, N. E.; Perez-Castillo, A. M.; Rubin, L. L.; Timchenko, L.; Gil, C.; Martinez, A., Subtly Modulating Glycogen Synthase Kinase 3 β: Allosteric Inhibitor Development and Their Potential for the Treatment of Chronic Diseases. J. Med. Chem. 2017, 60 (12), 4983-5001.
25. Goodwin, N. C.; Cianchetta, G.; Burgoon, H. A.; Healy, J.; Mabon, R.; Strobel, E. D.; Allen, J.; Wang, S.; Hamman, B. D.; Rawlins, D. B., Discovery of a Type III Inhibitor of LIM Kinase 2 That Binds in a DFG-Out Conformation. ACS Med. Chem. Lett. 2015, 6 (1), 53-57.
26. Lambert, S. A.; Jolma, A.; Campitelli, L. F.; Das, P. K.; Yin, Y.; Albu, M.; Chen, X.; Taipale, J.; Hughes, T. R.; Weirauch, M. T., The Human Transcription Factors. Cell (Cambridge, Mass., U. S.) 2018, 172 (4), 650-665.
27. Struntz, N. B.; Chen, A.; Deutzmann, A.; Wilson, R. M.; Stefan, E.; Evans, H. L.; Ramirez, M. A.; Liang, T.; Caballero, F.; Wildschut, M. H. E.; Neel, D. V.; Freeman, D. B.; Pop, M. S.; McConkey, M.; Muller, S.; Curtin, B. H.; Tseng, H.; Frombach, K. R.; Butty, V. L.; Levine, S. S.; Feau, C.; Elmiligy, S.; Hong, J. A.; Lewis, T. A.; Vetere, A.; Clemons, P. A.; Malstrom, S. E.; Ebert, B. L.; Lin, C. Y.; Felsher, D. W.; Koehler, A. N., Stabilization of the Max Homodimer with a Small Molecule Attenuates Myc-Driven Transcription. Cell Chem. Biol. 2019, 26 (5), 711-723.e14.
28. Hu, J.; Hu, B.; Wang, M.; Xu, F.; Miao, B.; Yang, C.-Y.; Wang, M.; Liu, Z.; Hayes, D. F.; Chinnaswamy, K.; Delproposto, J.; Stuckey, J.; Wang, S., Discovery of ERD-308 as a highly potent proteolysis targeting chimera (PROTAC) degrader of estrogen receptor (ER). J. Med. Chem. 2019, 62 (3), 1420-1442.
29. Ichwan, S. J. A.; Yamada, S.; Sumrejkanchanakij, P.; Ibrahim-Auerkari, E.; Eto, K.; Ikeda, M. A., Defect in serine 46 phosphorylation of p53 contributes to acquisition of p53 resistance in oral squamous cell carcinoma cells. Oncogene 2006, 25 (8), 1216-1224.
30. Issaeva, N.; Bozko, P.; Enge, M.; Protopopova, M.; Verhoef, L. G. G. C.; Masucci, M.; Pramanik, A.; Selivanova, G., Small molecule RITA binds to p53, blocks p53-HDM-2 interaction and activates p53 function in tumors. Nat. Med. (N. Y., NY, U. S.) 2004, 10 (12), 1321-1328.
31. Davidoff, A. M.; Humphrey, P. A.; Iglehart, J. D.; Marks, J. R., Genetic basis for p53 overexpression in human breast cancer. Proc. Natl. Acad. Sci. U.S.A 1991, 88 (11), 5006-10.
32. Kakeji, Y.; Korenaga, D.; Tsujitani, S.; Baba, H.; Anai, H.; Maehara, Y.; Sugimachi, K., Gastric cancer with p53 overexpression has high potential for metastasising to lymph nodes. Br J Cancer 1993, 67 (3), 589-93.
33. Clevers, H., Wnt/beta-catenin signaling in development and disease. Cell 2006, 127 (3), 469-80.
34. Cui, C.; Zhou, X.; Zhang, W.; Qu, Y.; Ke, X., Is β-Catenin a Druggable Target for Cancer Therapy? Trends Biochem. Sci. 2018, 43 (8), 623-634.
35. Li, Y.; Yang, J.; Aguilar, A.; McEachem, D.; Przybranowski, S.; Liu, L.; Yang, C.-Y.; Wang, M.; Han, X.; Wang, S., Discovery of MD-224 as a First-in-Class, Highly Potent, and Efficacious Proteolysis Targeting Chimera Murine Double Minute 2 Degrader Capable of Achieving Complete and Durable Tumor Regression. J. Med. Chem. 2019, 62 (2), 448-466.
36. Nagaraju, G. P.; Zakka, K. M.; Landry, J. C.; Shaib, W. L.; Lesinski, G. B.; El-Rayes, B. F., Inhibition of HSP90 overcomes resistance to chemotherapy and radiotherapy in pancreatic cancer. Int. J. Cancer 2019, 145 (6), 1529-1537.
37. Kim, H. S.; Hong, M.; Lee, S.-C.; Lee, H.-Y.; Suh, Y.-G.; Oh, D.-C.; Seo, J. H.; Choi, H.; Kim, J. Y.; Kim, K.-W.; Kim, J. H.; Kim, J.; Kim, Y.-M.; Park, S.-J.; Park, H.-J.; Lee, J., Ring-truncated deguelin derivatives as potent Hypoxia Inducible Factor-lu (HIF-lu) inhibitors. Eur. J. Med. Chem. 2015, 104, 157-164.
38. Humphrey, S. J.; Azimifar, S. B.; Mann, M., High-throughput phosphoproteomics reveals in vivo insulin signaling dynamics. Nat. Biotechnol. 2015, 33 (9), 990-995.
39. Choudhary, A.; He, K. H.; Mertins, P.; Udeshi, N. D.; Dancik, V.; Fomina-Yadlin, D.; Kubicek, S.; Clemons, P. A.; Schreiber, S. L.; Carr, S. A.; Wagner, B. K., Quantitative-proteomic comparison of alpha and beta cells to uncover novel targets for lineage reprogramming. PLoS One 2014, 9 (4), e95194/1-e95194/10, 10 pp.
40. Lim, W. A., Designing customized cell signalling circuits. Nat Rev Mol Cell Biol 2010, 11 (6), 393-403.
41. Hunter, T., The Age of Crosstalk: Phosphorylation, Ubiquitination, and Beyond. Molecular Cell 2007, 28 (5), 730-738.
42. Toure, M.; Crews, C. M., Small-Molecule PROTACS: New Approaches to Protein Degradation. Angewandte Chemie (International ed. in English) 2016, 55 (6), 1966-73.
43. Schneekloth, J. S.; Fonseca, F. N.; Koldobskiy, M.; Mandal, A.; Deshaies, R.; Sakamoto, K.; Crews, C. M., Chemical genetic control of protein levels: Selective in vivo targeted degradation. Journal of the American Chemical Society 2004, 126 (12), 3748-3754.
44. Bondeson, D. P.; Mares, A.; Smith, I. E. D.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M., Catalytic in vivo protein knockdown by small-molecule PROTACs. Nature Chemical Biology 2015, 11 (8), 611-U120.
45. Rodriguez-Gonzalez, A.; Cyrus, K.; Salcius, M.; Kim, K.; Crews, C. M.; Deshaies, R. J.; Sakamoto, K. M., Targeting steroid hormone receptors for ubiquitination and degradation in breast and prostate cancer. Oncogene 2008, 27 (57), 7201-11.
46. Winter, G. E.; Buckley, D. L.; Paulk, J.; Roberts, J. M.; Souza, A.; Dhe-Paganon, S.; Bradner, J. E., Phthalimide conjugation as a strategy for in vivo target protein degradation. Science (New York, N.Y.) 2015, 348 (6241), 1376-1381.
47. Guo, M.; Gorman, P. M.; Rico, M.; Chakrabartty, A.; Laurents, D. V., Charge substitution shows that repulsive electrostatic interactions impede the oligomerization of Alzheimer amyloid peptides. FEBS Letters 2005, 579 (17), 3574-3578.
48. Zhang, Y.-B.; Howitt, J.; McCorkle, S.; Lawrence, P.; Springer, K.; Freimuth, P., Protein aggregation during overexpression limited by peptide extensions with large net negative charge. Protein Expression and Purification 2004, 36 (2), 207-216.

### Example 6—Validation of Tyrosine Kinase Binders

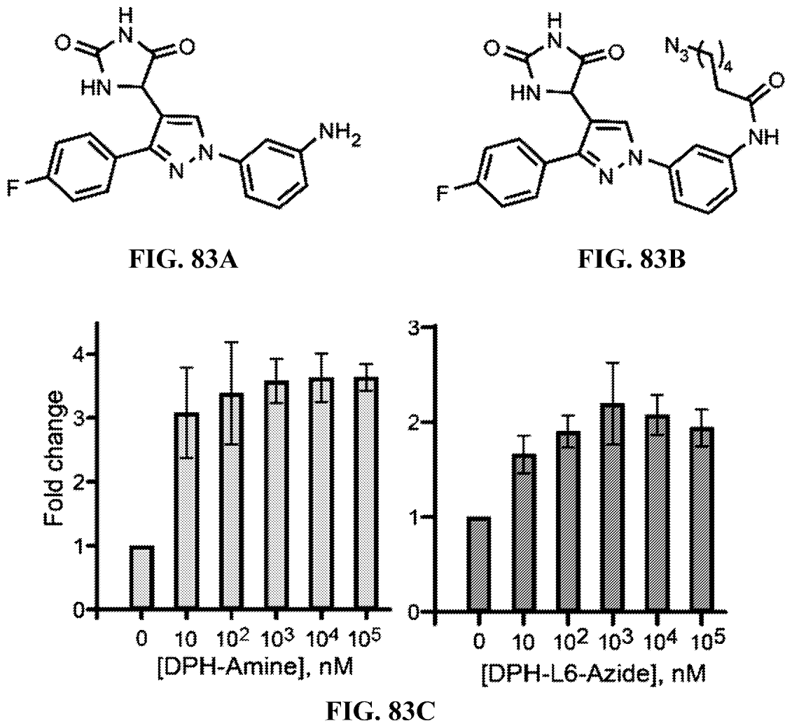

To expand the scope of PHICS beyond the Ser/Thr kinases (AMPK or PKC), Applicants will develop PHICS using the Abelson (ABL) family that are nonreceptor tyrosine kinases with SH3-SH2-TK (3-Src Homology 2-tyrosine kinase) domains (42, 43). An autoinhibitory mechanism involving intramolecular interactions stabilizes this kinase in a “locked” conformation, inhibiting kinase activity unless the SH3 domain interactions are disrupted by binding of an activator. The SH2 domain of ABL is responsible for substrate specificity: if the SH2 domain is exchanged for another protein's SH2 domain, then substrate specificity is altered (43). While a preferred proline-rich phosphorylation motif has been identified for ABL, many substrates lack that motif, suggesting that ABL kinase has flexible specificity requirements, which is beneficial for the development of the PHICS platform. Applicants have identified two ABL kinase activators (44, 45): the pyrazole activator DPH (FIG. 83A) and a pyrazoline activator that shows enhanced cellular permeability. Both DPH and DPH with linker (FIG. 83B) activated ABL in Athe DP-Glo assay (FIG. 83C).

### Methods

Synthesis of a library of PHICS. Design and synthesis of PHICS molecules for all six suggested targets will be straightforward since several proteolysis targeting chimeras (PROTACs) for these proteins already exist and choice of binder, linker type and side of its attachment were previously validated (46). Leveraging extended data, Applicants have selected six binders of target proteins (FIG. 84A), which will be connected to 3 different kinase activators (FIG. 84B) via at least 3 alkyl or PEG linkers with different lengths. All of the binders are selective reversible inhibitors of their targets, except for dasatinib and MRTX. The first one is a broad-spectrum tyrosine kinase inhibitor, which still induced selective degradation of ABL as a part of PROTAC (47). Second is a reversible covalent binder, which was successfully used to degrade KRASG12C when its irreversible analog failed to generate an efficient PROTAC (48). PHICS molecules from these binders will provide valuable information about the effect of polypharmacology and reversibility of covalent binding on neo-phosphorylation and resulting HLA-display. With regards to kinase binders, Applicants have chosen nanomolar allosteric activators of AMPK, PKC and ABL: PF-06409577 (Kd=7 nM for AMPK α1β1γ1) (21), benzolactam (Kd=4 nM for PKCα) (49) and diaryl pyrazolyl hydantoin (DPH, Kd=137 nM) (45) respectively. For all three scaffolds, linker attachment sites were chosen based on published crystal structures or SAR data and activity after functionalization was confirmed via ADP-Glo assay. To generate several PHICS molecules per protein target, Applicants will utilize a streamlined approach similar to how PROTACs are synthesized: the enzyme-recruiting amine-functionalized kinase activator will be conjugated to an alkyl or PEG linker of varying length containing a carboxylic acid or its NHS ester. The other end of the linker will be an amine or carboxylic acid which will be respectively coupled with free acids (in case of JQ1 and MI-1061, FIG. 84C) or amine (4 other binders, FIG. 84D) via a second amide bond. Only the synthesis of KRAS-targeting PHICS molecules will deviate from this general plan. Reversible-covalent PHICS require conjugation of the linker before the functionalization of the molecule with acrylamide due to the high reactivity of α,β-unsaturated amides toward nucleophiles.

Biochemical validation of PHICS. Following PHICS synthesis, Applicants will perform the ADP-Glo assay to determine target protein phosphorylation, leveraging the high-throughput nature of this assay. Next, Applicants will use AlphaScreen assay in the presence of PHICS with different linkers to identify the most optimal molecules for ternary complex formation between kinase and target protein. After this assay, inactive controls for selected PHICS will be synthesized based on the SAR (Structure-Activity Relationship) studies of the target protein binder. Further, ADP-Glo and AlphaScreen assays will be performed with active and inactive PHICS to confirm the kinase activation and ternary complex formation. The catalytic nature of these active PHICS molecules will be assessed by ADP-Glo assay. Western blotting will be performed to determine the phosphorylation of target protein in the presence of active and inactive PHICS using phospho-AMPK/phospho-PKC substrate motif or phospho-tyrosine antibodies. Also, dose and time dependency of PHICS-induced phosphorylation and sensitivity of such phosphorylations to phosphatase will be evaluated. Mass spectrometry studies will be performed in the presence of active or inactive PHICS to identify the global neo-phosphorylation sites on the target proteins.

Cellular validation of PHICS. After validating the synthesized PHICS molecules in biochemical assays, these molecules will be evaluated in various cancer cell lines based on the abundance of each target protein as well as kinase. Different target proteins and kinase isoforms show different subcellular localizations in cells and Applicants will take this into account while performing cell-based studies. Co-immunoprecipitation of the kinase and the corresponding target protein will be attempted after treating cells with the active or inactive PHICS molecules to confirm the ternary complex formation. Cells will be treated with active or inactive PHICS and phosphorylation of the target will be monitored after immunoprecipitation followed by immunoblotting with phosphor-S/T/Y antibodies. Immunoprecipitated samples will be subjected to mass spectrometry studies to identify the phosphorylation sites. Furthermore, competition experiments will be performed to validate the target engagement of PHICS in the presence of excess target protein binders. Reversibility of PHICS-induced phosphorylation will be characterized by wash-out experiments, and resistance of neo-phosphorylations towards the dephosphorylation by phosphatases will be evaluated by treatment with phosphatase inhibitors in the presence of PHICS. Finally, time and dose dependency of PHICS-induced phosphorylation will be evaluated in the same cancer cell line, and further characterization in different cancer cell lines will provide information on the cell type specificity of PHICS molecules.

Detection and analyses of HLA-displayed phosphopeptides by mass spectrometry. In collaboration with Dr. Steve Carr's laboratory, Applicants will follow the published protocol (18, 19) that identifies HLA-associated phosphopeptides to analyze the neo-phosphorylation induced by PHICS. Briefly, phosphopeptides generated from proteolysis of aberrantly phosphorylated proteins are loaded onto HLA class I molecules. HLA-associated peptide complexes are then shuttled to the cell surface and displayed to the immune system to elicit a specific cytotoxic T cell response. Here, FHIOSE cells will be stably transfected with the soluble HLA (sHLA)-A*0201 construct with a VLDLr sequence as a downstream purification tag. Subsequently, cells will be incubated with PHICS and the sHLA/peptide complexes will be extracted using an immunoaffinity column coupled with anti-VLDLr antibody. Upon phosphopeptide-enrichment using Fe(III)-IMAC columns, samples will be subjected to MS/MS.

Alternative approaches. Applicants have focused on isoform specific kinase binders. However, if some targets are not phosphorylated, Applicants will explore more pan-isoform kinase binders. For example, PKC activator indolactam (50) and pan-AMPK activator MK-872251 demonstrated a much broader scope of kinase specificity than benzolactam and PF-06409577, and PHICS designed based on these binders will have a broader range of potential targets. Selected targets are well-studied oncoproteins, and they also possess a diverse set of inhibitors with varied specificities. For instance, γ-Carboline is a pan-BET protein binder that was used by Wang et al. to create a PROTAC with picomolar potency against three isoforms of BRD protein (BRD2-4) (52, 53). Finally, different modes of target engagement will be utilized. A broad-spectrum binder, (vs the proposed specific binders), will help determine how phosphorylation of multiple targets effects HLA display.

Currently, most of the selected binders are non-covalent, except for a MRTX-based binder, which forms a reversible covalent bond with KRasG12C. Recently several reports indicated that covalent reversible PROTACs outperform their non-covalent and covalent irreversible analogs (54). Applicants will also apply the reversible covalent binding approach (for BTK or EGFR) in the case when non-covalent binders fail to provide a desired level of phosphorylation. Despite the fact that proposed types of linkers proved to be quite successful for most of the reported PROTACs, additional optimization might be required for individual targets. As an alternative to the bis-amidation strategy, Applicants will consider a click-chemistry based PHICS assembly platform (23), as well as reductive amination or alkylation of amine-containing binders with aldehyde/halide, functionalized linkers. Finally, c-MET and CDK4/6 with commercially available binders (foretinib and palbociclib, FIG. 84A) will be the alternative targets in case if any of the proposed six targets fail to work.

Future directions. During Phase I, Applicants will develop and apply PHICS to determine if they influence HLA-display. During phase II, Applicants will determine if the HLA display has functional consequences. For example, Applicants will determine if these HLA display trigger the activity of T cells in co-culture systems. Applicants will also develop some general rules for development of PHICS. For example, using global phosphoproteomics, Applicants will identify off-target phosphorylation induced by PHICS generated for various kinase activators and targets. Applicants will focus on off-target phosphorylation of not only the non-target proteins but also on phosphorylation sites within the target protein that do not match the kinase substrate motif.

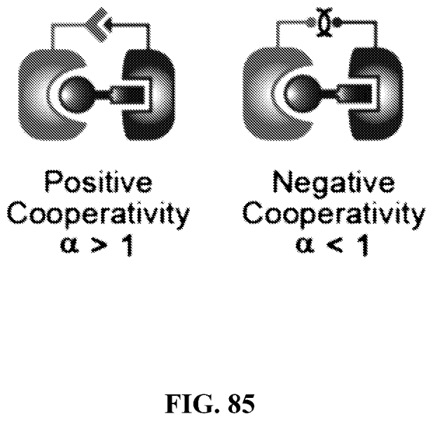

Protein-protein interactions between the kinase and target protein can enhance or attenuate the activity of PHICS molecules. Even if the binding affinities of the individual components comprising PHICS are very high, if the enforced orientation is incorrect, then productive complex will not be formed and phosphorylation will not occur effectively. The identity of the kinase/target protein binder, linker attachment sites, length and identity of the linkers, and exit vectors—how the linker exits the protein—can be altered to design PHICS that can promote cooperativity (FIG. 85). To determine the nature of cooperativity, Applicants will build a mathematical framework for 3-body equilibrium in collaboration with Prof. David Spiegel (Yale University) who has developed a mathematical model to understand ternary complex equilibria.

PHICS can be useful in several other scenarios. Several phosphorylation sites recruit ubiquitin ligase (55) and PHICS may enable targeted protein degradation (56-60). Applicants note that PHICS will complement PROTACS in multiple ways. For example, PHICS can potentially have several target sites (Ser, Thr, Tyr, and His) while PROTACs have only lysine. The efficiency of PROTAC depends on the efficiency of ubiquitination, which is a complex process compared to phosphorylation. Ubiquitination is a multistep modification involving appendage of a protein and often yields a heterogeneous mixture of poly-ubiquitinated species in substoichiometric amounts. Phosphorylation is relatively simple involving appendage of a small phosphoryl group (non-concatenate). Finally, ubiquitination involves large complexes compared to those of kinases.

Transcription factors and protein-protein interactions remain chemically intractable. Protein phosphorylation converts a neutral residue to a negatively charged residue and hyperphosphorylation may drastically affect the structure, binding interactions, or electrostatic surface. For example, hyperphosphorylation of the DNA-binding domain of the transcription factor, which is positively charged to facilitate DNA binding, will lower the transcription factor's binding affinity. Similarly, hyperphosphorylation may also inhibit protein-protein interaction. Thus, hyperphosphorylation may yield a general approach to target chemically intractable proteins.

### The Following References Relate to Example 6

1. Gerry, C. J. & Schreiber, S. L. Unifying principles of bifunctional, proximity-inducing small molecules. Nat Chem Biol 16, 369-378, doi:10.1038/s41589-020-0469-1 (2020).
2. Choudhary, A., Wu, P. & Ding, E. A. Compositions and methods for inducing protein phosphorylation. US patent (2016).
3. Fegan, A., White, B., Carlson, J. C. T. & Wagner, C. R. Chemically Controlled Protein Assembly: Techniques and Applications. Chem Rev 110, 3315-3336, doi:10.1021/cr8002888 (2010).
4. Spencer, D., Wandless, T., Schreiber, S. & Crabtree, G. Controlling signal transduction with synthetic ligands. Science 262, 1019-1024, doi:10.1126/science.7694365 (1993).
5. Tarrant, M. K. & Cole, P. A. The chemical biology of protein phosphorylation. Annu. Rev. Biochem. 78, 797-825, doi:10.1146/annurev.biochem.78.070907.103047 (2009).
6. Ferguson, F. M. & Gray, N. S. Kinase inhibitors: the road ahead. Nat. Rev. Drug Discov. 17, 353, doi:10.1038/nrd.2018.21 (2018).
7. McCormick, J. W., Pincus, D., Resnekov, O. & Reynolds, K. A. Strategies for Engineering and Rewiring Kinase Regulation. Trends Biochem Sci 45, 259-271, doi:10.1016/j.tibs.2019.11.005 (2020).
8. Ku, N. O., Azhar, S. & Omary, M. B. Keratin 8 phosphorylation by p38 kinase regulates cellular keratin filament reorganization: modulation by a keratin 1-like disease causing mutation. J Biol Chem 277, 10775-10782, doi:10.1074/jbc.M107623200 (2002).
9. McLaughlin, R. J., Spindler, M. P., van Lummel, M. & Roep, B. O. Where, How, and When: Positioning Posttranslational Modification Within Type 1 Diabetes Pathogenesis. Curr. Diab. Rep. 16, 63-63, doi:10.1007/s11892-016-0752-4 (2016).
10. Neugebauer, K. M., Merrill, J. T., Wener, M. H., Lahita, R. G. & Roth, M. B. SR proteins are autoantigens in patients with systemic lupus erythematosus. Importance of phosphoepitopes. Arthritis Rheum 43, 1768-1778, doi:10.1002/1529-0131(200008)43:8<1768::Aid-anr13>3.0.Co;2-9 (2000).
11. Thapar, R. Structural basis for regulation of RNA-binding proteins by phosphorylation. ACS Chem Biol 10, 652-666, doi:10.1021/cb500860x (2015).
12. Garcia-Garcia, T. et al. Role of Protein Phosphorylation in the Regulation of Cell Cycle and DNA-Related Processes in Bacteria. Front Microbiol 7, 184, doi:10.3389/fmicb.2016.00184 (2016).
13. Koehler, A. N. A complex task? Direct modulation of transcription factors with small molecules. Curr. Opin. Chem. Biol. 14, 331-340, doi:10.1016/j.cbpa.2010.03.022 (2010).
14. Hart, G. W., Slawson, C., Ramirez-Correa, G. & Lagerlof, O. Cross talk between 0-GlcNAcylation and phosphorylation: roles in signaling, transcription, and chronic disease. Ann Rev Biochem 80, 825-858, doi:10.1146/annurev-biochem-060608-102511 (2011).
15. Mukherjee, S. et al. Yersinia YopJ acetylates and inhibits kinase activation by blocking phosphorylation. Science 312, 1211-1214, doi:10.1126/science.1126867 (2006).
16. Hunter, T. The age of crosstalk: phosphorylation, ubiquitination, and beyond. Mol Cell 28, 730-738, doi:10.1016/j.molcel.2007.11.019 (2007).
17. Mohammed, F. et al. Phosphorylation-dependent interaction between antigenic peptides and MHC class I: a molecular basis for the presentation of transformed self. Nat Immunology 9, 1236-1243, doi:10.1038/ni.1660 (2008).
18. Abelin, J. G. et al. Complementary IMAC enrichment methods for HLA-associated phosphopeptide identification by mass spectrometry. Nat Prot 10, 1308-1318, doi:10.1038/nprot.2015.086 (2015).
19. Cobbold, M. et al. MHC class I-associated phosphopeptides are the targets of memory-like immunity in leukemia. Sci Trans Med 5, 203ra125, doi:10.1126/scitranslmed.3006061 (2013).
20. Filippakopoulos, P. et al. Selective inhibition of BET bromodomains. Nature 468, 1067-1073, doi:10.1038/nature09504 (2010).
21. Cameron, K. O. et al. Discovery and Preclinical Characterization of 6-Chloro-5-[4-(1-hydroxycyclobutyl)phenyl]-1H-indole-3-carboxylic Acid (PF-06409577), a Direct Activator of Adenosine Monophosphate-activated Protein Kinase (AMPK), for the Potential Treatment of Diabetic Nephropathy. J Med Chem 59, 8068-8081, doi:10.1021/acs.jmedchem.6b00866 (2016).
22. Zegzouti, H., Zdanovskaia, M., Hsiao, K. & Goueli, S. A. ADP-Glo: A Bioluminescent and homogeneous ADP monitoring assay for kinases. Assay Drug Dev. Technol. 7, 560-572, doi:10.1089/adt.2009.0222 (2009).
23. Wurz, R. P. et al. A “Click Chemistry Platform” for the Rapid Synthesis of Bispecific Molecules for Inducing Protein Degradation. J. Med. Chem. 61, 453-461, doi:10.1021/acs.jmedchem.6b01781 (2018).
24. Beaudet, L. et al. AlphaLISA immunoassays: the no-wash alternative to ELISAs for research and drug discovery. Nat. Meth. 5, A10, doi:10.1038/nmeth.f.230 (2008).
25. Douglass, E. F., Jr., Miller, C. J., Sparer, G., Shapiro, H. & Spiegel, D. A. A comprehensive mathematical model for three-body binding equilibria. J. Am. Chem. Soc. 135, 6092-6099, doi:10.1021/ja311795d (2013).
26. Rodbard, D., Feldman, Y., Jaffe, M. L. & Miles, L. E. Kinetics of two-site immunoradiometric (‘sandwich’) assays-II. Studies on the nature of the ‘high-dose hook effect’. Immunochemistry 15, 77-82 (1978).
27. Johanns, M. et al. AMPK antagonizes hepatic glucagon-stimulated cyclic AMP signalling via phosphorylation-induced activation of cyclic nucleotide phosphodiesterase 4B. Nat. Commun. 7, 10856, doi:10.1038/ncomms10856 (2016).
28. Wang, Z. et al. Quantitative phosphoproteomic analysis of the molecular substrates of sleep need. Nature 558, 435-439, doi:10.1038/s41586-018-0218-8 (2018).
29. Beausoleil, S. A., Villen, J., Gerber, S. A., Rush, J. & Gygi, S. P. A probability-based approach for high-throughput protein phosphorylation analysis and site localization. Nat Biotechnol 24, 1285-1292, doi:10.1038/nbtl240 (2006).
30. Kozikowski, A. P. et al. New Amide-Bearing Benzolactam-Based Protein Kinase C Modulators Induce Enhanced Secretion of the Amyloid Precursor Protein Metabolite sAPPa. J. Med. Chem. 46, 364-373, doi:10.1021/jm020350r (2003).
31. Kozikowski, A. P. et al. Modeling, Chemistry, and Biology of the Benzolactam Analogs of Indolactam V (ILV). 2. Identification of the Binding Site of the Benzolactams in the CRD2 Activator-Binding Domain of PKC6 and Discovery of an ILV Analog of Improved Isoenzyme Selectivity. J. Med. Chem. 40, 1316-1326, doi:10.1021/JM960875H (1997).
32. Mach, U. R., Lewin, N. E., Blumberg, P. M. & Kozikowski, A. P. Synthesis and pharmacological evaluation of 8- and 9-substituted benzolactam-V8 derivatives as potent ligands for protein kinase C, a therapeutic target for Alzheimer's disease. ChemMedChem 1, 307-314, doi:10.1002/cmdc.200500068 (2006).
33. Zhang, G., Kazanietz, M. G., Blumberg, P. M. & Hurley, J. H. Crystal structure of the Cys2 activator-binding domain of protein kinase Cδ in complex with phorbol ester. Cell 81, 917-924, doi:10.1016/0092-8674(95)90011-X (1995).
34. Ma, D., Tang, W., Kozikowski, A. P., Lewin, N. E. & Blumberg, P. M. General and Stereospecific Route to 9-Substituted, 8,9-Disubstituted, and 9,10-Disubstituted Analogues of Benzolactam-V8. J. Org. Chem. 64, 6366-6373, doi:10.1021/jo990605h (1999).
35. Wang, C. et al. Phosphorylation of ULKI affects autophagosome fusion and links chaperone-mediated autophagy to macroautophagy. Nat. Commun. 9, 1-15, doi:10.1038/s41467-018-05449-1 (2018).
36. Pal Singh, S., Dammeijer, F. & Hendriks, R. W. Role of Bruton's tyrosine kinase in B cells and malignancies. Mol Cancer 17, 57, doi:10.1186/s12943-018-0779-z (2018).
37. Burger, J. A. & Wiestner, A. Targeting B cell receptor signalling in cancer: preclinical and clinical advances. Nat Rev Cancer 18, 148-167, doi:10.1038/nrc.2017.121 (2018).
38. Liang, C. et al. The development of Bruton's tyrosine kinase (BTK) inhibitors from 2012 to 2017: A mini-review. Eur J Med Chem 151, 315-326, doi:10.1016/j.ejmech.2018.03.062 (2018).
39. Kang, S. W. et al. PKCbeta modulates antigen receptor signaling via regulation of Btk membrane localization. Emboj 20, 5692-5702, doi:10.1093/emboj/20.20.5692 (2001).
40. Johnson, A. R. et al. Battling Btk Mutants With Noncovalent Inhibitors That Overcome Cys481 and Thr474 Mutations. ACS Chem Biol 11, 2897-2907, doi:10.1021/acschembio.6b00480 (2016).
41. Bender, A. T. et al. Ability of Bruton's Tyrosine Kinase Inhibitors to Sequester Y551 and Prevent Phosphorylation Determines Potency for Inhibition of Fc Receptor but not B-Cell Receptor Signaling. Mol Pharmacol 91, 208-219, doi: 10.1124/mol.116.107037 (2017).
42. Colicelli, J. ABL tyrosine kinases: evolution of function, regulation, and specificity. Science signaling 3, re6, doi:10.1126/scisignal.3139re6 (2010).
43. Mayer, B. J. & Baltimore, D. Mutagenic analysis of the roles of SH2 and SH3 domains in regulation of the Abl tyrosine kinase. Mol Cell Biol 14, 2883-2894, doi:10.1128/mcb.14.5.2883 (1994).
44. Simpson, G. L. et al. Identification and Optimization of Novel Small c-Abl Kinase Activators Using Fragment and HTS Methodologies. J Med Chem 62, 2154-2171, doi:10.1021/acs.jmedchem.8b01872 (2019).
45. Yang, J. et al. Discovery and characterization of a cell-permeable, small-molecule c-Abl kinase activator that binds to the myristoyl binding site. Chem Biol 18, 177-186, doi:10.1016/j.chembiol.2010.12.013 (2011).
46. Sun, X. et al. PROTACs: great opportunities for academia and industry. Signal Transduct Target Ther 4, 64, doi:10.1038/s41392-019-0101-6 (2019).
47. Lai, A. C. et al. Modular PROTAC Design for the Degradation of Oncogenic BCR-ABL. Angew Chem Int Ed Engl 55, 807-810, doi:10.1002/anie.201507634 (2016).
48. Zeng, M. et al. Exploring Targeted Degradation Strategy for Oncogenic KRAS(G12C). Cell Chem Biol 27, 19-31.e16, doi:10.1016/j.chembiol.2019.12.006 (2020).
49. Kozikowski, A. P. et al. Modeling, chemistry, and biology of the benzolactam analogues of indolactam V (ILV). 2. Identification of the binding site of the benzolactams in the CRD2 activator-binding domain of PKCdelta and discovery of an ILV analogue of improved isozyme selectivity. J Med Chem 40, 1316-1326, doi:10.1021/jm960875h (1997).
50. Kazanietz, M. G. et al. Characterization of ligand and substrate specificity for the calcium-dependent and calcium-independent protein kinase C isozymes. Mol Pharmacol 44, 298-307 (1993).
51. Feng, D. et al. Discovery of MK-8722: A Systemic, Direct Pan-Activator of AMP-Activated Protein Kinase. ACS Med Chem Lett 9, 39-44, doi:10.1021/acsmedchemlett.7b00417 (2018).
52. Ran, X. et al. Structure-Based Design of γ-Carboline Analogues as Potent and Specific BET Bromodomain Inhibitors. J Med Chem 58, 4927-4939, doi:10.1021/acs.jmedchem.5b00613 (2015).
53. Zhou, B. et al. Discovery of a Small-Molecule Degrader of Bromodomain and Extra-Terminal (BET) Proteins with Picomolar Cellular Potencies and Capable of Achieving Tumor Regression. J Med Chem 61, 462-481, doi:10.1021/acs.jmedchem.6b01816 (2018).
54. Guo, W.-H. et al. Enhancing Intracellular Concentration and Target Engagement of PROTACs with Reversible Covalent Chemistry. bioRxiv, 2019.2012.2030.873588, doi:10.1101/2019.12.30.873588 (2019).
55. Hunter, T. The Age of Crosstalk: Phosphorylation, Ubiquitination, and Beyond. Mol Cell 28, 730-738, (2007).
56. Toure, M. & Crews, C. M. Small-Molecule PROTACS: New Approaches to Protein Degradation. Angew Chem Int Ed 55, 1966-1973, doi:10.1002/anie.201507978 (2016).
57. Schneekloth, J. S. et al. Chemical genetic control of protein levels: Selective in vivo targeted degradation. J Am Chem Soc 126, 3748-3754, doi:10.1021/ja039025z (2004).
58. Bondeson, D. P. et al. Catalytic in vivo protein knockdown by small-molecule PROTACs. Nat Chem Biol 11, 611-U120, doi:10.1038/nchembio.1858 (2015).
59. Rodriguez-Gonzalez, A. et al. Targeting steroid hormone receptors for ubiquitination and degradation in breast and prostate cancer. Oncogene 27, 7201-7211, doi:10.1038/onc.2008.320 (2008).
60. Winter, G. E. et al. Phthalimide conjugation as a strategy for in vivo target protein degradation. Science (New York, N.Y.) 348, 1376-1381, doi:10.1126/science.aab1433 (2015).

### Example 7—Localizing Moiety Kinase Inhibitors and their Residence Time

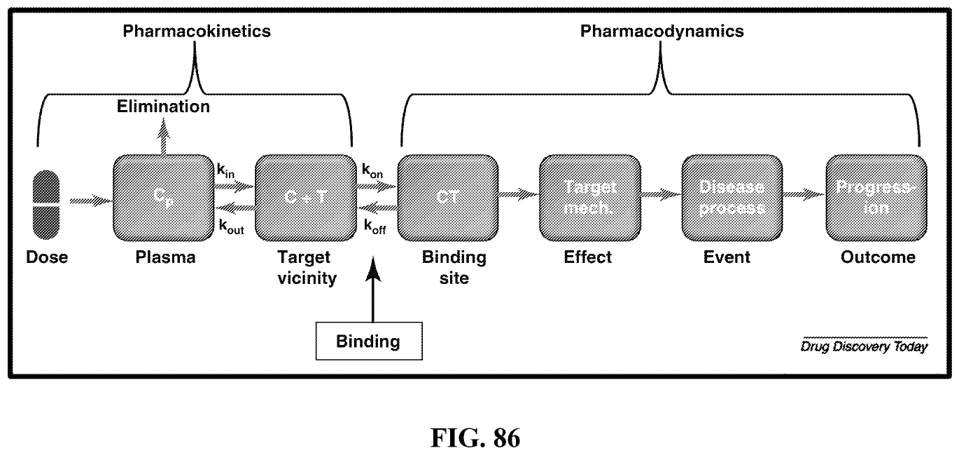

Residence time tau (t) is defined as the time a compound resides on its target:

$${K}_{d} = \frac{{k}_{off}}{{k}_{on}} \qquad {t}_{r} = \frac{1}{{k}_{off}}$$

where K_{d} is the equilibrium constant. A goal of the presently disclosed compositions is to achieve improved efficacy (extended contact, extended inhibition), longer pharmacological effects at lower doses, and reduced off-target effects. The binding of a compound to its target can be seen as the link between pharmacokinetics and pharmacodynamics (FIG. 86). Exploration of residence time, including identification of localizing moieties with a long target residence time allows discovery of moieties inhibitory at time points after the free compound has been eliminated from the cell. Exploration and discussion of a variety of compounds and their residence time, which can be utilized in design strategy for the multifunctional compounds of the present invention follows.

Willemsen-Seegers et al., explored residence time, association and dissociation constants of kinase inhibitor interactions and equilibrium affinity constants to compare inhibitor potency values (IC₅₀) in enzyme assays and target residence time as predictors of biological activity of kinase inhibitors. Kinetics of binding interactions between different kinases and their inhibitors as determined by surface plasma resonance (SPR) are shown in Table 4 (Willemsen-Seegers N, et al., J Mol Biol. 429(4):574-586 (2017), Table 1; doi:10.1016/j.jmb.2016.12.019).

TABLE 4
Equilibrium dissociation constant (K_{D}) and residence time (τ) of kinase-inhibitor interactions determined by SPR.

| Kinase | Inhibitor | kₐ(1/Ms) | log(kₐ) | SD log(kₐ) | k_{d} (1/s) | log(k_{d}) | SD log(k_{d}) | K_{D} (M) | r(s) | t_{1/2} (s) | No. exp.^{c} |
|---|---|---|---|---|---|---|---|---|---|---|---|
| ABL | Bosutinib | 7.21E+05 | 5.86 | 0.64 | 1.57E−03 | −2.80 | 0.25 | 2.17E−09 | 638 | 442 | 5 |
| AKT1 | GSK690693 | 3.84E+04 | 4.58 | | 2.95E−02 | −1.53 | | 7.67E−07 | 34 | 23 | 2 |
| AKT2 | GSK690693 | 3.59E+04 | 4.55 | | 2.93E−02 | −1.53 | | 8.15E−07 | 34 | 24 | 2 |
| ALK | Crizotinib | 2.29E+05 | 5.36 | | 1.14E−02 | −1.94 | | 5.00E−08 | 87 | 61 | 1 |
| ALK | Crizotinib | 3.18E+05 | 5.50 | | 7.91E−02 | −1.10 | | 2.49E−07 | 13 | 9 | 1 |
| [L1196M] | | | | | | | | | | | |
| AurA | MK5108 | 3.41E+06 | 6.53 | 0.24 | 1.55E−02 | −1.81 | 0.12 | 4.54E−09 | 65 | 45 | 3 |
| (AURKA) | | | | | | | | | | | |
| AurB | GSK1070916 | 1.40E+04 | 4.15 | 0.46 | 6.28E−05 | −4.20 | 0.31 | 4.48E−09 | 15,926 | 11,037 | 3 |
| (AURKB)^{a} | | | | | | | | | | | |
| AXL | Crizotinib | 4.82E+04 | 4.68 | | 9.68E−02 | −1.01 | | 2.01E−06 | 10 | 7 | 1 |
| BMX | Dasatinib | 6.65E+05 | 5.82 | | 2.17E−04 | −3.66 | | 3.26E−10 | 4613 | 3197 | 2 |
| BRAF | Dabrafenib | 4.67E+05 | 5.67 | | 2.88E−04 | −3.54 | | 6.16E−10 | 3471 | 2406 | 2 |
| BTK^{a} | Ponatinib | 2.15E+04 | 4.33 | 0.18 | 1.68E−02 | −1.77 | 0.17 | 7.84E−07 | 59 | 41 | 15 |
| CHK1 | Sunitinib | 9.16E+04 | 4.96 | | 1.98E−01 | −0.70 | | 2.16E−06 | 5 | 4 | 1 |
| (CHEK1) | | | | | | | | | | | |
| DDR2^{a} | Dasatinib | 3.93E+04 | 4.59 | | 1.00E−04 | −4.00 | | 2.55E−09 | 9990 | 6923 | 1 |
| EGFR | Gefitinib | 5.46E+06 | 6.74 | 0.79 | 7.63E−03 | −2.12 | 0.28 | 1.40E−09 | 131 | 91 | 12 |
| FAK | Crizotinib | 8.50E+06 | 6.93 | | 1.16E−01 | −0.94 | | 1.37E−08 | 9 | 6 | 1 |
| (PTK2) | | | | | | | | | | | |
| FGFR1^{a} | BGJ398 | 1.65E+05 | 5.22 | | 7.67E−05 | −4.12 | | 4.65E−10 | 13,041 | 9037 | 2 |
| FGFR3^{a} | BGJ398 | 1.48E+05 | 5.17 | | 2.58E−04 | −3.59 | | 1.75E−09 | 3873 | 2684 | 2 |
| FGFR4 | BGJ398 | 6.26E+05 | 5.80 | 0.33 | 1.21E−02 | −1.92 | 0.24 | 1.94E−08 | 82 | 57 | 4 |
| FLT3 | Dasatinib | 2.40E+03 | 3.38 | 0.07 | 5.93E−03 | −2.23 | 0.07 | 2.47E−06 | 168 | 117 | 4 |
| FMS | Dasatinib | 2.85E+05 | 5.46 | 0.12 | 1.10E−03 | −2.96 | 0.06 | 3.86E−09 | 908 | 629 | 8 |
| (CSF1R) | | | | | | | | | | | |
| FYN | Ponatinib | 8.49E+04 | 4.93 | 0.53 | 8.93E−05 | −4.05 | 0.56 | 1.05E−09 | 11,197 | 7760 | 5 |
| [isoform a] | | | | | | | | | | | |
| IGF1R^{a} | Staurosporine | 5.19E+05 | 5.71 | 0.04 | 3.97E−02 | −1.40 | 0.11 | 7.65E−08 | 25 | 17 | 3 |
| IRAK4 | Sunitinib | 7.17E+05 | 5.86 | 0.28 | 1.05E−01 | −0.98 | 0.26 | 1.46E−07 | 10 | 7 | 4 |
| ITK | Sunitinib | 3.98E+03 | 3.60 | | 2.77E−02 | −1.56 | | 6.96E−06 | 36 | 25 | 1 |
| KDR | Vandetanib | 2.56E+05 | 5.41 | 0.38 | 2.73E−02 | −1.56 | 0.26 | 1.07E−07 | 37 | 25 | 5 |
| (VEGFR2) | | | | | | | | | | | |
| KIT^{a} | Pazopanib | 3.69E+03 | 3.57 | 0.04 | 2.00E−04 | −3.70 | 0.02 | 5.42E−08 | 4995 | 3461 | 5 |
| LCK | Ponatinib | 1.27E+05 | 5.10 | 0.61 | 2.24E−04 | −3.65 | 0.23 | 1.76E−09 | 4466 | 3095 | 4 |
| LYN | Ponatinib | 1.10E+05 | 5.04 | 0.22 | 5.40E−05 | −4.27 | 0.27 | 4.88E−10 | 18,534 | 12,844 | 4 |
| [isoform a] | | | | | | | | | | | |
| MAP3K5 | Staurosporine | 4.45E+05 | 5.65 | 0.02 | 3.12E−03 | −2.51 | 0.03 | 7.00E−09 | 321 | 222 | 4 |
| MET | Crizotinib | 7.42E+04 | 4.87 | | 1.76E−03 | −2.76 | | 2.37E−08 | 569 | 394 | 2 |
| p38α | Sorafenib | 1.92E+04 | 4.28 | | 1.14E−02 | −1.94 | | 5.92E−07 | 88 | 61 | 2 |
| (MAPK14)^{b} | | | | | | | | | | | |
| p38α | Sorafenib | 1.75E+04 | 4.24 | | 6.69E−03 | −2.17 | | 3.81E−07 | 149 | 104 | 2 |
| (MAPK14) | | | | | | | | | | | |
| PAK4 | Staurosporine | 4.27E+07 | 7.63 | | 6.73E−03 | −2.17 | | 1.58E−10 | 149 | 103 | 2 |
| PIK3Cα/ | Apitolisib | 4.78E+06 | 6.68 | 0.10 | 5.41E−03 | −2.27 | 0.08 | 1.13E−09 | 185 | 128 | 5 |
| PIK3R1 | | | | | | | | | | | |
| PIK3Cδ/ | Apitolisib | 6.83E+ 06 | 6.83 | | 2.11E−02 | −1.68 | | 3.08E−09 | 47 | 33 | 2 |
| PIK3R1 | | | | | | | | | | | |
| PIK3Cγ | Apitolisib | 1.62E+06 | 6.21 | 0.18 | 2.02E−02 | −1.69 | 0.17 | 1.24E−08 | 49 | 34 | 6 |
| PKN1 | GSK690693 | 1.66E+05 | 5.22 | | 8.48E−03 | −2.07 | | 5.11E−08 | 118 | 82 | 2 |
| PLK1 | BI 2536 | 4.68E+04 | 4.67 | | 4.99E−04 | −3.30 | | 1.07E−08 | 2004 | 1389 | 1 |
| PYK2 | Crizotinib | 1.68E+05 | 5.23 | 0.79 | 1.71E−02 | −1.77 | 0.34 | 1.01E−07 | 59 | 41 | 3 |
| (PTK2B)^{a} | | | | | | | | | | | |
| RET | Sorafenib | 2.55E+04 | 4.41 | 0.07 | 1.08E−04 | −3.97 | 0.28 | 4.23E−09 | 9277 | 6429 | 6 |
| SRC | Ponatinib | 2.60E+04 | 4.41 | 0.51 | 1.57E−04 | −3.80 | 0.41 | 6.05E−09 | 6365 | 4411 | 5 |
| TIE2^{a} | Ponatinib | 4.02E+04 | 4.60 | 0.39 | 2.68E−03 | −2.57 | 0.39 | 6.66E−08 | 373 | 259 | 9 |
| TNIK | Bosutinib | 1.25E+06 | 6.10 | | 1.78E−02 | −1.75 | | 1.43E−08 | 56 | 39 | 2 |
| TRKA | AZ23 | 3.28E+05 | 5.52 | 0.12 | 1.74E−04 | −3.76 | 0.15 | 5.29E−10 | 5757 | 3990 | 9 |
| (NTRK1)^{a} | | | | | | | | | | | |
| TRKB | AZ23 | 4.21E+05 | 5.62 | 0.15 | 2.31E−04 | −3.64 | 0.20 | 5.48E−10 | 4334 | 3004 | 4 |
| (NTRK2)^{a} | | | | | | | | | | | |
| TRKC | AZ23 | 2.82E+05 | 5.45 | | 1.49E−04 | −3.83 | | 5.30E−10 | 6692 | 4637 | 2 |
| (NTRK3)^{a} | | | | | | | | | | | |

Table 5 (Willemsen-Seegers N, et al. J Mol Biol. 429(4):574-586 (2017), Table 2; doi:10.1016/j.jmb.2016.12.019) shows kinetic parameters and potency in an enzyme assay of three different reversible EGFR inhibitors: gefitinib, erlotinib, and lapatinib. The assay conducted and results are as disclosed in Willemsen-Seegers, et al. (2017), incorporated herein by reference.

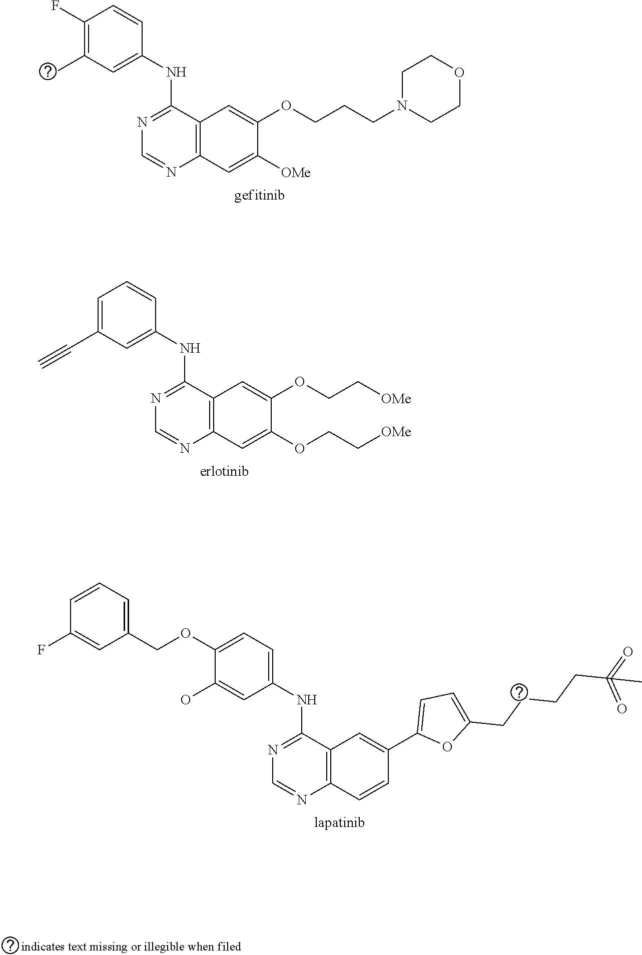

TABLE 5
Kinetic parameters and potency in enzyme assay of reversible EGFR inhibitors.

| Inhibitor | Kₐ (1/Ms) | log(kₐ) | SD log(kₐ) | k_{d} (1/s) | log(k_{d}) | SD log(k_{d}) | K_{D} (M) | r(s) | t_{1/2} (s) | No. exp | IC₅₀ (M) |
|---|---|---|---|---|---|---|---|---|---|---|---|
| Erlotinib | 4.74E+06 | 6.68 | 0.49 | 3.31E−03 | −2.48 | 0.23 | 6.99E−10 | 302 | 209 | 7 | 7.5E−10 |
| Gefitinib | 5.46E+06 | 6.74 | 0.79 | 7.63E−03 | −2.12 | 0.28 | 1.40E−09 | 131 | 91 | 12 | 5.1E−10 |
| Lapatinib | 1.11E+05 | 5.05 | | 4.70E−05 | −4.33 | | 4.22E−10 | 21.273 | 14,742 | 2 | 4.9E−09 |

Apparent kinetic parameters of irreversible tyrosine kinase inhibitors obtained with an induced fit binding model as examined in Willemsen-Seegers, et al. (2017) at Table S3 are shown in Table 6. Parameters are apparent (modeled) values and therefore indicated with an asterisk.

TABLE 6

| Enzyme | Inhibitor | kₐ₁*(1/Ms) | k_{d1}*(1/s) | kₐ₂*(1/s) | k_{d2}*(1/s) | k_{D}*(M) | τ*(s)^{a} |
|---|---|---|---|---|---|---|---|
| EGFR | afatinib | 2.78E+06 | 5.89E−03 | 2.24E−03 | 4.57E−05 | 4.20E−11 | 30389 |
| EGFR | ibrutinib | 4.30E+05 | 1.83E−02 | 7.20E−03 | 9.97E−05 | 5.82E−10 | 14031 |
| EGFR | neratinib | 1.74E+08 | 6.59E−02 | 5.46E−03 | 5.54E−05 | 5.81E−12 | 12692 |
| EGFR | pelitinib | 3.23E+06 | 8.25E−03 | 1.94E−03 | 1.87E−04 | 2.22E−10 | 6732 |
| FGFR4 | BLU9931 | 1.91E+04 | 1.23E−02 | 1.03E−02 | 8.22E−05 | 5.11E−06 | 22418 |
| BTK | ibrutinib | 3.42E+05 | 2.35E−03 | 2.26E−03 | 3.11E−04 | 8.29E−10 | 6746 |

Table 7 shows kinetic parameters of the binding of ponatinib to ten different kinases and IC₅₀ in enzyme assays (Willemsen-Seegers, et al., at Table S4).

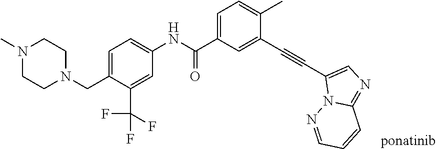 [SMILES: Cc1ccc(C(=O)Nc2ccc(CN3CCN(C)CC3)c(C(F)(F)F)c2)cc1C#Cc1cnc2cccnn12]

TABLE 7

| Kinase | kₐ(1/Ms) | log(kₐ) | SD log(kₐ) | k_{d}(1/s) | log (k_{d)} | SD log(k_{d)} | K_{D}(M) | _{T}(s) | t_{1/2} | nr. exp | IC₅₀ (M) |
|---|---|---|---|---|---|---|---|---|---|---|---|
| ABL | 4.35E+04 | 4.64 | 0.20 | 1.54E−06 | −5.81 | 1.43 | 3.55E−11 | 647628 | 448806 | 4 | 3.30E−09 |
| BTK^{a} | 2.15E+04 | 4.33 | 0.18 | 1.68E−02 | −1.77 | 0.17 | 7.84E−07 | 59 | 41 | 15 | 1.57E−07 |
| DDR2 | 2.98E+04 | 4.47 | 0.07 | 1.40E−04 | −3.85 | 0.31 | 4.68E−09 | 7160 | 4962 | 4 | 3.43E−09 |
| FGFR1 | 1.79E+04 | 4.25 | 0.08 | 1.19E−04 | −3.93 | 0.16 | 6.66E−09 | 8417 | 5833 | 4 | 3.88E−09 |
| FYN | 8.49E+04 | 4.93 | 0.53 | 8.93E−05 | −4.05 | 0.56 | 1.05E−09 | 11197 | 7760 | 5 | 2.39E−09 |
| [isoform a] | | | | | | | | | | | |
| LCK | 1.27E+05 | 5.10 | 0.61 | 2.24E−04 | −3.65 | 0.23 | 1.76E−09 | 4466 | 3095 | 4 | 4.76E−10 |
| LYN | 1.10E+05 | 5.04 | 0.22 | 5.40E−05 | −4.27 | 0.27 | 4.88E−10 | 18534 | 12844 | 4 | 7.57E−10 |
| [isoform a] | | | | | | | | | | | |
| RET | 1.18E+05 | 5.07 | 0.28 | 4.29E−05 | −4.37 | 0.30 | 3.64E−10 | 23294 | 16143 | 4 | 9.96E−10 |
| SRC | 2.60E+04 | 4.41 | 0.51 | 1.57E−04 | −3.80 | 0.41 | 6.05E−09 | 6365 | 4411 | 5 | 5.85E−09 |
| TIE2 | 4.02E+04 | 4.60 | 0.39 | 2.68E−03 | −2.57 | 0.39 | 6.66E−08 | 373 | 259 | 9 | 3.78E−09 |

Aurora kinases are serine/threonine kinases that are essential for cell proliferation. They are phosphotransferase enzymes that help the dividing cell dispense its genetic materials to its daughter cells. Aurora A functions during prophase of mitosis and is required for correct duplication and separation of the centrosomes. Aurora B functions in the attachment of the mitotic spindle to the centromere. Aurora A and B are ubiquitously expressed. Tables 8 and 9 list kinetic parameters of Aurora kinase inhibitors of Aurora A and B, respectively (from Willemsen-Seegers, et al. (2017, Tables 3A and 3B).

TABLE 8

| Inhibitor | kₐ (1/Ms) | log(kₐ) | SD log(kₐ) | K_{d} (1/s) | log(k_{d}) | SD log (k_{d}) | K_{D} (M) | r(s) | t_{1/2} (s) | No. exp |
|---|---|---|---|---|---|---|---|---|---|---|
| AMG900 | 2.20E+05 | 5.34 | | 2.61E−04 | −3.58 | | 1.19E−09 | 3824 | 2650 | 2 |
| Danusertib | 5.12E+05 | 5.71 | 0.11 | 8.68E−04 | −3.06 | 0.18 | 1.69E−09 | 1153 | 799 | 5 |
| GSK 1070916 | 1.80E+05 | 5.26 | 0.49 | 2.37E−03 | −2.62 | 0.18 | 1.32E−08 | 422 | 292 | 5 |
| MK5108 | 3.41E+06 | 6.56 | 0.24 | 1.55E−02 | −1.81 | 0.12 | 4.54E−09 | 65 | 45 | 3 |
| MLN8054 | 3.06E+06 | 6.49 | | 1.32E−02 | −1.88 | | 4.33E−09 | 76 | 52 | 2 |
| Tozasertib | 5.90E+05 | 5.77 | 0.03 | 1.77E−03 | −2.75 | 0.35 | 3.00E−09 | 566 | 392 | 3 |

TABLE 9

| Inhibitor | kₐ (1/Ms) | log(kₐ) | SD log(kₐ) | K_{d} (1/s) | log(k_{d}) | SD log (k_{d}) | K_{D} (M) | r(s) | t_{1/2} (s) | No. exp |
|---|---|---|---|---|---|---|---|---|---|---|
| AMG900 | 5.88E+04 | 4.77 | | 2.31E−05 | −4.64 | | 3.93E−10 | 43,300 | 30,007 | 2 |
| Danusertib | 4.34E+04 | 4.64 | 0.14 | 6.26E−05 | −4.20 | 0.25 | 1.44E−09 | 15,976 | 11,071 | 3 |
| GSK1070916 | 1.40E+04 | 4.15 | 0.46 | 6.28E−05 | −4.20 | 0.31 | 4.48E−09 | 15,926 | 11,037 | 3 |
| MK5108 | 9.38E+05 | 5.97 | 0.11 | 8.93E−03 | −2.05 | 0.12 | 9.52E−09 | 112 | 76 | 3 |
| MLN8054 | 6.53E+04 | 4.81 | | 1.72E−04 | −3.76 | | 2.63E−09 | 5820 | 4033 | 2 |
| Tozasertib | 3.15E+04 | 4.50 | 0.11 | 8.72E−05 | −4.06 | 0.29 | 2.77E−09 | 11,473 | 7951 | 5 |

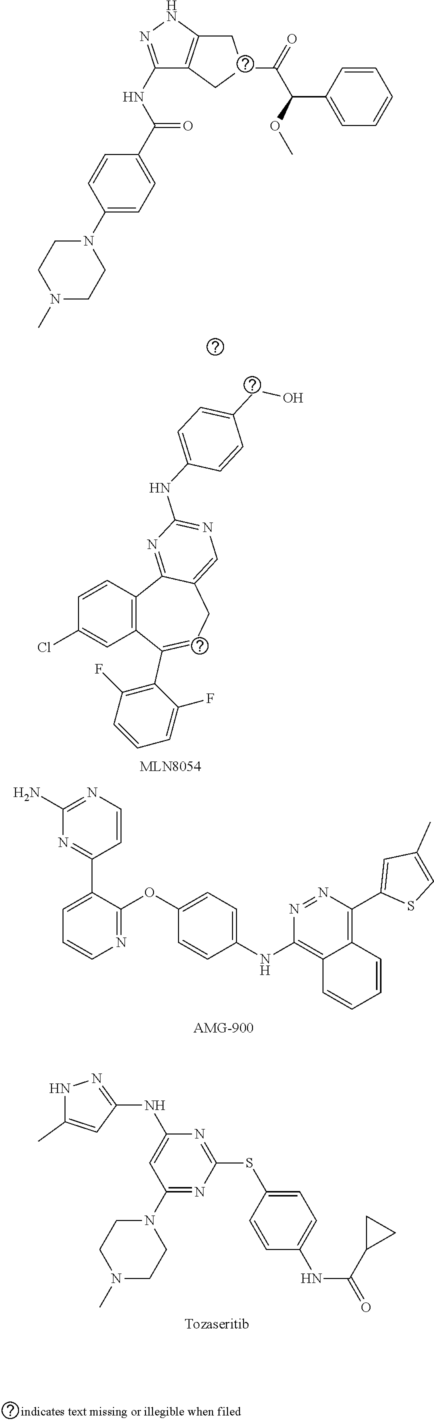 [SMILES: CO[C@@H](C(=O)C1Cc2[nH]nc(NC(=O)c3ccc(N4CCN(C)CC4)cc3)c2C1)c1ccccc1 ; OCc1ccc(Nc2ncc3c(n2)-c2ccc(Cl)cc2C(c2c(F)cccc2F)=CC3)cc1 ; Cc1csc(-c2nnc(Nc3ccc(Oc4ncccc4-c4ccnc(N)n4)cc3)c3ccccc23)c1 ; Cc1cc(Nc2cc(N3CCN(C)CC3)nc(Sc3ccc(NC(=O)C4CC4)cc3)n2)n[nH]1]

PI3K kinases are a family of enzymes involved in cellular functions such as cell growth, proliferation, differentiation, motility, survival and intracellular trafficking, which in turn are involved in cancer. In particular, α and β isoforms are expressed in many cell types and have been mainly targeted for oncology. The PI3K p110γ and p110δ catalytic subunits are expressed in hematopoietic cells and play a role in adaptive and innate immunity. Tables 10-12 list the kinetic parameters of PI3K inhibitors on PIK3Cα/PIK3R1, PIK3Cγ, and PIK3Cδ/PIK3R1.

TABLE 10

| Inhibitor | kₐ (1/Ms) | log (kₐ) | SD log (kₐ) | k_{d} (1/s) | log(k_{d}) | SD log (k_{d}) | K_{D} (M) | r(s) | t_{1/2} (s) | nr exp |
|---|---|---|---|---|---|---|---|---|---|---|
| Alpelisib | 5.28E+06 | 6.72 | | 7.41E−03 | −2.13 | | 1.40E−09 | 135 | 94 | 2 |
| Apitolisib | 4.78E+06 | 6.68 | 0.10 | 5.41E−03 | −2.27 | 0.08 | 1.13E−09 | 185 | 128 | 5 |
| AZD-8055 | 3.61E+06 | 6.56 | | 3.39E−02 | −1.47 | | 9.40E−09 | 29 | 20 | 2 |
| Buparlisib | 3.26E+06 | 6.51 | | 2.09E−02 | −1.68 | | 6.41E−09 | 48 | 33 | 2 |
| Dactolisib | 2.30E+06 | 6.36 | 0.30 | 2.28E−02 | −1.64 | 0.20 | 9.91E−09 | 44 | 30 | 4 |
| Duvelisib | 1.66E+06 | 6.22 | | 5.30E−02 | −1.28 | | 3.16E−08 | 19 | 13 | 2 |
| Idelalisib | 1.13E+06 | 6.05 | 0.36 | 1.37E−01 | −0.86 | 0.12 | 1.22E−07 | 7 | 5 | 3 |
| Pictilisib | 4.09E+06 | 6.61 | 0.13 | 4.49E−03 | −2.35 | 0.08 | 1.10E−09 | 223 | 154 | 4 |

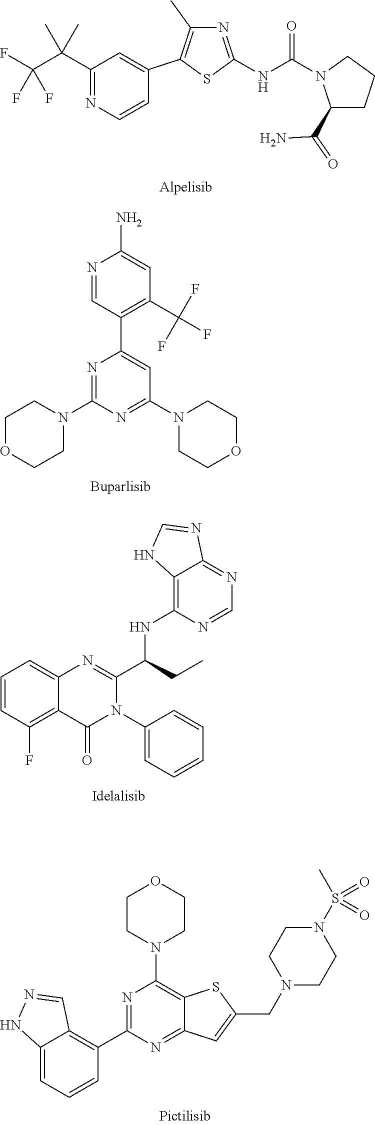 [SMILES: Cc1nc(NC(=O)N2CCC[C@H]2C(N)=O)sc1-c1ccnc(C(C)(C)C(F)(F)F)c1 ; Nc1cc(C(F)(F)F)c(-c2cc(N3CCOCC3)nc(N3CCOCC3)n2)cn1 ; CC[C@H](Nc1ncnc2nc[nH]c12)c1nc2cccc(F)c2c(=O)n1-c1ccccc1 ; CS(=O)(=O)N1CCN(Cc2cc3nc(-c4cccc5[nH]ncc45)nc(N4CCOCC4)c3s2)CC1]

TABLE 11

| Inhibitor | kₐ (1/Ms) | log (kₐ) | SD log (kₐ) | kₐ (1/s) | log(k_{d}) | SD log (k_{d}) | K_{D} (M) | r(s) | t_{1/2} (s) | nr exp |
|---|---|---|---|---|---|---|---|---|---|---|
| Alpelisib | 4.05E+06 | 6.61 | 0.35 | 3.95E−02 | −1.40 | 0.49 | 9.76E−09 | 25 | 16 | 4 |
| Apitolisib | 1.62E+06 | 6.21 | 0.18 | 2.02E−02 | −1.69 | 0.17 | 1.24E−06 | 49 | 34 | 6 |
| AZD-8055 | 1.59E+06 | 6.20 | | 1.47E−01 | −0.83 | | 9.25E−08 | 7 | 5 | 2 |
| Buparlisib | 1.49E+06 | 6.17 | | 1.49E−01 | −0.83 | | 9.99E−08 | 7 | 5 | 2 |
| Dactolisib | 5.89E+05 | 5.77 | 0.16 | 4.33E−02 | −1.36 | 0.12 | 7.35E−06 | 23 | 16 | 6 |
| Duvelisib | 1.63E+06 | 6.21 | 0.07 | 7.09E−04 | −3.15 | 0.16 | 4.35E−10 | 1411 | 978 | 5 |
| Idelalisib | 1.03E+06 | 6.01 | 0.09 | 1.71E−02 | −1.77 | 0.02 | 1.66E−08 | 59 | 41 | 4 |
| Pictilisib | 2.22E+06 | 6.35 | 0.32 | 5.39E−02 | −1.27 | 0.38 | 2.43E−06 | 19 | 13 | 4 |

TABLE 12

| Inhibitor | kₐ (1/Ms) | log (kₐ) | SD log (kₐ) | k_{d} (1/s) | log(k_{d}) | SD log (k_{d}) | K_{D} (M) | r(s) | t_{1/2} (s) | nr exp |
|---|---|---|---|---|---|---|---|---|---|---|
| Alpelisib | 6.16E+06 | 6.79 | | 6.41E−02 | −1.19 | | 1.04E−08 | 16 | 11 | 2 |
| Apitolisib | 6.83E+06 | 6.83 | | 2.11E−02 | −1.68 | | 3.08E−09 | 47 | 33 | 2 |
| AZD-8055 | 1.93E+06 | 6.29 | | 6.50E−02 | −1.19 | | 3.36E−08 | 15 | 11 | 2 |
| Buparlisib | 4.30E+06 | 6.63 | | 6.90E−02 | −1.16 | | 1.61E−06 | 14 | 10 | 2 |
| Dactolisib | 1.52E+06 | 6.16 | | 3.27E−02 | −1.49 | | 2.15E−08 | 31 | 21 | 2 |
| Duvelisib | 5.31E+06 | 6.72 | 0.03 | 1.21E−04 | −3.92 | 0.59 | 2.29E−11 | 8246 | 5715 | 3 |
| Idelalisib | 5.93E+06 | 6.77 | 0.19 | 4.32E−03 | −2.36 | 0.72 | 7.29E−10 | 231 | 160 | 7 |
| Pictilisib | 4.02E+06 | 6.60 | | 9.32E−03 | −2.03 | | 2.32E−09 | 107 | 74 | 2 |

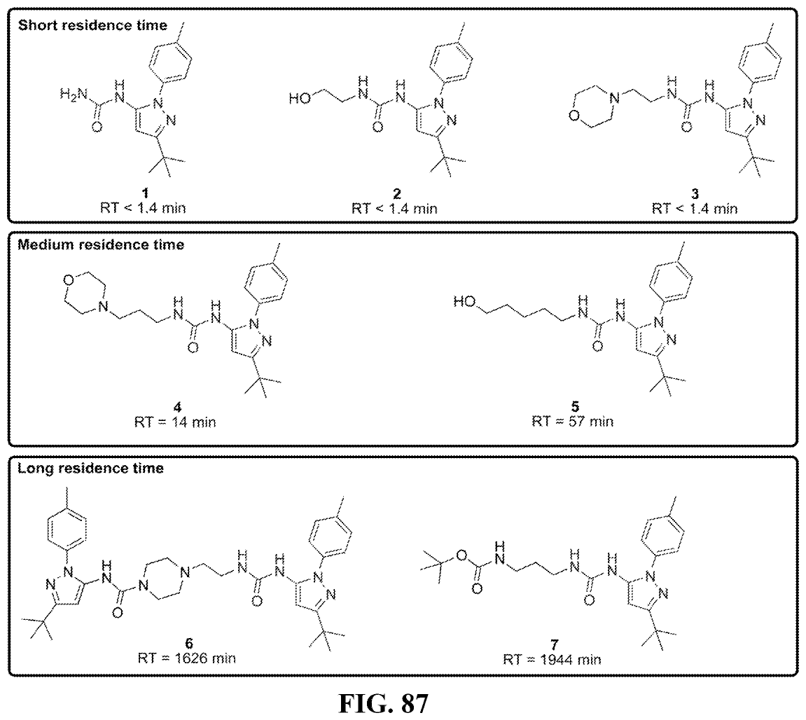

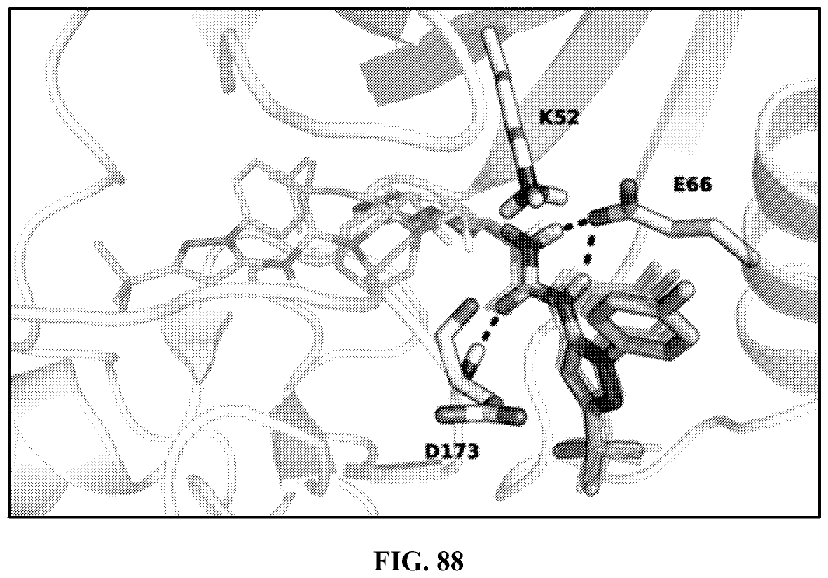

Cyclin-dependent protein kinase 8 (CDK8) regulates transcription by several mechanisms, such as by binding and/or phosphorylating several transcription factors, which can have an activating or inhibitory effect on transcription factor function. Inhibition of CDK8 suppresses cell growth and may have anticancer activity by causing selective and disproportionate upregulation of super-enhancer-associated genes including the cell identity genes CEBPA and IRF8. FIG. 87 shows the chemical structures of several CDK8 inhibitors with residence times of various lengths. FIG. 88 shows the active site of the crystal structure of human CDK8 in complex with compounds 1-7 in FIG. 87 (Callegari et al. J Chem Inf Model 57(2):386 (2017); Schneider et al. PNAS 110(20):8081-8086 (2013)).

p38α mitogen-activated protein kinases play a key role in regulating the proinflammatory cytokines biosynthesis and are therapeutic targets for the treatment of autoimmune and inflammatory diseases. Chemical structures of inhibitors of these kinases are shown in FIG. 89 and comparison of their experimental affinities, kinetics, and protein conformation states are listed in Table 13 Braka et al., “Residence Time Prediction of Type 1 and 2 Kinase Inhibitors from Unbinding Simulations, J. Chem. Inf Model. 2020, 60, 1, 342-348, doi:10.1021/acs.jcim,9b00497). B96, BMU, SB6, are structurally solved in complex with p38a and available under PDB codes 1KV2, 1KV1, and 1A9U, respectively. Protocolato predict relative ligand kinetic rates and residence times of the compounds as disclosed in Braka et al. can be used to identify candidate drugs for us in the multifunctional molecules of the present invention.

TABLE 13
Comparison of experimental affinities, kinetics, and
protein conformation states of the studied inhibitors.

| | | | | DFG | experimental |
|---|---|---|---|---|---|
| ligand [symbol] | K_{D} (M) | k [symbol] (s⁻¹) | RT exp. | orientation | method [symbol] |
| B96 | 6.1 × 10 [symbol] | 5.2 × 10 [symbol] | 5.3 h | out | SPR [symbol] |
| BMU | 1.1 × 10 [symbol] | 2.8 × 10 [symbol] | 35.7 s | out | SPR [symbol] |
| SBS | 4.0 × 10 [symbol] | 2.7 × 10 [symbol] | 37 s | in | SPR [symbol] |
| SB6 | 7.8 × 10 [symbol] | 1.3 × 10 [symbol] | 7.7 s | in | SPR [symbol] |
| SB7 | 5.3 × 10 [symbol] | 6.7 × 10 [symbol] | 14.9 s | in | SPR [symbol] |
| BR5 | 9.7 × 10 [symbol] | 1.5 × 10 [symbol] | 18.5 h | out | SPR [symbol] |
| BR8 | 2.3 × 10 [symbol] | 3.3 × 10 [symbol] | 5 min | out | SPR [symbol] |
| B12 | 1.6 × 10 [symbol] | 2.6 × 10 [symbol] | 10.6 h | out | SPR [symbol] |

[symbol]

indicates data missing or illegible when filed

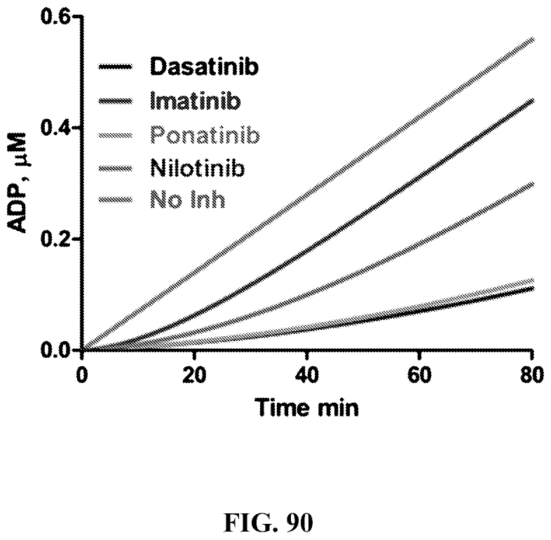

Abl1 is a tyrosine-protein kinase that is implicated in processes of cell differentiation, cell division, cell adhesion, and stress response. Chemical structures of inhibitors of this kinase are shown below and kinetics of inhibition are shown in FIG. 90 and Table 14 (Sigma Aldrich “Measuring Kinase Inhibitor Residence Times,” Application Note, available at sigmaaldrich.com/technical-documents). In the Application Note on the Sigma Aldrich page, a protocol for measuring kinase inhibitor residence times is also provided, allowing determination of residence time of a molecule during interaction with a kinase, allowing for calculations of candidate molecules with desired activity profiles.

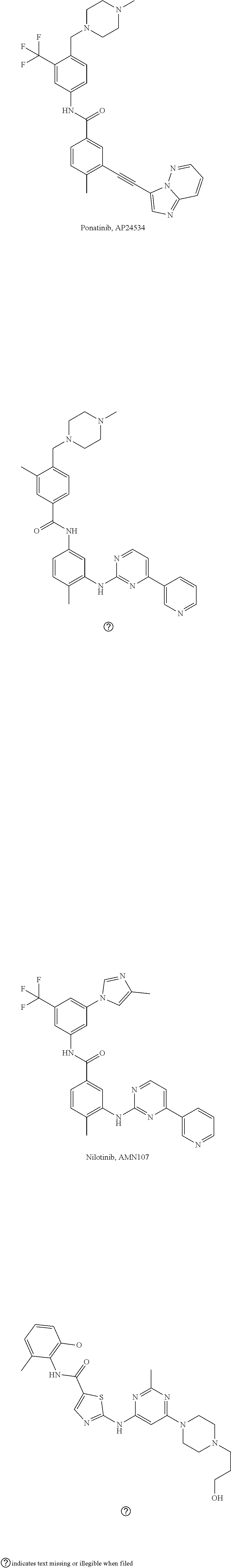 [SMILES: Cc1ccc(C(=O)Nc2ccc(CN3CCN(C)CC3)c(C(F)(F)F)c2)cc1C#Cc1cnc2cccnn12 ; Cc1cc(C(=O)Nc2ccc(C)c(Nc3nccc(-c4cccnc4)n3)c2)ccc1CN1CCN(C)CC1 ; Cc1cn(-c2cc(NC(=O)c3ccc(C)c(Nc4nccc(-c5cccnc5)n4)c3)cc(C(F)(F)F)c2)cn1 ; Cc1nc(Nc2ncc(C(=O)Nc3c(C)cccc3O)s2)cc(N2CCN(CCCO)CC2)n1]

TABLE 14

| | Koff, min − 1 | Tau, min |
|---|---|---|
| Dasatinib | 0.004011 | 249 |
| Imatinib | 0.05884 | 17 |
| Ponatinib | 0.004882 | 205 |
| Nilotinib | 0.0201 | 50 |

Table 15 (adapted from Roskoski R J. Pharmacol Res 103:26-48 (2016), at Table 5) shows the drug-target residence times of various selected drugs with their protein kinase target. The lapatinib binding pocket is much larger than that for the other drugs with EGFR as a protein kinase target, and gefitinib and erlotinib have a shorter residence time owing to their dissociation from an active enzyme form without requiring any changes in protein conformation. Dissociation of lapatinib from EGFR may require a receptor conformational change.

TABLE 15

| | Prcrein | intubi [symbol] or | Residence |
|---|---|---|---|
| Drug | kinase target | type | time |
| Sunitinib | VEGFR | [symbol] | <2.9 min |
| Gefitinib | EGfR | [symbol] | <10 min |
| Erlotiniib | EGFR | [symbol] | <10 min |
| Lenvatinib | VEGFR | [symbol] | 17 min |
| So [symbol] afenib | VEGFR | [symbol] | 64 min |
| Lapatinib | EGFR | [symbol] | 300 min |
| Sorafenib | B-Raf | [symbol] | 568 min |
| Sorafenib | CDK8 | [symbol] | 576 min |

[symbol]

indicates data missing or illegible when filed

### Example 8 Abl-BRD4 Chimeric Molecule

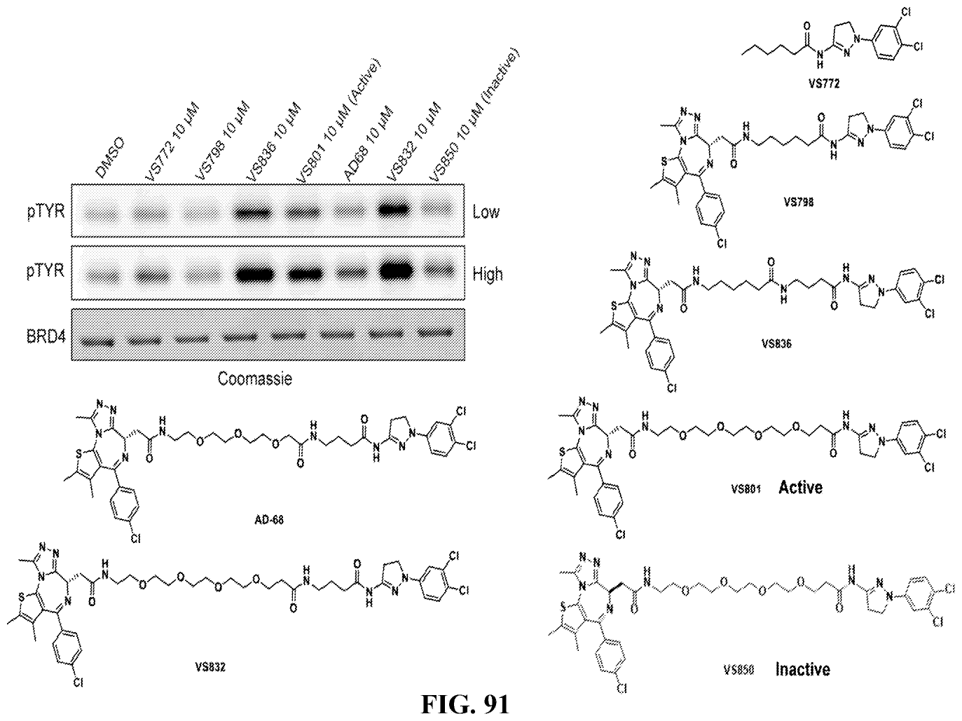

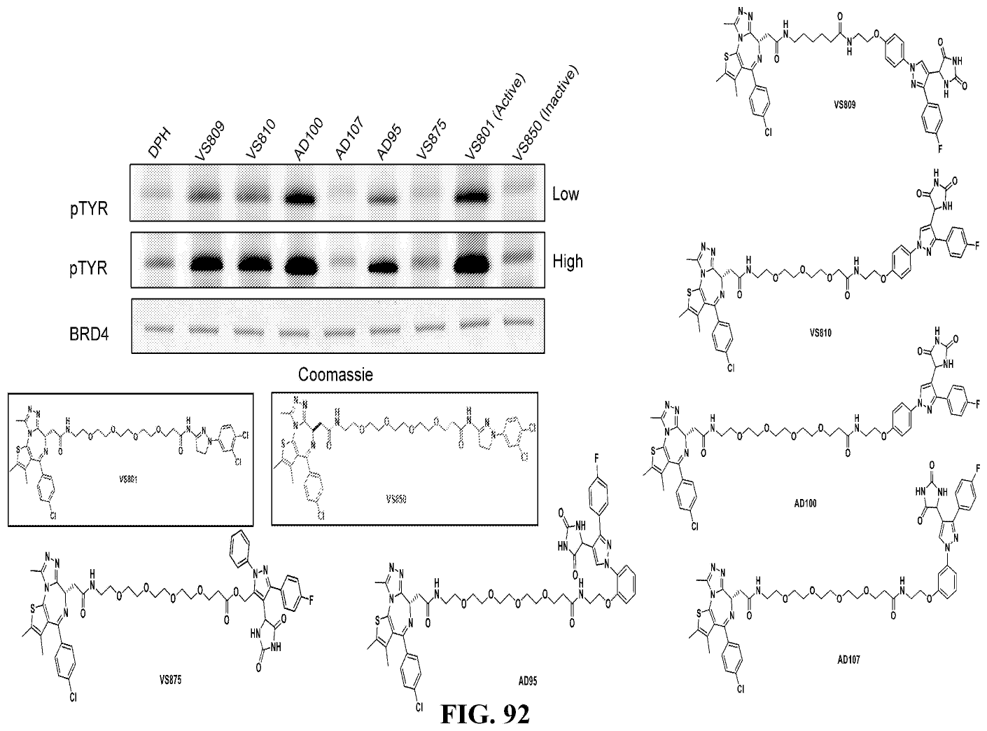

Multi-functional molecules comprising a BRD4 localizing moiety and an Abl activator moiety were assayed for tyrosine phosphorylation activity (Millipore)(FIG. 91). Active molecule VS801 showed high tyrosine phosphorylation enzymatic activity relative to control DMSO and inactive molecule VS850. DPH derived PHICS phosphorylation levels, and thus, Abl engagement, varied by attachment of the DPH derivative to the multifunctional chimeric molecule via orienting adaptor and linker (FIG. 92).

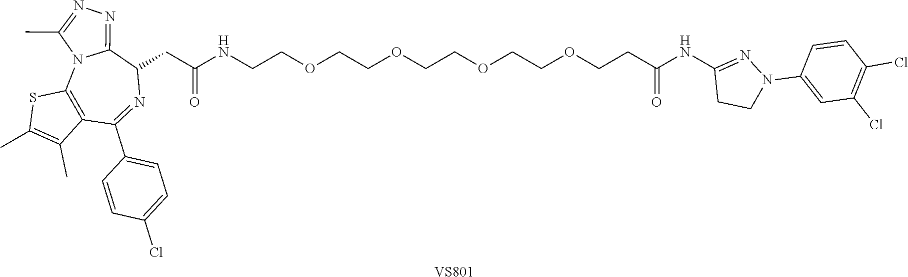

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A multi-functional chemical conjugation molecule, comprising a localizing moiety, a chemical linker moiety, an activator moiety, a first orienting adaptor interconnecting the chemical linker moiety on one end to the activator moiety, and optionally a second orienting adaptor interconnecting the chemical linker molecule on a different end to the localizing moiety.

2. The molecule of claim 1, represented by Formula I-A
Loc-L-(V₁-Act)ₙ (I-A),
wherein Loc comprises the localizing moiety, L is the chemical linker moiety, V₁ is the first orienting adaptor, and Act is the activator moiety and wherein n is at least one; or Formula I-B
Loc-V₂-L-(V₁-Act)ₙ (I-B)
wherein Loc comprises the localizing moiety, L is the chemical linker moiety, V₁ is the first orienting adaptor, V₂ is the second orienting adaptor, and Act is the activator moiety.

3. The molecule of claim 1 or claim 2, wherein the first and second orienting adaptor are independently selected from Table 2.

4. The molecule of claim 1, wherein the activator moiety binds and activates an enzyme that modifies a target substrate associated with the localizing moiety.

5. The molecule of claim 4, wherein the target substrate is not a natural substrate of the enzyme, or wherein activation of the enzyme by the activator molecule results in modification of the target substrate by the enzyme at one or more new modification sites that would otherwise remain unmodified by the enzyme when not activated by binding to the activator moiety.

6. The molecule of claim 1, wherein linker is selected from 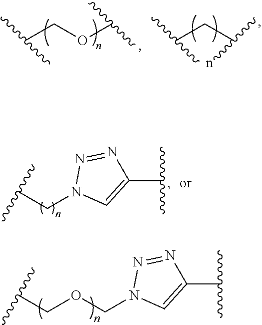
wherein n is between 1 and 50.

7. The molecule of claim 1, wherein the linker is a PEG molecule, alkyl, heterocycloalkyl, cycloalkyl, aryl, alkylene, alkenyl, heteroaryl, amide, amine, thiol or derivatives thereof.

8. The molecule of claim 1, wherein the linker is a multifunctional linker.

9. The molecule of claim 8, wherein the linker is a multifunctional PEG linker.

10. The molecule of claim 2, wherein n is between 2 and 5.

11. The molecule of claim 1, wherein the activator moiety is capable of finding and activating an enzyme.

12. The molecule of claim 11, wherein the enzyme is a kinase, phosphatase, transferase or ligase.

13. The molecule of claim 12, wherein the kinase a serine/threonine kinase, a tyrosine kinase, or a dual-specificity protein kinase that phosphorylates protein serine/threonine and protein tyrosine.

14. The molecule of claim 13, wherein the kinase is AMP-activated protein kinase (AMPK), a Glucokinase (GK), or an AGC kinase.

15. The molecule of claim 1, wherein the activator moiety binds and activates a protein kinase C (PKC).

16. The molecule of claim 15, wherein the activator moiety binds and activates a PKC isoform selected from: PKC-α, PKC-βI, PKC-βII, PKC-γ, PKC-ε, PKC-δ, PKC-η, or PKC-ξ.

17. The molecule of claim 15, wherein the activator moiety is selected from Table 2.

18. The molecule of claim 1, wherein the localizing moiety targets a nucleic acid, polypeptide, or polysaccharide.

19. The molecule of claim 1, wherein the localizing moiety is a target polypeptide binding moiety.

20. The molecule of claim 19, wherein the target polypeptide binding moiety binds a target polypeptide comprising a bromodomain and an extra-terminal motif (BET).

21. The molecule of claim 20, wherein the target polypeptide is a bromodomain-containing protein 4 (BRD4), BRD3, BRD2, BRDT.

22. The molecule of claim 21, wherein the target polypeptide binding moiety is (+)-JQ1.

23. The molecule of claim 1, according to the formula 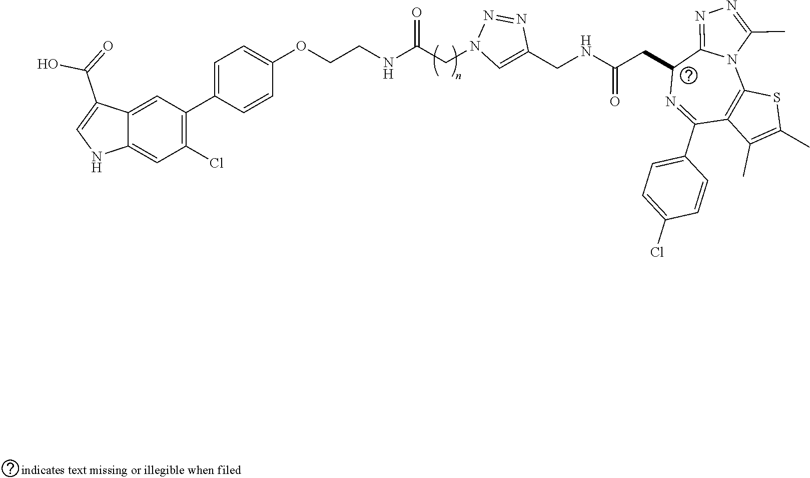
wherein n=3, 5, 7, 9, 11 [PHICS 1.1-1.5].

24. The molecule of claim 1, according to the formula 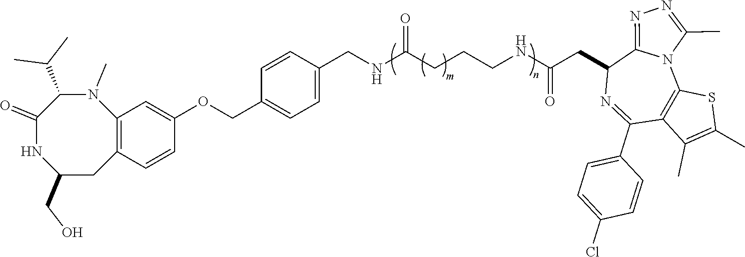
wherein n=0 and m=0, n=1 and m=1, n=2 and m=3, n=2 and m=1, n=2 and m=3.

25. The molecule of claim 24 selected from

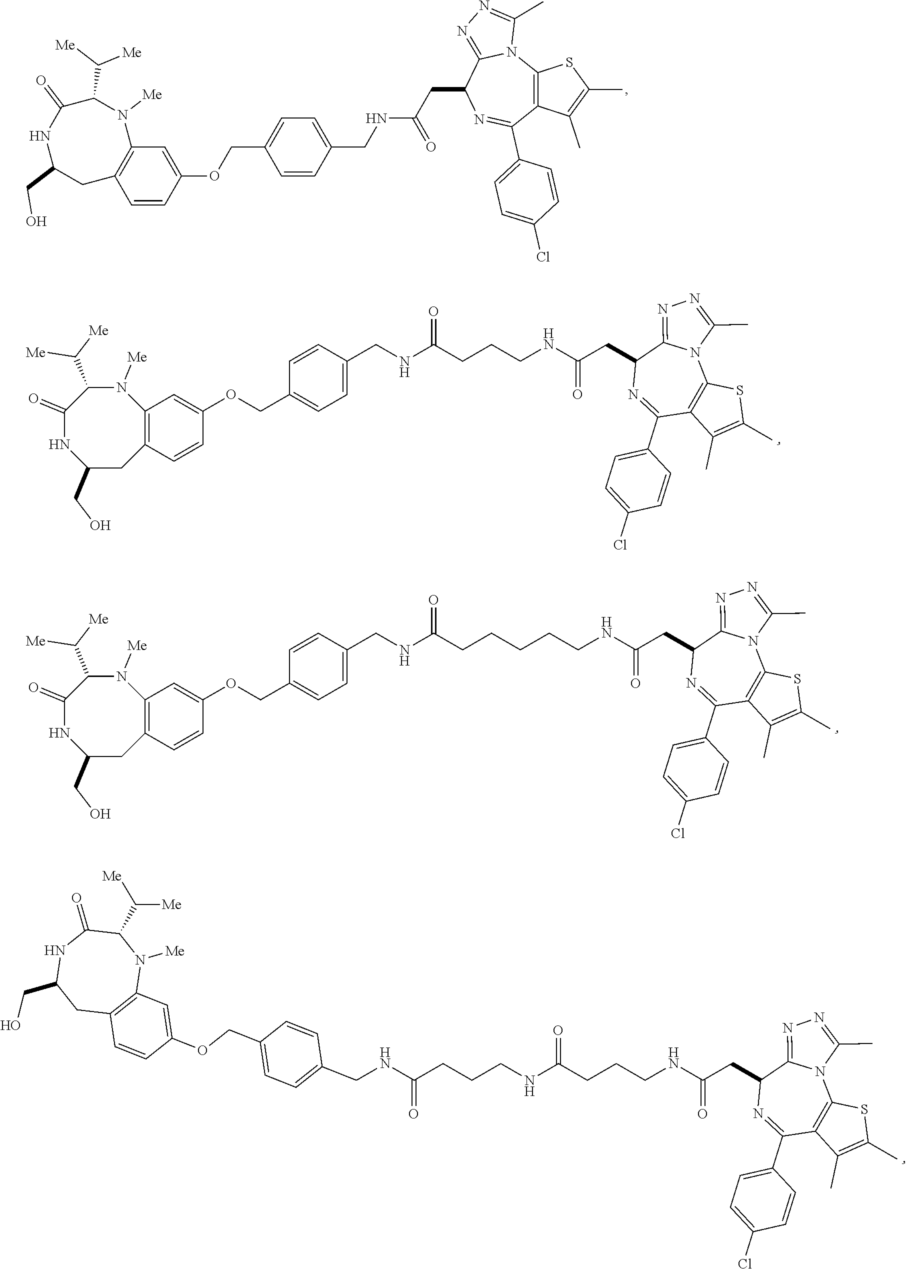

26. The molecule of claim 1, wherein the localizing moiety is independently selected from Table Y, the activating moiety is independently selected from Table Z, the first and second orienting adaptor are independently selected from Table 1 and the linker is independently selected from Table 2.

27. The molecule of claim 1 according to the formula 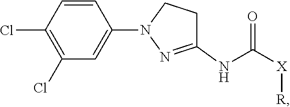
wherein X is selected from (CH₂)ₙ, (CH₂)ₙNHC(O)(CH₂)ₙ, (CH₂)ₙ(OC₂H₄)ₙ, (CH₂)ₙNHC(O)CH₂(OC₂H₄)ₙ, and (CH₂)ₙNHC(O)(CH₂)ₙ(OC₂H₄)ₙ, wherein each n is independently selected from 0, 1, 2, 3, 4, 5, 6 or 7 and R is

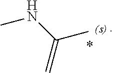

28. The molecule of claim 1 according to the formula

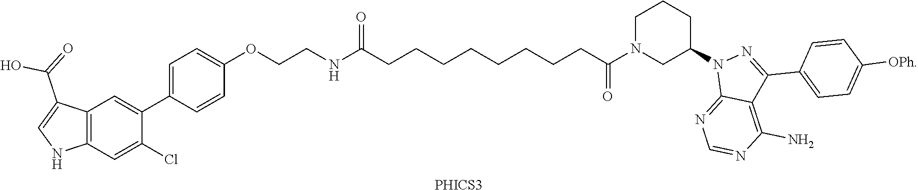 [SMILES: Nc1ncnc2c1c(-c1ccc(Oc3ccccc3)cc1)nn2[C@@H]1CCCN(C(=O)CCCCCCCCC(=O)NCCOc2ccc(-c3cc4c(C(=O)O)c[nH]c4cc3Cl)cc2)C1 ; "PHICS3"]

29. The molecule of claim 1, according to the formula 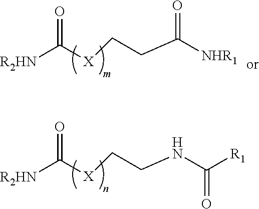
wherein R₁ is selected from JQ1, Ibrutinib, Dasatinib, MRTX, MI-1061, Gefitinib, Palbociclib, or Foretinib, and R₂ is selected from PF-06409577, Benzolactam or DPH, wherein X is CH₂ or (CH₂)₂O, and when X=CH₂, n=1 or 5 or m=0 or 4 and wherein when X=(CH₂)₂O, n=3, or m=3.

30. A pharmaceutical composition comprising the molecule according to any one of the preceding claims and one or more pharmaceutically acceptable salts, carriers, or diluents.

31. The pharmaceutical composition of claim 30, further comprising AMP.

32. A method of modifying a target substrate in a cell, comprising contacting the cell with the molecule of any of claims 1-32.

33. The method of claim 33, wherein the modifying comprises a post translational modification.

34. The method of claim 33, wherein the post-translation modification comprises phosphorylation, hydroxylation, acetylation, methylation, glycosylation, prenylation, amidation, eliminylation, lipidation, acylation, lipoylation, deacetylation, formylation, S-nitrosylation, S-sulfenylation, sulfonylation, sulfinylation, succinylation, sulfation, carbonylation, or alkylation.

35. The method of claim 34, wherein the modifying comprises inducing phosphorylation of a protein in the cell.

36. A method of phosphorylating a protein comprising contacting the protein with the molecule of any one of claims 1-32 wherein the protein is in proximity to a kinase specific to the activator moiety of the molecule.

37. The method of claim 36, wherein the phosphorylating of the protein comprises phosphorylation of a plurality of proteins that are not a substrate of the kinase.

38. The method of claim 36, wherein the protein is BRD4.

39. The method of claim 38, wherein the protein is phosphorylated between BD1 and BD2 of the BRD4.

40. A method of modifying a target substrate in a subject in need thereof, the method comprising administering a molecule of any of claims 1-32 to the subject.

41. The method of claim 40, wherein the subject has cancer.

42. A method for modifying a protein of interest, the method comprising contacting the protein of interest with a compound according to any one of claims 1-32 in an environment comprising one or more activators.

43. A method for the treatment of a disease, disorder, or condition in a subject in need thereof comprising administering a molecule according to any one of claims 1-32 the subject.

44. A method of making a multifunctional conjugation molecule, comprising binding a localizing moiety and an activator moiety to different ends of a linker molecule, the localizing moiety and activator moiety optionally bound to the linker molecule via orienting adaptors wherein the linker molecule links the activator molecule such that both the activator molecule and localizing moiety is active in a cell.

45. The method of claim 42, wherein the compound is selected from compounds from Tables 4, 5, 6, 8, 9, 10, 11, 12, or 14.

46. The composition of claim 1 according to the formula: [chemical structure]


SEQUENCE LISTING (17 sequences)

SEQ ID NO: 1 - protein, 8 aa, Artificial Sequence
  Synthetic
GPLGIAGQ

SEQ ID NO: 2 - protein, 15 aa, Homo sapiens
GQRRESSRPVKPPRR

SEQ ID NO: 3 - protein, 15 aa, Homo sapiens
ELPTEETEIMIVQRR

SEQ ID NO: 4 - protein, 15 aa, Homo sapiens
KKDVPDSQQHPAPRR

SEQ ID NO: 5 - protein, 15 aa, Homo sapiens
EQLKCCSGILKEMRR

SEQ ID NO: 6 - DNA, 20 bp, Artificial Sequence
  Synthetic
acctgggagtgctcaccgga

SEQ ID NO: 7 - DNA, 24 bp, Artificial Sequence
  Synthetic
tttaagcttccattcctgttctcc

SEQ ID NO: 8 - DNA, 23 bp, Artificial Sequence
  Synthetic
cgtggccatcaggatgatcaaag

SEQ ID NO: 9 - DNA, 18 bp, Artificial Sequence
  Synthetic
tcgtactggcctctccat

SEQ ID NO: 10 - DNA, 29 bp, Artificial Sequence
  Synthetic
cttcatcatcgctgagtacatggccaatg

SEQ ID NO: 11 - DNA, 18 bp, Artificial Sequence
  Synthetic
atggggcgctgcttggtg

SEQ ID NO: 12 - DNA, 24 bp, Artificial Sequence
  Synthetic
taaagtatctaatttcggcctgtc

SEQ ID NO: 13 - DNA, 20 bp, Artificial Sequence
  Synthetic
acaactccttgatcgtttac

SEQ ID NO: 14 - protein, 15 aa, Homo sapiens
HMRSAMSGLHLVKRR

SEQ ID NO: 15 - protein, 458 aa, Homo sapiens
NAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDS

SEQ ID NO: 16 - protein, 434 aa, Homo sapiens
TPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSS

SEQ ID NO: 17 - protein, 451 aa, Homo sapiens
QLTPANPPPPEVSNPKKPGRVTNQLQYLHKVVMKALWKHQFAWPFRQPVDAVKLGLPDYHKIIKQPMDMGTIKRRLENNYYWAASECMQDFNTMFTNCYIYNKPTDDIVLMAQTLEKIFLQKVASMPQEEQELVVTIPKNSHKKGAKLAALQGSVTSAHQVPAVSSVSHTALYTPPPEIPTTVLNIPHPSVISSPLLKSLHSAGPPLLAVTAAPPAQPLAKKKGVKRKADTTTPTPTAILAPGSPASPPGSLEPKAARLPPMRRESGRPIKPPRKDLPDSQQQHQSSKKGKLSEQLKHCNGILKELLSKKHAAYAWPFYKPVDASALGLHDYHDIIKHPMDLSTVKRKMENRDYRDAQEFAADVRLMFSNCYKYNPPDHDVVAMARKLQDVFEFRYAKMPDEPLEPGPLPVSTAMPPGLAKSSSESSSEESSSESSSEEEEEEDEEDEEEE